(19) United States
(12) Patent Application Publication
(10) Pub. No.: US 2024/0366776 A1
(43) Pub. Date: Nov. 7, 2024

(54) COMPOSITIONS OF PROTEIN COMPLEXES AND METHODS OF USE THEREOF

(71) Applicant: Hoffmann-La Roche Inc., Little Falls, NJ (US)

(72) Inventors: John Thomas MULLIGAN, Seattle, WA (US); Shannon Lee Okada, Seattle, WA (US); Justin Richard Killebrew, Seattle, WA (US); Diane Louise Hollenbaugh, Seattle, WA (US)

(21) Appl. No.: 18/665,678

(22) Filed: May 16, 2024

Related U.S. Application Data
(63) Continuation of application No. PCT/US2022/080043, filed on Nov. 17, 2022.
(60) Provisional application No. 63/280,494, filed on Nov. 17, 2021.

(51) Int. Cl. A61K 47/68; A61P 35/00; A61P 37/04

(52) U.S. Cl. CPC A61K 47/6813; A61K 47/6845; A61K 47/6849; A61K 47/6889; A61P 35/00; A61P 37/04

(57) ABSTRACT

Provided herein are protein complexes comprising a sensor domain and a therapeutic domain linked by a linker, and methods of use thereof. In aspects of the present disclosure, activity of the therapeutic domain comprises a dependence on sensor domain binding to target markers.

### SEQUENCE LISTING

The instant application contains a Sequence Listing which has been submitted electronically in XML format and is hereby incorporated by reference in its entirety. Said XML copy, created on May 8, 2024, is named 51177-046003_Sequence_Listing_5_8_24 and is 634,151 bytes in size.

### BACKGROUND

Interleukin 2 (IL-2) is a potent cytokine that exhibits toxicity upon systemic administration. There is a need for a version of IL-2 that can be delivered systemically but can be regulated to exhibit therapeutic activity on an effective subset of T cells.

### SUMMARY

In some aspects, the present disclosure provides for a complex comprising: (a) a therapeutic domain comprising an IL-2 peptide and (b) a sensor domain comprising an antibody, wherein said sensor domain is configured to bind PD-1 and IL-2 in a mutually exclusive manner. In some embodiments, the complex further comprises a linker linking the therapeutic domain to the sensor domain. In some embodiments, the sensor domain is configured: (i) to bind IL-2 in the absence of PD-1; and (ii) to not bind IL-2 in the presence of PD-1. In some embodiments, the antibody is an antibody fragment or an antibody derivative. In some embodiments, the sensor domain comprises a single dual binding antibody (DBA) configured to bind PD-1 and IL-2. In some embodiments, the DBA comprises a heavy chain CDR3 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 11-20, 154-156, 168-173, 114-119, 415, 421, 433, 439, 445, 451, 457, 463, 469, 475, 481, 487, 493, 499, 505, 511, 517, 523, 529, 535, 541, 547, 553, 559, 565, 571, 577, 583, 589, 595, 601, 607, 613, 619, 625, 631, 637, 643, 649, 655, 661, or 667. In some embodiments, the DBA comprises a heavy chain CDR1, CDR2, or CDR3 comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any of the sequences recited in Table 3, Table 7, Table 8, or Table 19. In some embodiments, the DBA comprises a V_{H} or a V_{L} comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any of the sequences recited in Table 18. In some embodiments, the complex comprises an Fc domain. In some embodiments, the Fc domain is from IgG. In some embodiments, the Fc domain is homodimeric. In some embodiments, the Fc domain is heterodimeric. In some embodiments, the Fc domain comprises: (a) a first polypeptide comprising a knob mutation and (b) a second polypeptide comprising a hole mutation. In some embodiments, the knob mutation or the hole mutation comprises mutations of any one of following pairs of residues relative to IgG: 366 and 407, 405 and 394, or 407 and 366. In some embodiments, the knob mutation comprises an arginine residue, a phenylalanine residue, a tyrosine residue or a tryptophan residue and the hole mutation comprises an alanine residue, a serine residue, a threonine residue, or a valine residue. In some embodiments, the complex comprises a sensor domain comprising a full-length DBA, wherein the IL-2 peptide is linked to an N-terminus of a heavy chain of said full-length DBA or wherein the IL-2 peptide is linked to an N-terminus of a light chain of said full-length DBA. In some embodiments, the complex comprises a sensor domain comprising a full-length DBA, wherein the IL-2 peptide is linked to a C-terminus of a heavy chain of said full-length DBA. In some embodiments, the complex comprises: (a) a first polypeptide according to N-[IL-2]-[linker]-[V_{H}]-[C_{H}]-[hinge]-Fc-C; and a second polypeptide according to N-[V_{L}]-[C_{L}]-C, or (b) a first polypeptide according to N-[V_{H}]-[C_{H}]-[hinge]-Fc-C; and a second polypeptide according to N-[IL-2]-[linker]-[V_{L}]-[C_{L}]-C, wherein N-denotes a peptide N-terminus, C-denotes a peptide C-terminus, [linker] denotes said linker, V_{H} indicates a heavy chain variable domain of said DBA, Cu indicates a heavy chain constant domain of an immunoglobulin, V_{L} denotes a light chain variable domain of said DBA, [hinge] denotes a hinge region of an immunoglobulin, Fc denotes an Fc region of an immunoglobulin, and C_{L} denotes a light chain constant domain of an immunoglobulin. In some embodiments, the complex comprises any one of AF003345, AF003243, AF003246, AF003247, AF003341, AF003644, AF003651, AF003657, or AF003934. In some embodiments, the complex comprises: (a) a first polypeptide according to N-[IL-2]-[linker]-[V_{H}]-[C_{H}]-[hinge]-Fc[knob]-C; a second polypeptide according to N-[V_{L}]-[C_{L}]-C; and a third polypeptide according to N-[V_{H}]-[C_{H}]-[hinge]-Fc[hole]-C, or (b) a first polypeptide according to N-[IL-2]-[linker]-[V_{H}]-[C_{H}]-[hinge]-Fc[hole]-C; a second polypeptide according to N-[V_{L}]-[C_{L}]-C; and a third polypeptide according to N-[V_{H}]-[C_{H}]-[hinge]-Fc[knob]-C, wherein N-denotes a peptide N-terminus, C-denotes a peptide C-terminus, [linker] denotes said linker, V_{H} indicates a heavy chain variable domain of said DBA, C_{H} indicates a heavy chain constant domain of an immunoglobulin, V_{L} denotes a light chain variable domain of said DBA, [hinge] denotes a hinge region of an immunoglobulin, Fc[knob] denotes an Fc of an immunoglobulin comprising a knob mutation, Fc[hole] denotes an Fc region of an immunoglobulin comprising a hole mutation, and C_{L} denotes a light chain constant domain of an immunoglobulin. In some embodiments, the knob mutation or the hole mutation comprises mutations of any one of following pairs of residues relative to IgG: 366 and 407, 405 and 394, or 407 and 366. In some embodiments, the complex comprises any one of AF003229, AF003230, AF003232, AF003740, AF003747, AF003749, AF003753, AF003945, AF003947, AF003951, AF003952, AF003953, AF003955, AF003956, or AF003941. In some embodiments, the complex comprises: (a) a first polypeptide according to N-[IL-2]-[linker]-[V_{H}]-[C_{H}]-[hinge]-Fc-[scFv]-C; and (b) a second polypeptide according to N-[V_{L}]-[C_{L}]-C, wherein N-denotes a peptide N-terminus, C-denotes a peptide C-terminus, [linker] denotes said linker, V_{H} indicates a heavy chain variable domain of an anti-PD-1 monoselective antibody, C_{H} indicates a heavy chain constant domain of an immunoglobulin, V_{L} denotes a light chain variable domain of an anti-PD-1 monoselective antibody, [hinge] denotes a hinge region of an immunoglobulin, Fc denotes an Fc region of an immunoglobulin, C_{L} denotes a light chain constant domain of an immunoglobulin, and [scFv] denotes an scFv comprising V_{H} and V_{L} domains of said DBA. In some embodiments, said scFv is oriented according to N-[V_{H}]-[linker2]-[V_{L}]-C. In some embodiments, said scFv comprising V_{H} and V_{L} domains of said DBA comprises: (a) a V_{H} domain comprising a sequence having at least 80% identity to a V_{H} domain of any one of AB002022_2B07v1, AB002328_2B07v4, AB002360_7A04v1, AB002413_2A11v3, AB002342_2B07v5, AB002345_2B07v6, or AB002365_7A04v2; or (b) a V_{L} domain comprising a sequence having at least 80% identity to a V_{L} domain of any one of AB002022_2B07v1, AB002328_2B07v4, AB002360_7A04v1, AB002413_2A11v3, AB002342_2B07v5, AB002345_2B07v6, or AB002365_7A04v2. In some embodiments, said scFv comprising V_{H} and V_{L} domains of said DBA comprises: (a) heavy chain CDRs of any one of AB002022_2B07v1, AB002328_2B07v4, AB002360_7A04v1, AB002413_2A11v3, AB002342_2B07v5, AB002345_2B07v6, or AB002365_7A04v2; or (b) light chain CDRs of any one of AB002022_2B07v1, AB002328_2B07v4, AB002360_7A04v1, AB002413_2A11v3, AB002342_2B07v5, AB002345_2B07v6, or AB002365_7A04v2. In some embodiments, the complex comprises any one of AF003864, AF003871, AF003872, AF003913, AF003918, AF003923, AF003927, AF004502, AF004503, AF004504, AF004505, AF004892, or AF004893. In some embodiments, the complex comprises: (a) a first polypeptide according to N-[V_{H}]-[C_{H}]-[hinge]-Fc[knob]-[linker]-[IL-2]-C; a second polypeptide according to N-[V_{L}]-[C_{L}]-C; and a third polypeptide according to N-[V_{H}]-[C_{H}]-[hinge]-Fc[hole]-[linker]-[scFv]-C, or (b) a first polypeptide according to N-[V_{H}]-[C_{H}]-[hinge]-Fc[hole]-[linker]-[IL-2]-C; a second polypeptide according to N-[V_{L}]-[C_{L}]-C; and a third polypeptide according to N-[V_{H}]-[C_{H}]-[hinge]-Fc[knob]-[linker]-[scFv]-C; wherein N-denotes a peptide N-terminus, C-denotes a peptide C-terminus, [linker] denotes said linker, V_{H} indicates a heavy chain variable domain of said DBA, Cu indicates a heavy chain constant domain of an immunoglobulin, V_{L} denotes a light chain variable domain of said DBA, [hinge] denotes a hinge region of an immunoglobulin, Fc[knob] denotes an Fc of an immunoglobulin comprising a knob mutation, Fc[hole] denotes an Fc region of an immunoglobulin comprising a hole mutation, C_{L} denotes a light chain constant domain of an immunoglobulin, and [scFv] denotes an scFv of said DBA. In some embodiments, the complex comprises any one of AF004693, AF004695, AF004696, AF005416, AF005418, or AF005419. In some embodiments, the complex comprises: (a) a first polypeptide according to N-[V_{H}]-[C_{H}]-[het-hinge]-Fc[knob]-[linker]-[IL-2]-C; a second polypeptide according to N-[V_{L}]-[C_{L}]-C; and a third polypeptide according to N-[V_{H}]-[C_{H}]-[het-hinge]-Fc[hole]-C, or (b) a first polypeptide according to N-[V_{H}]-[C_{H}]-[het-hinge]-Fc[hole]-[linker]-[IL-2]-C; a second polypeptide according to N-[V_{L}]-[C_{L}]-C; and a third polypeptide according to N-[V_{H}]-[C_{H}]-[het-hinge]-Fc[knob]-C, wherein N-denotes a peptide N-terminus, C-denotes a peptide C-terminus, [linker] denotes said linker, V_{H} indicates a heavy chain variable domain of said DBA, C_{H} indicates a heavy chain constant domain of an immunoglobulin, V_{L} denotes a light chain variable domain of said DBA, [het hinge] denotes a hinge region heterologous to said Fc region, Fc[knob] denotes an Fc of an immunoglobulin comprising a knob mutation, Fc[hole] denotes an Fc region of an immunoglobulin comprising a hole mutation, and C_{L} denotes a light chain constant domain of an immunoglobulin. In some embodiments, the hinge region heterologous to said Fc region is: (a) a hinge region derived from an IgG3 antibody, or (b) a G4S-based linker. In some embodiments, the complex comprises AF003632 or AF003634. In some embodiments, the IL-2 peptide comprises a wild-type human IL-2 peptide. In some embodiments, the IL-2 peptide comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or substantially 100% sequence identity to human IL-2. In some embodiments, the IL-2 peptide comprises a mutation at least one of R38, K43, E61, F42, Y45, L72, T3, or C125 of human IL-2. In some embodiments, the complex comprises any one of AF003232, AF003243, AF003246, AF003247, AF003341, AF003345, AF003632, AF003634, AF003644, AF003651, AF003652, AF003653, AF003657, AF003740, AF003744, AF003747, AF003749, AF003753, AF003864, AF003873, AF003876, AF003877, AF003913, AF003918, AF003923, AF003927, AF003930, AF003931, AF003933, AF003934, AF003935, AF003941, AF003945, AF003946, AF003947, AF003948, AF003951, AF003952, AF003953, AF003955, AF003956, AF004262, AF004265, AF004273, AF004276, AF004284, AF004287, AF004295, AF004298, AF004385, AF004386, AF004387, AF004388, AF004389, AF004404, AF004405, AF004413, AF004414, AF004415, AF004416, AF004504, AF004505, AF004693, AF004695, AF004696, AF004771, AF004773, AF004892, or AF004893.

In some aspects, the present disclosure provides for a method of enhancing T-cell reactivity to heterologous cells, comprising administering any of the complexes described herein to a subject in need thereof. In some embodiments, the heterologous cells are cancer cells.

In some aspects, the present disclosure provides for a method of treating a subject in need thereof comprising administering the complex of any one of claims Error! Reference source not found.-Error! Reference source not found.to the subject in need thereof. In some embodiments, the administering comprises intravenous, intramuscular, or subcutaneous administration. In some embodiments, the subject in need thereof has cancer. In some embodiments, the therapeutic domain treats the subject in need thereof. In some embodiments, the subject in need thereof is a mammal. In some embodiments, the subject in need thereof is a human.

In some aspects, the present disclosure provides for a composition comprising a recombinant nucleic acid encoding any of the complexes described herein. In some aspects, the present disclosure provides for a host cell comprising any of the recombinant nucleic acids encoding any of the complexes described herein.

In some aspects, the present disclosure provides for a pharmaceutical composition comprising any of the complexes described herein and a pharmaceutically acceptable excipient.

In various aspects, the present disclosure provides a complex comprising: a) a therapeutic domain; b) a linker; and c) a sensor domain, wherein the therapeutic domain is an IL-2 agonist, the therapeutic domain is linked to the sensor domain by the linker, and wherein the sensor domain is dual-binding antibody (DBA) that is capable of binding the therapeutic domain (the IL-2 agonist domain) and a marker, wherein the marker is PD-1.

In some aspects, the sensor domain is bound to the therapeutic domain in an absence of the marker. In some aspects, the therapeutic domain is blocked from binding the sensor domain upon binding of the sensor domain to the marker. In some aspects, the activity of the therapeutic domain is reduced upon binding of the therapeutic domain to the sensor domain. In some aspects, the therapeutic domain is capable of exhibiting therapeutic activity upon binding of the sensor domain to the marker. In some aspects, the therapeutic domain is therapeutically active upon binding of the sensor domain to the marker.

In some aspects, the sensor domain comprises an antibody. In some aspects, the antibody is an antibody fragment or antibody derivative. In some aspects, the complex comprises an Fc domain. In some aspects, the complex comprises a domain that improves kinetic properties. In some aspects, the complex includes two heavy chains and two light chains.

In some aspects, the complex comprises two therapeutic domains. In some aspects, the complex comprises two sensor domains. In some aspects, the complex is a regulated therapeutic protein. In some aspects, the antibody or the antibody fragment comprises an IgG, a single domain antibody fragment, a nanobody, or a single chain variable fragment (scFv).

In some aspects, the therapeutic domain is an IL-2 receptor agonist. In some aspects, the IL-2 receptor agonist is IL-2, IL-15, or variants or fusions thereof. In some aspects, the therapeutic domain binds to the sensor domain.

In some aspects, the linker is a polypeptide linker. In some aspects, the linker comprises from 2 to 200 amino acids in length. In some aspects, the linker is: attached to a heavy chain of the sensor domain, attached to a light chain of the sensor domain, is a fusion with an N-terminus of the sensor domain, or is a fusion with a C-terminus of the sensor domain. In some aspects, the linker is: attached to a heavy chain of the therapeutic domain, attached to a light chain of the therapeutic domain, is a fusion with an N-terminus of the therapeutic domain, or is a fusion with a C-terminus of the therapeutic domain.

In some aspects, the activity of the therapeutic domain is reduced when bound to the sensor domain. In some aspects, the therapeutic domain is inactive when bound to the sensor domain. In some aspects, the sensor domain blocks the activity of the therapeutic domain when bound to the therapeutic domain. In some aspects, the therapeutic domain is active when the sensor domain is bound to the marker. In some aspects, an affinity of the sensor domain for the marker is equal to or greater than an affinity of the sensor domain for the therapeutic domain.

In some aspects, an affinity of the sensor domain for the marker is at least 2 times, 5 times, 10 times, 100 times, 1000 times, 10000, or 100000 times greater than an affinity of the sensor domain for the therapeutic domain.

In some aspects, the sensor domain is an antibody or a fragment thereof. In some aspects, the sensor domain comprises one or both antigen binding domains of a bispecific antibody. In some aspects, the bispecific antibody comprises a first antigen binding domain that is capable of binding to the therapeutic domain and is capable of binding to the marker, and a second antigen binding domain that is capable of binding to the marker. In some aspects, the bispecific antibody comprises a single therapeutic domain.

In some aspects, the sensor antibody binds to an IL-2 receptor agonist and to PD-1. In some aspects, the IL-2 receptor agonist is IL-2, IL-15, or variants or fusions thereof.

In some aspects, the sensor domain comprises a complementarity determining region (CDR) selected from Table 3, Table 7, Table 8, or Table 19. In some aspects, the sensor domain is selected from TABLE 8 or TABLE 18. In some aspects, the complex is selected from TABLE 15 or TABLE 2A. In some aspects, the sensor domain comprises a complementarity determining region having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any of the complementary determining regions selected from Table 3, Table 7, Table 8, or Table 19. In some aspects, the sensor domain comprises a V_{H} or V_{L} domain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any of the V_{H} or V_{L} domains recited in TABLE 8 OR TABLE 18. In some aspects, the sensor domain comprises a complementarity determining region having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NO: 1-SEQ ID NO: 20 or SEQ ID NO: 142-SEQ ID NO: 173, or SEQ ID NO: 238-252. In some aspects, the sensor domain has at least 80%, at least 85%, at least 90%, at least 95%, at least 97%, at least 99%, or 100% sequence identity to any one of SEQ ID NO: 21-SEQ ID NO: 27, SEQ ID NO: 31-SEQ ID NO: 39, or SEQ ID NO: 127-SEQ ID NO: 141. In some aspects, the protein complex has at least 80%, at least 85%, at least 90%, at least 95%, at least 97%, at least 99%, or 100% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, SEQ ID NO: 289-293, or a fragment thereof.

In various aspects, the present disclosure provides a method comprising administering any of the above complexes to a subject in need thereof. In various aspects, the present disclosure provides a method of treating a subject in need thereof comprising administering any of the above complexes to the subject in need thereof. In some aspects, the administering comprises intravenous, intramuscular, or subcutaneous administration. In some aspects, the subject in need thereof has cancer. In some aspects, the therapeutic domain treats the subject in need thereof. In some aspects, the subject in need thereof is a mammal. In some aspects, the subject in need thereof is a human.

In some aspects, the present disclosure provides IL-2 conjugates that can be delivered systemically but may exhibit diminished systemic toxicity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a schematic of the protein complexes of the present disclosure. FIG. 1A shows an exemplary dual binding protein complex in an inactive state. The protein complex has a sensor domain and a therapeutic domain. The sensor domain and therapeutic domain are linked by a linker. The sensor domain is shown bound to the therapeutic domain, rendering the therapeutic domain inactive. FIG. 1B shows an exemplary dual binding protein complex in an active state. The protein complex has a sensor domain and a therapeutic domain. The sensor domain and therapeutic domain are linked by a linker. The sensor domain is shown bound to the marker, rendering the therapeutic domain active.

FIG. 2 shows schematics of protein complexes of the present disclosure comprising one or more sensor domains and one or more therapeutic domain. FIGS. 2A, 2B, 2C, 2D, 2E, 2F, 2G, 2H, and 2I show example schematics of arrangements of protein complexes detailed in the Examples.

FIG. 3 shows that IL-2 signaling by five exemplary PD-1/IL-2 DBA-cytokine protein complexes (2_A08, 2_A11, 2_B05, 2_B07, and 7_A04, SEQ ID NO: 67-SEQ ID NO: 68, SEQ ID NO: 69-SEQ ID NO: 70, SEQ ID NO: 71-SEQ ID NO: 72, SEQ ID NO: 73-SEQ ID NO: 74 and SEQ ID NO: 75-SEQ ID NO: 76 respectively) is reduced as compared to a control IL-2-Anti-HER2 protein complex (SEQ ID NO: 65-SEQ ID NO: 66).

FIGS. 4A, 4B, 4C, 4D, 4E, and 4F provide IL-2 activity of protein complexes comprising the structure depicted in FIG. 2B in wells coated with PD-1-Fc or an IgG1 control protein. Activity was measured as growth of a 630 nm signal from HEK-BLUE™ IL-2 reporter cells. FIGS. 4A-C provide the IL-2 activities of three different PD-1/IL-2 DBA-IL-2 complexes. FIG. 4D provides the activity of an anti-PD-1 antibody-IL-2 complex. FIG. 4E provides the activity of an anti-Her-2 antibody-IL-2 complex. FIG. 4F provides the activity of an anti-IL-2 antibody-IL-2 complex.

FIGS. 5A, 5B, 5C, 5D, 5E, 5F, 5G, and 5H provide IL-2 activity of protein complexes comprising the structure depicted in FIG. 2H in wells coated with PD-1-Fc or an IgG1 control protein. Activity was measured as growth of a 630 nm signal from HEK-BLUE™ IL-2 reporter cells. FIG. 5B-5D provide results for two PD-1/IL-2 DBA complexes comprising anti-PD-1 domains in the Fab arms and a PD-1/IL-2 DBA scFv on the Fc arm. FIGS. 5A, 5C, and 5E-H provide results for control protein complexes.

FIG. 6 provides rates of serum concentration decreases in the blood of wild-type mice of a PD-1/IL-2 DBA-cytokine complex (‘2B07 IL-2 mut’) and two control complexes.

FIGS. 7A, 7B, 7C, and 7D provide CD8⁺ T cell and NK cell counts in blood and spleen tissue collected from wild-type mice 5 days following treatment with a PD-1/IL-2 DBA-cytokine complex (‘2B07 IL-2 mut’) and two control complexes.

FIG. 8 provides tumor volume measurements as a function of the number of days post tumor cell implant in mice. Mice received various intravenous doses of a PD-1/IL-2 DBA-IL-2 complex, a PD-1/IL-2 DBA complex lacking IL-2, or an isotype control.

FIG. 9 provides the IL-2RBG binding for symmetric complexes comprising the structure depicted in FIG. 2E.

FIG. 10 provides the IL-2RBG binding for asymmetric complexes comprising the structure depicted in FIG. 2B.

FIG. 11 shows a plot depicting in vitro IL-2RBG binding by PD-1/IL-2 DBA-cytokine complexes and non-regulated control complexes and compares complexes made with two different forms of IL-2 including WT IL-2 and IL-2 3x. FIG. 11A shows IL-2RBG binding for complexes with wild-type IL-2 and FIG. 11B shows IL-2RBG binding for complexes with the IL-2 3× mutant.

FIGS. 12A, 12B, 13A, 13B, 14A, 14B, 15, and 16 show plots depicting IL-2 activity of IL-2-linked protein complexes comprising the structures depicted in FIGS. 2E, 2B, 2H, 2G and 2I, respectively in cells treated with PD-1-Fc or an IgG1 control protein. Activity was measured as growth of a 630 nm signal from HEK-BLUE™ IL-2 reporter cells (an engineered human kidney cell line which generates a detectable color change in upon activation of its IL-2 receptor).

FIG. 17 shows plots depicting IL-2 activity of IL-2-linked protein complexes comprising the structures depicted in FIG. 2H with a human Fc domain in cells treated with PD-1-Fc or an IgG1 control protein. Activity was measured as growth of a 630 nm signal from HEK-BLUE™ IL-2 reporter cells (an engineered human kidney cell line which generates a detectable color change in upon activation of its IL-2 receptor).

FIG. 18 shows plots depicting IL-2 activity in protein complexes comprising the structure depicted in in FIG. 2B with the IL-2 3× variant.

FIGS. 19A, 19B, and 19C show plots depicting IL-2 activity of IL-2-3×-linked protein complexes comprising the structure depicted in in FIG. 2H with three different anti-PD-1 Fab arms (Nivolumab, 4C10 and Knd respectively) in cells plated in wells coated with PD-1-Fc or an IgG1 control protein. Activity was measured as growth of a 630 nm signal from HEK-BLUE™ IL-2 reporter cells (an engineered human kidney cell line which generates a detectable color change in upon activation of its IL-2 receptor).

FIG. 20 shows plots depicting IL-2 activity of IL-2-linked protein complexes comprising the structure depicted in FIG. 2B with linkers of varying lengths. Complexes were tested in wells coated with PD-1-Fc or an IgG1 control protein. Activity was measured as growth of a 630 nm signal from HEK-BLUE™ IL-2 reporter cells (an engineered human kidney cell line which generates a detectable color change in upon activation of its IL-2 receptor).

FIGS. 21A and 21B show plots depicting PD-1 dependent induction of STAT5 phosphorylation by PD-1/IL-2 DBA-Cytokine Complexes in Human Primary CD8+ T Cells as measured by flow cytometry.

FIGS. 22A and 22B show plots depicting PD-1/IL-2 DBA-cytokine complex modulation of human T cell activation in a mixed lymphocyte reaction as assessed by Granzyme B release.

FIGS. 23A and 23B show plots depicting PD-1/IL-2 DBA-Cytokine Complex Modulation of Anti-Tumor Immunity in a Syngenetic Tumor Model. 500,000 MC38 tumor cells were implanted subcutaneously in human PD-1 knock-in mice, mice were treated intravenously with complexes, and tumor volume was assessed.

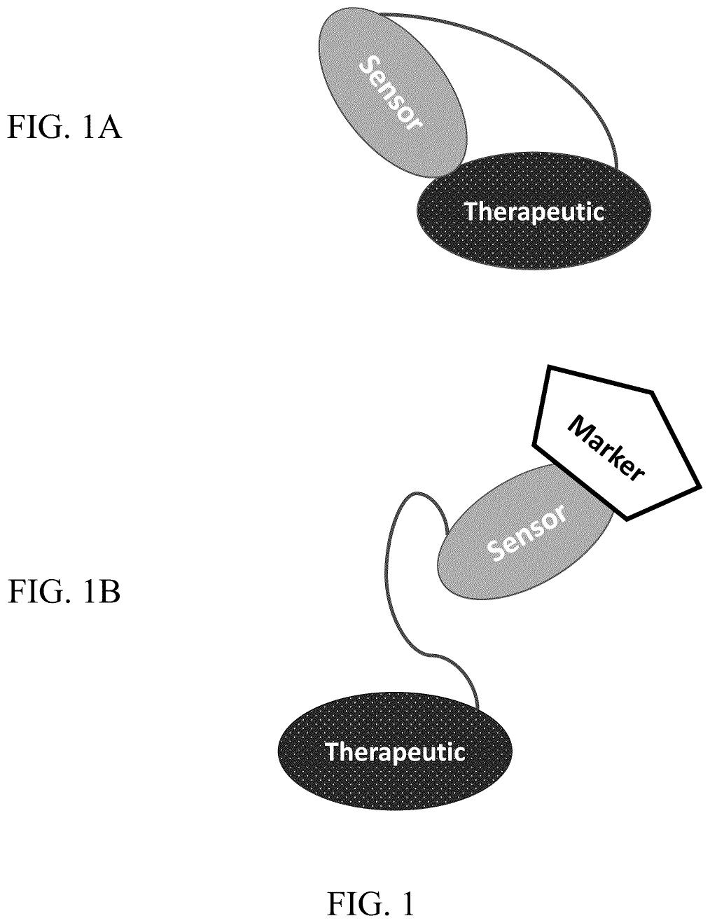

### DETAILED DESCRIPTION

The present disclosure provides compositions of protein complexes and methods of use thereof. Interleukin 2 therapeutics are often unable to be realized due to systemic on-target toxicity. Provided herein are protein complexes, which specifically exhibit therapeutic efficacy on PD-1 positive cells, specifically antigen-experienced T cells. Moreover, protein complexes of the present disclosure are self-regulated, remaining inactive in the absence of a PD-1 and activating when bound to PD-1. The protein complexes disclosed herein may include a sensor domain (e.g., an antibody, Fab or scFv) that is linked to an IL-2 receptor agonist (the therapeutic domain) via a linker. The sensor domain may be a dual binding antibody that has affinity for the therapeutic domain and for PD-1, such that the PD-1 and the therapeutic domain compete for binding to the sensor domain. In the absence of PD-1, the sensor domain binds the therapeutic domain, rendering the therapeutic domain unable to exert activity on IL-2 receptors. When the sensor domain is bound to PD-1, the therapeutic domain is unbound and may exert activity. In some embodiments, regulation of IL-2 receptor agonist activity by the complex may be reversible, that is, when the sensor domain disassociates from PD-1, the sensor domain may bind the therapeutic domain, rendering the therapeutic domain once again unable to exert activity. Thus, the protein complexes of the present disclosure comprise sensor domains that regulate IL-2 receptor agonist domains in the presence of PD-1, bind the PD-1, and render the IL-2 receptor agonist domain active. Various structures and compositions of protein complexes are disclosed herein, including pharmaceutical formulations. Also provided herein are methods for treating a subject in need thereof by administering the protein complex to the subject.

As used herein, a “sensor domain” generally refers to a dual-binding antibody capable of binding PD-1 and of binding an IL-2 receptor agonist.

As used herein, a “therapeutic domain” generally refers to an IL-2 receptor agonist. Non-limiting examples of a therapeutic domain include IL-2, IL-15 or any other molecule that acts on an IL-2 receptor in a manner similar to IL-2.

As used herein, a “marker” generally refers to PD-1 protein.

As used herein, an “antibody” generally refers to an antibody, an antibody derivative, or fragment(s) thereof that contains part or all of an antibody variable domain.

The term “recombinant nucleic acid” generally refers to synthetic nucleic acid having a nucleotide sequence that is not naturally occurring. A recombinant nucleic acid may be synthesized in the laboratory. A recombinant nucleic acid is prepared by using recombinant DNA technology by using enzymatic modification of DNA, such as enzymatic restriction digestion, ligation, and DNA cloning. A recombinant nucleic acid as used herein can be DNA, or RNA. A recombinant DNA may be transcribed in vitro, to generate a messenger RNA (mRNA), the recombinant mRNA may be isolated, purified and used to transfect a cell. A recombinant nucleic acid may encode a protein or a polypeptide. A recombinant nucleic acid, under suitable conditions, can be incorporated into a living cell, and can be expressed inside the living cell. As used herein, “expression” of a nucleic acid usually refers to transcription and/or translation of the nucleic acid. The product of a nucleic acid expression is usually a protein but can also be an mRNA. Detection of an mRNA encoded by a recombinant nucleic acid in a cell that has incorporated the recombinant nucleic acid, is considered positive proof that the nucleic acid is “expressed” in the cell.

As used herein, the term “therapeutic domain” generally refers to a protein domain having the minimum sequence features to activate a given therapeutic activity in a cell or organism. In the case where a therapeutic domain is a ligand of a ligand-receptor pair, the ligand has the minimum sequence and/or structural features to allow for binding to or activation of the receptor.

The process of inserting or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the forced introduction of small molecules such as DNA, RNA, or antibodies into eukaryotic cells. Just to make life confusing, ‘transfection’ also refers to the introduction of bacteriophage into bacterial cells. ‘Transduction’ is mostly used to describe the introduction of recombinant viral vector particles into target cells, while ‘infection’ refers to natural infections of humans or animals with wild-type viruses.

### Protein Complexes

The present disclosure provides complexes that may self-regulate IL-2 receptor agonist activity. Protein complexes of the present disclosure may include a dual-binding antibody with affinity for PD-1 and affinity for an IL-2 receptor agonist (the “sensor domain”) and an IL-2 receptor agonist (the “therapeutic domain”). The sensor domain and therapeutic domain may be linked by a linker. The sensor domain may regulate the activity of the therapeutic domain. Regulation of the activity of the therapeutic domain may include binding of the sensor domain to the therapeutic domain, rendering the therapeutic domain unable to exert activity on the IL-2 receptor. Regulation of the activity of the therapeutic domain may further include unbinding, or release, of the therapeutic domain by the sensor domain upon binding of the sensor domain to PD-1. Thus, the protein complexes of the present disclosure are superior drug candidates as the sensor domain-dependent activity of the IL-2 receptor agonist allows for cell-specific activity, even upon systemic administration of the protein complex. Compared to IL-2 receptor agonists administered on their own, the protein complexes of the present disclosure exhibit regulated therapeutic activity. As a result, compared to free IL-2 receptor agonists administered on their own, the protein complexes of the present disclosure exhibit reduced systemic on-target toxicity.

The protein complexes of the present disclosure can have an Fc region. The protein complexes of the present disclosure can have a domain that improves kinetic properties. For example, the protein complexes of the present disclosure may be further coupled to a half-life extender, such as an Fc region, albumin, PEG, or another zwitterionic polymer. The protein complexes of the present disclosure may have two heavy chains and two light chains. The protein complexes of the present disclosure may have two heavy chains and one light chain. The protein complexes of the present disclosure may include multiple sensor domains and multiple therapeutic domains. For example, a protein complex of the present disclosure may include two sensor domains and two therapeutic domains, all of which are linked and in which the two therapeutic domains are bound to the two sensor domains. In some embodiments, a protein complex of the present disclosure may include two sensor domains and one therapeutic domain, all of which are linked and in which the therapeutic domain may bind to both sensor domains or only one of the two sensor domains.

In some embodiments, the PD-1 may be a surface protein, such as a cell surface protein. In some embodiments, the PD-1 may be expressed on antigen-experienced T cells.

In some embodiments the IL-2 receptor agonist may be IL-2, a variant of IL-2 or a truncated version of IL-2. In some embodiments the IL-2 receptor agonist may be IL-15, IL-15-sushi, a variant of Il-15 or a variant of IL-15-sushi. In some embodiments the IL-2 receptor agonist may be an engineered or designed peptide that binds IL2 receptor beta and IL-2 receptor gamma. In some embodiments the IL-2 receptor agonist may be an antibody that binds IL2 receptor beta and IL-2 receptor gamma.

In some embodiments, binding of the sensor domain to the therapeutic domain versus binding of the sensor domain to PD-1 is regulated by the relative affinity of the sensor domain for the therapeutic domain. In some embodiments, the sensor domain may have a dissociation constant (Kd) for PD-1 that is lower than the dissociation constant of the sensor domain for the therapeutic domain. Thus, the sensor may have a higher affinity (lower Kd) for PD-1 than for the therapeutic domain. The sensor domains of the present disclosure may be engineered, for example by affinity maturation, to have a higher affinity (lower dissociation constant) for PD-1 than the therapeutic domain. In the absence of the marker, the sensor domain of the present disclosure may have a sufficiently high affinity for the therapeutic domain such that the therapeutic domain is bound by the sensor domain. In the presence of the marker, the affinity of the sensor domain for PD-1 is sufficiently high (low dissociation constant), such that PD-1 outcompetes the therapeutic domain for binding to the sensor domain. As a result, the equilibrium binding shifts from a state in which the sensor domain is bound to the IL-2 receptor agonist domain to a state in which the IL-2 receptor agonist domain is unbound, and the sensor domain binds PD-1.

The sensor domain may have an affinity for PD-1 that is at least 2-fold higher than an affinity for the therapeutic domain. The sensor domain may have an affinity for PD-1 that is at least 5-fold higher than an affinity for the therapeutic domain. The sensor domain may have an affinity for PD-1 that is at least 10-fold higher than an affinity for the therapeutic domain. The sensor domain may have an affinity for PD-1 that is at least 15-fold higher than an affinity for the therapeutic domain. The sensor domain may have an affinity for PD-1 that is at least 20-fold higher than an affinity for the therapeutic domain. The sensor domain may have an affinity for PD-1 that is at least 25-fold higher than an affinity for the therapeutic domain. The sensor domain may have an affinity for PD-1 that is at least 30-fold higher than an affinity for the therapeutic domain. The sensor domain may have an affinity for PD-1 that is at least 35-fold higher than an affinity for the therapeutic domain. The sensor domain may have an affinity for PD-1 that is at least 40-fold higher than an affinity for the therapeutic domain. The sensor domain may have an affinity for PD-1 that is at least 45-fold higher than an affinity for the therapeutic domain. The sensor domain may have an affinity for PD-1 that is at least 50-fold higher than an affinity for the therapeutic domain. The sensor domain may have an affinity for PD-1 that is at least 60-fold higher than an affinity for the therapeutic domain. The sensor domain may have an affinity for PD-1 that is at least 70-fold higher than an affinity for the therapeutic domain. The sensor domain may have an affinity for PD-1 that is at least 80-fold higher than an affinity for the therapeutic domain. The sensor domain may have an affinity for PD-1 that is at least 90-fold higher than an affinity for the therapeutic domain. The sensor domain may have an affinity for PD-1 that is at least 100-fold higher than an affinity for the therapeutic domain. The sensor domain may have an affinity for PD-1 that is at least 150-fold higher than an affinity for the therapeutic domain. The sensor domain may have an affinity for PD-1 that is at least 200-fold higher than an affinity for the therapeutic domain. The sensor domain may have an affinity for PD-1 that is at least 250-fold higher than an affinity for the therapeutic domain. The sensor domain may have an affinity for PD-1 that is at least 300-fold higher than an affinity for the therapeutic domain. The sensor domain may have an affinity for PD-1 that is at least 350-fold higher than an affinity for the therapeutic domain. The sensor domain may have an affinity for PD-1 that is at least 400-fold higher than an affinity for the therapeutic domain. The sensor domain may have an affinity for PD-1 that is at least 450-fold higher than an affinity for the therapeutic domain. The sensor domain may have an affinity for PD-1 that is at least 500-fold higher than an affinity for the therapeutic domain. The sensor domain may have an affinity for PD-1 that is at least 1000-fold higher than an affinity for the therapeutic domain. The sensor domain may have an affinity for PD-1 that is at least 10000-fold higher than an affinity for the therapeutic domain. The sensor domain may have an affinity for PD-1 that is at least 100000-fold higher than an affinity for the therapeutic domain. The sensor domain may have an affinity for PD-1 that is from 2 to 10-fold higher than an affinity for the therapeutic domain. The sensor domain may have an affinity for PD-1 that is from 10 to 20-fold higher than an affinity for the therapeutic domain. The sensor domain may have an affinity for PD-1 that is from 20 to 30-fold higher than an affinity for the therapeutic domain. The sensor domain may have an affinity for PD-1 that is from 30 to 40-fold higher than an affinity for the therapeutic domain. The sensor domain may have an affinity for PD-1 that is from 40 to 50-fold higher than an affinity for the therapeutic domain. The sensor domain may have an affinity for PD-1 that is from 50 to 100-fold higher than an affinity for the therapeutic domain. The sensor domain may have an affinity for PD-1 that is from 100 to 150-fold higher than an affinity for the therapeutic domain. The sensor domain may have an affinity for PD-1 that is from 150 to 200-fold higher than an affinity for the therapeutic domain. The sensor domain may have an affinity for PD-1 that is from 200 to 250-fold higher than an affinity for the therapeutic domain. The sensor domain may have an affinity for PD-1 that is from 250 to 300-fold higher than an affinity for the therapeutic domain. The sensor domain may have an affinity for PD-1 that is from 300 to 350-fold higher than an affinity for the therapeutic domain. The sensor domain may have an affinity for PD-1 that is from 350 to 400-fold higher than an affinity for the therapeutic domain. The sensor domain may have an affinity for PD-1 that is from 400 to 450-fold higher than an affinity for the therapeutic domain. The sensor domain may have an affinity for PD-1 that is from 450 to 500-fold higher than an affinity for the therapeutic domain. The sensor domain may have an affinity for PD-1 that is from 500 to 1000-fold higher than an affinity for the therapeutic domain. The sensor domain may have an affinity for PD-1 that is from 10 to 80-fold higher than an affinity for the therapeutic domain. The sensor domain may have an affinity for PD-1 that is from 30 to 70-fold higher than an affinity for the therapeutic domain. The sensor domain may have an affinity for PD-1 that is from 40 to 60-fold higher than an affinity for the therapeutic domain. The sensor domain may have an affinity for PD-1 that is from 20 to 50-fold higher than an affinity for the therapeutic domain. The sensor domain may have an affinity for PD-1 that is from 10 to 1000-fold higher than an affinity for the therapeutic domain. The sensor domain may have an affinity for PD-1 that is from 70 to 500-fold higher than an affinity for the therapeutic domain. The sensor domain may have an affinity for PD-1 that is from 100 to 500-fold higher than an affinity for the therapeutic domain. The sensor domain may have an affinity for PD-1 that is from 500 to 750-fold higher than an affinity for the therapeutic domain. The sensor domain may have an affinity for PD-1 that is from 250 to 750-fold higher than an affinity for the therapeutic domain. The sensor domain may have an affinity for PD-1 that is from 1000 to 100000-fold higher than an affinity for the therapeutic domain. The sensor domain may have an affinity for PD-1 that is from 2 to 100000-fold higher than an affinity for the therapeutic domain.

A protein complex of the present disclosure, or a fragment thereof, may comprise one or more complementary determining regions (CDRs) having have at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or substantially 100% sequence identity to any one of the CDRs disclosed herein. A protein complex of the present disclosure, or a fragment thereof, may comprise one or more heavy chain or light chain variable regions having have at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or substantially 100% sequence identity to any one of the heavy chain or light chain variable regions described herein. For example, a protein complex of the present disclosure, or a fragment thereof, may comprise one or more CDRs having at least 80% sequence identity to any one of SEQ ID NOs: 1-20, SEQ ID NOs: 142-173, or SEQ ID NOs: 238-252. A protein complex of the present disclosure, or a fragment thereof, may comprise one or more CDRs having at least 85% sequence identity to any one of SEQ ID NO: 1-SEQ ID NO: 20, SEQ ID NO: 142-173, or SEQ ID NO: 238-252. A protein complex of the present disclosure, or a fragment thereof, may comprise one or more CDRs having at least 90% sequence identity to any one of SEQ ID NO: 1-SEQ ID NO: 20, SEQ ID NO: 142-173, or SEQ ID NO: 238-252. A protein complex of the present disclosure, or a fragment thereof, may comprise one or more CDRs having at least 92% sequence identity to any one of SEQ ID NO: 1-SEQ ID NO: 20, SEQ ID NO: 142-173, or SEQ ID NO: 238-252. A protein complex of the present disclosure, or a fragment thereof, may comprise one or more CDRs having at least 95% sequence identity to any one of SEQ ID NO: 1-SEQ ID NO: 20, SEQ ID NO: 142-173, or SEQ ID NO: 238-252. A protein complex of the present disclosure, or a fragment thereof, may comprise one or more CDRs having at least 97% sequence identity to any one of SEQ ID NO: 1-SEQ ID NO: 20, SEQ ID NO: 142-173, or SEQ ID NO: 238-252. A protein complex of the present disclosure, or a fragment thereof, may comprise one or more CDRs having at least 99% sequence identity to any one of SEQ ID NO: 1-SEQ ID NO: 20, SEQ ID NO: 142-173, or SEQ ID NO: 238-252. A protein complex of the present disclosure, or a fragment thereof, may comprise one or more CDRs having any one of SEQ ID NO: 1-SEQ ID NO: 20, SEQ ID NO: 142-173, or SEQ ID NO: 238-252.

A protein complex, or a fragment thereof, can have at least 80% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof. A protein complex can have at least 85% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof. A protein complex can have at least 90% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof. A protein complex can have at least 92% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof. A protein complex can have at least 95% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof. A protein complex can have at least 97% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof. A protein complex can have at least 99% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof. A protein complex is any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof.

A protein complex of the present disclosure may have at least 95% sequence identity to any one of SEQ ID NO: 41, SEQ ID NO: 44, SEQ ID NO: 80-SEQ ID NO: 112, SEQ ID NO: 174-175, SEQ ID NO: 181-182, SEQ ID NO: 195-196, SEQ ID NO: 205-206, SEQ ID NO: 210-212, SEQ ID NO: 220-223, SEQ ID NO: 226-231, SEQ ID NO: 259-261, SEQ ID NO: 266-282, or SEQ ID NO: 289-293, or a fragment thereof and have one or more CDRs with at least 80% sequence identity to any one SEQ ID NO: 1-SEQ ID NO: 20, SEQ ID NO: 142-173, or SEQ ID NO: 238-252. The protein complexes of the present disclosure can have CDRs selected from SEQ ID NO: 1-SEQ ID NO: 20, SEQ ID NO: 142-173, or SEQ ID NO: 238-252 arranged in any combination or order.

A fragment of any of the above may retain the functional binding domains of the sensor or any functional therapeutic domains of the therapeutic. For example, a dual binding antibody protein complex can include the entire antibody or a fragment having regions of the antibody that are capable of binding to a marker and the therapeutic domain. In the latter case, the fragment may be an scFv that can bind to a marker and the therapeutic domain. Exemplary sequence of protein complexes of the present disclosure is shown below in TABLE 1.

TABLE 1
Exemplary Protein Complexes

| SEQ ID NO | Sequence | Description |
|---|---|---|
| SEQ ID NO: | QVQLVESGGGVVQPGRSLRLDCKASGITFSN | PD1- |
| 769 | SGMHWVRQAPGKGLEWVAVIWYDGSKRYY | IL2_3x_Cterm_Nivo_ |
| | ADSVKGRFTISRDNSKNTLFLQMNSLRAEDT | 2B07_H_H37Y_L_ |
| | AVYYCATNDDYWGQGTLVTVSSAKTTAPSV | A107Y_S109R; |
| | YPLAPVCGDTTGSSVTLGCLVKGYFPEPVTL | AF4695_pep2 |
| | TWNSGSLSSGVHTFPAVLQSDLYTLSSSVTV | |
| | TSSTWPSQSITCNVAHPASSTKVDKKIEPRGP | |
| | TIKPCPPCKCPAPNAAGGPSVFIFPPKIKDVLM | |
| | ISLSPIVTCVVVDVSEDDPDVQISWFVNNVEV | |
| | HTAQTQTHREDYNSTLRVVSALPIQHQDWM | |
| | SGKEFKCKVNNKDLGAPIERTISKPKGSVRAP | |
| | QVYVLPPPEKEMTKKQVSLTCLVKDFMPEDI | |
| | YVEWTNNGKTELNYKNTEPVLKSDGSYFMY | |
| | SKLTVEKKNWVERNSYSCSVVHEGLHNHHT | |
| | TKSFSRTPGGGGGSGGGGSGGGGSGGGGSQ | |
| | VQLVQSGAEVKKPGASVKVSCKASGDTFTR | |
| | YYVHWVRQAPGQGLEWMGIINPSGGYASYA | |
| | QKFQGRVTMTRDTSTSTVYMELSSLRSEDTA | |
| | VYYCAAGLFIWGQGTLVTVSSASGGGGSGG | |
| | GGSGGGGSHASDIQMTQSPSSLSASVGDRVTI | |
| | TCRASQSIGRWLAWYQQKPGKAPKLLIYSAS | |
| | NLETGVPSRFSGSGSGTDFTLTISSLQPEDFAT | |
| | YYCQQYNRFPVTFGPGTKVDIK | |
| SEQ ID NO: | QVQLVESGGGVVQPGRSLRLDCKASGITFSN | PD1- |
| 759 | SGMHWVRQAPGKGLEWVAVIWYDGSKRYY | IL2_3x_Cterm_Nivo_ |
| | ADSVKGRFTISRDNSKNTLFLQMNSLRAEDT | 2B07_H_H37Y_L_ |
| | AVYYCATNDDYWGQGTLVTVSSAKTTAPSV | A107Y_S109R; |
| | YPLAPVCGDTTGSSVTLGCLVKGYFPEPVTL | AF4695_pep1 |
| | TWNSGSLSSGVHTFPAVLQSDLYTLSSSVTV | AND |
| | TSSTWPSQSITCNVAHPASSTKVDKKIEPRGP | AF4696_pep1 |
| | TIKPCPPCKCPAPNAAGGPSVFIFPPKIKDVLM | |
| | ISLSPIVTCVVVDVSEDDPDVQISWFVNNVEV | |
| | HTAQTQTHREDYNSTLRVVSALPIQHQDWM | |
| | SGKEFKCKVNNKDLGAPIERTISKPKGSVRAP | |
| | QVYVLPPPEEEMTKKQVTLTCMVTDFMPEDI | |
| | YVEWTNNGKTELNYKNTEPVLDSDGSYFMY | |
| | SDLRVEKKNWVERNSYSCSVVHEGLHNHHT | |
| | TESFSRTPGGGGGSGGGGSGGGGSAPTSSST | |
| | KKTQLQLEHLLLDLQMILNGINNYKNPKLTD | |
| | MLTFEFYMPKKATELKHLQCLERELKPLEEV | |
| | LNLAQSKNFHLRPRDLISNINVIVLELKGSETT | |
| | FMCEYADETATIVEFLNRWITFCQSIISTLT | |
| SEQ ID NO: | EIVLTQSPATLSLSPGERATLSCRASQSVSSYL | PD1- |
| 745 | AWYQQKPGQAPRLLIYDASNRATGIPARFSG | IL2_3x_Cterm_Nivo_ |
| | SGSGTDFTLTISSLEPEDFAVYYCQQSSNWPR | 2B07_H_H37Y_L_ |
| | TFGQGTKVEIKRADAAPTVSIFPPSSEQLTSG | A107Y_S109R; |
| | GASVVCFLNNFYPKDINVKWKIDGSERQNG | AF4695_pep3 |
| | VLNSWTDQDSKDSTYSMSSTLTLTKDEYERH | AND |
| | NSYTCEATHKTSTSPIVKSFNRNEC | AF4695_pep3 |
| SEQ ID NO: | QVQLVESGGGVVQPGRSLRLDCKASGITFSN | PD1- |
| 770 | SGMHWVRQAPGKGLEWVAVIWYDGSKRYY | IL2_3x_Cterm_Nivo_ |
| | ADSVKGRFTISRDNSKNTLFLQMNSLRAEDT | 704var |
| | AVYYCATNDDYWGQGTLVTVSSAKTTAPSV | AF4696_pep2 |
| | YPLAPVCGDTTGSSVTLGCLVKGYFPEPVTL | |
| | TWNSGSLSSGVHTFPAVLQSDLYTLSSSVTV | |
| | TSSTWPSQSITCNVAHPASSTKVDKKIEPRGP | |
| | TIKPCPPCKCPAPNAAGGPSVFIFPPKIKDVLM | |
| | ISLSPIVTCVVVDVSEDDPDVQISWFVNNVEV | |
| | HTAQTQTHREDYNSTLRVVSALPIQHQDWM | |
| | SGKEFKCKVNNKDLGAPIERTISKPKGSVRAP | |
| | QVYVLPPPEKEMTKKQVSLTCLVKDFMPEDI | |
| | YVEWTNNGKTELNYKNTEPVLKSDGSYFMY | |
| | SKLTVEKKNWVERNSYSCSVVHEGLHNHHT | |
| | TKSFSRTPGGGGGSGGGGSGGGGSGGGGSQ | |
| | VQLVQSGAEVKKPGASVKVSCKASGYTFTR | |
| | YYMHWVRQAPGQGLEWMGIINPRAGYTSY | |
| | ALKFQGRVTMTRDTSTSTVYMELSSLRSEDT | |
| | AVYYCTSGWDVWGQGTLVTVSSASGGGGS | |
| | GGGGSGGGGSHASDIQMTQSPSSLSASVGDR | |
| | VTITCRASQSISTWLAWYQQKPGKAPKLLIY | |
| | AASSLDSGVPSRFSGSGSGTDFTLTISSLQPED | |
| | FATYYCQQSYSFPVTFGQGTKVEIK | |
| SEQ ID NO: | APTSSSTKKTQLQLEHLLLDLQMILNGINNYK | PD1- |
| 181 | NPKLTDMLTFEFYMPKKATELKHLQCLEREL | IL2_3x_Asym_PD1- |
| | KPLEEVLNLAQSKNFHLRPRDLISNINVIVLEL | IL2_2B07_H_H37Y_ |
| | KGSETTFMCEYADETATIVEFLNRWITFCQSII | L_W38Y_A107Y |
| | STLTGGGGSGGGGSGGGGSGGGGSQVQLVQ | AF4386_pep1 |
| | SGAEVKKPGASVKVSCKASGDTFTRYYVHW | |
| | VRQAPGQGLEWMGIINPSGGYASYAQKFQG | |
| | RVTMTRDTSTSTVYMELSSLRSEDTAVYYCA | |
| | AGLFIWGQGTLVTVSSAKTTAPSVYPLAPVC | |
| | GDTTGSSVTLGCLVKGYFPEPVTLTWNSGSL | |
| | SSGVHTFPAVLQSDLYTLSSSVTVTSSTWPSQ | |
| | SITCNVAHPASSTKVDKKIEPRGPTIKPCPPCK | |
| | CPAPNAAGGPSVFIFPPKIKDVLMISLSPIVTC | |
| | VVVDVSEDDPDVQISWFVNNVEVHTAQTQT | |
| | HREDYNSTLRVVSALPIQHQDWMSGKEFKC | |
| | KVNNKDLGAPIERTISKPKGSVRAPQVYVLPP | |
| | PEEEMTKKQVTLTCMVTDFMPEDIYVEWTN | |
| | NGKTELNYKNTEPVLDSDGSYFMYSDLRVE | |
| | KKNWVERNSYSCSVVHEGLHNHHTTESFSR | |
| | TPGK | |
| SEQ ID NO: | QVQLVQSGAEVKKPGASVKVSCKASGDTFTRYYVHWV | PD1- |
| 182 | RQAPGQGLEWMGIINPSGGYASYAQKFQGRVTMTRD | IL2_3x_Asym_PD1- |
| | TSTSTVYMELSSLRSEDTAVYYCAAGLFIWGQGTLVTVS | IL2_2B07_H_H37YL_ |
| | SAKTTAPSVYPLAPVCGDTTGSSVTLGCLVKGYFPEPVT | W38Y_A107Y |
| | LTWNSGSLSSGVHTFPAVLQSDLYTLSSSVTVTSSTWPS | AF4386_pep2 |
| | QSITCNVAHPASSTKVDKKIEPRGPTIKPCPPCKCPAPN | |
| | AAGGPSVFIFPPKIKDVLMISLSPIVTCVVVDVSEDDPDV | |
| | QISWFVNNVEVHTAQTQTHREDYNSTLRVVSALPIQH | |
| | QDWMSGKEFKCKVNNKDLGAPIERTISKPKGSVRAPQ | |
| | VYVLPPPEKEMTKKQVSLTCLVKDFMPEDIYVEWTNN | |
| | GKTELNYKNTEPVLKSDGSYFMYSKLTVEKKNWVERNS | |
| | YSCSVVHEGLHNHHTTKSFSRTPGGGGSGGGSHHHHH | |
| | H | |
| SEQ ID NO: | DIQMTQSPSSLSASVGDRVTITCRASQSIGRY | PD1- |
| 212 | LAWYQQKPGKAPKLLIYSASNLETGVPSRFS | IL2_3x_Asym_PD1- |
| | GSGSGTDFTLTISSLQPEDFATYYCQQYNSFP | IL2_2B07_H_H37YL_ |
| | VTFGPGTKVDIKRADAAPTVSIFPPSSEQLTS | W38Y_A107Y |
| | GGASVVCFLNNFYPKDINVKWKIDGSERQN | AF4386_pep3 |
| | GVLNSWTDQDSKDSTYSMSSTLTLTKDEYER | |
| | HNSYTCEATHKTSTSPIVKSFNRNEC | |
| SEQ ID NO: | APTSSSTKKTQLQLEHLLLDLQMILNGINNYKNPKLTDM | PD1- |
| 183 | LTFEFYMPKKATELKHLQCLERELKPLEEVLNLAQSKNF | IL2_3x_Asym_PD1- |
| | HLRPRDLISNINVIVLELKGSETTFMCEYADETATIVEFLN | IL2_7A04_H_M115W_ |
| | RWITFCQSIISTLTGGGGSGGGGSGGGGSGGGGSQVQ | L_Q68D |
| | LVQSGAEVKKPGASVKVSCKASGYTFTDYYMHWVRQ | AF4387_pep1 |
| | APGQGLEWMGIINPRAGYTSYALKFQGRVTMTRDTST | |
| | STVYMELSSLRSEDTAVYYCTSGWDVWGQGTLVTVSS | |
| | AKTTAPSVYPLAPVCGDTTGSSVTLGCLVKGYFPEPVTL | |
| | TWNSGSLSSGVHTFPAVLQSDLYTLSSSVTVTSSTWPS | |
| | QSITCNVAHPASSTKVDKKIEPRGPTIKPCPPCKCPAPN | |
| | AAGGPSVFIFPPKIKDVLMISLSPIVTCVVVDVSEDDPDV | |
| | QISWFVNNVEVHTAQTQTHREDYNSTLRVVSALPIQH | |
| | QDWMSGKEFKCKVNNKDLGAPIERTISKPKGSVRAPQ | |
| | VYVLPPPEEEMTKKQVTLTCMVTDFMPEDIYVEWTNN | |
| | GKTELNYKNTEPVLDSDGSYFMYSDLRVEKKNWVERN | |
| | SYSCSVVHEGLHNHHTTESFSRTPGK | |
| SEQ ID NO: | QVQLVQSGAEVKKPGASVKVSCKASGYTFTDYYMHW | PD1- |
| 184 | VRQAPGQGLEWMGIINPRAGYTSYALKFQGRVTMTR | IL2_3x_Asym_PD1- |
| | DTSTSTVYMELSSLRSEDTAVYYCTSGWDVWGQGTLV | IL2_7A04_H_M115W_ |
| | TVSSAKTTAPSVYPLAPVCGDTTGSSVTLGCLVKGYFPE | L_Q68D |
| | PVTLTWNSGSLSSGVHTFPAVLQSDLYTLSSSVTVTSST | AF4387_pep2 |
| | WPSQSITCNVAHPASSTKVDKKIEPRGPTIKPCPPCKCP | |
| | APNAAGGPSVFIFPPKIKDVLMISLSPIVTCVVVDVSED | |
| | DPDVQISWFVNNVEVHTAQTQTHREDYNSTLRVVSAL | |
| | PIQHQDWMSGKEFKCKVNNKDLGAPIERTISKPKGSVR | |
| | APQVYVLPPPEKEMTKKQVSLTCLVKDFMPEDIYVEWT | |
| | NNGKTELNYKNTEPVLKSDGSYFMYSKLTVEKKNWVE | |
| | RNSYSCSVVHEGLHNHHTTKSFSRTPGGGGSGGGSHH | |
| | HHHH | |
| SEQ ID NO: | DIQMTQSPSSLSASVGDRVTITCRASQSISTW | PD1- |
| 185 | LAWYQQKPGKAPKLLIYAASSLDSGVPSRFS | IL2_3x_Asym_PD1- |
| | GSGSGTDFTLTISSLQPEDFATYYCQQSYSFP | IL2_7A04_H_M115W_ |
| | VTFGQGTKVEIKRADAAPTVSIFPPSSEQLTS | L_Q68D |
| | GGASVVCFLNNFYPKDINVKWKIDGSERQN | AF4387_pep3 |
| | GVLNSWTDQDSKDSTYSMSSTLTLTKDEYER | |
| | HNSYTCEATHKTSTSPIVKSFNRNEC | |

### A. Sensor Domains

Protein complexes of the present disclosure include sensor domains comprised of a dual-binding antibody with affinity for PD-1 and affinity for an IL-2 receptor agonist. A sensor domain may be any protein that is capable of sensing the presence of a first moiety and regulating a second moiety, where the first moiety is PD-1 and the second moiety is an IL-2 receptor agonist. For example, the present disclosure provides a sensor domain that may be an antibody or antibody fragment capable of binding a first moiety and binding and blocking the activity of a second moiety, wherein the first moiety is PD-1 and the second moiety is IL-2 or another IL-2 receptor agonist. In the absence of the first moiety, the sensor domain binds the second moiety. If the first moiety is introduced into the system, the sensor domain binds the first moiety and unbinds the second moiety. Thus, the binding and unbinding of the second moiety is reversible. The sensor domain inactivates or blocks the activity of the IL-2 receptor agonist domain by binding the IL-2 receptor agonist domain and preventing it from binding to its target (the IL-2 receptor). The sensor domain regulates the IL-2 receptor agonist domain by releasing it to act on its target upon binding of PD-1.

In some embodiments, the sensor domain is a dual binding antibody. A dual binding antibody may be capable of binding PD-1 and the IL-2 receptor agonist domain. A dual binding antibody of the present disclosure may be selected or engineered to bind PD-1 and the therapeutic domain. The dual binding protein may have a higher affinity for PD-1 as compared to the IL-2 receptor agonist domain. The dual binding protein may be affinity matured to have a higher affinity for PD-1 as compared to the IL-2 receptor agonist domain.

In some embodiments, the sensor domain is an antibody. The sensor domain may also be a fragment of an antibody. A fragment of an antibody consistent with the sensor domains disclosed herein retains its ability to exhibit dual binding to both PD-1 and an IL-2 receptor agonist domain. One or both domains of a bispecific antibody may be sensor domains of the protein complexes of the present disclosure. In the instance that bispecific antibodies are used, the bispecific antibody may include a first antigen binding domain that may bind an IL-2 receptor agonist domain and PD-1 and may also include a second antigen binding domain capable of binding PD-1.

In some embodiments, the sensor domain is an anti-PD1 antibody or fragment thereof (e.g., an scFv that binds PD1 or PD-L1).

### B. Therapeutic Domains

Protein complexes of the present disclosure include therapeutic domains comprised of an IL-2 receptor agonist. A therapeutic domain of the present disclosure is linked to a sensor domain via a linker to form a protein complex. The therapeutic domain may exert therapeutic activity by binding to an IL-2 receptor.

In some embodiments, the protein complexes of the present disclosure comprise a therapeutic domain comprising IL-2, or variants or fusions of this cytokine. The therapeutic domain may also be a fragment of the above-mentioned moiety. A fragment retains functional regions of the moiety needed for binding to its target (e.g., IL-2 receptor) and any functional regions needed for activity.

In some embodiments, the protein complexes of the present disclosure comprise a therapeutic domain comprising IL-15, or variants or fusions of this cytokine. The therapeutic domain may also be a fragment of the above-mentioned moiety. A fragment retains functional regions of the moiety needed for binding to its target (e.g., IL-2 receptor) and any functional regions needed for activity.

In some embodiments, the protein complexes of the present disclosure comprise a therapeutic domain comprising a peptide, and engineered protein or an antibody capable of binding IL-2 receptor beta and IL-2 receptor gamma. The therapeutic domain may also be a fragment of the above-mentioned moiety. A fragment retains functional regions of the moiety needed for binding to its target (e.g., IL-2 receptor) and any functional regions needed for activity.

### C. Linkers

A protein complex disclosed herein may comprise a linker. The linker may connect two domains, such as a sensor domain and a therapeutic domain. Various linkers are consistent with the protein complexes of the present disclosure. In some embodiments, the linker may be an amino acid linker or a chemical linker.

The linker may be a stable linker. For example, a linker may maintain a connection between a therapeutic domain and a sensor domain even upon binding of the sensor domain to a marker and, thereby, unbinding of the therapeutic domain from the sensor domain. For example, although the sensor domain may unbind the therapeutic domain, the therapeutic domain may remain linked to the sensor domain via the linker. Examples of linkers that are consistent with this activity may include non-cleavable linkers.

The linker may also be a flexible linker. A flexible linker is a linker that is long enough to allow for the therapeutic domain to bind to the IL-2 receptor, once it is unbound from the sensor domain. Flexibility of the linker may affect therapeutic efficacy. For example, upon binding of the sensor domain to PD-1 and unbinding of the therapeutic domain, the therapeutic domain needs to be able to encounter and bind its target, the IL-2 receptor. If the linker is not flexible enough to allow for the therapeutic domain to bind the IL-2 receptor, therapeutic efficacy may be reduced or not exerted. When the linker is flexible, therapeutic domains may be able to bind the IL-2 receptor and exert high therapeutic efficacy. Flexibility of a linker may arise from the length of the linker. For example, short linkers may sterically hinder the therapeutic domain from binding the IL-2 receptor. Longer linkers may allow for the protein complex to be more flexible and allow for therapeutic domains to bind the IL-2 receptor. In some embodiments, a linker that is too long may impact the ability of the sensor domain to bind the therapeutic domain and inhibit activity in the absence of PD-1. In some embodiments, a linker that is too long may impact the stability of a protein therapeutic domain or the half-life of the protein therapeutic domain in vivo.

In some embodiments, the linker may be attached to a heavy chain of the sensor domain or a light chain of the sensor domain. A linker may be fused to the N-terminus or C-terminus of the sensor domain. In some embodiments, the linker may be fused with the N-terminus or C-terminus of the IL-2 receptor agonist domain. For example, a linker may be attached to an N-terminus or C-terminus of an scFV or an ScFab.

Amino Acid Linkers. An amino acid linker may comprise any amino acid residues. In some embodiments, favored amino acid residues are amino acid residues that are entropically flexible. Favored amino acid residues in an amino acid linker of the present disclosure may include glycine and serine. Other preferred amino acid residues may include alanine, proline, threonine, and glutamic acid. In preferred embodiments, the amino acid linker may comprise from 3 to 60 amino acid residues in length. In some embodiments, the amino acid linker may comprise 20 amino acid residues. In some embodiments, the amino acid linker may comprise 40 amino acid residues. In some embodiments, the amino acid linker may comprise 60 amino acid residues. In some embodiments, the amino acid linker may comprise 80 amino acid residues. An amino acid linker may comprise at least 5 amino acid residues. An amino acid linker may comprise at least 10 amino acid residues. An amino acid linker may comprise at least 15 amino acid residues. An amino acid linker may comprise at least 20 amino acid residues. An amino acid linker may comprise at least 25 amino acid residues. An amino acid linker may comprise at least 30 amino acid residues. An amino acid linker may comprise at least 35 amino acid residues. An amino acid linker may comprise at least 40 amino acid residues. An amino acid linker may comprise at least 45 amino acid residues. An amino acid linker may comprise at least 50 amino acid residues. An amino acid linker may comprise at least 55 amino acid residues. An amino acid linker may comprise at least 60 amino acid residues. An amino acid linker may comprise at least 65 amino acid residues. An amino acid linker may comprise at least 70 amino acid residues. An amino acid linker may comprise at least 75 amino acid residues. An amino acid linker may comprise at least 80 amino acid residues. An amino acid linker may comprise at least 85 amino acid residues. An amino acid linker may comprise at least 90 amino acid residues. An amino acid linker may comprise at least 95 amino acid residues. An amino acid linker may comprise at least 100 amino acid residues. An amino acid linker may comprise at least 110 amino acid residues. An amino acid linker may comprise at least 120 amino acid residues. An amino acid linker may comprise at least 130 amino acid residues. An amino acid linker may comprise at least 140 amino acid residues. An amino acid linker may comprise at least 150 amino acid residues. An amino acid linker may comprise at least 160 amino acid residues. An amino acid linker may comprise at least 170 amino acid residues. An amino acid linker may comprise at least 180 amino acid residues. An amino acid linker may comprise at least 190 amino acid residues. An amino acid linker may comprise at least 200 amino acid residues. An amino acid linker may comprise at least 300 amino acid residues. An amino acid linker may comprise at least 400 amino acid residues. An amino acid linker may comprise at least 500 amino acid residues. An amino acid linker may comprise from 5 to 10 amino acid residues. An amino acid linker may comprise from 10 to 15 amino acid residues. An amino acid linker may comprise from 15 to 20 amino acid residues. An amino acid linker may comprise from 20 to 25 amino acid residues. An amino acid linker may comprise from 25 to 30 amino acid residues. An amino acid linker may comprise from 30 to 35 amino acid residues. An amino acid linker may comprise from 35 to 40 amino acid residues. An amino acid linker may comprise from 40 to 45 amino acid residues. An amino acid linker may comprise from 45 to 50 amino acid residues. An amino acid linker may comprise from 50 to 55 amino acid residues. An amino acid linker may comprise from 55 to 60 amino acid residues. An amino acid linker may comprise from 60 to 65 amino acid residues. An amino acid linker may comprise from 65 to 70 amino acid residues. An amino acid linker may comprise from 70 to 75 amino acid residues. An amino acid linker may comprise from 75 to 80 amino acid residues. An amino acid linker may comprise from 80 to 85 amino acid residues. An amino acid linker may comprise from 85 to 90 amino acid residues. An amino acid linker may comprise from 90 to 95 amino acid residues. An amino acid linker may comprise from 95 to 100 amino acid residues. An amino acid linker may comprise from 5 to 80 amino acid residues. An amino acid linker may comprise from 20 to 40 amino acid residues. An amino acid linker may comprise from 20 to 80 amino acid residues. An amino acid linker may comprise from 30 to 60 amino acid residues. An amino acid linker may comprise from 40 to 50 amino acid residues. An amino acid linker may comprise from 10 to 30 amino acid residues. An amino acid linker may comprise from 10 to 20 amino acid residues. An amino acid linker may comprise from 5 to 25 amino acid residues. An amino acid linker may comprise from 25 to 75 amino acid residues. An amino acid linker may comprise from 100 to 500 amino acid residues. An amino acid linker may comprise from 100 to 300 amino acid residues. An amino acid linker may comprise from 5 to 500 amino acid residues. An amino acid linker may comprise no more than 100 amino acid residues. An amino acid linker may comprise no more than 90 amino acid residues. An amino acid linker may comprise no more than 80 amino acid residues. An amino acid linker may comprise no more than 70 amino acid residues. An amino acid linker may comprise no more than 60 amino acid residues. An amino acid linker may comprise no more than 50 amino acid residues. An amino acid linker may comprise no more than 40 amino acid residues. An amino acid linker may comprise no more than 30 amino acid residues. An amino acid linker may comprise no more than 20 amino acid residues. An amino acid linker may comprise no more than 10 amino acid residues. An amino acid linker may comprise no more than 95 amino acid residues. An amino acid linker may comprise no more than 90 amino acid residues. An amino acid linker may comprise no more than 85 amino acid residues. An amino acid linker may comprise no more than 80 amino acid residues. An amino acid linker may comprise no more than 75 amino acid residues. An amino acid linker may comprise no more than 70 amino acid residues. An amino acid linker may comprise no more than 65 amino acid residues. An amino acid linker may comprise no more than 60 amino acid residues. An amino acid linker may comprise no more than 55 amino acid residues. An amino acid linker may comprise no more than 50 amino acid residues. An amino acid linker may comprise no more than 45 amino acid residues. An amino acid linker may comprise no more than 40 amino acid residues. An amino acid linker may comprise no more than 35 amino acid residues. An amino acid linker may comprise no more than 30 amino acid residues. An amino acid linker may comprise no more than 25 amino acid residues. An amino acid linker may comprise no more than 20 amino acid residues. An amino acid linker may comprise no more than 15 amino acid residues. An amino acid linker may comprise no more than 10 amino acid residues. An amino acid linker may comprise no more than 200 amino acid residues. An amino acid linker may comprise no more than 300 amino acid residues. An amino acid linker may comprise no more than 400 amino acid residues. An amino acid linker may comprise no more than 500 amino acid residues.

### Non-Cleavable Linkers.

A non-cleavable linker may include a non-proteolytically cleavable peptide. A non-proteolytically cleavable peptide may be inert to proteases present in a given sample or organism. For example, a peptide may be inert to all human protease cleavage sequences, and thereby may comprise a high degree of stability within humans and human samples. Such a peptide may also comprise a secondary structure which renders a protease cleavage site inert or inaccessible to a protease. A non-cleavable linker of the present disclosure may comprise a half-life for cleavage of at least 1 hour, at least 2 hours, at least 4 hours, at least 8 hours, at least 12 hours, at least 16 hours, at least 1 day, at least 2 days, at least 3 days, at least 1 week, at least 2 weeks, or at least 1 month in the presence of human proteases at 25° C. in pH 7 buffer.

### D. Protein Complex Structures

The present disclosure provides a wide variety of protein complexes spanning a range of structures. A protein complex of the present disclosure may comprise an IL-2 receptor agonist domain and a sensor domain expressed as a single unit. An IL-2 receptor agonist domain may be expressed as an N-terminal extension of a sensor domain, as a C-terminal extension of a sensor domain, or disposed within a sensor domain. For example, a protein complex may comprise a peptide which comprises, from N-terminus to C-terminus, an IL-2 receptor agonist domain, a peptide linker, an scFv domain, and optionally a tag, such as a purification tag (e.g., a V5 or myc tag) or a localization signal.

A protein complex may comprise a plurality of protein subunits. The plurality of protein subunits (e.g., an IL-2 receptor agonist domain and a sensor domain, two sensor domains, or two subunits of a sensor domain) may be chemically or physically coupled following expression. The plurality of protein subunits may comprise a plurality of sensor and/or therapeutic domains. A sensor and/or a therapeutic domain may be comprised of a single protein subunit, of multiple protein subunits, or by portions thereof. For example, a sensor domain may comprise an antibody Fab region comprising portions of an immunoglobulin light chain and an immunoglobulin heavy chain.

A plurality of protein subunits may comprise physical handles which facilitate their selective coupling. The physical handles may enable spontaneous, irreversible, and/or non-mediated (e.g., not requiring a chaperone protein or a catalytic complex) coupling between the protein subunits, thereby enabling complex and asymmetric protein complexes. For example, two distinct protein complex subunits expressed in a single Chinese hamster ovary (CHO) cell, may comprise physical handles which spontaneously and irreversibly couple prior to cellular export. Such physical handles may comprise a ‘knob-into-hole’ (KIH) construct or a charge-swap construct, in which two protein subunits comprise physical structures with mutual binding affinities and specificities. Such physical handles may comprise a covalently binding pair, such as a plurality of thiols configured to form disulfide bonds. Physical handles may enable facile production of protein complexes comprising identical or distinct domains.

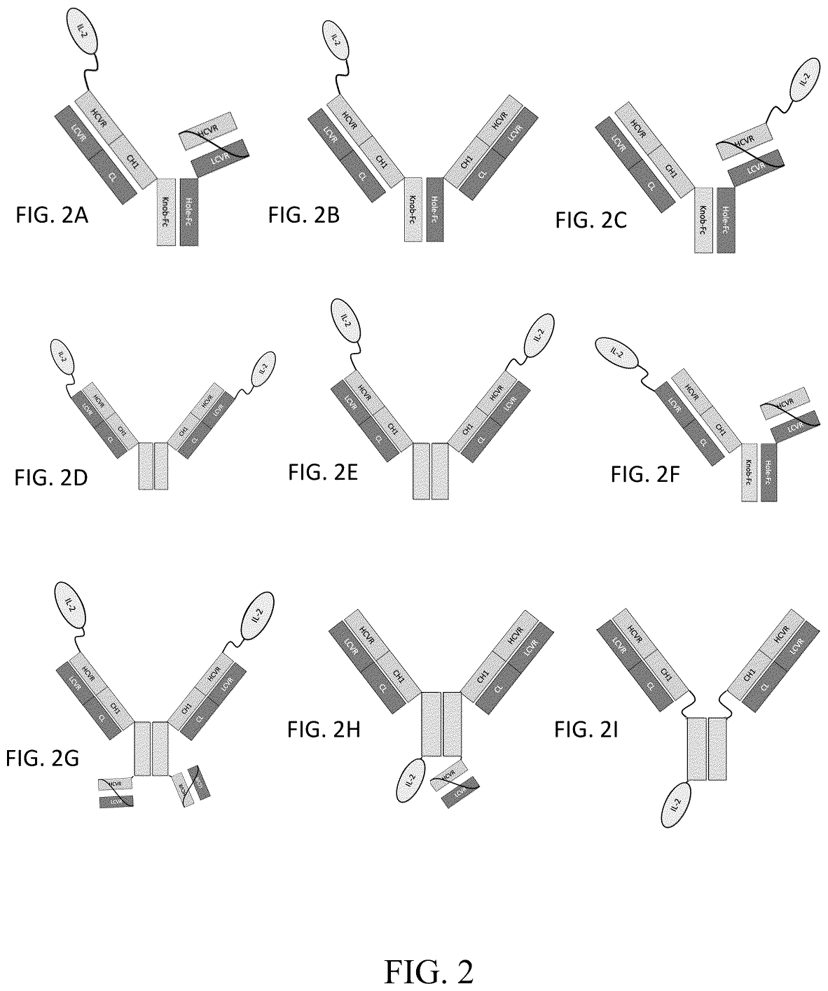

A protein complex may comprise two or more identical domains. An example of such a protein complex is provided in FIG. 2E, which illustrates an antibody (multi-sensor domain) coupled to two IL-2 therapeutic domains. In this example, the protein complex comprises two protein immunoglobulin light chain subunits and two immunoglobulin heavy chain subunits complexed to form a competent antibody. The two immunoglobulin heavy chain subunits comprise N-terminal linkers coupled to IL-2 therapeutic domains. Each immunoglobulin heavy chain is coupled to an immunoglobulin light chain, such that the protein complex comprises two Fab regions, each separately coupled to a therapeutic domain by a linker. A second example of such a protein complex is provided in FIG. 2D, which illustrates an antibody (multi-sensor domain) coupled to two IL-2 therapeutic domains. In this example, the protein complex comprises two protein immunoglobulin light chain subunits and two immunoglobulin heavy chain subunits complexed to form a competent antibody. The two immunoglobulin light chain subunits comprise N-terminal linkers coupled to IL-2 therapeutic domains. Each immunoglobulin heavy chain is coupled to an immunoglobulin light chain, such that the protein complex comprises two Fab regions, each separately coupled to a therapeutic domain by a linker. A third example of such a protein complex is provided in FIG. 2G, which illustrates an antibody (multi-sensor domain) coupled to two IL-2 therapeutic domains and four sensor domains. In this example, the protein complex comprises two protein immunoglobulin light chain subunits and two immunoglobulin heavy chain subunits complexed to form a competent antibody. The two immunoglobulin heavy chain subunits comprise N-terminal linkers coupled to IL-2 therapeutic domains and comprise C-terminal linkers coupled to a sensor domain which does not target a therapeutic domain (an anti-PD-1 scFv domain). Each immunoglobulin heavy chain is coupled to an immunoglobulin light chain, such that the protein complex comprises two Fab regions, each separately coupled to a therapeutic domain by a linker and two Fc domains, each separately coupled to a sensor by a linker.

While the above example provides a symmetric protein complex with two identical sensor domains and two identical therapeutic domains, a protein complex may also comprise a plurality of distinct sensor and/or therapeutic domains. Such a protein complex may comprise an immunoglobulin unit with a first arm comprised of a heavy chain-light chain pair, and a second arm comprised of an antibody fragment such as an scFv, an scFab, a VH, or a fragment thereof. In such cases, the heavy chain, the antibody fragment, or the light chain may comprise an N-terminal extension with a linker and a therapeutic domain, as illustrated in FIGS. 2A, C, and F, respectively. Alternatively, the heavy chain, the antibody fragment, or the light chain may comprise a C-terminal extension with a linker and a therapeutic domain. A protein complex may also comprise a symmetric immunoglobulin unit with a single therapeutic domain. For example, as shown in FIG. 2B, an immunoglobulin unit may comprise an N-terminal linker and therapeutic unit on a single heavy chain. Alternatively, an immunoglobulin unit may comprise an N-terminal linker and therapeutic unit on a single light chain. An immunoglobulin unit may also comprise a pair of antibody fragments coupled to a single Fc region. An immunoglobulin unit may comprise a nanobody. An immunoglobulin unit may comprise a diabody.

A protein complex may comprise flexible linker between the Fab arm and the Fc domain of a competent antibody, such that the Fab sensor domain is capable of binding a therapeutic domain linked to the C-term of the Fc domain, as shown in FIG. 51. In this example, the protein complex comprises two protein immunoglobulin light chain subunits and two immunoglobulin heavy chain subunits complexed to form the sensor antibody domains.

A protein complex may comprise a sensor domain which does not target a therapeutic domain. Such a sensor domain may aid in target localization, or may enhance the binding of a separate sensor domain to PD-1. An example of a protein complex comprising a sensor domain which does not target a therapeutic domain is provided in FIG. 2H. This system comprises a monospecific anti-PD-1 antibody, wherein a first heavy chain comprises a C-terminal linker coupled to a therapeutic domain, and a second heavy chain comprises a C-terminal linker coupled to a sensor domain with dual specificity for the IL-2 receptor agonist domain and for PD-1.

A protein complex may comprise a range of sensor-to-therapeutic domain ratios. A protein complex may comprise equal numbers of sensor domains and therapeutic domains, examples of which are provided by FIGS. 2D and 2E, which illustrate protein complexes with 2 sensor domains and 2 therapeutic domains. A protein complex may comprise a greater number of sensor domains than therapeutic domains, such as the protein complexes of FIGS. 2A, 2B, 2C, 2F and 2I, which each comprise two sensor domains and one therapeutic domain, or such as FIG. 2G, which comprises four sensor domains and two therapeutic domains and FIG. 2H, which comprises three sensor domains and one therapeutic domain. In such cases, a therapeutic domain may be capable of interacting with multiple sensor domains, or may be constrained from interacting with more than one sensor domain. The number of therapeutic domains with which a sensor domain may interact may depend on its linker. A linker may be sufficiently short so as to prevent a therapeutic domain from interacting with a sensor domain, or may be sufficiently long so as to allow a therapeutic domain to interact with multiple sensor domains.

In specific cases, a protein complex may comprise an antibody with Fc-coupled therapeutic and sensor domains. As illustrated in FIG. 2H, a protein complex may comprise an antibody with a first heavy chain C-terminal extension comprising a linker and a therapeutic domain, and a second heavy chain C-terminal extension comprising a linker and a sensor domain. An antibody of this design may comprise common targets across its Fab and C-terminal extension sensor domain. For example, the antibody Fab regions and C-terminal extension sensor domain may each target the same epitope on PD-1. Conversely, an antibody of this design may comprise separate targets across its Fab regions and C-terminal extension sensor domain. For example, the antibody Fab regions and C-terminal extension sensor domain may each target a different epitope on PD-1. As illustrated in FIG. 2I, a protein complex may comprise an antibody with a first heavy chain comprising a flexible linker the CH1 and CH2 domains (between the Fab arm and the Fc domain) and a heavy chain C-terminal extension comprising a linker and a therapeutic domain, and a second first heavy chain comprising a flexible linker the CH1 and CH2 domains (between the Fab arm and the Fc domain) with no C-terminal extension.

In some embodiments, an amino acid in the protein complex described herein may comprise a conservative substitution. A conservative substitution may comprise a substitution of one amino acid with a different amino acid with similar biochemical properties (e.g. charge, hydrophobicity, and size). Examples of conservative substitutions, as well as substitutions that may be, but are not necessarily, preferred, are provided in TABLE 2 below.

TABLE 2
Example Conservative Substitutions

| Original | | Preferred |
|---|---|---|
| Residue | Example Substitutions | Substitutions |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

In some embodiments, the present disclosure describes a recombinant nucleic acid that encodes the protein complex disclosed herein. In some embodiments, the recombinant nucleic acid comprises a plasmid or a vector that encodes the entire protein complex. In some embodiments, the recombinant nucleic acid comprises plasmids or vectors that encode the therapeutic domain, the sensor domain, and the linker respectively. In some embodiments, the recombinant nucleic acid comprises plasmids or vectors that encode any two of the therapeutic domain, the sensor domain, and the linker together.

### Pharmaceutical Formulations

A protein complex or a recombinant nucleic acid encoding the protein complex of the present disclosure may be formulated as a pharmaceutical composition. A pharmaceutical composition may comprise a pharmaceutically acceptable carrier or excipient. As used herein “pharmaceutically acceptable” or “pharmacologically acceptable” includes molecular entities and compositions that do not produce an adverse, allergic or other untoward reaction when administered to a subject, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients are often also incorporated into the compositions.

### Applications

A protein complex of the present disclosure may be used for various therapeutic applications. A protein complex of the present disclosure may be used as a therapeutic to administer to a subject in need thereof. The subject may be a human or non-human mammal. The subject may have a disease. The disease may be cancer. The cancer may be acute lymphoblastic leukemia (ALL); acute myeloid leukemia (AML); cancer in adolescents; adrenocortical carcinoma; aids-related cancers; Kaposi sarcoma (soft tissue sarcoma); aids-related lymphoma (lymphoma); primary CNS lymphoma (lymphoma); anal cancer; appendix cancer—see gastrointestinal carcinoid tumors; astrocytomas, childhood (brain cancer); atypical teratoid/rhabdoid tumor, childhood, central nervous system (brain cancer); basal cell carcinoma of the skin-see skin cancer; bile duct cancer; bladder cancer; bone cancer (includes Ewing sarcoma and osteosarcoma and malignant fibrous histiocytoma); brain tumors; breast cancer; bronchial tumors (lung cancer); Burkitt lymphoma—see non-Hodgkin lymphoma; carcinoid tumor (gastrointestinal); carcinoma of unknown primary; cardiac (heart) tumors, childhood; central nervous system; atypical teratoid/rhabdoid tumor, childhood (brain cancer); medulloblastoma and other CNS embryonal tumors, childhood (brain cancer); germ cell tumor, childhood (brain cancer); primary CNS lymphoma; cervical cancer; childhood cancers; cancers of childhood, unusual; cholangiocarcinoma-see bile duct cancer; chordoma, childhood (bone cancer); chronic lymphocytic leukemia (CLL); chronic myelogenous leukemia (CML); chronic myeloproliferative neoplasms; colorectal cancer; craniopharyngioma, childhood (brain cancer); cutaneous t-cell lymphoma-see lymphoma (mycosis fungoides and Sezary syndrome); ductal carcinoma in situ (DCIS)-see breast cancer; embryonal tumors, medulloblastoma and other central nervous system, childhood (brain cancer); endometrial cancer (uterine cancer); cpendymoma, childhood (brain cancer); esophageal cancer; esthesioneuroblastoma (head and neck cancer); Ewing sarcoma (bone cancer); extracranial germ cell tumor, childhood; extragonadal germ cell tumor; eye cancer; intraocular melanoma; retinoblastoma; fallopian tube cancer; fibrous histiocytoma of bone, malignant, and osteosarcoma; gallbladder cancer; gastric (stomach) cancer; gastrointestinal carcinoid tumor; gastrointestinal stromal tumors (GIST) (soft tissue sarcoma); germ cell tumors; childhood central nervous system germ cell tumors (brain cancer); childhood extracranial germ cell tumors; extragonadal germ cell tumors; ovarian germ cell tumors; testicular cancer; gestational trophoblastic disease; hairy cell leukemia; head and neck cancer; heart tumors, childhood; hepatocellular (liver) cancer; histiocytosis, Langerhans cell; Hodgkin lymphoma; hypopharyngeal cancer (head and neck cancer); intraocular melanoma; islet cell tumors, pancreatic neuroendocrine tumors; Kaposi sarcoma (soft tissue sarcoma); kidney (renal cell) cancer; Langerhans cell histiocytosis; laryngeal cancer (head and neck cancer); leukemia; lip and oral cavity cancer (head and neck cancer); liver cancer; lung cancer (non-small cell, small cell, pleuropulmonary blastoma, and tracheobronchial tumor); lymphoma; male breast cancer; malignant fibrous histiocytoma of bone and osteosarcoma; melanoma; melanoma, intraocular (eye); merkel cell carcinoma (skin cancer); mesothelioma, malignant; metastatic cancer; metastatic squamous neck cancer with occult primary (head and neck cancer); midline tract carcinoma with nut gene changes; mouth cancer (head and neck cancer); multiple endocrine neoplasia syndromes; multiple myeloma/plasma cell neoplasms; mycosis fungoides (lymphoma); myelodysplastic syndromes, myelodysplastic/myeloproliferative neoplasms; myelogenous leukemia, chronic (CML); myeloid leukemia, acute (AML); myeloproliferative neoplasms, chronic; nasal cavity and paranasal sinus cancer (head and neck cancer); nasopharyngeal cancer (head and neck cancer); neuroblastoma; non-Hodgkin lymphoma; non-small cell lung cancer; oral cancer, lip and oral cavity cancer and oropharyngeal cancer (head and neck cancer); osteosarcoma and malignant fibrous histiocytoma of bone; ovarian cancer; pancreatic cancer; pancreatic neuroendocrine tumors (islet cell tumors); papillomatosis (childhood laryngeal); paraganglioma; paranasal sinus and nasal cavity cancer (head and neck cancer); parathyroid cancer; penile cancer; pharyngeal cancer (head and neck cancer); phcochromocytoma; pituitary tumor; plasma cell neoplasm/multiple myeloma; pleuropulmonary blastoma (lung cancer); pregnancy and breast cancer; primary central nervous system (CNS) lymphoma; primary peritoneal cancer; prostate cancer; rectal cancer; recurrent cancer; renal cell (kidney) cancer; retinoblastoma; rhabdomyosarcoma, childhood (soft tissue sarcoma); salivary gland cancer (head and neck cancer); sarcoma; childhood rhabdomyosarcoma (soft tissue sarcoma); childhood vascular tumors (soft tissue sarcoma); Ewing sarcoma (bone cancer); Kaposi sarcoma (soft tissue sarcoma); osteosarcoma (bone cancer); soft tissue sarcoma; uterine sarcoma; Sezary syndrome (lymphoma); skin cancer; small cell lung cancer; small intestine cancer; soft tissue sarcoma; squamous cell carcinoma of the skin—see skin cancer; squamous neck cancer with occult primary, metastatic (head and neck cancer); stomach (gastric) cancer; t-cell lymphoma, cutaneous—see lymphoma (mycosis fungoides and Sezary syndrome); testicular cancer; throat cancer (head and neck cancer); nasopharyngeal cancer; oropharyngeal cancer; hypopharyngeal cancer; thymoma and thymic carcinoma; thyroid cancer; tracheobronchial tumors (lung cancer); transitional cell cancer of the renal pelvis and ureter (kidney (renal cell) cancer); unknown primary carcinoma; unusual cancers of childhood; ureter and renal pelvis, transitional cell cancer (kidney (renal cell) cancer; urethral cancer; uterine cancer, endometrial; uterine sarcoma; vaginal cancer; vascular tumors (soft tissue sarcoma); vulvar cancer; Wilms tumor and other childhood kidney tumors; or cancer in young adults.

A protein complex may be administered as a pharmaceutical composition. A pharmaceutical composition of the disclosure can be a combination of any protein complex described herein with other chemical components, such as carriers, stabilizers, diluents, dispersing agents, suspending agents, thickening agents, and/or excipients. The pharmaceutical composition facilitates administration of a protein complex described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, inhalation, dermal, intra-articular, intrathecal, intranasal, and topical administration. A pharmaceutical composition can be administered in a local or systemic manner, for example, via injection of the protein complex described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a protein complex described herein in water-soluble form. Suspensions of protein complexes described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduces the aggregation of such protein complexes described herein to allow for the preparation of highly concentrated solutions. Alternatively, the protein complexes described herein can be lyophilized or in powder form for re-constitution with a suitable vehicle, e.g., sterile pyrogen-free water, before use. In some embodiments, a purified protein complex is administered intravenously. A protein complex of the present disclosure may comprise a sufficiently long serum half-life (e.g., as demonstrated herein, e.g., in EXAMPLE 7) to enable dosing regimens comprising daily, alternating day, twice weekly, weekly, biweekly, or monthly dosing frequencies. A protein complex of the present disclosure may comprise a serum half-life of at least 12 hours, at least 24 hours, at least 36 hours, at least 48 hours, at least 72 hours, at least 96 hours, at least 120 hours, at least 168 hours, at least 250 hours, at least 320 hours, or at least 400 hours. The serum half-life may be a human serum half-life, a murine serum half-life, a porcine serum-half life, a bovine serum half-life, a canine scrum half-life, a feline serum half-life, or a leporine serum half-life.

A protein complex of the disclosure can be applied directly to an organ, or an organ tissue or cells, during a surgical procedure, or via transdermal, subcutaneous, intramuscular, intratumoral, intrathecal, topical, or local delivery. In some embodiments, a protein complex may be applied directly to a cancerous tissue (e.g., a tumor). The protein complexes described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives. The protein complexes may be expressed in spirulina and delivered orally.

In practicing the methods of treatment or use provided herein, therapeutically-effective amounts of the protein complex described herein are administered in pharmaceutical compositions to a subject suffering from cancer. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a protein complex described herein can be manufactured, for example, by expressing the protein complex in a recombinant system, purifying the protein complex, lyophilizing the protein complex, mixing, or dissolving. The pharmaceutical compositions can include at least one pharmaceutically acceptable carrier, diluent, or excipient and compounds described herein as free-base or pharmaceutically-acceptable salt form.

Methods for the preparation of protein complexes described herein include formulating the protein complex described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Certain methods described herein comprise administering to the subject an intravenous pharmaceutical composition comprising a protein complex of the present disclosure, for example, as described herein. Intravenous pharmaceutical compositions of protein complexes include any formulation suitable for administration to a subject via any intravenous method, including a bolus, an infusion which occurs over time or any other intravenous method known in the art. In some aspects, the rate of infusion is such that the dose is administered over a period of less than five minutes, more than five minutes but less than 15 minutes or greater than 15 minutes. In other aspects, the rate of infusion is such that the dose is administered over a period of less than 5 minutes. In other aspects, the rate of infusion is such that the dose is administered over a period of greater than 5 minutes and less than 15 minutes. In some other aspects, the rate of infusion is such that the dose is administered over a period of greater than 15 minutes.

“Product” or “dosage form” as used herein refers to any solid, semi-solid, lyophilized, aqueous, liquid or frozen formulation or preparation used for administration. Upon administration, the rate of release of an active moiety from a product is often greatly influenced by the excipients and/or product characteristics which make up the product itself. For example, an enteric coat on a tablet is designed to separate that tablet's contents from the stomach contents to prevent, for example, degradation of the stomach which often induces gastrointestinal discomfort or injury. According to the currently accepted conventional understanding, systemic exposure of the active moiety will be relatively insensitive to the small formulation changes.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A protein complex of the present disclosure may be administered to a patient in an effective amount. The term “effective amount,” as used herein, can refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case can be determined using techniques, such as a dose escalation study.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal or a lab animal afflicted with a disease or condition) with a protein complex of the disclosure. Protein complexes of the present disclosure may be administered to treat a disease in a subject. The subject can be a human. A subject can be a human; a non-human primate such as a chimpanzee, or other ape or monkey species; a farm animal such as a cattle, horse, sheep, goat, swine; a domestic animal such as a rabbit, dog, and cat; a laboratory animal including a rodent, such as a rat, mouse and guinea pig, or the like. A subject can be of any age. A subject can be, for example, an elderly adult, adult, adolescent, pre-adolescent, child, toddler, infant, or fetus in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment may be provided to the subject for more than I day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment can also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering a protein complex of the disclosure to a subject, either intravenously, subcutancously, intramuscularly, by inhalation, dermally, intra-articular injection, orally, intrathecally, transdermally, intranasally, via a peritoneal route, or directly onto or into a diseased tissue, e.g., via topical, intra-articular injection route or injection route of application.

In some embodiments, the present disclosure provides a method for treating a cancer, the method comprising administering to a subject in need thereof an effective amount of a protein complex of the present disclosure.

In some embodiments, the present disclosure provides a method for treating a cancer, the method comprising administering to a patient in need thereof an effective amount of a pharmaceutical composition comprising a protein complex of the present disclosure and a pharmaceutically acceptable carrier.

### Kits

A protein complex of the present disclosure may be provided in various kits. In some embodiments, pharmaceutical compositions comprising a protein complex of the present disclosure may be supplied as a kit. A kit may comprise a container that comprises a protein complex. Therapeutic protein complexes can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic protein complexes. Such a kit may further comprise written information on indications and usage of the pharmaceutical composition.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

TABLE 2A
Example Peptide Components of Multipeptide Immunocytokine
Designs Described herein

| | | | SEQ |
|---|---|---|---|
| peptideID | Sequence | Comments | ID NO: |
| PEP000113 | DIQMTQSPSSLSASVGDRVTITCRASQDVNT | | 671 |
| | AVAWYQQKPGKAPKLLIYSASFLYSGVPSR | | |
| | FSGSRSGTDFTLTISSLQPEDFATYYCQQHY | | |
| | TTPPTFGQGTKVEIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP000169 | DIQMTQSPSSLSASVGDRVTITCRASQSISS | | 672 |
| | WLAWYQQKPGKAPKLLIYAASTLQSGVPS | | |
| | RFSGSGSGTDFTLTISSLQPEDFATYYCQQS | | |
| | YSTPLTFGGGTKLEIKRADAAPTVSIFPPSSE | | |
| | QLTSGGASVVCFLNNFYPKDINVKWKIDGS | | |
| | ERQNGVLNSWTDQDSKDSTYSMSSTLTLTK | | |
| | DEYERHNSYTCEATHKTSTSPIVKSFNRNEC | | |
| PEP000243 | DIVMTQSPDSLAVSLGERATINCKASESVDT | | 673 |
| | SDNSFIHWYQQKPGQSPKLLIYRSSTLESGV | | |
| | PDRFSGSGSGTDFTLTISSLQAEDVAVYYCQ | | |
| | QNYDVPWTFGQGTKVEIKRADAAPTVSIFP | | |
| | PSSEQLTSGGASVVCFLNNFYPKDINVKWKI | | |
| | DGSERQNGVLNSWTDQDSKDSTYSMSSTL | | |
| | TLTKDEYERHNSYTCEATHKTSTSPIVKSEN | | |
| | RNEC | | |
| PEP000244 | DIVMTQSPDSLAVSLGERATINCKASESVDT | | 674 |
| | SDNSFIHWYQQKPGQSPKLLIYRSSTLESGV | | |
| | PDRFSGSGSGTDFTLTISSLQAEDVAVYYCQ | | |
| | QNYDVPWTFGQGTKVEIKRTVAAPSVFIFPP | | |
| | SDEQLKSGTASVVCLLNNFYPREAKVQWK | | |
| | VDNALQSGNSQESVTEQDSKDSTYSLSSTL | | |
| | TLSKADYEKHKVYACEVTHQGLSSPVTKSF | | |
| | NRGEC | | |
| PEP000245 | DIVMTQSPDSLAVSLGERATINCKASQSLLH | | 675 |
| | SSSNKNYLAWYQQKPGQPPKLLIYWASTRE | | |
| | SGVPDRFSGSGSGTDFTLTISSLQAEDVAVY | | |
| | YCQQYYSTPITFGPGTKVDIKRADAAPTVSI | | |
| | FPPSSEQLTSGGASVVCFLNNFYPKDINVK | | |
| | WKIDGSERQNGVLNSWTDQDSKDSTYSMS | | |
| | STLTLTKDEYERHNSYTCEATHKTSTSPIVK | | |
| | SFNRNEC | | |
| PEP000288 | EVQLLESGGGLVQPGGSLRLSCAASGFSFSS | | 676 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SASTKGPSVFPLAPSSKSTSGGTAALGCLVK | | |
| | DYFPEPVTVSWNSGALTSGVHTFPAVLQSS | | |
| | GLYSLSSVVTVPSSSLGTQTYICNVNHKPSN | | |
| | TKVDKKVEPKSCDKTHTCPPCPAPEAAGGP | | |
| | SVFLFPPKPKDTLMISRTPEVTCVVVDVSHE | | |
| | DPEVKFNWYVDGVEVHNAKTKPREEQYNS | | |
| | TYRVVSVLTVLHQDWLNGKEYKCKVSNK | | |
| | ALGAPIEKTISKAKGQPREPQVYTLPPCRDE | | |
| | LTKNQVSLWCLVKGFYPSDIAVEWESNGQP | | |
| | ENNYKTTPPVLDSDGSFFLYSKLTVDKSRW | | |
| | QQGNVFSCSVMHEALHNHYTQKSLSLSPG | | |
| | GGGGSGGGGSGGGGSAPASSSTKKTQLQLE | | |
| | HLLLDLQMILNGINNYKNPKLTRMLTAKFA | | |
| | MPKKATELKHLQCLEEELKPLEEVLNGAQS | | |
| | KNFHLRPRDLISNINVIVLELKGSETTFMCE | | |
| | YADETATIVEFLNRWITFAQSIISTLT | | |
| PEP001315 | MSTSTEQKLISEEDLQVQLVQSGAEVKKPG | PD1_R04_C10- | 677 |
| | ASVKVSCKASGYTFTTYYMHWVRQAPGQ | Internal | |
| | GLEWMGIINPSGGGTLYAQKFQGRVTMTR | anti-PD1 | |
| | DTSTSTVYMELSSLRSEDTAVYYCAAGLFI | mAb | |
| | WGQGTTVTVSSASGGGGSGGGGSGGGGSH | | |
| | ASDIVMTQSPDSLAVSLGERATINCKASQSL | | |
| | LHSSSNKNYLAWYQQKPGQPPKLLIYWAST | | |
| | RESGVPDRFSGSGSGTDFTLTISSLQAEDVA | | |
| | VYYCQQYYSTPITFGPGTKVDIKGSGLNDIF | | |
| | EAQKIEWHEGKPIPNPLLGLDST | | |
| PEP003213 | MSTSTEQKLISEEDLQVQLVQSGAEVKKPG | AB002022_ | 678 |
| | ASVKVSCKASGDTFTRHYVHWVRQAPGQG | 2B07v1 | |
| | LEWMGIINPSGGYASYAQKFQGRVTMTRD | | |
| | TSTSTVYMELSSLRSEDTAVYYCAAGLFIW | | |
| | GQGTLVTVSSASGGGGSGGGGSGGGGSHA | | |
| | SDIQMTQSPSSLSASVGDRVTITCRASQSIGR | | |
| | WLAWYQQKPGKAPKLLIYSASNLETGVPSR | | |
| | FSGSGSGTDFTLTISSLQPEDFATYYCQQYE | | |
| | SFPVTFGPGTKVDIKGKPIPNPLLGLDST | | |
| PEP003626 | EVQLLESGGGLVQPGGSLRLSCAASGFSFSS | | 679 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SAKTTAPSVYPLAPVCGDTTGSSVTLGCLV | | |
| | KGYFPEPVTLTWNSGSLSSGVHTFPAVLQS | | |
| | DLYTLSSSVTVTSSTWPSQSITCNVAHPASS | | |
| | TKVDKKIEPRGPTIKPCPPCKCPAPNAAGGP | | |
| | SVFIFPPKIKDVLMISLSPIVTCVVVDVSEDD | | |
| | PDVQISWFVNNVEVHTAQTQTHREDYNSTL | | |
| | RVVSALPIQHQDWMSGKEFKCKVNNKDLG | | |
| | APIERTISKPKGSVRAPQVYVLPPPEEEMTK | | |
| | KQVTLTCMVTDFMPEDIYVEWTNNGKTEL | | |
| | NYKNTEPVLDSDGSYFMYSDLRVEKKNWV | | |
| | ERNSYSCSVVHEGLHNHHTTESFSRTPGGG | | |
| | GGSGGGGSGGGGSAPASSSTKKTQLQLEHL | | |
| | LLDLQMILNGINNYKNPKLTRMLTAKFAMP | | |
| | KKATELKHLQCLEEELKPLEEVLNGAQSKN | | |
| | FHLRPRDLISNINVIVLELKGSETTFMCEYA | | |
| | DETATIVEFLNRWITFAQSIISTLT | | |
| PEP003639 | APASSSTKKTQLQLEHLLLDLQMILNGINNY | | 680 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGDTF | | |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGK | | |
| PEP003641 | DIQMTQSPSSLSASVGDRVSITCKASQNVGT | | 681 |
| | NVGWYQQKPGKAPKALIYSASFRYSGVPSR | | |
| | FSGSGSGTDFTLTISSLQPEDFATYFCQQYY | | |
| | TYPYTFGGGTKLEIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP003642 | DIQMTQSPSSLSASVGDRVTITCRASQSIGR | | 682 |
| | WLAWYQQKPGKAPKLLIYSASNLETGVPSR | | |
| | FSGSGSGTDFTLTISSLQPEDFATYYCQQYE | | |
| | SFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP003648 | QVQLVQSGAEVKKPGASVKVSCKASGDTF | | 683 |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGSGGG | | |
| | SHHHHHH | | |
| PEP003654 | APASSSTKKTQLQLEHLLLDLQMILNGINNY | | 684 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGSE | | |
| | VQLVESGGGLVKPGGSLRLSCAASGFTFSS | | |
| | YTLAWVRQAPGKGLEWVAAIDSSSYTYSP | | |
| | DTVRGRFTISRDNAKNSLYLQMNSLRAEDT | | |
| | AVYYCARDSNWDALDYWGQGTLVTVSSA | | |
| | KTTAPSVYPLAPVCGDTTGSSVTLGCLVKG | | |
| | YFPEPVTLTWNSGSLSSGVHTFPAVLQSDL | | |
| | YTLSSSVTVTSSTWPSQSITCNVAHPASSTK | | |
| | VDKKIEPRGPTIKPCPPCKCPAPNAAGGPSV | | |
| | FIFPPKIKDVLMISLSPIVTCVVVDVSEDDPD | | |
| | VQISWFVNNVEVHTAQTQTHREDYNSTLR | | |
| | VVSALPIQHQDWMSGKEFKCKVNNKDLGA | | |
| | PIERTISKPKGSVRAPQVYVLPPPEEEMTKK | | |
| | QVTLTCMVTDFMPEDIYVEWTNNGKTELN | | |
| | YKNTEPVLDSDGSYFMYSKLRVEKKNWVE | | |
| | RNSYSCSVVHEGLHNHHTTKSFSRTPGK | | |
| PEP003655 | APASSSTKKTQLQLEHLLLDLQMILNGINNY | | 685 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGSE | | |
| | VQLVESGGGLVQPGGSLRLSCAASGFNIKD | | |
| | TYIHWVRQAPGKGLEWVARIYPTNGYTRY | | |
| | ADSVKGRFTISADTSKNTAYLQMNSLRAED | | |
| | TAVYYCSRWGGDGFYAMDYWGQGTLVTV | | |
| | SSAKTTAPSVYPLAPVCGDTTGSSVTLGCL | | |
| | VKGYFPEPVTLTWNSGSLSSGVHTFPAVLQ | | |
| | SDLYTLSSSVTVTSSTWPSQSITCNVAHPAS | | |
| | STKVDKKIEPRGPTIKPCPPCKCPAPNAAGG | | |
| | PSVFIFPPKIKDVLMISLSPIVTCVVVDVSED | | |
| | DPDVQISWFVNNVEVHTAQTQTHREDYNS | | |
| | TLRVVSALPIQHQDWMSGKEFKCKVNNKD | | |
| | LGAPIERTISKPKGSVRAPQVYVLPPPEEEM | | |
| | TKKQVTLTCMVTDFMPEDIYVEWTNNGKT | | |
| | ELNYKNTEPVLDSDGSYFMYSKLRVEKKN | | |
| | WVERNSYSCSVVHEGLHNHHTTKSFSRTPG | | |
| | K | | |
| PEP003657 | APASSSTKKTQLQLEHLLLDLQMILNGINNY | | 686 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGDTF | | |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSKLRVEKKNWVERNSY | | |
| | SCSVVHEGLHNHHTTKSFSRTPGK | | |
| PEP003780 | APASSSTKKTQLQLEHLLLDLQMILNGINNY | | 687 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGDTF | | |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSASTKG | | |
| | PSVFPLAPSSKSTSGGTAALGCLVKDYFPEP | | |
| | VTVSWNSGALTSGVHTFPAVLQSSGLYSLS | | |
| | SVVTVPSSSLGTQTYICNVNHKPSNTKVDK | | |
| | KVEPKSCDKTHTCPPCPAPEAAGGPSVFLFP | | |
| | PKPKDTLMISRTPEVTCVVVDVSHEDPEVK | | |
| | FNWYVDGVEVHNAKTKPREEQYNSTYRVV | | |
| | SVLTVLHQDWLNGKEYKCKVSNKALPAPIE | | |
| | KTISKAKGQPREPQVYTLPPSREEMTKNQV | | |
| | SLTCLVKGFYPSDIAVEWESNGQPENNYKT | | |
| | TPPVLDSDGSFFLYSKLTVDKSRWQQGNVF | | |
| | SCSVMHEALHNHYTQKSLSLSPGK | | |
| PEP003782 | DIQMTQSPSSLSASVGDRVTITCRASQSIGR | | 688 |
| | WLAWYQQKPGKAPKLLIYSASNLETGVPSR | | |
| | FSGSGSGTDFTLTISSLQPEDFATYYCQQYE | | |
| | SFPVTFGPGTKVDIKRTVAAPSVFIFPPSDEQ | | |
| | LKSGTASVVCLLNNFYPREAKVQWKVDNA | | |
| | LQSGNSQESVTEQDSKDSTYSLSSTLTLSKA | | |
| | DYEKHKVYACEVTHQGLSSPVTKSFNRGEC | | |
| PEP003786 | APASSSTKKTQLQLEHLLLDLQMILNGINNY | | 689 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | DIQMTQSPSSLSASVGDRVTITCRASQSIGR | | |
| | WLAWYQQKPGKAPKLLIYSASNLETGVPSR | | |
| | FSGSGSGTDFTLTISSLQPEDFATYYCQQYE | | |
| | SFPVTFGPGTKVDIKRTVAAPSVFIFPPSDEQ | | |
| | LKSGTASVVCLLNNFYPREAKVQWKVDNA | | |
| | LQSGNSQESVTEQDSKDSTYSLSSTLTLSKA | | |
| | DYEKHKVYACEVTHQGLSSPVTKSFNRGEC | | |
| PEP003791 | QVQLVQSGAEVKKPGASVKVSCKASGDTF | | 690 |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSASTKG | | |
| | PSVFPLAPSSKSTSGGTAALGCLVKDYFPEP | | |
| | VTVSWNSGALTSGVHTFPAVLQSSGLYSLS | | |
| | SVVTVPSSSLGTQTYICNVNHKPSNTKVDK | | |
| | KVEPKSCDKTHTCPPCPAPEAAGGPSVFLFP | | |
| | PKPKDTLMISRTPEVTCVVVDVSHEDPEVK | | |
| | FNWYVDGVEVHNAKTKPREEQYNSTYRVV | | |
| | SVLTVLHQDWLNGKEYKCKVSNKALPAPIE | | |
| | KTISKAKGQPREPQVYTLPPSREEMTKNQV | | |
| | SLTCLVKGFYPSDIAVEWESNGQPENNYKT | | |
| | TPPVLDSDGSFFLYSKLTVDKSRWQQGNVF | | |
| | SCSVMHEALHNHYTQKSLSLSPGK | | |
| PEP004129 | EVQLLESGGGLVQPGGSLRLSCAASGFSFSS | | 691 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SASTKGPSVFPLAPSSKSTSGGTAALGCLVK | | |
| | DYFPEPVTVSWNSGALTSGVHTFPAVLQSS | | |
| | GLYSLSSVVTVPSSSLGTQTYICNVNHKPSN | | |
| | TKVDKKVEPKSCDKTHTSPPSPEPKSSDTPP | | |
| | PSPRSPEPKSSDTPPPSPRSPEPKSCDTPPPCP | | |
| | RAPEAAGGPSVFLFPPKPKDTLMISRTPEVT | | |
| | CVVVDVSHEDPEVKFNWYVDGVEVHNAK | | |
| | TKPREEQYNSTYRVVSVLTVLHQDWLNGK | | |
| | EYKCKVSNKALGAPIEKTISKAKGQPREPQ | | |
| | VCTLPPSRDELTKNQVSLSCAVKGFYPSDIA | | |
| | VEWESNGQPENNYKTTPPVLDSDGSFFLVS | | |
| | KLTVDKSRWQQGNVFSCSVMHEALHNHYT | | |
| | QKSLSLSP | | |
| PEP004130 | EVQLLESGGGLVQPGGSLRLSCAASGFSFSS | | 692 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SASTKGPSVFPLAPSSKSTSGGTAALGCLVK | | |
| | DYFPEPVTVSWNSGALTSGVHTFPAVLQSS | | |
| | GLYSLSSVVTVPSSSLGTQTYICNVNHKPSN | | |
| | TKVDKKVEPKSCDKTHTSPPSPEPKSSDTPP | | |
| | PSPRSPEPKSSDTPPPSPRSPEPKSCDTPPPCP | | |
| | RAPEAAGGPSVFLFPPKPKDTLMISRTPEVT | | |
| | CVVVDVSHEDPEVKFNWYVDGVEVHNAK | | |
| | TKPREEQYNSTYRVVSVLTVLHQDWLNGK | | |
| | EYKCKVSNKALGAPIEKTISKAKGQPREPQ | | |
| | VYTLPPCRDELTKNQVSLWCLVKGFYPSDI | | |
| | AVEWESNGQPENNYKTTPPVLDSDGSFFLY | | |
| | SKLTVDKSRWQQGNVFSCSVMHEALHNHY | | |
| | TQKSLSLSPGGGGGSGGGGSGGGGSAPASS | | |
| | STKKTQLQLEHLLLDLQMILNGINNYKNPK | | |
| | LTRMLTAKFAMPKKATELKHLQCLEEELKP | | |
| | LEEVLNGAQSKNFHLRPRDLISNINVIVLEL | | |
| | KGSETTFMCEYADETATIVEFLNRWITFAQS | | |
| | IISTLT | | |
| PEP004133 | QVQLVQSGAEVKKPGASVKVSCKASGDTF | | 693 |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSASTKG | | |
| | PSVFPLAPSSKSTSGGTAALGCLVKDYFPEP | | |
| | VTVSWNSGALTSGVHTFPAVLQSSGLYSLS | | |
| | SVVTVPSSSLGTQTYICNVNHKPSNTKVDK | | |
| | KVEPKSCDKTHTSPPSPEPKSSDTPPPSPRSP | | |
| | EPKSSDTPPPSPRSPEPKSCDTPPPCPRAPEA | | |
| | AGGPSVFLFPPKPKDTLMISRTPEVTCVVVD | | |
| | VSHEDPEVKFNWYVDGVEVHNAKTKPREE | | |
| | QYNSTYRVVSVLTVLHQDWLNGKEYKCK | | |
| | VSNKALGAPIEKTISKAKGQPREPQVCTLPP | | |
| | SRDELTKNQVSLSCAVKGFYPSDIAVEWES | | |
| | NGQPENNYKTTPPVLDSDGSFFLVSKLTVD | | |
| | KSRWQQGNVFSCSVMHEALHNHYTQKSLS | | |
| | LSP | | |
| PEP004134 | QVQLVQSGAEVKKPGASVKVSCKASGDTF | | 694 |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSASTKG | | |
| | PSVFPLAPSSKSTSGGTAALGCLVKDYFPEP | | |
| | VTVSWNSGALTSGVHTFPAVLQSSGLYSLS | | |
| | SVVTVPSSSLGTQTYICNVNHKPSNTKVDK | | |
| | KVEPKSCDKTHTSPPSPEPKSSDTPPPSPRSP | | |
| | EPKSSDTPPPSPRSPEPKSCDTPPPCPRAPEA | | |
| | AGGPSVFLFPPKPKDTLMISRTPEVTCVVVD | | |
| | VSHEDPEVKFNWYVDGVEVHNAKTKPREE | | |
| | QYNSTYRVVSVLTVLHQDWLNGKEYKCK | | |
| | VSNKALGAPIEKTISKAKGQPREPQVYTLPP | | |
| | CRDELTKNQVSLWCLVKGFYPSDIAVEWES | | |
| | NGQPENNYKTTPPVLDSDGSFFLYSKLTVD | | |
| | KSRWQQGNVFSCSVMHEALHNHYTQKSLS | | |
| | LSPGGGGGSGGGGSGGGGSAPASSSTKKTQ | | |
| | LQLEHLLLDLQMILNGINNYKNPKLTRMLT | | |
| | AKFAMPKKATELKHLQCLEEELKPLEEVLN | | |
| | GAQSKNFHLRPRDLISNINVIVLELKGSETTF | | |
| | MCEYADETATIVEFLNRWITFAQSIISTLT | | |
| PEP004153 | APASSSTKKTQLQLEHLLLDLQMILNGINNY | | 695 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGDTF | | |
| | TRYYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSKLRVEKKNWVERNSY | | |
| | SCSVVHEGLHNHHTTKSFSRTPGK | | |
| PEP004158 | DIQMTQSPSSLSASVGDRVTITCRASQSIGR | | 696 |
| | WLAWYQQKPGKAPKLLIYSASNLETGVPSR | | |
| | FSGSGSGTDFTLTISSLQPEDFATYYCQQYN | | |
| | RFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004159 | DIQMTQSPSSLSASVGDRVTITCRASQSIGR | | 697 |
| | WLAWYQQKPGKAPKLLIYSASNLETGVPSR | | |
| | FSGSGSGTDFTLTISSLQPEDFATYYCQQYN | | |
| | SFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004161 | DIQMTQSPSSLSASVGDRVTITCRASQSIGR | | 698 |
| | YLAWYQQKPGKAPKLLIYSASNLETGVPSR | | |
| | FSGSGSGTDFTLTISSLQPEDFATYYCQQYN | | |
| | RFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004162 | DIQMTQSPSSLSASVGDRVTITCRASQSIGR | | 699 |
| | YLAWYQQKPGKAPKLLIYSASNLETGVPSR | | |
| | FSGSGSGTDFTLTISSLQPEDFATYYCQQYN | | |
| | SFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004163 | DIQMTQSPSSLSASVGDRVTITCRASQSIGR | | 700 |
| | YLAWYQQKPGKAPKLLIYYASNLETGVPSR | | |
| | FSGSGSGTDFTLTISSLQPEDFATYYCQQYN | | |
| | SFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004191 | MSTSTQVQLVQSGAEVKKPGASVKVSCKA | AB002342_ | 701 |
| | SGDTFTRYYVHWVRQAPGQGLEWMGIINP | 2B07v5 | |
| | SGGYASYAQKFQGRVTMTRDTSTSTVYME | | |
| | LSSLRSEDTAVYYCAAGLFIWGQGTLVTVS | | |
| | SASGGGGSGGGGSGGGGSHASDIQMTQSPS | | |
| | SLSASVGDRVTITCRASQSIGRWLA WYQQK | | |
| | PGKAPKLLIYSASNLETGVPSRFSGSGSGTD | | |
| | FTLTISSLQPEDFATYYCQQYERFPVTFGPG | | |
| | TKVDIKAAAGSGSEQKLISEEDLGKPIPNPL | | |
| | LGLDST | | |
| PEP004192 | MSTSTQVQLVQSGAEVKKPGASVKVSCKA | AB002353_ | 702 |
| | SGDTFTRYYVHWVRQAPGQGLEWMGIINP | 2B07v9 | |
| | SGGYASYAQKFQGRVTMTRDTSTSTVYME | | |
| | LSSLRSEDTAVYYCAAGLFIWGQGTLVTVS | | |
| | SASGGGGSGGGGSGGGGSHASDIQMTQSPS | | |
| | SLSASVGDRVTITCRASQSIGRWLAWYQQK | | |
| | PGKAPKLLIYSASNLETGVPSRFSGSGSGTD | | |
| | FTLTISSLQPEDFATYYCQQYESFPVTFGPGT | | |
| | KVDIKAAAGSGSEQKLISEEDLGKPIPNPLL | | |
| | GLDST | | |
| PEP004195 | MSTSTQVQLVQSGAEVKKPGASVKVSCKA | AB002328_ | 703 |
| | SGDTFTRYYVHWVRQAPGQGLEWMGIINP | 2B07v4 | |
| | SGGYASYAQKFQGRVTMTRDTSTSTVYME | | |
| | LSSLRSEDTAVYYCAAGLFIWGQGTLVTVS | | |
| | SASGGGGSGGGGSGGGGSHASDIQMTQSPS | | |
| | SLSASVGDRVTITCRASQSIGRWLAWYQQK | | |
| | PGKAPKLLIYSASNLETGVPSRFSGSGSGTD | | |
| | FTLTISSLQPEDFATYYCQQYNRFPVTFGPG | | |
| | TKVDIKAAAGSGSEQKLISEEDLGKPIPNPL | | |
| | LGLDST | | |
| PEP004198 | MSTSTQVQLVQSGAEVKKPGASVKVSCKA | AB002293_ | 704 |
| | SGDTFTRYYVHWVRQAPGQGLEWMGIINP | 2B07v2 | |
| | SGGYASYAQKFQGRVTMTRDTSTSTVYME | | |
| | LSSLRSEDTAVYYCAAGLFIWGQGTLVTVS | | |
| | SASGGGGSGGGGSGGGGSHASDIQMTQSPS | | |
| | SLSASVGDRVTITCRASQSIGRWLAWYQQK | | |
| | PGKAPKLLIYSASNLETGVPSRFSGSGSGTD | | |
| | FTLTISSLQPEDFATYYCQQYNSFPVTFGPG | | |
| | TKVDIKAAAGSGSEQKLISEEDLGKPIPNPL | | |
| | LGLDST | | |
| PEP004207 | MSTSTQVQLVQSGAEVKKPGASVKVSCKA | AB002347_ | 705 |
| | SGDTFTRYYVHWVRQAPGQGLEWMGIINP | 2B07v7 | |
| | SGGYASYAQKFQGRVTMTRDTSTSTVYME | | |
| | LSSLRSEDTAVYYCAAGLFIWGQGTLVTVS | | |
| | SASGGGGGGGGSGGGGSHASDIQMTQSPS | | |
| | SLSASVGDRVTITCRASQSIGRYLAWYQQK | | |
| | PGKAPKLLIYSASNLETGVPSRFSGSGSGTD | | |
| | FTLTISSLQPEDFATYYCQQYNRFPVTFGPG | | |
| | TKVDIKAAAGSGSEQKLISEEDLGKPIPNPL | | |
| | LGLDST | | |
| PEP004208 | MSTSTQVQLVQSGAEVKKPGASVKVSCKA | AB002345_ | 706 |
| | SGDTFTRYYVHWVRQAPGQGLEWMGIINP | 2B07v6 | |
| | SGGYASYAQKFQGRVTMTRDTSTSTVYME | | |
| | LSSLRSEDTAVYYCAAGLFIWGQGTLVTVS | | |
| | SASGGGGSGGGGSGGGGSHASDIQMTQSPS | | |
| | SLSASVGDRVTITCRASQSIGRYLAWYQQK | | |
| | PGKAPKLLIYSASNLETGVPSRFSGSGSGTD | | |
| | FTLTISSLQPEDFATYYCQQYNSFPVTFGPG | | |
| | TKVDIKAAAGSGSEQKLISEEDLGKPIPNPL | | |
| | LGLDST | | |
| PEP004210 | MSTSTQVQLVQSGAEVKKPGASVKVSCKA | AB002348_ | 707 |
| | SGDTFTRYYVHWVRQAPGQGLEWMGIINP | 2B07v8 | |
| | SGGYASYAQKFQGRVTMTRDTSTSTVYME | | |
| | LSSLRSEDTAVYYCAAGLFIWGQGTLVTVS | | |
| | SASGGGGSGGGGSGGGGSHASDIQMTQSPS | | |
| | SLSASVGDRVTITCRASQSIGRYLAWYQQK | | |
| | PGKAPKLLIYYASNLETGVPSRFSGSGSGTD | | |
| | FTLTISSLQPEDFATYYCQQYNSFPVTFGPG | | |
| | TKVDIKAAAGSGSEQKLISEEDLGKPIPNPL | | |
| | LGLDST | | |
| PEP004211 | MSTSTQVQLVQSGAEVKKPGASVKVSCKA | AB002326_ | 708 |
| | SGDTFTRYYVHWVRQAPGQGLEWMGIINP | 2B07v3 | |
| | SGGYASYAQKFQGRVTMTRDTSTSTVYME | | |
| | LSSLRSEDTAVYYCAAGLFIWGQGTLVTVS | | |
| | SASGGGGSGGGGSGGGGSHASDIQMTQSPS | | |
| | SLSASVGDRVTITCRASQSIGSWLAWYQQK | | |
| | PGKAPKLLIYSASNLETGVPSRFSGSGSGTD | | |
| | FTLTISSLQPEDFATYYCQQYNSFPVTFGPG | | |
| | TKVDIKAAAGSGSEQKLISEEDLGKPIPNPL | | |
| | LGLDST | | |
| PEP004243 | APASSSTKKTQLQLEHLLLDLQMILNGINNY | | 709 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGDTF | | |
| | TRYYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGK | | |
| PEP004247 | QVQLVQSGAEVKKPGASVKVSCKASGDTF | | 710 |
| | TRYYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGK | | |
| PEP004251 | APASSSTKKTQLQLEHLLLDLQMILNGINNY | | 711 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGYTF | | |
| | TKNYMHWVRQAPGQGLEWLGWVSPDSGY | | |
| | TGYAQKFQGRVTMTRDTSTSTVYMELSSLR | | |
| | SEDTAVYYCTTDLLSLELDDAFDIWGQGTM | | |
| | VTVSSAKTTAPSVYPLAPVCGDTTGSSVTL | | |
| | GCLVKGYFPEPVTLTWNSGSLSSGVHTFPA | | |
| | VLQSDLYTLSSSVTVTSSTWPSQSITCNVAH | | |
| | PASSTKVDKKIEPRGPTIKPCPPCKCPAPNA | | |
| | AGGPSVFIFPPKIKDVLMISLSPIVTCVVVDV | | |
| | SEDDPDVQISWFVNNVEVHTAQTQTHRED | | |
| | YNSTLRVVSALPIQHQDWMSGKEFKCKVN | | |
| | NKDLGAPIERTISKPKGSVRAPQVYVLPPPE | | |
| | EEMTKKQVTLTCMVTDFMPEDIYVEWTNN | | |
| | GKTELNYKNTEPVLDSDGSYFMYSDLRVE | | |
| | KKNWVERNSYSCSVVHEGLHNHHTTESFS | | |
| | RTPGK | | |
| PEP004261 | MSTSTQVQLVQSGAEVKKPGASVKVSCKA | AB002381_ | 712 |
| | SGDTFTDYYMHWVRQAPGQGLEWMGIINP | 7A04v4 | |
| | RAGYTSYALKFQGRVTMTRDTSTSTVYME | | |
| | LSSLRSEDTAVYYCTSGWDVWGQGTLVTV | | |
| | SSASGGGGSGGGGSGGGGSHASDIQMTQSP | | |
| | SSLSASVGDRVTITCRASQSISTWLAWYQQ | | |
| | KPGKAPKLLIYAASSLDSGVPSRFSGSGSGT | | |
| | DFTLTISSLQPEDFATYYCQQSYSFPVTFGQ | | |
| | GTKVEIKAAAGSGSEQKLISEEDLGKPIPNP | | |
| | LLGLDST | | |
| PEP004271 | MSTSTQVQLVQSGAEVKKPGASVKVSCKA | AB002427_ | 713 |
| | SGHTFTRYYMHWVRQAPGQGLEWMGIINP | 2A11v5 | |
| | SGGYATYAQKFQGRVTMTRDTSTSTVYME | | |
| | LSSLRSEDTAVYYCASGLFIWGQGTLVTVS | | |
| | SASGGGGSGGGGSGGGGSHASDIQMTQSPS | | |
| | SLSASVGDRVTITCRASQSINSWLAWYQQK | | |
| | PGKAPKLLIYATSTLESGVPSRFSGSGSGTD | | |
| | FTLTISSLQPEDFATYYCQQSYRFPVTFGQG | | |
| | TKVEIKAAAGSGSEQKLISEEDLGKPIPNPLL | | |
| | GLDST | | |
| PEP004272 | MSTSTQVQLVQSGAEVKKPGASVKVSCKA | AB002413_ | 714 |
| | SGHTFTRYYMHWVRQAPGQGLEWMGIINP | 2A11v3 | |
| | SGGYATYAQKFQGRVTMTRDTSTSTVYME | | |
| | LSSLRSEDTAVYYCASGLFIWGQGTLVTVS | | |
| | SASGGGGSGGGGSGGGGSHASDIQMTQSPS | | |
| | SLSASVGDRVTITCRASQSINSWLAWYQQK | | |
| | PGKAPKLLIYATSTLESGVPSRFSGSGSGTD | | |
| | FTLTISSLQPEDFATYYCQQYYRFPVTFGQG | | |
| | TKVEIKAAAGSGSEQKLISEEDLGKPIPNPLL | | |
| | GLDST | | |
| PEP004273 | MSTSTQVQLVQSGAEVKKPGASVKVSCKA | AB002417_ | 715 |
| | SGHTFTRYYMHWVRQAPGQGLEWMGIINP | 2A11v4 | |
| | SGGYATYAQKFQGRVTMTRDTSTSTVYME | | |
| | LSSLRSEDTAVYYCASGLFIWGQGTLVTVS | | |
| | SASGGGGSGGGGSGGGGSHASDIQMTQSPS | | |
| | SLSASVGDRVTITCRASQSINSWLAWYQQK | | |
| | PGKAPKLLIYATSTLESGVPSRFSGSGSGTD | | |
| | FTLTISSLQPEDFATYYCQQYYSFPVTFGQG | | |
| | TKVEIKAAAGSGSEQKLISEEDLGKPIPNPLL | | |
| | GLDST | | |
| PEP004281 | MSTSTQVQLVQSGAEVKKPGASVKVSCKA | AB002410_ | 716 |
| | SGHTFTRYYMHWVRQAPGQGLEWMGIINP | 2A11v2 | |
| | SGGYATYAQKFQGRVTMTRDTSTSTVYME | | |
| | LSSLRSEDTAVYYCASGWFVWGQGTLVTV | | |
| | SSASGGGGSGGGGSGGGGSHASDIQMTQSP | | |
| | SSLSASVGDRVTITCRASQSINSWLAWYQQ | | |
| | KPGKAPKLLIYDTSTLESGVPSRFSGSGSGT | | |
| | DFTLTISSLQPEDFATYYCQQSYSFPVTFGQ | | |
| | GTKVEIKAAAGSGSEQKLISEEDLGKPIPNP | | |
| | LLGLDST | | |
| PEP004299 | MSTSTQVQLVQSGAEVKKPGASVKVSCKA | AB002360_ | 717 |
| | SGYTFTDYYMHWVRQAPGQGLEWMGIINP | 7A04v1 | |
| | RAGYTSYALKFQGRVTMTRDTSTSTVYME | | |
| | LSSLRSEDTAVYYCTSGWDVWGQGTLVTV | | |
| | SSASGGGGSGGGGSGGGGSHASDIQMTQSP | | |
| | SSLSASVGDRVTITCRASQSISTWLAWYQQ | | |
| | KPGKAPKLLIYAASSLDSGVPSRFSGSGSGT | | |
| | DFTLTISSLQPEDFATYYCQQSYSFPVTFGQ | | |
| | GTKVEIKAAAGSGSEQKLISEEDLGKPIPNP | | |
| | LLGLDST | | |
| PEP004340 | MSTSTQVQLVQSGAEVKKPGASVKVSCKA | AB002365_ | 718 |
| | SGYTFTRYYMHWVRQAPGQGLEWMGIINP | 7A04v2 | |
| | RAGYTSYALKFQGRVTMTRDTSTSTVYME | | |
| | LSSLRSEDTAVYYCTSGWDVWGQGTLVTV | | |
| | SSASGGGGSGGGGSGGGGSHASDIQMTQSP | | |
| | SSLSASVGDRVTITCRASQSISTWLAWYQQ | | |
| | KPGKAPKLLIYAASSLDSGVPSRFSGSGSGT | | |
| | DFTLTISSLQPEDFATYYCQQSYSFPVTFGQ | | |
| | GTKVEIKAAAGSGSEQKLISEEDLGKPIPNP | | |
| | LLGLDST | | |
| PEP004349 | MSTSTQVQLVQSGAEVKKPGASVKVSCKA | AB002370_ | 719 |
| | SGYTFTTYYMHWVRQAPGQGLEWMGIINP | 7A04v3 | |
| | RAGYTSYALKFQGRVTMTRDTSTSTVYME | | |
| | LSSLRSEDTAVYYCTSGWDVWGQGTLVTV | | |
| | SSASGGGGSGGGGSGGGGSHASDIQMTQSP | | |
| | SSLSASVGDRVTITCRASQSISTWLAWYQQ | | |
| | KPGKAPKLLIYAASSLDSGVPSRFSGSGSGT | | |
| | DFTLTISSLQPEDFATYYCQQSYSFPVTFGQ | | |
| | GTKVEIKAAAGSGSEQKLISEEDLGKPIPNP | | |
| | LLGLDST | | |
| PEP004356 | EVQLLESGGGLVQPGGSLRLSCAASGFSFSS | | 720 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SAKTTAPSVYPLAPVCGDTTGSSVTLGCLV | | |
| | KGYFPEPVTLTWNSGSLSSGVHTFPAVLQS | | |
| | DLYTLSSSVTVTSSTWPSQSITCNVAHPASS | | |
| | TKVDKKIEPRGPTIKPCPPCKCPAPNAAGGP | | |
| | SVFIFPPKIKDVLMISLSPIVTCVVVDVSEDD | | |
| | PDVQISWFVNNVEVHTAQTQTHREDYNSTL | | |
| | RVVSALPIQHQDWMSGKEFKCKVNNKDLG | | |
| | APIERTISKPKGSVRAPQVYVLPPPEKEMTK | | |
| | KQVSLTCLVKDFMPEDIYVEWTNNGKTEL | | |
| | NYKNTEPVLKSDGSYFMYSKLTVEKKNWV | | |
| | ERNSYSCSVVHEGLHNHHTTKSFSRTPGGG | | |
| | GGSGGGGSGGGGSGGGGSEVQLVESGGGL | | |
| | VKPGGSLRLSCAASGFTFSSYTLAWVRQAP | | |
| | GKGLEWVAAIDSSSYTYSPDTVRGRFTISRD | | |
| | NAKNSLYLQMNSLRAEDTAVYYCARDSN | | |
| | WDALDYWGQGTLVTVSSASGGGGSGGGG | | |
| | SGGGGSHASDIQMTQSPSSLSASVGDRVSIT | | |
| | CKASQNVGTNVGWYQQKPGKAPKALIYSA | | |
| | SFRYSGVPSRFSGSGSGTDFTLTISSLQPEDF | | |
| | ATYFCQQYYTYPYTFGGGTKLEIKGGGSGG | | |
| | GSHHHHHH | | |
| PEP004361 | APASSSTKKTQLQLEHLLLDLQMILNGINNY | | 721 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGDTF | | |
| | TRHYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSKLRVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGYTFTTYYMHWVRQAPGQGL | | |
| | EWMGIINPSGGGTLYAQKFQGRVTMTRDTS | | |
| | TSTVYMELSSLRSEDTAVYYCAAGLFIWGQ | | |
| | GTTVTVSSASGGGGSGGGGSGGGGSHASDI | | |
| | VMTQSPDSLAVSLGERATINCKASQSLLHSS | | |
| | SNKNYLAWYQQKPGQPPKLLIYWASTRES | | |
| | GVPDRFSGSGSGTDFTLTISSLQAEDVAVYY | | |
| | CQQYYSTPITFGPGTKVDIK | | |
| PEP004363 | APASSSTKKTQLQLEHLLLDLQMILNGINNY | | 722 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGDTF | | |
| | TRYYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSKLRVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGYTFTTYYMHWVRQAPGQGL | | |
| | EWMGIINPSGGGTLYAQKFQGRVTMTRDTS | | |
| | TSTVYMELSSLRSEDTAVYYCAAGLFIWGQ | | |
| | GTTVTVSSASGGGGSGGGGSGGGGSHASDI | | |
| | VMTQSPDSLAVSLGERATINCKASQSLLHSS | | |
| | SNKNYLAWYQQKPGQPPKLLIYWASTRES | | |
| | GVPDRFSGSGSGTDFTLTISSLQAEDVAVYY | | |
| | CQQYYSTPITFGPGTKVDIK | | |
| PEP004395 | EVQLLESGGGLVQPGGSLRLSCAASGFSFSS | | 723 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SAKTTAPSVYPLAPVCGDTTGSSVTLGCLV | | |
| | KGYFPEPVTLTWNSGSLSSGVHTFPAVLQS | | |
| | DLYTLSSSVTVTSSTWPSQSITCNVAHPASS | | |
| | TKVDKKIEPRGPTIKPCPPCKCPAPNAAGGP | | |
| | SVFIFPPKIKDVLMISLSPIVTCVVVDVSEDD | | |
| | PDVQISWFVNNVEVHTAQTQTHREDYNSTL | | |
| | RVVSALPIQHQDWMSGKEFKCKVNNKDLG | | |
| | APIERTISKPKGSVRAPQVYVLPPPEKEMTK | | |
| | KQVSLTCLVKDFMPEDIYVEWTNNGKTEL | | |
| | NYKNTEPVLKSDGSYFMYSKLTVEKKNWV | | |
| | ERNSYSCSVVHEGLHNHHTTKSFSRTPGGG | | |
| | GGSGGGGSGGGGSGGGGSQVQLVQSGAEV | | |
| | KKPGASVKVSCKASGDTFTRHYVHWVRQA | | |
| | PGQGLEWMGIINPSGGYASYAQKFQGRVT | | |
| | MTRDTSTSTVYMELSSLRSEDTAVYYCAAG | | |
| | LFIWGQGTLVTVSSASGGGGSGGGGSGGG | | |
| | GSHASDIQMTQSPSSLSASVGDRVTITCRAS | | |
| | QSIGRWLAWYQQKPGKAPKLLIYSASNLET | | |
| | GVPSRFSGSGSGTDFTLTISSLQPEDFATYYC | | |
| | QQYESFPVTFGPGTKVDIKGGGSGGGSHHH | | |
| | HHH | | |
| PEP004398 | EVQLLESGGGLVQPGGSLRLSCAASGFSFSS | | 724 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SAKTTAPSVYPLAPVCGDTTGSSVTLGCLV | | |
| | KGYFPEPVTLTWNSGSLSSGVHTFPAVLQS | | |
| | DLYTLSSSVTVTSSTWPSQSITCNVAHPASS | | |
| | TKVDKKIEPRGPTIKPCPPCKCPAPNAAGGP | | |
| | SVFIFPPKIKDVLMISLSPIVTCVVVDVSEDD | | |
| | PDVQISWFVNNVEVHTAQTQTHREDYNSTL | | |
| | RVVSALPIQHQDWMSGKEFKCKVNNKDLG | | |
| | APIERTISKPKGSVRAPQVYVLPPPEKEMTK | | |
| | KQVSLTCLVKDFMPEDIYVEWTNNGKTEL | | |
| | NYKNTEPVLKSDGSYFMYSKLTVEKKNWV | | |
| | ERNSYSCSVVHEGLHNHHTTKSFSRTPGGG | | |
| | GGSGGGGSGGGGSGGGGSQVQLVQSGAEV | | |
| | KKPGASVKVSCKASGDTFTRYYVHWVRQA | | |
| | PGQGLEWMGIINPSGGYASYAQKFQGRVT | | |
| | MTRDTSTSTVYMELSSLRSEDTAVYYCAAG | | |
| | LFIWGQGTLVTVSSASGGGGSGGGGSGGG | | |
| | GSHASDIQMTQSPSSLSASVGDRVTITCRAS | | |
| | QSIGRWLAWYQQKPGKAPKLLIYSASNLET | | |
| | GVPSRFSGSGSGTDFTLTISSLQPEDFATYYC | | |
| | QQYNRFPVTFGPGTKVDIKGGGSGGGSHHH | | |
| | HHH | | |
| PEP004404 | EVQLLESGGGLVQPGGSLRLSCAASGFSFSS | | 725 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SAKTTAPSVYPLAPVCGDTTGSSVTLGCLV | | |
| | KGYFPEPVTLTWNSGSLSSGVHTFPAVLQS | | |
| | DLYTLSSSVTVTSSTWPSQSITCNVAHPASS | | |
| | TKVDKKIEPRGPTIKPCPPCKCPAPNAAGGP | | |
| | SVFIFPPKIKDVLMISLSPIVTCVVVDVSEDD | | |
| | PDVQISWFVNNVEVHTAQTQTHREDYNSTL | | |
| | RVVSALPIQHQDWMSGKEFKCKVNNKDLG | | |
| | APIERTISKPKGSVRAPQVYVLPPPEKEMTK | | |
| | KQVSLTCLVKDFMPEDIYVEWTNNGKTEL | | |
| | NYKNTEPVLKSDGSYFMYSKLTVEKKNWV | | |
| | ERNSYSCSVVHEGLHNHHTTKSFSRTPGGG | | |
| | GGSGGGGSGGGGSGGGGSQVQLVQSGAEV | | |
| | KKPGASVKVSCKASGHTFTRYYMHWVRQ | | |
| | APGQGLEWMGIINPSGGYATYAQKFQGRV | | |
| | TMTRDTSTSTVYMELSSLRSEDTAVYYCAS | | |
| | GLFIWGQGTLVTVSSASGGGGSGGGGSGG | | |
| | GGSHASDIQMTQSPSSLSASVGDRVTITCRA | | |
| | SQSINSWLAWYQQKPGKAPKLLIYATSTLE | | |
| | SGVPSRFSGSGSGTDFTLTISSLQPEDFATYY | | |
| | CQQYYRFPVTFGQGTKVEIKGGGSGGGSHH | | |
| | HHHH | | |
| PEP004406 | EVQLLESGGGLVQPGGSLRLSCAASGFSFSS | | 726 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SAKTTAPSVYPLAPVCGDTTGSSVTLGCLV | | |
| | KGYFPEPVTLTWNSGSLSSGVHTFPAVLQS | | |
| | DLYTLSSSVTVTSSTWPSQSITCNVAHPASS | | |
| | TKVDKKIEPRGPTIKPCPPCKCPAPNAAGGP | | |
| | SVFIFPPKIKDVLMISLSPIVTCVVVDVSEDD | | |
| | PDVQISWFVNNVEVHTAQTQTHREDYNSTL | | |
| | RVVSALPIQHQDWMSGKEFKCKVNNKDLG | | |
| | APIERTISKPKGSVRAPQVYVLPPPEKEMTK | | |
| | KQVSLTCLVKDFMPEDIYVEWTNNGKTEL | | |
| | NYKNTEPVLKSDGSYFMYSKLTVEKKNWV | | |
| | ERNSYSCSVVHEGLHNHHTTKSFSRTPGGG | | |
| | GGSGGGGSGGGGSGGGGSQVQLVQSGAEV | | |
| | KKPGASVKVSCKASGYTFTDYYMHWVRQ | | |
| | APGQGLEWMGIINPRAGYTSYALKFQGRVT | | |
| | MTRDTSTSTVYMELSSLRSEDTAVYYCTSG | | |
| | WDVWGQGTLVTVSSASGGGGSGGGGSGG | | |
| | GGSHASDIQMTQSPSSLSASVGDRVTITCRA | | |
| | SQSISTWLAWYQQKPGKAPKLLIYAASSLD | | |
| | SGVPSRFSGSGSGTDFTLTISSLQPEDFATYY | | |
| | CQQSYSFPVTFGQGTKVEIKGGGSGGGSHH | | |
| | HHHH | | |
| PEP004416 | APASSSTKKTQLQLEHLLLDLQMILNGINNY | | 727 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGYTF | | |
| | TDYYMHWVRQAPGQGLEWMGIINPRAGY | | |
| | TSYALKFQGRVTMTRDTSTSTVYMELSSLR | | |
| | SEDTAVYYCTSGWDVWGQGTLVTVSSAKT | | |
| | TAPSVYPLAPVCGDTTGSSVTLGCLVKGYF | | |
| | PEPVTLTWNSGSLSSGVHTFPAVLQSDLYTL | | |
| | SSSVTVTSSTWPSQSITCNVAHPASSTKVDK | | |
| | KIEPRGPTIKPCPPCKCPAPNAAGGPSVFIFP | | |
| | PKIKDVLMISLSPIVTCVVVDVSEDDPDVQI | | |
| | SWFVNNVEVHTAQTQTHREDYNSTLRVVS | | |
| | ALPIQHQDWMSGKEFKCKVNNKDLGAPIE | | |
| | RTISKPKGSVRAPQVYVLPPPEEEMTKKQV | | |
| | TLTCMVTDFMPEDIYVEWTNNGKTELNYK | | |
| | NTEPVLDSDGSYFMYSKLRVEKKNWVERN | | |
| | SYSCSVVHEGLHNHHTTKSFSRTPGK | | |
| PEP004418 | APASSSTKKTQLQLEHLLLDLQMILNGINNY | | 728 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGYTF | | |
| | TRYYMHWVRQAPGQGLEWMGIINPRAGYT | | |
| | SYALKFQGRVTMTRDTSTSTVYMELSSLRS | | |
| | EDTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSKLRVEKKNWVERNSY | | |
| | SCSVVHEGLHNHHTTKSFSRTPGK | | |
| PEP004419 | APASSSTKKTQLQLEHLLLDLQMILNGINNY | | 729 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGYTF | | |
| | TTYYMHWVRQAPGQGLEWMGIINPRAGYT | | |
| | SYALKFQGRVTMTRDTSTSTVYMELSSLRS | | |
| | EDTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSKLRVEKKNWVERNSY | | |
| | SCSVVHEGLHNHHTTKSFSRTPGK | | |
| PEP004420 | DIQMTQSPSSLSASVGDRVTITCRASQSIGR | | 730 |
| | WLAWYQQKPGKAPKLLIYSASNLETGVPSR | | |
| | FSGSGSGTDFTLTISSLQPEDFATYYCQQYE | | |
| | RFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004421 | DIQMTQSPSSLSASVGDRVTITCRASQSIGS | | 731 |
| | WLAWYQQKPGKAPKLLIYSASNLETGVPSR | | |
| | FSGSGSGTDFTLTISSLQPEDFATYYCQQYN | | |
| | SFPVTFGPGTKVDIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004423 | DIQMTQSPSSLSASVGDRVTITCRASQSINS | | 732 |
| | WLAWYQQKPGKAPKLLIYATSTLESGVPSR | | |
| | FSGSGSGTDFTLTISSLQPEDFATYYCQQYY | | |
| | RFPVTFGQGTKVEIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004426 | DIQMTQSPSSLSASVGDRVTITCRASQSIST | | 733 |
| | WLAWYQQKPGKAPKLLIYAASSLDSGVPS | | |
| | RFSGSGSGTDFTLTISSLQPEDFATYYCQQS | | |
| | YRFPVTFGQGTKVEIKRADAAPTVSIFPPSSE | | |
| | QLTSGGASVVCFLNNFYPKDINVKWKIDGS | | |
| | ERQNGVLNSWTDQDSKDSTYSMSSTLTLTK | | |
| | DEYERHNSYTCEATHKTSTSPIVKSFNRNEC | | |
| PEP004427 | DIQMTQSPSSLSASVGDRVTITCRASQSIST | | 734 |
| | WLAWYQQKPGKAPKLLIYAASSLDSGVPS | | |
| | RFSGSGSGTDFTLTISSLQPEDFATYYCQQS | | |
| | YSFPVTFGQGTKVEIKRADAAPTVSIFPPSSE | | |
| | QLTSGGASVVCFLNNFYPKDINVKWKIDGS | | |
| | ERQNGVLNSWTDQDSKDSTYSMSSTLTLTK | | |
| | DEYERHNSYTCEATHKTSTSPIVKSFNRNEC | | |
| PEP004431 | APASSSTKKTQLQLEHLLLDLQMILNGINNY | | 735 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGHTF | | |
| | TRYYMHWVRQAPGQGLEWMGIINPSGGYA | | |
| | TYAQKFQGRVTMTRDTSTSTVYMELSSLRS | | |
| | EDTAVYYCASGLFIWGQGTLVTVSSAKTTA | | |
| | PSVYPLAPVCGDTTGSSVTLGCLVKGYFPEP | | |
| | VTLTWNSGSLSSGVHTFPAVLQSDLYTLSSS | | |
| | VTVTSSTWPSQSITCNVAHPASSTKVDKKIE | | |
| | PRGPTIKPCPPCKCPAPNAAGGPSVFIFPPKI | | |
| | KDVLMISLSPIVTCVVVDVSEDDPDVQISWF | | |
| | VNNVEVHTAQTQTHREDYNSTLRVVSALPI | | |
| | QHQDWMSGKEFKCKVNNKDLGAPIERTIS | | |
| | KPKGSVRAPQVYVLPPPEEEMTKKQVTLTC | | |
| | MVTDFMPEDIYVEWTNNGKTELNYKNTEP | | |
| | VLDSDGSYFMYSDLRVEKKNWVERNSYSC | | |
| | SVVHEGLHNHHTTESFSRTPGK | | |
| PEP004433 | APASSSTKKTQLQLEHLLLDLQMILNGINNY | | 736 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGYTF | | |
| | TDYYMHWVRQAPGQGLEWMGIINPRAGY | | |
| | TSYALKFQGRVTMTRDTSTSTVYMELSSLR | | |
| | SEDTAVYYCTSGWDVWGQGTLVTVSSAKT | | |
| | TAPSVYPLAPVCGDTTGSSVTLGCLVKGYF | | |
| | PEPVTLTWNSGSLSSGVHTFPAVLQSDLYTL | | |
| | SSSVTVTSSTWPSQSITCNVAHPASSTKVDK | | |
| | KIEPRGPTIKPCPPCKCPAPNAAGGPSVFIFP | | |
| | PKIKDVLMISLSPIVTCVVVDVSEDDPDVQI | | |
| | SWFVNNVEVHTAQTQTHREDYNSTLRVVS | | |
| | ALPIQHQDWMSGKEFKCKVNNKDLGAPIE | | |
| | RTISKPKGSVRAPQVYVLPPPEEEMTKKQV | | |
| | TLTCMVTDFMPEDIYVEWTNNGKTELNYK | | |
| | NTEPVLDSDGSYFMYSDLRVEKKNWVERN | | |
| | SYSCSVVHEGLHNHHTTESFSRTPGK | | |
| PEP004435 | APASSSTKKTQLQLEHLLLDLQMILNGINNY | | 737 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGYTF | | |
| | TRYYMHWVRQAPGQGLEWMGIINPRAGYT | | |
| | SYALKFQGRVTMTRDTSTSTVYMELSSLRS | | |
| | EDTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGK | | |
| PEP004436 | APASSSTKKTQLQLEHLLLDLQMILNGINNY | | 738 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | QVQLVQSGAEVKKPGASVKVSCKASGYTF | | |
| | TTYYMHWVRQAPGQGLEWMGIINPRAGYT | | |
| | SYALKFQGRVTMTRDTSTSTVYMELSSLRS | | |
| | EDTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGK | | |
| PEP004438 | QVQLVQSGAEVKKPGASVKVSCKASGDTF | | 739 |
| | TRYYVHWVRQAPGQGLEWMGIINPSGGYA | | |
| | SYAQKFQGRVTMTRDTSTSTVYMELSSLRS | | |
| | EDTAVYYCAAGLFIWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGSGGG | | |
| | SHHHHHH | | |
| PEP004440 | QVQLVQSGAEVKKPGASVKVSCKASGHTF | | 740 |
| | TRYYMHWVRQAPGQGLEWMGIINPSGGYA | | |
| | TYAQKFQGRVTMTRDTSTSTVYMELSSLRS | | |
| | EDTAVYYCASGLFIWGQGTLVTVSSAKTTA | | |
| | PSVYPLAPVCGDTTGSSVTLGCLVKGYFPEP | | |
| | VTLTWNSGSLSSGVHTFPAVLQSDLYTLSSS | | |
| | VTVTSSTWPSQSITCNVAHPASSTKVDKKIE | | |
| | PRGPTIKPCPPCKCPAPNAAGGPSVFIFPPKI | | |
| | KDVLMISLSPIVTCVVVDVSEDDPDVQISWF | | |
| | VNNVEVHTAQTQTHREDYNSTLRVVSALPI | | |
| | QHQDWMSGKEFKCKVNNKDLGAPIERTIS | | |
| | KPKGSVRAPQVYVLPPPEKEMTKKQVSLTC | | |
| | LVKDFMPEDIYVEWTNNGKTELNYKNTEP | | |
| | VLKSDGSYFMYSKLTVEKKNWVERNSYSC | | |
| | SVVHEGLHNHHTTKSFSRTPGGGGSGGGSH | | |
| | HHHHH | | |
| PEP004442 | QVQLVQSGAEVKKPGASVKVSCKASGYTF | | 741 |
| | TDYYMHWVRQAPGQGLEWMGIINPRAGY | | |
| | TSYALKFQGRVTMTRDTSTSTVYMELSSLR | | |
| | SEDTAVYYCTSGWDVWGQGTLVTVSSAKT | | |
| | TAPSVYPLAPVCGDTTGSSVTLGCLVKGYF | | |
| | PEPVTLTWNSGSLSSGVHTFPAVLQSDLYTL | | |
| | SSSVTVTSSTWPSQSITCNVAHPASSTKVDK | | |
| | KIEPRGPTIKPCPPCKCPAPNAAGGPSVFIFP | | |
| | PKIKDVLMISLSPIVTCVVVDVSEDDPDVQI | | |
| | SWFVNNVEVHTAQTQTHREDYNSTLRVVS | | |
| | ALPIQHQDWMSGKEFKCKVNNKDLGAPIE | | |
| | RTISKPKGSVRAPQVYVLPPPEKEMTKKQV | | |
| | SLTCLVKDFMPEDIYVEWTNNGKTELNYK | | |
| | NTEPVLKSDGSYFMYSKLTVEKKNWVERN | | |
| | SYSCSVVHEGLHNHHTTKSFSRTPGGGGSG | | |
| | GGSHHHHHH | | |
| PEP004443 | QVQLVQSGAEVKKPGASVKVSCKASGYTF | | 742 |
| | TKNYMHWVRQAPGQGLEWLGWVSPDSGY | | |
| | TGYAQKFQGRVTMTRDTSTSTVYMELSSLR | | |
| | SEDTAVYYCTTDLLSLELDDAFDIWGQGTM | | |
| | VTVSSAKTTAPSVYPLAPVCGDTTGSSVTL | | |
| | GCLVKGYFPEPVTLTWNSGSLSSGVHTFPA | | |
| | VLQSDLYTLSSSVTVTSSTWPSQSITCNVAH | | |
| | PASSTKVDKKIEPRGPTIKPCPPCKCPAPNA | | |
| | AGGPSVFIFPPKIKDVLMISLSPIVTCVVVDV | | |
| | SEDDPDVQISWFVNNVEVHTAQTQTHRED | | |
| | YNSTLRVVSALPIQHQDWMSGKEFKCKVN | | |
| | NKDLGAPIERTISKPKGSVRAPQVYVLPPPE | | |
| | KEMTKKQVSLTCLVKDFMPEDIYVEWTNN | | |
| | GKTELNYKNTEPVLKSDGSYFMYSKLTVEK | | |
| | KNWVERNSYSCSVVHEGLHNHHTTKSFSR | | |
| | TPGGGGSGGGSHHHHHH | | |
| PEP004445 | QVQLVQSGAEVKKPGASVKVSCKASGYTF | | 743 |
| | TRYYMHWVRQAPGQGLEWMGIINPRAGYT | | |
| | SYALKFQGRVTMTRDTSTSTVYMELSSLRS | | |
| | EDTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGSGGG | | |
| | SHHHHHH | | |
| PEP004446 | QVQLVQSGAEVKKPGASVKVSCKASGYTF | | 744 |
| | TTYYMHWVRQAPGQGLEWMGIINPRAGYT | | |
| | SYALKFQGRVTMTRDTSTSTVYMELSSLRS | | |
| | EDTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGSGGG | | |
| | SHHHHHH | | |
| PEP004729 | EIVLTQSPATLSLSPGERATLSCRASQSVSSY | | 745 |
| | LAWYQQKPGQAPRLLIYDASNRATGIPARF | | |
| | SGSGSGTDFTLTISSLEPEDFAVYYCQQSSN | | |
| | WPRTFGQGTKVEIKRADAAPTVSIFPPSSEQ | | |
| | LTSGGASVVCFLNNFYPKDINVKWKIDGSE | | |
| | RQNGVLNSWTDQDSKDSTYSMSSTLTLTK | | |
| | DEYERHNSYTCEATHKTSTSPIVKSFNRNEC | | |
| PEP004810 | APASSSTKKTQLQLEHLLLDLQMILNGINNY | | 746 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSQVQLVQSGAEVKKPG | | |
| | ASVKVSCKASGDTFTRYYVHWVRQAPGQG | | |
| | LEWMGIINPSGGYASYAQKFQGRVTMTRD | | |
| | TSTSTVYMELSSLRSEDTAVYYCAAGLFIW | | |
| | GQGTLVTVSSAKTTAPSVYPLAPVCGDTTG | | |
| | SSVTLGCLVKGYFPEPVTLTWNSGSLSSGV | | |
| | HTFPAVLQSDLYTLSSSVTVTSSTWPSQSIT | | |
| | CNVAHPASSTKVDKKIEPRGPTIKPCPPCKC | | |
| | PAPNAAGGPSVFIFPPKIKDVLMISLSPIVTC | | |
| | VVVDVSEDDPDVQISWFVNNVEVHTAQTQ | | |
| | THREDYNSTLRVVSALPIQHQDWMSGKEF | | |
| | KCKVNNKDLGAPIERTISKPKGSVRAPQVY | | |
| | VLPPPEEEMTKKQVTLTCMVTDFMPEDIYV | | |
| | EWTNNGKTELNYKNTEPVLDSDGSYFMYS | | |
| | DLRVEKKNWVERNSYSCSVVHEGLHNHHT | | |
| | TESFSRTPGK | | |
| PEP004813 | APASSSTKKTQLQLEHLLLDLQMILNGINNY | | 747 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSQVQLVQSGAEVKKPG | | |
| | ASVKVSCKASGYTFTRYYMHWVRQAPGQ | | |
| | GLEWMGIINPRAGYTSYALKFQGRVTMTR | | |
| | DTSTSTVYMELSSLRSEDTAVYYCTSGWDV | | |
| | WGQGTLVTVSSAKTTAPSVYPLAPVCGDTT | | |
| | GSSVTLGCLVKGYFPEPVTLTWNSGSLSSG | | |
| | VHTFPAVLQSDLYTLSSSVTVTSSTWPSQSI | | |
| | TCNVAHPASSTKVDKKIEPRGPTIKPCPPCK | | |
| | CPAPNAAGGPSVFIFPPKIKDVLMISLSPIVT | | |
| | CVVVDVSEDDPDVQISWFVNNVEVHTAQT | | |
| | QTHREDYNSTLRVVSALPIQHQDWMSGKE | | |
| | FKCKVNNKDLGAPIERTISKPKGSVRAPQV | | |
| | YVLPPPEEEMTKKQVTLTCMVTDFMPEDIY | | |
| | VEWTNNGKTELNYKNTEPVLDSDGSYFMY | | |
| | SDLRVEKKNWVERNSYSCSVVHEGLHNHH | | |
| | TTESFSRTPGK | | |
| PEP004818 | QVQLVQSGAEVKKPGASVKVSCKASGYTF | | 748 |
| | TRYYMHWVRQAPGQGLEWMGIINPRAGYT | | |
| | SYALKFQGRVTMTRDTSTSTVYMELSSLRS | | |
| | EDTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGK | | |
| PEP004822 | APASSSTKKTQLQLEHLLLDLQMILNGINNY | | 749 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSQVQLVQSGAE | | |
| | VKKPGASVKVSCKASGDTFTRYYVHWVRQ | | |
| | APGQGLEWMGIINPSGGYASYAQKFQGRV | | |
| | TMTRDTSTSTVYMELSSLRSEDTAVYYCAA | | |
| | GLFIWGQGTLVTVSSAKTTAPSVYPLAPVC | | |
| | GDTTGSSVTLGCLVKGYFPEPVTLTWNSGS | | |
| | LSSGVHTFPAVLQSDLYTLSSSVTVTSSTWP | | |
| | SQSITCNVAHPASSTKVDKKIEPRGPTIKPCP | | |
| | PCKCPAPNAAGGPSVFIFPPKIKDVLMISLSP | | |
| | IVTCVVVDVSEDDPDVQISWFVNNVEVHTA | | |
| | QTQTHREDYNSTLRVVSALPIQHQDWMSG | | |
| | KEFKCKVNNKDLGAPIERTISKPKGSVRAPQ | | |
| | VYVLPPPEEEMTKKQVTLTCMVTDFMPEDI | | |
| | YVEWTNNGKTELNYKNTEPVLDSDGSYFM | | |
| | YSDLRVEKKNWVERNSYSCSVVHEGLHNH | | |
| | HTTESFSRTPGK | | |
| PEP004825 | APASSSTKKTQLQLEHLLLDLQMILNGINNY | | 750 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSQVQLVQSGAE | | |
| | VKKPGASVKVSCKASGYTFTRYYMHWVR | | |
| | QAPGQGLEWMGIINPRAGYTSYALKFQGR | | |
| | VTMTRDTSTSTVYMELSSLRSEDTAVYYCT | | |
| | SGWDVWGQGTLVTVSSAKTTAPSVYPLAP | | |
| | VCGDTTGSSVTLGCLVKGYFPEPVTLTWNS | | |
| | GSLSSGVHTFPAVLQSDLYTLSSSVTVTSST | | |
| | WPSQSITCNVAHPASSTKVDKKIEPRGPTIK | | |
| | PCPPCKCPAPNAAGGPSVFIFPPKIKDVLMIS | | |
| | LSPIVTCVVVDVSEDDPDVQISWFVNNVEV | | |
| | HTAQTQTHREDYNSTLRVVSALPIQHQDW | | |
| | MSGKEFKCKVNNKDLGAPIERTISKPKGSV | | |
| | RAPQVYVLPPPEEEMTKKQVTLTCMVTDF | | |
| | MPEDIYVEWTNNGKTELNYKNTEPVLDSD | | |
| | GSYFMYSDLRVEKKNWVERNSYSCSVVHE | | |
| | GLHNHHTTESFSRTPGK | | |
| PEP004829 | APASSSTKKTQLQLEHLLLDLQMILNGINNY | | 751 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSQVQLV | | |
| | QSGAEVKKPGASVKVSCKASGDTFTRYYV | | |
| | HWVRQAPGQGLEWMGIINPSGGYASYAQK | | |
| | FQGRVTMTRDTSTSTVYMELSSLRSEDTAV | | |
| | YYCAAGLFIWGQGTLVTVSSAKTTAPSVYP | | |
| | LAPVCGDTTGSSVTLGCLVKGYFPEPVTLT | | |
| | WNSGSLSSGVHTFPAVLQSDLYTLSSSVTV | | |
| | TSSTWPSQSITCNVAHPASSTKVDKKIEPRG | | |
| | PTIKPCPPCKCPAPNAAGGPSVFIFPPKIKDV | | |
| | LMISLSPIVTCVVVDVSEDDPDVQISWFVNN | | |
| | VEVHTAQTQTHREDYNSTLRVVSALPIQHQ | | |
| | DWMSGKEFKCKVNNKDLGAPIERTISKPKG | | |
| | SVRAPQVYVLPPPEEEMTKKQVTLTCMVT | | |
| | DFMPEDIYVEWTNNGKTELNYKNTEPVLDS | | |
| | DGSYFMYSDLRVEKKNWVERNSYSCSVVH | | |
| | EGLHNHHTTESFSRTPGK | | |
| PEP004832 | APASSSTKKTQLQLEHLLLDLQMILNGINNY | | 752 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSQVQLV | | |
| | QSGAEVKKPGASVKVSCKASGYTFTRYYM | | |
| | HWVRQAPGQGLEWMGIINPRAGYTSYALK | | |
| | FQGRVTMTRDTSTSTVYMELSSLRSEDTAV | | |
| | YYCTSGWDVWGQGTLVTVSSAKTTAPSVY | | |
| | PLAPVCGDTTGSSVTLGCLVKGYFPEPVTLT | | |
| | WNSGSLSSGVHTFPAVLQSDLYTLSSSVTV | | |
| | TSSTWPSQSITCNVAHPASSTKVDKKIEPRG | | |
| | PTIKPCPPCKCPAPNAAGGPSVFIFPPKIKDV | | |
| | LMISLSPIVTCVVVDVSEDDPDVQISWFVNN | | |
| | VEVHTAQTQTHREDYNSTLRVVSALPIQHQ | | |
| | DWMSGKEFKCKVNNKDLGAPIERTISKPKG | | |
| | SVRAPQVYVLPPPEEEMTKKQVTLTCMVT | | |
| | DFMPEDIYVEWTNNGKTELNYKNTEPVLDS | | |
| | DGSYFMYSDLRVEKKNWVERNSYSCSVVH | | |
| | EGLHNHHTTESFSRTPGK | | |
| PEP004836 | APASSSTKKTQLQLEHLLLDLQMILNGINNY | | 753 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | GGGGSQVQLVQSGAEVKKPGASVKVSCKA | | |
| | SGDTFTRYYVHWVRQAPGQGLEWMGIINP | | |
| | SGGYASYAQKFQGRVTMTRDTSTSTVYME | | |
| | LSSLRSEDTAVYYCAAGLFIWGQGTLVTVS | | |
| | SAKTTAPSVYPLAPVCGDTTGSSVTLGCLV | | |
| | KGYFPEPVTLTWNSGSLSSGVHTFPAVLQS | | |
| | DLYTLSSSVTVTSSTWPSQSITCNVAHPASS | | |
| | TKVDKKIEPRGPTIKPCPPCKCPAPNAAGGP | | |
| | SVFIFPPKIKDVLMISLSPIVTCVVVDVSEDD | | |
| | PDVQISWFVNNVEVHTAQTQTHREDYNSTL | | |
| | RVVSALPIQHQDWMSGKEFKCKVNNKDLG | | |
| | APIERTISKPKGSVRAPQVYVLPPPEEEMTK | | |
| | KQVTLTCMVTDFMPEDIYVEWTNNGKTEL | | |
| | NYKNTEPVLDSDGSYFMYSDLRVEKKNWV | | |
| | ERNSYSCSVVHEGLHNHHTTESFSRTPGK | | |
| PEP004839 | APASSSTKKTQLQLEHLLLDLQMILNGINNY | | 754 |
| | KNPKLTRMLTAKFAMPKKATELKHLQCLE | | |
| | EELKPLEEVLNGAQSKNFHLRPRDLISNINVI | | |
| | VLELKGSETTFMCEYADETATIVEFLNRWIT | | |
| | FAQSIISTLTGGGGSGGGGSGGGGSGGGGS | | |
| | GGGGSQVQLVQSGAEVKKPGASVKVSCKA | | |
| | SGYTFTRYYMHWVRQAPGQGLEWMGIINP | | |
| | RAGYTSYALKFQGRVTMTRDTSTSTVYME | | |
| | LSSLRSEDTAVYYCTSGWDVWGQGTLVTV | | |
| | SSAKTTAPSVYPLAPVCGDTTGSSVTLGCL | | |
| | VKGYFPEPVTLTWNSGSLSSGVHTFPAVLQ | | |
| | SDLYTLSSSVTVTSSTWPSQSITCNVAHPAS | | |
| | STKVDKKIEPRGPTIKPCPPCKCPAPNAAGG | | |
| | PSVFIFPPKIKDVLMISLSPIVTCVVVDVSED | | |
| | DPDVQISWFVNNVEVHTAQTQTHREDYNS | | |
| | TLRVVSALPIQHQDWMSGKEFKCKVNNKD | | |
| | LGAPIERTISKPKGSVRAPQVYVLPPPEEEM | | |
| | TKKQVTLTCMVTDFMPEDIYVEWTNNGKT | | |
| | ELNYKNTEPVLDSDGSYFMYSDLRVEKKN | | |
| | WVERNSYSCSVVHEGLHNHHTTESFSRTPG | | |
| | K | | |
| PEP004957 | APTSSSTKKTQLQLEHLLLDLQMILNGINNY | | 755 |
| | KNPKLTDMLTFEFYMPKKATELKHLQCLER | | |
| | ELKPLEEVLNLAQSKNFHLRPRDLISNINVIV | | |
| | LELKGSETTFMCEYADETATIVEFLNRWITF | | |
| | CQSIISTLTGGGGSGGGGSGGGGSGGGGSQ | | |
| | VQLVQSGAEVKKPGASVKVSCKASGDTFT | | |
| | RYYVHWVRQAPGQGLEWMGIINPSGGYAS | | |
| | YAQKFQGRVTMTRDTSTSTVYMELSSLRSE | | |
| | DTAVYYCAAGLFIWGQGTLVTVSSAKTTAP | | |
| | SVYPLAPVCGDTTGSSVTLGCLVKGYFPEP | | |
| | VTLTWNSGSLSSGVHTFPAVLQSDLYTLSSS | | |
| | VTVTSSTWPSQSITCNVAHPASSTKVDKKIE | | |
| | PRGPTIKPCPPCKCPAPNAAGGPSVFIFPPKI | | |
| | KDVLMISLSPIVTCVVVDVSEDDPDVQISWF | | |
| | VNNVEVHTAQTQTHREDYNSTLRVVSALPI | | |
| | QHQDWMSGKEFKCKVNNKDLGAPIERTIS | | |
| | KPKGSVRAPQVYVLPPPEEEMTKKQVTLTC | | |
| | MVTDFMPEDIYVEWTNNGKTELNYKNTEP | | |
| | VLDSDGSYFMYSDLRVEKKNWVERNSYSC | | |
| | SVVHEGLHNHHTTESFSRTPGK | | |
| PEP004958 | APTSSSTKKTQLQLEHLLLDLQMILNGINNY | | 756 |
| | KNPKLTDMLTFEFYMPKKATELKHLQCLER | | |
| | ELKPLEEVLNLAQSKNFHLRPRDLISNINVIV | | |
| | LELKGSETTFMCEYADETATIVEFLNRWITF | | |
| | CQSIISTLTGGGGSGGGGSGGGGSGGGGSQ | | |
| | VQLVQSGAEVKKPGASVKVSCKASGHTFT | | |
| | RYYMHWVRQAPGQGLEWMGIINPSGGYAT | | |
| | YAQKFQGRVTMTRDTSTSTVYMELSSLRSE | | |
| | DTAVYYCASGLFIWGQGTLVTVSSAKTTAP | | |
| | SVYPLAPVCGDTTGSSVTLGCLVKGYFPEP | | |
| | VTLTWNSGSLSSGVHTFPAVLQSDLYTLSSS | | |
| | VTVTSSTWPSQSITCNVAHPASSTKVDKKIE | | |
| | PRGPTIKPCPPCKCPAPNAAGGPSVFIFPPKI | | |
| | KDVLMISLSPIVTCVVVDVSEDDPDVQISWF | | |
| | VNNVEVHTAQTQTHREDYNSTLRVVSALPI | | |
| | QHQDWMSGKEFKCKVNNKDLGAPIERTIS | | |
| | KPKGSVRAPQVYVLPPPEEEMTKKQVTLTC | | |
| | MVTDFMPEDIYVEWTNNGKTELNYKNTEP | | |
| | VLDSDGSYFMYSDLRVEKKNWVERNSYSC | | |
| | SVVHEGLHNHHTTESFSRTPGK | | |
| PEP004959 | APTSSSTKKTQLQLEHLLLDLQMILNGINNY | | 757 |
| | KNPKLTDMLTFEFYMPKKATELKHLQCLER | | |
| | ELKPLEEVLNLAQSKNFHLRPRDLISNINVIV | | |
| | LELKGSETTFMCEYADETATIVEFLNRWITF | | |
| | CQSIISTLTGGGGSGGGGSGGGGSGGGGSQ | | |
| | VQLVQSGAEVKKPGASVKVSCKASGYTFT | | |
| | DYYMHWVRQAPGQGLEWMGIINPRAGYTS | | |
| | YALKFQGRVTMTRDTSTSTVYMELSSLRSE | | |
| | DTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGK | | |
| PEP004960 | APTSSSTKKTQLQLEHLLLDLQMILNGINNY | | 758 |
| | KNPKLTDMLTFEFYMPKKATELKHLQCLER | | |
| | ELKPLEEVLNLAQSKNFHLRPRDLISNINVIV | | |
| | LELKGSETTFMCEYADETATIVEFLNRWITF | | |
| | CQSIISTLTGGGGSGGGGSGGGGSGGGGSQ | | |
| | VQLVQSGAEVKKPGASVKVSCKASGYTFT | | |
| | TYYMHWVRQAPGQGLEWMGIINPRAGYTS | | |
| | YALKFQGRVTMTRDTSTSTVYMELSSLRSE | | |
| | DTAVYYCTSGWDVWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGK | | |
| PEP004967 | QVQLVESGGGVVQPGRSLRLDCKASGITFS | | 759 |
| | NSGMHWVRQAPGKGLEWVAVIWYDGSKR | | |
| | YYADSVKGRFTISRDNSKNTLFLQMNSLRA | | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGGGGGSGG | | |
| | GGSGGGGSAPTSSSTKKTQLQLEHLLLDLQ | | |
| | MILNGINNYKNPKLTDMLTFEFYMPKKATE | | |
| | LKHLQCLERELKPLEEVLNLAQSKNFHLRP | | |
| | RDLISNINVIVLELKGSETTFMCEYADETATI | | |
| | VEFLNRWITFCQSIISTLT | | |
| PEP004973 | QVQLVESGGGVVQPGRSLRLDCKASGITFS | | 760 |
| | NSGMHWVRQAPGKGLEWVAVIWYDGSKR | | |
| | YYADSVKGRFTISRDNSKNTLFLQMNSLRA | | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGHTFTRYYMHWVRQAPGQGL | | |
| | EWMGIINPSGGYATYAQKFQGRVTMTRDT | | |
| | STSTVYMELSSLRSEDTAVYYCASGLFIWG | | |
| | QGTLVTVSSASGGGGSGGGGSGGGGSHAS | | |
| | DIQMTQSPSSLSASVGDRVTITCRASQSINS | | |
| | WLAWYQQKPGKAPKLLIYATSTLESGVPSR | | |
| | FSGSGSGTDFTLTISSLQPEDFATYYCQQYY | | |
| | RFPVTFGQGTKVEIKGGGSGGGSHHHHHH | | |
| PEP004974 | QVQLVESGGGVVQPGRSLRLDCKASGITFS | | 761 |
| | NSGMHWVRQAPGKGLEWVAVIWYDGSKR | | |
| | YYADSVKGRFTISRDNSKNTLFLQMNSLRA | | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGYTFTDYYMHWVRQAPGQG | | |
| | LEWMGIINPRAGYTSYALKFQGRVTMTRDT | | |
| | STSTVYMELSSLRSEDTAVYYCTSGWDVW | | |
| | GQGTLVTVSSASGGGGSGGGGSGGGGSHA | | |
| | SDIQMTQSPSSLSASVGDRVTITCRASQSIST | | |
| | WLAWYQQKPGKAPKLLIYAASSLDSGVPS | | |
| | RFSGSGSGTDFTLTISSLQPEDFATYYCQQS | | |
| | YSFPVTFGQGTKVEIKGGGSGGGSHHHHHH | | |
| PEP004978 | QVQLVQSGAEVKKPGASVKVSCKASGYTF | | 762 |
| | TTYYMHWVRQAPGQGLEWMGIINPSGGGT | | |
| | LYAQKFQGRVTMTRDTSTSTVYMELSSLRS | | |
| | EDTAVYYCAAGLFIWGQGTTVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGGGGGSGG | | |
| | GGSGGGGSAPTSSSTKKTQLQLEHLLLDLQ | | |
| | MILNGINNYKNPKLTDMLTFEFYMPKKATE | | |
| | LKHLQCLERELKPLEEVLNLAQSKNFHLRP | | |
| | RDLISNINVIVLELKGSETTFMCEYADETATI | | |
| | VEFLNRWITFCQSIISTLT | | |
| PEP004981 | QVQLVQSGAEVKKPGASVKVSCKASGYTF | | 763 |
| | TTYYMHWVRQAPGQGLEWMGIINPSGGGT | | |
| | LYAQKFQGRVTMTRDTSTSTVYMELSSLRS | | |
| | EDTAVYYCAAGLFIWGQGTTVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGDTFTRYYVHWVRQAPGQGL | | |
| | EWMGIINPSGGYASYAQKFQGRVTMTRDT | | |
| | STSTVYMELSSLRSEDTAVYYCAAGLFIWG | | |
| | QGTLVTVSSASGGGGSGGGGSGGGGSHAS | | |
| | DIQMTQSPSSLSASVGDRVTITCRASQSIGR | | |
| | WLAWYQQKPGKAPKLLIYSASNLETGVPSR | | |
| | FSGSGSGTDFTLTISSLQPEDFATYYCQQYE | | |
| | RFPVTFGPGTKVDIKGGGSGGGSHHHHHH | | |
| PEP004982 | QVQLVQSGAEVKKPGASVKVSCKASGYTF | | 764 |
| | TTYYMHWVRQAPGQGLEWMGIINPSGGGT | | |
| | LYAQKFQGRVTMTRDTSTSTVYMELSSLRS | | |
| | EDTAVYYCAAGLFIWGQGTTVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGDTFTRYYVHWVRQAPGQGL | | |
| | EWMGIINPSGGYASYAQKFQGRVTMTRDT | | |
| | STSTVYMELSSLRSEDTAVYYCAAGLFIWG | | |
| | QGTLVTVSSASGGGGSGGGGSGGGGSHAS | | |
| | DIQMTQSPSSLSASVGDRVTITCRASQSIGR | | |
| | YLAWYQQKPGKAPKLLIYSASNLETGVPSR | | |
| | FSGSGSGTDFTLTISSLQPEDFATYYCQQYN | | |
| | SFPVTFGPGTKVDIKGGGSGGGSHHHHHH | | |
| PEP004983 | QVQLVQSGAEVKKPGASVKVSCKASGYTF | | 765 |
| | TTYYMHWVRQAPGQGLEWMGIINPSGGGT | | |
| | LYAQKFQGRVTMTRDTSTSTVYMELSSLRS | | |
| | EDTAVYYCAAGLFIWGQGTTVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGHTFTRYYMHWVRQAPGQGL | | |
| | EWMGIINPSGGYATYAQKFQGRVTMTRDT | | |
| | STSTVYMELSSLRSEDTAVYYCASGLFIWG | | |
| | QGTLVTVSSASGGGGSGGGGSGGGGSHAS | | |
| | DIQMTQSPSSLSASVGDRVTITCRASQSINS | | |
| | WLAWYQQKPGKAPKLLIYATSTLESGVPSR | | |
| | FSGSGSGTDFTLTISSLQPEDFATYYCQQYY | | |
| | RFPVTFGQGTKVEIKGGGSGGGSHHHHHH | | |
| PEP004984 | QVQLVQSGAEVKKPGASVKVSCKASGYTF | | 766 |
| | TTYYMHWVRQAPGQGLEWMGIINPSGGGT | | |
| | LYAQKFQGRVTMTRDTSTSTVYMELSSLRS | | |
| | EDTAVYYCAAGLFIWGQGTTVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGYTFTDYYMHWVRQAPGQG | | |
| | LEWMGIINPRAGYTSYALKFQGRVTMTRDT | | |
| | STSTVYMELSSLRSEDTAVYYCTSGWDVW | | |
| | GQGTLVTVSSASGGGGSGGGGSGGGGSHA | | |
| | SDIQMTQSPSSLSASVGDRVTITCRASQSIST | | |
| | WLAWYQQKPGKAPKLLIYAASSLDSGVPS | | |
| | RFSGSGSGTDFTLTISSLQPEDFATYYCQQS | | |
| | YSFPVTFGQGTKVEIKGGGSGGGSHHHHHH | | |
| PEP005089 | QVQLVESGGGVVQPGRSLRLDCKASGITFS | | 767 |
| | NSGMHWVRQAPGKGLEWVAVIWYDGSKR | | |
| | YYADSVKGRFTISRDNSKNTLFLQMNSLRA | | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGGGGGSGG | | |
| | GGSGGGGSAPASSSTKKTQLQLEHLLLDLQ | | |
| | MILNGINNYKNPKLTRMLTAKFAMPKKAT | | |
| | ELKHLQCLEEELKPLEEVLNGAQSKNFHLR | | |
| | PRDLISNINVIVLELKGSETTFMCEYADETA | | |
| | TIVEFLNRWITFAQSIISTLT | | |
| PEP005090 | QVQLVESGGGVVQPGRSLRLDCKASGITFS | AB001203_ | 768 |
| | NSGMHWVRQAPGKGLEWVAVIWYDGSKR | trastuzumab | |
| | YYADSVKGRFTISRDNSKNTLFLQMNSLRA | control | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSEVQLVESGGGLVQPGG | | |
| | SLRLSCAASGFNIKDTYIHWVRQAPGKGLE | | |
| | WVARIYPTNGYTRYADSVKGRFTISADTSK | | |
| | NTAYLQMNSLRAEDTAVYYCSRWGGDGF | | |
| | YAMDYWGQGTLVTVSSASGGGGSGGGGS | | |
| | GGGGSHASDIQMTQSPSSLSASVGDRVTITC | | |
| | RASQDVNTAVAWYQQKPGKAPKLLIYSAS | | |
| | FLYSGVPSRFSGSRSGTDFTLTISSLQPEDFA | | |
| | TYYCQQHYTTPPTFGQGTKVEIK | | |
| PEP005091 | QVQLVESGGGVVQPGRSLRLDCKASGITFS | | 769 |
| | NSGMHWVRQAPGKGLEWVAVIWYDGSKR | | |
| | YYADSVKGRFTISRDNSKNTLFLQMNSLRA | | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGDTFTRYYVHWVRQAPGQGL | | |
| | EWMGIINPSGGYASYAQKFQGRVTMTRDT | | |
| | STSTVYMELSSLRSEDTAVYYCAAGLFIWG | | |
| | QGTLVTVSSASGGGGSGGGGSGGGGSHAS | | |
| | DIQMTQSPSSLSASVGDRVTITCRASQSIGR | | |
| | WLAWYQQKPGKAPKLLIYSASNLETGVPSR | | |
| | FSGSGSGTDFTLTISSLQPEDFATYYCQQYN | | |
| | RFPVTFGPGTKVDIK | | |
| PEP005092 | QVQLVESGGGVVQPGRSLRLDCKASGITFS | | 770 |
| | NSGMHWVRQAPGKGLEWVAVIWYDGSKR | | |
| | YYADSVKGRFTISRDNSKNTLFLQMNSLRA | | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEKEMTKKQVSL | | |
| | TCLVKDFMPEDIYVEWTNNGKTELNYKNT | | |
| | EPVLKSDGSYFMYSKLTVEKKNWVERNSY | | |
| | SCSVVHEGLHNHHTTKSFSRTPGGGGGSGG | | |
| | GGSGGGGSGGGGSQVQLVQSGAEVKKPGA | | |
| | SVKVSCKASGYTFTRYYMHWVRQAPGQGL | | |
| | EWMGIINPRAGYTSYALKFQGRVTMTRDTS | | |
| | TSTVYMELSSLRSEDTAVYYCTSGWDVWG | | |
| | QGTLVTVSSASGGGGSGGGGSGGGGSHAS | | |
| | DIQMTQSPSSLSASVGDRVTITCRASQSIST | | |
| | WLAWYQQKPGKAPKLLIYAASSLDSGVPS | | |
| | RFSGSGSGTDFTLTISSLQPEDFATYYCQQS | | |
| | YSFPVTFGQGTKVEIK | | |
| PEP005469 | DIQMTQSPSSLSASVGDRVTITCRASQTISRY | | 771 |
| | LNWYQQKPGKAPKLLIYAASSLQSGVPSRF | | |
| | SGSGSGTDFTLTISSLQPEDFATYYCQQSYS | | |
| | TPRTFGQGTKLEIKRADAAPTVSIFPPSSEQL | | |
| | TSGGASVVCFLNNFYPKDINVKWKIDGSER | | |
| | QNGVLNSWTDQDSKDSTYSMSSTLTLTKD | | |
| | EYERHNSYTCEATHKTSTSPIVKSFNRNEC | | |
| PEP005470 | QVQLVQSGAEVKKPGSSVKVSCKASGYTFT | | 772 |
| | AYYIHWVRQAPGQGLEFMGWIHPYSGGTN | | |
| | YAQKFQGRVTITADESTSTAYMELSSLRSE | | |
| | DTAVYYCAIGYYYGKFDYWGQGTLVTVSS | | |
| | AKTTAPSVYPLAPVCGDTTGSSVTLGCLVK | | |
| | GYFPEPVTLTWNSGSLSSGVHTFPAVLQSD | | |
| | LYTLSSSVTVTSSTWPSQSITCNVAHPASST | | |
| | KVDKKIEPRGPTIKPCPPCKCPAPNAAGGPS | | |
| | VFIFPPKIKDVLMISLSPIVTCVVVDVSEDDP | | |
| | DVQISWFVNNVEVHTAQTQTHREDYNSTL | | |
| | RVVSALPIQHQDWMSGKEFKCKVNNKDLG | | |
| | APIERTISKPKGSVRAPQVYVLPPPEEEMTK | | |
| | KQVTLTCMVTDFMPEDIYVEWTNNGKTEL | | |
| | NYKNTEPVLDSDGSYFMYSDLRVEKKNWV | | |
| | ERNSYSCSVVHEGLHNHHTTESFSRTPGGG | | |
| | GGSGGGGSGGGGSAPTSSSTKKTQLQLEHL | | |
| | LLDLQMILNGINNYKNPKLTDMLTFEFYMP | | |
| | KKATELKHLQCLERELKPLEEVLNLAQSKN | | |
| | FHLRPRDLISNINVIVLELKGSETTFMCEYA | | |
| | DETATIVEFLNRWITFCQSIISTLT | | |
| PEP005471 | QVQLVQSGAEVKKPGSSVKVSCKASGYTFT | | 773 |
| | AYYIHWVRQAPGQGLEFMGWIHPYSGGTN | | |
| | YAQKFQGRVTITADESTSTAYMELSSLRSE | | |
| | DTAVYYCAIGYYYGKFDYWGQGTLVTVSS | | |
| | AKTTAPSVYPLAPVCGDTTGSSVTLGCLVK | | |
| | GYFPEPVTLTWNSGSLSSGVHTFPAVLQSD | | |
| | LYTLSSSVTVTSSTWPSQSITCNVAHPASST | | |
| | KVDKKIEPRGPTIKPCPPCKCPAPNAAGGPS | | |
| | VFIFPPKIKDVLMISLSPIVTCVVVDVSEDDP | | |
| | DVQISWFVNNVEVHTAQTQTHREDYNSTL | | |
| | RVVSALPIQHQDWMSGKEFKCKVNNKDLG | | |
| | APIERTISKPKGSVRAPQVYVLPPPEKEMTK | | |
| | KQVSLTCLVKDFMPEDIYVEWTNNGKTEL | | |
| | NYKNTEPVLKSDGSYFMYSKLTVEKKNWV | | |
| | ERNSYSCSVVHEGLHNHHTTKSFSRTPGGG | | |
| | GGSGGGGSGGGGSGGGGSQVQLVQSGAEV | | |
| | KKPGASVKVSCKASGDTFTRYYVHWVRQA | | |
| | PGQGLEWMGIINPSGGYASYAQKFQGRVT | | |
| | MTRDTSTSTVYMELSSLRSEDTAVYYCAAG | | |
| | LFIWGQGTLVTVSSASGGGGSGGGGSGGG | | |
| | GSHASDIQMTQSPSSLSASVGDRVTITCRAS | | |
| | QSIGRWLAWYQQKPGKAPKLLIYSASNLET | | |
| | GVPSRFSGSGSGTDFTLTISSLQPEDFATYYC | | |
| | QQYNRFPVTFGPGTKVDIK | | |
| PEP005473 | DIVMTQSPLSLPVTPGEPASISCRSSQSLLHS | | 774 |
| | NGYNYLDWYLQKPGQSPQLLIYLGSNRAS | | |
| | GVPDRFSGSGSGTDFTLKISRVEAEDVGVY | | |
| | YCMQGTHWPPTFGQGTKVEIKRADAAPTV | | |
| | SIFPPSSEQLTSGGASVVCFLNNFYPKDINVK | | |
| | WKIDGSERQNGVLNSWTDQDSKDSTYSMS | | |
| | STLTLTKDEYERHNSYTCEATHKTSTSPIVK | | |
| | SFNRNEC | | |
| PEP005474 | QVQLVQSGAEVKKPGASVKVSCKASGYIFN | | 775 |
| | GYDIHWVRQAPGQGLEWMGWMNPDNGN | | |
| | TGLAQKFQGRVTITADESTSTAYMELSSLRS | | |
| | EDTAVYYCARGMATRFPYYYYGMDVWGQ | | |
| | GTLVTVSSAKTTAPSVYPLAPVCGDTTGSS | | |
| | VTLGCLVKGYFPEPVTLTWNSGSLSSGVHT | | |
| | FPAVLQSDLYTLSSSVTVTSSTWPSQSITCN | | |
| | VAHPASSTKVDKKIEPRGPTIKPCPPCKCPA | | |
| | PNAAGGPSVFIFPPKIKDVLMISLSPIVTCVV | | |
| | VDVSEDDPDVQISWFVNNVEVHTAQTQTH | | |
| | REDYNSTLRVVSALPIQHQDWMSGKEFKC | | |
| | KVNNKDLGAPIERTISKPKGSVRAPQVYVL | | |
| | PPPEEEMTKKQVTLTCMVTDFMPEDIYVEW | | |
| | TNNGKTELNYKNTEPVLDSDGSYFMYSDL | | |
| | RVEKKNWVERNSYSCSVVHEGLHNHHTTE | | |
| | SFSRTPGGGGGSGGGGSGGGGSAPTSSSTK | | |
| | KTQLQLEHLLLDLQMILNGINNYKNPKLTD | | |
| | MLTFEFYMPKKATELKHLQCLERELKPLEE | | |
| | VLNLAQSKNFHLRPRDLISNINVIVLELKGS | | |
| | ETTFMCEYADETATIVEFLNRWITFCQSIIST | | |
| | LT | | |
| PEP005475 | QVQLVQSGAEVKKPGASVKVSCKASGYIFN | | 776 |
| | GYDIHWVRQAPGQGLEWMGWMNPDNGN | | |
| | TGLAQKFQGRVTITADESTSTAYMELSSLRS | | |
| | EDTAVYYCARGMATRFPYYYYGMDVWGQ | | |
| | GTLVTVSSAKTTAPSVYPLAPVCGDTTGSS | | |
| | VTLGCLVKGYFPEPVTLTWNSGSLSSGVHT | | |
| | FPAVLQSDLYTLSSSVTVTSSTWPSQSITCN | | |
| | VAHPASSTKVDKKIEPRGPTIKPCPPCKCPA | | |
| | PNAAGGPSVFIFPPKIKDVLMISLSPIVTCVV | | |
| | VDVSEDDPDVQISWFVNNVEVHTAQTQTH | | |
| | REDYNSTLRVVSALPIQHQDWMSGKEFKC | | |
| | KVNNKDLGAPIERTISKPKGSVRAPQVYVL | | |
| | PPPEKEMTKKQVSLTCLVKDFMPEDIYVEW | | |
| | TNNGKTELNYKNTEPVLKSDGSYFMYSKLT | | |
| | VEKKNWVERNSYSCSVVHEGLHNHHTTKS | | |
| | FSRTPGGGGGSGGGGSGGGGSGGGGSQVQ | | |
| | LVQSGAEVKKPGASVKVSCKASGDTFTRY | | |
| | YVHWVRQAPGQGLEWMGIINPSGGYASYA | | |
| | QKFQGRVTMTRDTSTSTVYMELSSLRSEDT | | |
| | AVYYCAAGLFIWGQGTLVTVSSASGGGGS | | |
| | GGGGSGGGGSHASDIQMTQSPSSLSASVGD | | |
| | RVTITCRASQSIGRWLAWYQQKPGKAPKLL | | |
| | IYSASNLETGVPSRFSGSGSGTDFTLTISSLQ | | |
| | PEDFATYYCQQYNRFPVTFGPGTKVDIK | | |
| PEP005631 | EVQLLESGGGLVQPGGSLRLSCAASGFSFSS | | 777 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SASTKGPSVFPLAPSSKSTSGGTAALGCLVK | | |
| | DYFPEPVTVSWNSGALTSGVHTFPAVLQSS | | |
| | GLYSLSSVVTVPSSSLGTQTYICNVNHKPSN | | |
| | TKVDKKVEPKSCDKTHTCPPCPAPEAAGGP | | |
| | SVFLFPPKPKDTLMISRTPEVTCVVVDVSHE | | |
| | DPEVKFNWYVDGVEVHNAKTKPREEQYNS | | |
| | TYRVVSVLTVLHQDWLNGKEYKCKVSNK | | |
| | ALGAPIEKTISKAKGQPREPQVCTLPPSRDE | | |
| | LTKNQVSLSCAVKGFYPSDIAVEWESNGQP | | |
| | ENNYKTTPPVLDSDGSFFLVSKLTVDKSRW | | |
| | QQGNVFSCSVMHEALHNHYTQKSLSLSPG | | |
| | GGGSGGGGSGGGGSGGGGSQVQLVQSGAE | | |
| | VKKPGASVKVSCKASGDTFTRYYVHWVRQ | | |
| | APGQGLEWMGIINPSGGYASYAQKFQGRV | | |
| | TMTRDTSTSTVYMELSSLRSEDTAVYYCAA | | |
| | GLFIWGQGTLVTVSSASGGGGSGGGGSGG | | |
| | GGSHASDIQMTQSPSSLSASVGDRVTITCRA | | |
| | SQSIGRWLAWYQQKPGKAPKLLIYSASNLE | | |
| | TGVPSRFSGSGSGTDFTLTISSLQPEDFATYY | | |
| | CQQYNRFPVTFGPGTKVDIK | | |
| PEP005634 | EVQLLESGGGLVQPGGSLRLSCAASGFSFSS | | 778 |
| | YTMSWVRQAPGKGLEWVATISGGGRDIYY | | |
| | PDSVKGRFTISRDNSKNTLYLQMNSLRAED | | |
| | TAVYYCVLLTGRVYFALDSWGQGTLVTVS | | |
| | SASTKGPSVFPLAPSSKSTSGGTAALGCLVK | | |
| | DYFPEPVTVSWNSGALTSGVHTFPAVLQSS | | |
| | GLYSLSSVVTVPSSSLGTQTYICNVNHKPSN | | |
| | TKVDKKVEPKSCDKTHTCPPCPAPEAAGGP | | |
| | SVFLFPPKPKDTLMISRTPEVTCVVVDVSHE | | |
| | DPEVKFNWYVDGVEVHNAKTKPREEQYNS | | |
| | TYRVVSVLTVLHQDWLNGKEYKCKVSNK | | |
| | ALGAPIEKTISKAKGQPREPQVCTLPPSRDE | | |
| | LTKNQVSLSCAVKGFYPSDIAVEWESNGQP | | |
| | ENNYKTTPPVLDSDGSFFLVSKLTVDKSRW | | |
| | QQGNVFSCSVMHEALHNHYTQKSLSLSPG | | |
| | GGGSGGGGSGGGGSGGGGSQVQLVQSGAE | | |
| | VKKPGASVKVSCKASGYTFTRYYMHWVR | | |
| | QAPGQGLEWMGIINPRAGYTSYALKFQGR | | |
| | VTMTRDTSTSTVYMELSSLRSEDTAVYYCT | | |
| | SGWDVWGQGTLVTVSSASGGGGSGGGGS | | |
| | GGGGSHASDIQMTQSPSSLSASVGDRVTITC | | |
| | RASQSISTWLAWYQQKPGKAPKLLIYAASS | | |
| | LDSGVPSRFSGSGSGTDFTLTISSLQPEDFAT | | |
| | YYCQQSYSFPVTFGQGTKVEIK | | |
| PEP005789 | MSTSTQVQLVESGGGVVQPGRSLRLDCKAS | Nivolumab- | 779 |
| | GITFSNSGMHWVRQAPGKGLEWVAVIWYD | scFV | |
| | GSKRYYADSVKGRFTISRDNSKNTLFLQMN | | |
| | SLRAEDTAVYYCATNDDYWGQGTLVTVSS | | |
| | ASGGGGSGGGGSGGGGSHASEIVLTQSPAT | | |
| | LSLSPGERATLSCRASQSVSSYLAWYQQKP | | |
| | GQAPRLLIYDASNRATGIPARFSGSGSGTDF | | |
| | TLTISSLEPEDFAVYYCQQSSNWPRTFGQGT | | |
| | KVEIKEQKLISEEDLGSGLNDIFEAQKIEWH | | |
| | EGKPIPNPLLGLDSTNA | | |
| PEP005790 | MSTSTQVQLVQSGVEVKKPGASVKVSCKA | Pembrolizumab- | 780 |
| | SGYTFTNYYMYWVRQAPGQGLEWMGGIN | scFv | |
| | PSNGGTNFNEKFKNRVTLTTDSSTTTAYME | | |
| | LKSLQFDDTAVYYCARRDYRFDMGFDYW | | |
| | GQGTTVTVSSASGGGGSGGGGSGGGGSHA | | |
| | SEIVLTQSPATLSLSPGERATLSCRASKGVST | | |
| | SGYSYLHWYQQKPGQAPRLLIYLASYLESG | | |
| | VPARFSGSGSGTDFTLTISSLEPEDFAVYYC | | |
| | QHSRDLPLTFGGGTKVEIKEQKLISEEDLGS | | |
| | GLNDIFEAQKIEWHEGKPIPNPLLGLDSTNA | | |
| PEP005794 | MSTSTQVQLVQSGAEVKKPGASVKVSCKA | AB003178_ | 781 |
| | SGDEFTRYYVHWVRQAPGQGLEWMGIINP | 2B07v12 | |
| | SGGYASYAQKFQGRVTMTRDTSTSTVYME | | |
| | LSSLRSEDTAVYYCAAGLFIWGQGTLVTVS | | |
| | SGGGGSGGGGSGGGGSGGGGSDIQMTQSPS | | |
| | SLSASVGDRVTITCRASQSIGRWLAWYQQK | | |
| | PGKAPKLLIYSASNLETGVPSRFSGSGSGTD | | |
| | FTLTISSLQPEDFATYYCQQYNRFPVTFGPG | | |
| | TKVDIKEQKLISEEDLGSGLNDIFEAQKIEW | | |
| | HEGKPIPNPLLGLDST | | |
| PEP005797 | MSTSTQVQLVQSGAEVKKPGASVKVSCKA | AB003183_ | 782 |
| | SGDEFTRYYVHWVRQAPGQGLEWMGIQNP | 2B07v14 | |
| | SGGYASYAQKFQGRVTMTRDTSTSTVYME | | |
| | LSSLRSEDTAVYYCAAGLFIWGQGTLVTVS | | |
| | SGGGGSGGGGSGGGGSGGGGSDIQMTQSPS | | |
| | SLSASVGDRVTITCRASQSIGRELAWYQQK | | |
| | PGKAPKLLIYDASNLETGVPSRFSGSGSGTD | | |
| | FTLTISSLQPEDFATYYCQQYNRFPVTFGPG | | |
| | TKVDIKEQKLISEEDLGSGLNDIFEAQKIEW | | |
| | HEGKPIPNPLLGLDST | | |
| PEP005798 | MSTSTQVQLVQSGAEVKKPGASVKVSCKA | AB003180_ | 783 |
| | SGDEFTRYYVHWVRQAPGQGLEWMGIQNP | 2B07v13 | |
| | SGGYASYAQKFQGRVTMTRDTSTSTVYME | | |
| | LSSLRSEDTAVYYCAAGLFIWGQGTLVTVS | | |
| | SGGGGSGGGGSGGGGSGGGGSDIQMTQSPS | | |
| | SLSASVGDRVTITCRASQSIGRWLAWYQQK | | |
| | PGKAPKLLIYSASNLETGVPSRFSGSGSGTD | | |
| | FTLTISSLQPEDFATYYCQQYNRFPVTFGPG | | |
| | TKVDIKEQKLISEEDLGSGLNDIFEAQKIEW | | |
| | HEGKPIPNPLLGLDST | | |
| PEP005801 | MSTSTQVQLVQSGAEVKKPGASVKVSCKA | AB003174_ | 784 |
| | SGDTFTRYYVHWVRQAPGQGLEWMGIINP | 2B07v11 | |
| | SGGYASYAQKFQGRVTMTRDTSTSTVYME | | |
| | LSSLRSEDTAVYYCAAGLFIWGQGTLVTVS | | |
| | SGGGGSGGGGSGGGGSGGGGSDIQMTQSPS | | |
| | SLSASVGDRVTITCRASQSIGRELAWYQQK | | |
| | PGKAPKLLIYSASNLETGVPSRFSGSGSGTD | | |
| | FTLTISSLQPEDFATYYCQQYNRFPVTFGPG | | |
| | TKVDIKEQKLISEEDLGSGLNDIFEAQKIEW | | |
| | HEGKPIPNPLLGLDST | | |
| PEP005803 | MSTSTQVQLVQSGAEVKKPGASVKVSCKA | AB002328_ | 785 |
| | SGDTFTRYYVHWVRQAPGQGLEWMGIINP | 2B07v4 | |
| | SGGYASYAQKFQGRVTMTRDTSTSTVYME | | |
| | LSSLRSEDTAVYYCAAGLFIWGQGTLVTVS | | |
| | SGGGGSGGGGSGGGGSGGGGSDIQMTQSPS | | |
| | SLSASVGDRVTITCRASQSIGRWLAWYQQK | | |
| | PGKAPKLLIYSASNLETGVPSRFSGSGSGTD | | |
| | FTLTISSLQPEDFATYYCQQYNRFPVTFGPG | | |
| | TKVDIKEQKLISEEDLGSGLNDIFEAQKIEW | | |
| | HEGKPIPNPLLGLDST | | |
| PEP005817 | MSTSTQVQLVQSGAEVKKPGASVKVSCKA | AB003155_ | 786 |
| | SGETFTRYYVHWVRQAPGQGLEWMGIINPS | 2B07v10 | |
| | GGYASYAQKFQGRVTMTRDTSTSTVYMEL | | |
| | SSLRSEDTAVYYCAAGLFIWGQGTLVTVSS | | |
| | GGGGSGGGGSGGGGSGGGGSDIQMTQSPSS | | |
| | LSASVGDRVTITCRASQSIGRWLAWYQQKP | | |
| | GKAPKLLIYSASNLETGVPSRFSGSGSGTDF | | |
| | TLTISSLQPEDFATYYCQQYNRFPVTFGPGT | | |
| | KVDIKEQKLISEEDLGSGLNDIFEAQKIEWH | | |
| | EGKPIPNPLLGLDST | | |
| PEP005840 | MSTSTQVQLVQSGAEVKKPGASVKVSCKA | AB003012_ | 787 |
| | SGYTFTRYYMHWVRQAPGQGLEWMGIINP | 7A04v7 | |
| | RAGYTSYALKFQGRVTMTRDTSTSTVYME | | |
| | LSSLRSEDTAVYYCTSGWDVWGQGTLVTV | | |
| | SSGGGGSGGGGSGGGGSGGGGSDIQMTQSP | | |
| | SSLSASVGDRVTITCRASQSISTWLAWYQQ | | |
| | KPGKAPKLLIYAASSLDEGVPSRFSGSGSGT | | |
| | DFTLTISSLQPEDFATYYCQQSYSFPVTFGQ | | |
| | GTKVEIKEQKLISEEDLGSGLNDIFEAQKIE | | |
| | WHEGKPIPNPLLGLDST | | |
| PEP005842 | MSTSTQVQLVQSGAEVKKPGASVKVSCKA | AB002365_ | 788 |
| | SGYTFTRYYMHWVRQAPGQGLEWMGIINP | 7A04v2 | |
| | RAGYTSYALKFQGRVTMTRDTSTSTVYME | | |
| | LSSLRSEDTAVYYCTSGWDVWGQGTLVTV | | |
| | SSGGGGSGGGGSGGGGSGGGGSDIQMTQSP | | |
| | SSLSASVGDRVTITCRASQSISTWLAWYQQ | | |
| | KPGKAPKLLIYAASSLDSGVPSRFSGSGSGT | | |
| | DFTLTISSLQPEDFATYYCQQSYSFPVTFGQ | | |
| | GTKVEIKEQKLISEEDLGSGLNDIFEAQKIE | | |
| | WHEGKPIPNPLLGLDST | | |
| PEP005843 | MSTSTQVQLVQSGAEVKKPGASVKVSCKA | AB003007_ | 789 |
| | SGYTFTRYYMHWVRQAPGQGLEWMGIINP | 7A04v6 | |
| | RAGYTSYALKFQGRVTMTRDTSTSTVYME | | |
| | LSSLRSEDTAVYYCTSGWEVWGQGTLVTV | | |
| | SSGGGGSGGGGSGGGGSGGGGSDIQMTQSP | | |
| | SSLSASVGDRVTITCRASQSISTWLAWYQQ | | |
| | KPGKAPKLLIYAASSLDEGVPSRFSGSGSGT | | |
| | DFTLTISSLQPEDFATYYCQQSYSFPVTFGQ | | |
| | GTKVEIKEQKLISEEDLGSGLNDIFEAQKIE | | |
| | WHEGKPIPNPLLGLDST | | |
| PEP005845 | MSTSTQVQLVQSGAEVKKPGASVKVSCKA | AB003005_ | 790 |
| | SGYTFTRYYMHWVRQAPGQGLEWMGIIQP | 7A04v5 | |
| | RAGYTSYALKFQGRVTMTRDTSTSTVYME | | |
| | LSSLRSEDTAVYYCTSGWDVWGQGTLVTV | | |
| | SSGGGGSGGGGSGGGGSGGGGSDIQMTQSP | | |
| | SSLSASVGDRVTITCRASQSISTWLAWYQQ | | |
| | KPGKAPKLLIYAASSLDEGVPSRFSGSGSGT | | |
| | DFTLTISSLQPEDFATYYCQQSYSFPVTFGQ | | |
| | GTKVEIKEQKLISEEDLGSGLNDIFEAQKIE | | |
| | WHEGKPIPNPLLGLDST | | |
| PEP005847 | MSTSTQVQLVQSGAEVKKPGASVKVSCKA | AB003209_ | 791 |
| | SGETFTNYYIHWVRQAPGQGLEWMGIIDPR | 2B05v4 | |
| | AGYTSYALKFQGRVTMTRDTSTSTVYMEL | | |
| | SSLRSEDTAVYYCAGGWEDWGQGTLVTVS | | |
| | SGGGGSGGGGSGGGGSGGGGSDIQMTQSPS | | |
| | SLSASVGDRVTITCRISQSISSFLAWYQQKP | | |
| | GKAPKLLIYSASSLQSGVPSRFSGSGSGTDF | | |
| | TLTISSLQPEDFATYYCQQSFTSPITFGQGTR | | |
| | LEIKEQKLISEEDLGSGLNDIFEAQKIEWHE | | |
| | GKPIPNPLLGLDST | | |
| PEP005853 | MSTSTQVQLVQSGAEVKKPGASVKVSCKA | AB003200_ | 792 |
| | SGETFTNYYIHWVRQAPGQGLEWMGVINP | 2B05v1 | |
| | RAGYTSYALKFQGRVTMTRDTSTSTVYME | | |
| | LSSLRSEDTAVYYCAGGWEDWGQGTLVTV | | |
| | SSGGGGSGGGGSGGGGSGGGGSDIQMTQSP | | |
| | SSLSASVGDRVTITCRISQSISSWLAWYQQK | | |
| | PGKAPKLLIYSASSLQSGVPSRFSGSGSGTD | | |
| | FTLTISSLQPEDFATYYCQQSFTSPITFGQGT | | |
| | RLEIKEQKLISEEDLGSGLNDIFEAQKIEWHE | | |
| | GKPIPNPLLGLDST | | |
| PEP005860 | MSTSTQVQLVQSGAEVKKPGASVKVSCKA | AB003021_ | 793 |
| | SGFTFTRYYMHWVRQAPGQGLEWMGVINP | 2A11v7 | |
| | SGGYATYAQKFQGRVTMTRDTSTSTVYME | | |
| | LSSLRSEDTAVYYCASGWDVWGQGTLVTV | | |
| | SSGGGGSGGGGSGGGGSGGGGSDIQMTQSP | | |
| | SSLSASVGDRVTITCRASQSISSWLAWYQQ | | |
| | KPGKAPKLLIYATSTLESGVPSRFSGSGSGT | | |
| | DFTLTISSLQPEDFATYYCQQSYSSPVTFGQ | | |
| | GTKVEIKEQKLISEEDLGSGLNDIFEAQKIE | | |
| | WHEGKPIPNPLLGLDST | | |
| PEP005863 | MSTSTQVQLVQSGAEVKKPGASVKVSCKA | AB003201_ | 794 |
| | SGGTFTNYYIHWVRQAPGQGLEWMGIIDPR | 2B05v2 | |
| | AGYTSYALKFQGRVTMTRDTSTSTVYMEL | | |
| | SSLRSEDTAVYYCAGGWEDWGQGTLVTVS | | |
| | SGGGGSGGGGSGGGGSGGGGSDIQMTQSPS | | |
| | SLSASVGDRVTITCRISQSISSWLAWYQQKP | | |
| | GKAPKLLIYSASSLQSGVPSRFSGSGSGTDF | | |
| | TLTISSLQPEDFATYYCQQSFTSPITFGQGTR | | |
| | LEIKEQKLISEEDLGSGLNDIFEAQKIEWHE | | |
| | GKPIPNPLLGLDST | | |
| PEP005867 | MSTSTQVQLVQSGAEVKKPGASVKVSCKA | AB002427_ | 795 |
| | SGHTFTRYYMHWVRQAPGQGLEWMGIINP | 2A11v5 | |
| | SGGYATYAQKFQGRVTMTRDTSTSTVYME | | |
| | LSSLRSEDTAVYYCASGLFIWGQGTLVTVS | | |
| | SGGGGSGGGGSGGGGSGGGGSDIQMTQSPS | | |
| | SLSASVGDRVTITCRASQSINSWLAWYQQK | | |
| | PGKAPKLLIYATSTLESGVPSRFSGSGSGTD | | |
| | FTLTISSLQPEDFATYYCQQSYRFPVTFGQG | | |
| | TKVEIKEQKLISEEDLGSGLNDIFEAQKIEW | | |
| | HEGKPIPNPLLGLDST | | |
| PEP005868 | MSTSTQVQLVQSGAEVKKPGASVKVSCKA | AB002413_ | 796 |
| | SGHTFTRYYMHWVRQAPGQGLEWMGIINP | 2A11v3 | |
| | SGGYATYAQKFQGRVTMTRDTSTSTVYME | | |
| | LSSLRSEDTAVYYCASGLFIWGQGTLVTVS | | |
| | SGGGGSGGGGSGGGGSGGGGSDIQMTQSPS | | |
| | SLSASVGDRVTITCRASQSINSWLAWYQQK | | |
| | PGKAPKLLIYATSTLESGVPSRFSGSGSGTD | | |
| | FTLTISSLQPEDFATYYCQQYYRFPVTFGQG | | |
| | TKVEIKEQKLISEEDLGSGLNDIFEAQKIEW | | |
| | HEGKPIPNPLLGLDST | | |
| PEP005869 | MSTSTQVQLVQSGAEVKKPGASVKVSCKA | AB002417_ | 797 |
| | SGHTFTRYYMHWVRQAPGQGLEWMGIINP | 2A11v4 | |
| | SGGYATYAQKFQGRVTMTRDTSTSTVYME | | |
| | LSSLRSEDTAVYYCASGLFIWGQGTLVTVS | | |
| | SGGGGSGGGGSGGGGSGGGGSDIQMTQSPS | | |
| | SLSASVGDRVTITCRASQSINSWLAWYQQK | | |
| | PGKAPKLLIYATSTLESGVPSRFSGSGSGTD | | |
| | FTLTISSLQPEDFATYYCQQYYSFPVTFGQG | | |
| | TKVEIKEQKLISEEDLGSGLNDIFEAQKIEW | | |
| | HEGKPIPNPLLGLDST | | |
| PEP005880 | MSTSTQVQLVQSGAEVKKPGASVKVSCKA | AB002864_ | 798 |
| | SGHTFTRYYMHWVRQAPGQGLEWMGVIN | 2A11v6 | |
| | PSGGYATYAQKFQGRVTMTRDTSTSTVYM | | |
| | ELSSLRSEDTAVYYCASGWDVWGQGTLVT | | |
| | VSSGGGGSGGGGSGGGGSGGGGSDIQMTQ | | |
| | SPSSLSASVGDRVTITCRASQSINSWLAWYQ | | |
| | QKPGKAPKLLIYATSTLESGVPSRFSGSGSG | | |
| | TDFTLTISSLQPEDFATYYCQQSYSSPVTFG | | |
| | QGTKVEIKEQKLISEEDLGSGLNDIFEAQKIE | | |
| | WHEGKPIPNPLLGLDST | | |
| PEP005887 | MSTSTQVQLVQSGAEVKKPGASVKVSCKA | AB003202_ | 799 |
| | SGSTFTNYYIHWVRQAPGQGLEWMGIIDPR | 2B05v3 | |
| | AGYTSYALKFQGRVTMTRDTSTSTVYMEL | | |
| | SSLRSEDTAVYYCAGGWEDWGQGTLVTVS | | |
| | SGGGGSGGGGSGGGGSGGGGSDIQMTQSPS | | |
| | SLSASVGDRVTITCRISQSISSFLAWYQQKP | | |
| | GKAPKLLIYSASSLQSGVPSRFSGSGSGTDF | | |
| | TLTISSLQPEDFATYYCQQSFTSPITFGQGTR | | |
| | LEIKEQKLISEEDLGSGLNDIFEAQKIEWHE | | |
| | GKPIPNPLLGLDST | | |
| PEP006177 | QVQLVESGGGVVQPGRSLRLDCKASGITFS | | 800 |
| | NSGMHWVRQAPGKGLEWVAVIWYDGSKR | | |
| | YYADSVKGRFTISRDNSKNTLFLQMNSLRA | | |
| | EDTAVYYCATNDDYWGQGTLVTVSSAKTT | | |
| | APSVYPLAPVCGDTTGSSVTLGCLVKGYFP | | |
| | EPVTLTWNSGSLSSGVHTFPAVLQSDLYTLS | | |
| | SSVTVTSSTWPSQSITCNVAHPASSTKVDKK | | |
| | IEPRGPTIKPCPPCKCPAPNAAGGPSVFIFPP | | |
| | KIKDVLMISLSPIVTCVVVDVSEDDPDVQIS | | |
| | WFVNNVEVHTAQTQTHREDYNSTLRVVSA | | |
| | LPIQHQDWMSGKEFKCKVNNKDLGAPIERT | | |
| | ISKPKGSVRAPQVYVLPPPEEEMTKKQVTL | | |
| | TCMVTDFMPEDIYVEWTNNGKTELNYKNT | | |
| | EPVLDSDGSYFMYSDLRVEKKNWVERNSY | | |
| | SCSVVHEGLHNHHTTESFSRTPGGGGGSGG | | |
| | GGSGGGGSAPTSSSTKKTQLQLEHLLLDLQ | | |
| | MILNGINNYKNPKLTRMLTFKFYMPKKATE | | |
| | LKHLQCLEEELKPLEEVLNLAQSKNFHLRP | | |
| | RDLISNINVIVLELKGSETTFMCEYADETATI | | |
| | VEFLNRWITFCQSIISTLT | | |

### EXAMPLES

The following examples are illustrative and non-limiting to the scope of the devices, methods, systems, and kits described herein.

### Example 1

### Isolation of a Set of Dual-Binding Antibodies (DBAs) that Bind Human PD-1 and Human IL-2

This example describes the isolation of sensor domains of the present disclosure, specifically, a set of DBAs that bind human PD-1 and human IL-2. Anti-PD-1 and anti-IL-2 DBAs were isolated from a TUMBLER™ antibody phage display library (Distributed Bio, Inc.). The antibody phage display library was constructed to incorporate the heavy chain CDR1, heavy chain CDR2, and light chain diversity of the SUPERHUMAN™ 2.0 antibody library combined with 10 heavy chain CDR3 sequences from PD-1 binding antibodies (SEQ ID NO: 11-SEQ ID NO: 20).

TABLE 3
HC-CDR3 of PD-1 binders

| SEQ ID NO | Sequence | Description |
|---|---|---|
| SEQ ID NO: 11 | CAAGLFIW | HC-CDR3 of PD-1 binder |
| SEQ ID NO: 12 | CAGGWLDW | HC-CDR3 of PD-1 binder |
| SEQ ID NO: 13 | CARDHLGGSYQPW | HC-CDR3 of PD-1 binder |
| SEQ ID NO: 14 | CARDLVGVSPGIN | HC-CDR3 of PD-1 binder |
| | YVPRYYYYYYGMD | |
| | VW | |
| SEQ ID NO: 15 | CARDTGLGYYYGS | HC-CDR3 of PD-1 binder |
| | GDFDYW | |
| SEQ ID NO: 16 | CARSGYSYGYYFD | HC-CDR3 of PD-1 binder |
| | YW | |
| SEQ ID NO: 17 | CARTGGYPAIDSW | HC-CDR3 of PD-1 binder |
| SEQ ID NO: 18 | CASGWDVW | HC-CDR3 of PD-1 binder |
| SEQ ID NO: 19 | CASSPLQWVDVW | HC-CDR3 of PD-1 binder |
| SEQ ID NO: 20 | CTSGMDVW | HC-CDR3 of PD-1 binder |

This library was subjected to four rounds of selection with standard protocols. In brief, the phage library was incubated with the antigen, then captured on magnetic beads and washed on a KINGFISHER™ magnetic particle processor, eluted form the magnetic beads and amplified by passaging in E. coli. Round 1 was incubated with 50 nM human PD-1-His fusion (R&D Systems, Prod. Num. 8986-PD) and captured with TRIS NTA Biotin (Sigma-Aldrich Prod. Num. 75543) and streptavidin magnetic beads. Round 2 was incubated with 100 nM biotinylated IL-2 (Creative Biomart, Prod. Num. IL2-501H, biotinylated using standard protocols) and captured on streptavidin magnetic beads. Round 3 was incubated with 50 nM cynomolgus PD-1-Fc fusion (R&D Systems, Prod. Num. 8578-PD) and captured on protein G magnetic beads. Round 4 was incubated with 50 nM biotinylated human IL-2 and captured on streptavidin magnetic beads. The final selection was plated as single colonies and 380 colonies picked for Sanger sequencing. One hundred and fifty-one unique clones were chosen for expression. The scFv sequence for each clone was codon-optimized for E. coli expression and the corresponding DNA sequences sent to Integrated DNA Technologies, Inc. (IDT) for synthesis as GBLOCKS® with a T7 promoter, a translation initiation site and a T7 terminator. Protein from each GBLOCK® encoding an scFv was expressed using the PUREXPRESS® In vitro Protein Synthesis Kit (New England Biolabs, Inc., Prod. Num. E6800). The PUREXPRESS® scFv proteins were used directly in homogeneous time-resolved fluorescence (HTRF®) binding assays and cell-based functional assays. Each scFv was tested for binding to PD-1 and to human IL-2. Eighty-one of the antibodies showed dual-binding activity for both PD-1 and IL-2 and a summary of fluorescence signal values of binding curves is shown in TABLE 5. To examine the ability of DBA binding domains to block IL-2 receptor binding, V5-tagged DBA scFvs were serially diluted in a 384 well plate. Europium-labeled Streptavidin, biotin-labeled IL-2 (Acro Biosystems, Prod. Num. IL2-H82E4), IL-2 Receptor beta (Fc-IL2RB) (Acro Biosystems, Prod. Num. ILB-H5253), and APC-labeled anti-Fc antibody. Plates were incubated at room temperature for 2 hours, and the HTRF® signal was read on an ENVISION™ (Perkin Elmer) as a measure of IL-2: IL2RB binding. Four scFvs (SEQ ID NO: 31-SEQ ID NO: 34) bound PD-1, bound IL-2 and blocked binding of IL-2 to IL-2RB (TABLE 4).

TABLE 4
DBAs and Controls

| SEQ ID | | |
|---|---|---|
| NO | Sequence | Description |
| SEQ ID | QVQLVQSGAEVKKPGVSVKVSCKASGYTF | DBA capable |
| NO: 31 | PRSYIHWVRQAPGQGLEWMGWINPHSGDT | of binding |
| | YYAQNFQGRVTMTRDTSTSTVYMELSSLR | a PD-1 |
| | SEDTAVYYCARDTGLGYYYGSGDFDYWGQ | marker and |
| | GTLVTVSSASGGGGSGGGGSGGGGSHASD | IL-2 thera- |
| | IQMTQSPSSLSASVGDRVTITCRASQSIS | peutic |
| | RYLNWYQQKPGKAPKLLIYTASSLQSGVP | |
| | SRFSGSGSGTDFTLTISSLQPEDFATYYC | |
| | QQANRFPLTFGPGTKVDIK | |
| SEQ ID | QVQLVQSGAEVKKPGASVKVSCKASGYTF | DBA capable |
| NO: 32 | PRYHIHWVRQAPGQGLEWMGMINPSGGTT | of binding |
| | TYAQKFQGRVTMTRDTSTSTVYMELSSLR | a PD-1 |
| | SEDTAVYYCARDTGLGYYYGSGDFDYWGQ | marker and |
| | GTLVTVSSASGGGGSGGGGSGGGGSHASD | IL-2 thera- |
| | IQMTQSPSSLSASVGDRVTITCRASQSIS | peutic |
| | SWLAWYQQKPGKAPKLLIYAASSLQSGVP | |
| | SRFSGSGSGTDFTLTISSLQPEDFATYYC | |
| | QQSHSFPLTFGGGTKVEIK | |
| SEQ ID | QVQLVQSGAEVKKPGASVKVSCKASGYTF | DBA capable |
| NO: 33 | TRYYIHWVRQAPGQGLEWMGWINAYNGDT | of binding |
| | NYAQKLQGRVTMTRDTSTSTVYMELSSLR | a PD-1 |
| | SEDTAVYYCARDSYYYDSFDYWGQGTLVT | marker and |
| | VSSASGGGGSGGGGSGGGGSHASDIQMTQ | IL-2 thera- |
| | SPSSLSASVGDRVTITCRASQTITDWLAW | peutic |
| | YQQKPGKAPKLLIYGASNLQGGVPSRFSG | |
| | SGSGTDFTLTISSLQPEDFATYYCQQYYS | |
| | SWTFGQGTKVEIK | |
| SEQ ID | QVQLVQSGAEVKKPGASVKVSCKASGYTF | DBA capable |
| NO: 34 | TSYYMHWVRQAPGQGLEWMGIINPSDGST | of binding |
| | TYAQSFQGRVTMTRDTSTSTVYMELSSLR | a PD-1 |
| | SEDTAVYYCASGWDVWGQGTLVTVSSASG | marker and |
| | GGGSGGGGSGGGGSHASDIVMTQSPDSLA | IL-2 thera- |
| | VSLGERATINCKSSQSVFSSANNKNYLAW | peutic |
| | YQQKPGQPPKLLIYWASTRESGVPDRESG | |
| | SGSGTDFTLTISSLQAEDVAVYYCQQYFG | |
| | TPVTFGGGTKVEIK | |

TABLE 5

| Name | PD1 Binding | IL2 Binding | IL2RB Blocking |
|---|---|---|---|
| No DNA | 5 | 6 | 1,490 |
| PD1-IL2-R01-H08 | 576 | −9 | 1,829 |
| PD1-IL2-R01-H09 | 1,015 | 131 | 1,772 |
| PD1-IL2-R02-A03 | 1,508 | 635 | 1,714 |
| PD1-IL2-R02-A04 | 909 | 978 | 1,618 |
| PD1-IL2-R02-A05 | 1,557 | 23 | 1,735 |
| PD1-IL2-R02-A06 | 357 | 515 | 1,772 |
| PD1-IL2-R02-A08 | 995 | 520 | 1,612 |
| PD1-IL2-R02-A09 | 1,421 | 1,470 | 1,495 |
| PD1-IL2-R02-A10 | 500 | 838 | 1,847 |
| PD1-IL2-R02-A11 | 1,625 | 1,559 | 1,783 |
| PD1-IL2-R02-A12 | 1,725 | 130 | 1,586 |
| PD1-IL2-R02-B01 | 746 | 1,077 | 1,516 |
| PD1-IL2-R02-B02 | 1,740 | 1,107 | 1,849 |
| PD1-IL2-R02-B04 | 11 | 2,346 | 1,536 |
| PD1-IL2-R02-B05 | 1,665 | 2,489 | 1,613 |
| PD1-IL2-R02-B06 | 1,527 | 32 | 1,605 |
| PD1-IL2-R02-B07 | 1,685 | 628 | 1,814 |
| PD1-IL2-R02-B08 | 1,446 | 92 | 1,680 |
| PD1-IL2-R02-B10 | 211 | 343 | 1,607 |
| PD1-IL2-R02-B11 | 1,426 | 915 | 1,509 |
| PD1-IL2-R02-B12 | 1,264 | 316 | 1,762 |
| PD1-IL2-R02-C01 | 1,463 | 296 | 1,743 |
| PD1-IL2-R02-C02 | 1,299 | 298 | 1,069 |
| (SEQ ID NO: 31) | | | |
| PD1-IL2-R02-C03 | 1,383 | 293 | 1,211 |
| (SEQ ID NO: 32) | | | |
| PD1-IL2-R02-C04 | 1,622 | 575 | 1,857 |
| PD1-IL2-R02-C06 | 1,376 | 34 | 1,684 |
| PD1-IL2-R02-C07 | 34 | 87 | 1,607 |
| PD1-IL2-R02-C08 | 1,468 | 619 | 1,671 |
| PD1-IL2-R02-C10 | 174 | 256 | 1,757 |
| PD1-IL2-R02-C12 | 1,367 | 340 | 1,723 |
| PD1-IL2-R02-D01 | 1,421 | 68 | 1,614 |
| PD1-IL2-R02-D02 | 1,473 | 539 | 1,726 |
| PD1-IL2-R06-A10 | 1,269 | 9 | 1,796 |
| PD1-IL2-R06-A11 | 1,376 | 34 | 1,762 |
| PD1-IL2-R06-A12 | 1,305 | 7 | 1,681 |
| PD1-IL2-R06-B01 | 10 | 2,109 | 1,307 |
| PD1-IL2-R06-B02 | 1,666 | 15 | 1,799 |
| PD1-IL2-R06-B03 | 923 | 4 | 1,661 |
| PD1-IL2-R06-B04 | 1,782 | 28 | 1,666 |
| PD1-IL2-R06-B06 | 1,223 | 17 | 1,648 |
| PD1-IL2-R06-B08 | 1,777 | 1,160 | 1,738 |
| PD1-IL2-R06-B10 | 13 | 31 | 1,847 |
| PD1-IL2-R06-B11 | 1,534 | 24 | 1,699 |
| PD1-IL2-R06-B12 | 822 | 1,125 | 1,604 |
| PD1-IL2-R06-C02 | 1,667 | 26 | 1,671 |
| PD1-IL2-R06-C04 | 1,491 | 7 | 1,759 |
| PD1-IL2-R06-C08 | 1,448 | 8 | 1,693 |
| PD1-IL2-R06-C09 | 1,158 | 1,525 | 1,602 |
| PD1-IL2-R06-C11 | 1,879 | −2 | 1,785 |
| PD1-IL2-R06-C12 | 1,669 | 1,998 | 1,033 |
| PD1-IL2-R06-D02 | 280 | 432 | 1,677 |
| PD1-IL2-R06-D03 | 9 | 93 | 1,606 |
| PD1-IL2-R06-D05 | 505 | −3 | 1,786 |
| PD1-IL2-R06-D07 | 1,577 | 24 | 1,820 |
| PD1-IL2-R06-D10 | 1,751 | 49 | 1,719 |
| PD1-IL2-R06-D11 | 405 | 593 | 1,576 |
| PD1-IL2-R06-D12 | 1,024 | 1,423 | 1,649 |
| PD1-IL2-R06-E01 | 1,628 | 3 | 1,724 |
| PD1-IL2-R06-E02 | 1,554 | 16 | 1,598 |
| PD1-IL2-R06-E04 | 50 | 247 | 1,108 |
| (SEQ ID NO: 33) | | | |
| PD1-IL2-R06-E05 | 1,364 | 14 | 1,734 |
| PD1-IL2-R06-E06 | 1,627 | 15 | 1,735 |
| PD1-IL2-R06-E07 | 1,801 | 12 | 1,698 |
| PD1-IL2-R06-E09 | 1,467 | 11 | 1,511 |
| PD1-IL2-R06-E11 | 1,805 | 294 | 1,767 |
| PD1-IL2-R06-E12 | 4 | −7 | 1,735 |
| PD1-IL2-R06-F01 | 196 | 280 | 1,629 |
| PD1-IL2-R06-F03 | 1,377 | 28 | 1,642 |
| PD1-IL2-R06-F04 | 26 | 779 | 1,726 |
| PD1-IL2-R06-F05 | 1,493 | 18 | 1,625 |
| PD1-IL2-R06-F06 | 1,577 | 46 | 1,595 |
| PD1-IL2-R06-F07 | 1,544 | 335 | 1,682 |
| PD1-IL2-R06-F08 | 1,570 | 9 | 1,780 |
| PD1-IL2-R06-F09 | 30 | 41 | 1,776 |
| PD1-IL2-R06-F10 | 1,745 | 24 | 1,607 |
| PD1-IL2-R06-F11 | 1,586 | 12 | 1,574 |
| PD1-IL2-R06-F12 | 623 | 8 | 1,645 |
| PD1-IL2-R06-G01 | 130 | 184 | 1,640 |
| PD1-IL2-R06-G02 | 1,754 | 20 | 1,623 |
| PD1-IL2-R06-G04 | 1,348 | 13 | 1,596 |
| PD1-IL2-R06-G05 | 1,382 | 10 | 1,846 |
| PD1-IL2-R06-G06 | 1,383 | 4 | 1,744 |
| PD1-IL2-R06-G08 | 1,708 | 124 | 1,533 |
| PD1-IL2-R06-G09 | 557 | 756 | 1,527 |
| PD1-IL2-R06-G10 | 1,595 | 35 | 1,703 |
| PD1-IL2-R06-G11 | 1,469 | 17 | 1,709 |
| PD1-IL2-R06-G12 | 1,281 | 1,479 | 1,713 |
| PD1-IL2-R06-H01 | 381 | 4 | 1,647 |
| PD1-IL2-R06-H02 | 1,501 | 20 | 1,748 |
| PD1-IL2-R06-H03 | 1,132 | 1,449 | 1,617 |
| PD1-IL2-R06-H04 | 355 | 1 | 1,677 |
| PD1-IL2-R06-H05 | 1,409 | 21 | 1,561 |
| PD1-IL2-R06-H06 | 1,491 | 23 | 1,650 |
| PD1-IL2-R06-H07 | 12 | 13 | 1,701 |
| PD1-IL2-R06-H08 | 847 | 1,118 | 1,746 |
| PD1-IL2-R06-H09 | 1,732 | 22 | 1,662 |
| PD1-IL2-R06-H10 | 830 | 1,151 | 1,569 |
| PD1-IL2-R07-A03 | 1,786 | 28 | 1,511 |
| PD1-IL2-R07-A04 | 730 | 973 | 1,613 |
| PD1-IL2-R07-A05 | 477 | 663 | 1,327 |
| PD1-IL2-R07-A08 | 1,628 | 841 | 1,618 |
| PD1-IL2-R07-A09 | 1,235 | 2,040 | 910 |
| (SEQ ID NO: 34) | | | |
| PD1-IL2-R07-A10 | 1,716 | 63 | 1,518 |
| PD1-IL2-R07-B01 | 1,397 | 32 | 1,565 |
| PD1-IL2-R07-B02 | 192 | 321 | 1,634 |
| PD1-IL2-R07-B03 | 65 | 202 | 1,604 |
| PD1-IL2-R07-B04 | 1,862 | 410 | 1,527 |
| PD1-IL2-R07-B05 | 965 | 351 | 1,389 |
| PD1-IL2-R07-B06 | 1,882 | 44 | 1,497 |
| PD1-IL2-R07-B07 | 6 | 2,549 | 1,517 |
| PD1-IL2-R07-B08 | 906 | 1,047 | 1,475 |
| PD1-IL2-R07-B09 | 1,788 | 27 | 1,384 |
| PD1-IL2-R07-B10 | 18 | 19 | 1,635 |
| PD1-IL2-R07-B11 | 1,765 | 9 | 1,641 |
| PD1-IL2-R07-C01 | 230 | 367 | 1,536 |
| PD1-IL2-R07-C02 | 236 | 304 | 1,500 |
| PD1-IL2-R07-C03 | 20 | 1,347 | 1,536 |
| PD1-IL2-R07-C07 | 15 | 275 | 1,665 |
| PD1-IL2-R07-C10 | 1,064 | 317 | 1,550 |
| PD1-IL2-R07-C11 | 1,523 | 642 | 1,460 |
| PD1-IL2-R07-C12 | 1,377 | 49 | 1,707 |
| PD1-IL2-R07-D01 | 1,541 | 79 | 1,657 |
| PD1-IL2-R07-D03 | 1,483 | 33 | 1,481 |
| PD1-IL2-R07-D04 | 923 | 1,104 | 1,517 |
| PD1-IL2-R07-D06 | 1,664 | 416 | 1,734 |
| PD1-IL2-R07-D07 | 6 | 835 | 1,512 |
| PD1-IL2-R07-D10 | 1,580 | 193 | 1,572 |
| PD1-IL2-R07-D11 | 1,401 | 798 | 1,614 |
| PD1-IL2-R07-E02 | 1,473 | 992 | 1,830 |
| PD1-IL2-R07-E03 | 1,459 | 422 | 1,683 |
| PD1-IL2-R07-E05 | 512 | 913 | 1,513 |
| PD1-IL2-R07-E06 | 1,483 | 1,178 | 1,526 |
| PD1-IL2-R07-E07 | 1,181 | 1,060 | 1,524 |
| PD1-IL2-R07-E08 | 1,604 | 472 | 1,717 |
| PD1-IL2-R07-E09 | 1,733 | 23 | 1,569 |
| PD1-IL2-R07-E10 | 1,472 | 251 | 1,545 |
| PD1-IL2-R07-E11 | 1,146 | 56 | 1,777 |
| PD1-IL2-R07-E12 | 1,698 | 106 | 1,764 |
| PD1-IL2-R07-F01 | 3 | 17 | 1,529 |
| PD1-IL2-R07-F02 | 348 | 752 | 1,537 |
| PD1-IL2-R07-F03 | 1,788 | 520 | 1,750 |
| PD1-IL2-R07-F04 | 1,416 | 145 | 1,767 |
| PD1-IL2-R07-F06 | 1,422 | 438 | 1,579 |
| PD1-IL2-R07-F09 | 1,589 | 17 | 1,456 |
| PD1-IL2-R07-F10 | 24 | 19 | 1,778 |
| PD1-IL2-R07-F12 | 505 | 196 | 1,553 |
| PD1-IL2-R07-G01 | 4 | 214 | 1,560 |
| PD1-IL2-R07-G02 | 1,610 | 61 | 1,735 |
| PD1-IL2-R07-G04 | 82 | 147 | 1,600 |
| PD1-IL2-R07-G05 | 981 | 216 | 1,475 |
| PD1-IL2-R07-G06 | 860 | 512 | 1,655 |
| PD1-R04-C10 | 1,552 | 4 | 1,550 |
| PD1-R07-A05 | 653 | 19 | 1,730 |
| PD1-R07-A10 | 484 | 25 | 2,290 |
| PD1-R07-C09 | 1,911 | 20 | 2,080 |
| PD1-R07-D03 | 1,733 | 22 | 2,208 |
| PD1-R07-D05 | 1,760 | 16 | 1,578 |
| PD1-R07-D06 | 1,997 | 22 | 1,749 |
| PD1-R07-E05 | 633 | 24 | 2,246 |
| PD1-R07-G12 | 907 | 11 | 1,577 |
| PD1-R15-B02 | 1,671 | 28 | 1,797 |
| PDL1-DB03-H02 | 18 | 11 | 1,725 |
| Anti-Her2 (SEQ ID | 4 | 20 | 1,636 |
| NO: 28) | | | |

### Example 2

### Dual Binding Antibody (DBA)-Cytokine Protein Complexes

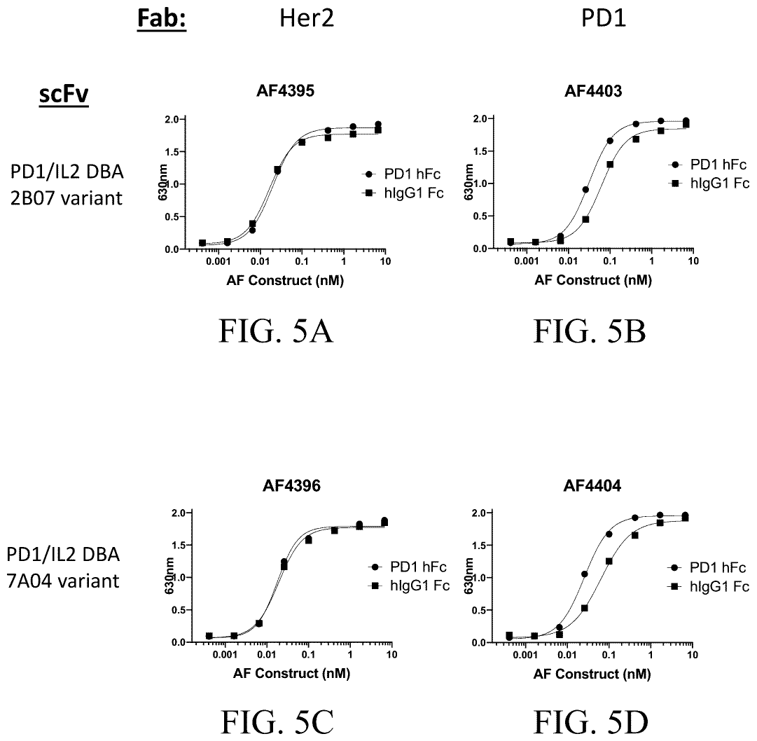

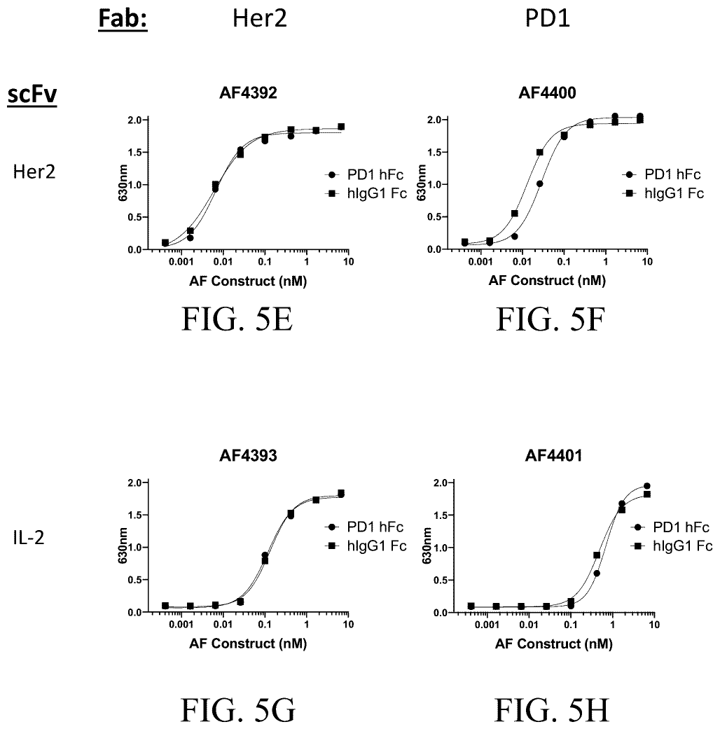

This example describes dual binding antibody (DBA)-cytokine protein complexes of the present disclosure. Various DBA-cytokine protein complexes of the present disclosure were designed to include a cytokine, a linker, and one or more dual binding antibody domains. Pictorial representations of exemplary constructs are shown in FIG. 5.

A series of DBA-cytokine protein complexes may be designed with two marker binding domains and one therapeutic domain. The DBAs used in this series, provided in TABLE 6 with sequences provided in TABLE 8, exhibit a range of affinities for the marker and the therapeutic domain. Exemplary DBA complexes are provided in TABLE 6, TABLE 9, and TABLE 10.

TABLE 6
Exemplary DBA Cytokine Protein Complexes

| SEQ ID NO | Sequences | Description |
|---|---|---|
| SEQ ID NO: 51 | QVQLVQSGAEVKKPGASVKVSCKASGYTFST | PD1- |
| | YYIHWVRQAPGQGLEWMGIINPSGGGTVYAQ | IL2_6C12_N36T_ |
| | KFQGRVTMTRDTSTSTVYMELSSLRSEDTAV | Sym_L_Long_Pep1; |
| | YYCAAGLFIWGQGTLVTVSSAKTTAPSVYPL | Symmetric DBA- |
| | APVCGDTTGSSVTLGCLVKGYFPEPVTLTWN | Cytokine Complex |
| | SGSLSSGVHTFPAVLQSDLYTLSSSVTVTSS | IgG format |
| | WTPSQSITCNVAHPASSTKVDKKIEPRGPTI | |
| | KPCPPCKCPAPNAAGGPSVFIFPPKIKDVLM | |
| | ISLSPIVTCVVVDVSEDDPDVQISWFVNNVE | |
| | VHTAQTQTHREDYNSTLRVVSALPIQHQDWM | |
| | SGKEFKCKVNNKDLGAPIERTISKPKGSVRA | |
| | PQVYVLPPPEEEMTKKQVTLTCMVTDFMPED | |
| | IYVEWTNNGKTELNYKNTEPVLDSDGSYFMY | |
| | SKLRVEKKNWVERNSYSCSVVHEGLHNHHTT | |
| | KSFSRTPGK | |
| SEQ ID NO: 52 | APTSSSTKKTQLQLEHLLLDLQMILNGINNY | PD1- |
| | KNPKLTRMLTFKFYMPKKATELKHLQCLEEE | IL2_6C12_N36T_ |
| | LKPLEEVLNLAQSKNFHLRPRDLISNINVIV | Sym_L_Long_Pep2; |
| | LELKGSETTFMCEYADETATIVEFLNRWITF | Symmetric DBA- |
| | CQSIISTLTVPGVGVPGAGVPGVGVPGGGVP | Cytokine Complex |
| | GVGVPGGGVPGAGVPGGGVPGVGVPGAGVPG | IgG format |
| | VGVPGGGDIQMTQSPSSLSASVGDRVTITCR | |
| | ASQYISSGLAWYQQKPGKAPKLLIYKASSLD | |
| | NGVPSRFSGSGSGTDFTLTISSLQPEDFATY | |
| | YCQQYERLPLTFGGGTKVEIKRADAAPTVSI | |
| | FPPSSEQLTSGGASVVCFLNNFYPKDINVKW | |
| | KIDGSERQNGVLNSWTDQDSKDSTYSMSSTL | |
| | TLTKDEYERHNSYTCEATHKTSTSPIVKSFN | |
| | RNEC | |
| SEQ ID NO: 53 | APTSSSTKKTQLQLEHLLLDLQMILNGINNY | PD1- |
| | KNPKLTRMLTFKFYMPKKATELKHLQCLEEE | IL2_6C12_N36T_ |
| | LKPLEEVLNLAQSKNFHLRPRDLISNINVIV | D68E_Sym_H_ |
| | LELKGSETTFMCEYADETATIVEFLNRWITF | Short_Pep1; |
| | CQSIISTLTGGGGSGGGGSGGGGSGGGGSQV | Symmetric DBA- |
| | QLVQSGAEVKKPGASVKVSCKASGYTFSTYY | Cytokine Complex |
| | IHWVRQAPGQGLEWMGIINPSGGGTVYAQKF | IgG format |
| | QGRVTMTRDTSTSTVYMELSSLRSEDTAVYY | |
| | CAAGLFIWGQGTLVTVSSAKTTAPSVYPLAP | |
| | VCGDTTGSSVTLGCLVKGYFPEPVTLTWNSG | |
| | SLSSGVHTFPAVLQSDLYTLSSSVTVTSSTW | |
| | PSQSITCNVAHPASSTKVDKKIEPRGPTIKP | |
| | CPPCKCPAPNAAGGPSVFIFPPKIKDVLMIS | |
| | LSPIVTCVVVDVSEDDPDVQISWFVNNVEVH | |
| | TAQTQTHREDYNSTLRVVSALPIQHQDWMSG | |
| | KEFKCKVNNKDLGAPIERTISKPKGSVRAPQ | |
| | VYVLPPPEEEMTKKQVTLTCMVTDFMPEDIY | |
| | VEWTNNGKTELNYKNTEPVLDSDGSYFMYSK | |
| | LRVEKKNWVERNSYSCSVVHEGLHNHHTTKS | |
| | FSRTPGK | |
| SEQ ID NO: 54 | DIQMTQSPSSLSASVGDRVTITCRASQYISS | PD1- |
| | GLAWYQQKPGKAPKLLIYKASSLENGVPSRF | IL2_6C12_N36T_ |
| | SGSGSGTDFTLTISSLQPEDFATYYCQQYER | D68E_Sym_H_ |
| | LPLTFGGGTKVEIKRADAAPTVSIFPPSSEQ | Short_Pep2; |
| | LTSGGASVVCFLNNFYPKDINVKWKIDGSER | Symmetric |
| | QNGVLNSWTDQDSKDSTYSMSSTLTLTKDEY | DBA-Cytokine |
| | ERHNSYTCEATHKTSTSPIVKSFNRNEC | Complex IgG |
| | | format |
| SEQ ID NO: 77 | QVQLVQSGAEVKKPGASVKVSCKASGYTFTT | PD1- |
| | YYVHWVRQAPGQGLEWMGIINPSGGSTSYAQ | IL2_L_7A05scFv_ |
| | NFQGRVTMTRDTSTSTVYMELSSLRSEDTAV | PD1-R07- |
| | YYCASGWDVWGQGTTVTVSSAKTTAPSVYPL | A05_Pep1; |
| | APVCGDTTGSSVTLGCLVKGYFPEPVTLTWN | Asymmetric DBA- |
| | SGSLSSGVHTFPAVLQSDLYTLSSSVTVTSS | Cytokine Complex |
| | TWPSQSITCNVAHPASSTKVDKKIEPRGPTI | IgG-scFv format |
| | KPCPPCKCPAPNAAGGPSVFIFPPKIKDVLM | |
| | ISLSPIVTCVVVDVSEDDPDVQISWFVNNVE | |
| | VHTAQTQTHREDYNSTLRVVSALPIQHQDWM | |
| | SGKEFKCKVNNKDLGAPIERTISKPKGSVRA | |
| | PQVYVLPPPEEEMTKKQVTLTCMVTDFMPED | |
| | IYVEWTNNGKTELNYKNTEPVLDSDGSYFMY | |
| | SDLRVEKKNWVERNSYSCSVVHEGLHNHHTT | |
| | ESFSRTPGK | |
| SEQ ID NO: 78 | QVQLVQSGAEVKKPGASVKVSCKASGYTFTT | PD1- |
| | YYVHWVRQAPGQGLEWMGIINPSGGSTSYAQ | IL2_L_7A05scFv_ |
| | NFQGRVTMTRDTSTSTVYMELSSLRSEDTAV | PD1-R07- |
| | YYCASGWDVWGQGTTVTVSSASGGGGSGGGG | A05_Pep2; |
| | SGGGGSHASEIVMTQSPATLSVSPGERATLS | Asymmetric DBA- |
| | CRASQSVNTYLAWYQQKPGQAPRLLIYGAST | Cytokine Complex |
| | RATGIPARFSGSGSGTEFTLTISSLQSEDFA | IgG-scFv format |
| | VYYCQQYGSSPVTFGQGTRLEIKPRGPTIKP | |
| | CPPCKCPAPNAAGGPSVFIFPPKIKDVLMIS | |
| | LSPIVTCVVVDVSEDDPDVQISWFVNNVEVH | |
| | TAQTQTHREDYNSTLRVVSALPIQHQDWMSG | |
| | KEFKCKVNNKDLGAPIERTISKPKGSVRAPQ | |
| | VYVLPPPEKEMTKKQVSLTCLVKDFMPEDIY | |
| | VEWTNNGKTELNYKNTEPVLKSDGSYFMYSK | |
| | LTVEKKNWVERNSYSCSVVHEGLHNHHTTKS | |
| | FSRTPGGGGSGGGSHHHHHH | |
| SEQ ID NO: 79 | APTSSSTKKTQLQLEHLLLDLQMILNGINNY | PD1- |
| | KNPKLTDMLTFKFYMPKKATELKHLQCLEEE | IL2_L_7A05scFv_ |
| | LKPLEEVLNLAQSKNFHLRPRDLISNINVIV | PD1-R07- |
| | LELKGSETTFMCEYADETATIVEFLNRWITF | A05_Pep3; |
| | CQSIISTLTGGGGSGGGGSGGGGSGGGGSEI | Asymmetric DBA- |
| | VMTQSPATLSVSPGERATLSCRASQSVNTYL | Cytokine Complex |
| | AWYQQKPGQAPRLLIYGASTRATGIPARFSG | IgG-scFv format |
| | SGSGTEFTLTISSLQSEDFAVYYCQQYGSSP | |
| | VTFGQGTRLEIKRADAAPTVSIFPPSSEQLT | |
| | SGGASVVCFLNNFYPKDINVKWKIDGSERQN | |
| | GVLNSWTDQDSKDSTYSMSSTLTLTKDEYER | |
| | HNSYTCEATHKTSTSPIVKSFNRNEC | |

TABLE 7
Dual-Binding Antibodies (DBAs)

| | | | HV* | LV** | | | | | | |
|---|---|---|---|---|---|---|---|---|---|---|
| Dual- | | | SEQ | SEQ | HV_cdr1 | HV_cdr2 | HV_cdr3 | LV_cdr1 | LV_cdr2 | LV_cdr3 |
| Binding | | | ID | ID | SEQ ID | SEQ ID | SEQ ID | SEQ ID | SEQ ID | SEQ ID |
| Antibody | Marker | Therapeutic | NO: | NO: | NO: | NO: | NO: | NO: | NO: | NO: |
| AB001718 | PD-1 | IL-2 | 127 | 135 | 142 | 148 | 154 | 157 | 163 | 168 |
| AB001744 | PD-1 | IL-2 | 128 | 136 | 143 | 148 | 154 | 158 | 163 | 169 |
| AB002022 | PD-1 | IL-2 | 129 | 137 | 144 | 149 | 154 | 159 | 164 | 170 |

*HV refers to the heavy chain variable region of the respective antibodies
**LV refers to the light chain variable region of the respective antibodies

TABLE 8
Sequences of DBA Protein Components

| SEQ ID | DBA Protein | |
|---|---|---|
| NO: | Component | Sequence |
| SEQ ID | AB001718_HV | QVQLVQSGAEVKKPGASVKVSCKA |
| NO: 127 | | SGDTFSTYYVHWVRQAPGQGLEWM |
| | | GIINPSGGGTVYAQKFQGRVTMTR |
| | | DTSTSTVYMELSSLRSEDTAVYYC |
| | | AAGLFIWGQGTLVTVSS |
| SEQ ID | AB001744_HV | QVQLVQSGAEVKKPGASVKVSCKA |
| NO: 128 | | SGYTFSNYYIHWVRQAPGQGLEWM |
| | | GIINPSGGGTVYAQKFQGRVTMTR |
| | | DTSTSTVYMELSSLRSEDTAVYYC |
| | | AAGLFIWGQGTLVTVSS |
| SEQ ID | AB002022_HV | QVQLVQSGAEVKKPGASVKVSCKA |
| NO: 129 | | SGDTFTRHYVHWVRQAPGQGLEWM |
| | | GIINPSGGYASYAQKFQGRVTMTR |
| | | DTSTSTVYMELSSLRSEDTAVYYC |
| | | AAGLFIWGQGTLVTVSS |
| SEQ ID | AB001718_LV | DIQMTQSPSSLSASVGDRVTITCR |
| NO: 135 | | ASQYISSGLAWYQQKPGKAPKLLI |
| | | YKASSLDNGVPSRFSGSGSGTDFT |
| | | LTISSLQPEDFATYYCQQYERLPL |
| | | TFGGGTKVEIK |
| SEQ ID | AB001744_LV | DIQMTQSPSSLSASVGDRVTITCR |
| NO: 136 | | ASQSIGTGLAWYQQKPGKAPKLLI |
| | | YKASSLDNGVPSRFSGSGSGTDFT |
| | | LTISSLQPEDFATYYCQQYNRAPL |
| | | TFGGGTKVEIK |
| SEQ ID | AB002022_LV | DIQMTQSPSSLSASVGDRVTITCR |
| NO: 137 | | ASQSIGRWLAWYQQKPGKAPKLLI |
| | | YSASNLETGVPSRFSGSGSGTDFT |
| | | LTISSLQPEDFATYYCQQYESFPV |
| | | TFGPGTKVDIK |
| SEQ ID | AB001718_HV_cdr1 | GDTFSTYYVH |
| NO: 142 | | |
| SEQ ID | AB001843_HV_cdr1 | GYTFSGYYIH |
| NO: 146 | | |
| SEQ ID | AB001866_HV_cdr1 | GYTFSNYYVH |
| NO: 147 | | |
| SEQ ID | AB001718_HV_cdr2 | IINPSGGGTVYAQKFQG |
| NO: 148 | | |
| | AB001744_HV_cdr2 | IINPSGGGTVYAQKFQG |
| SEQ ID | AB002022_HV_cdr2 | IINPSGGYASYAQKFQG |
| NO: 149 | | |
| SEQ ID | AB001718_HV_cdr3 | AAGLFI |
| NO: 154 | | |
| SEQ ID | AB001718_LV_cdr1 | RASQYISSGLA |
| NO: 157 | | |
| SEQ ID | AB001744_LV_cdr1 | RASQSIGTGLA |
| NO: 158 | | |
| SEQ ID | AB002022_LV_cdr1 | RASQSIGRWLA |
| NO: 159 | | |
| SEQ ID | AB001609_LV_cdr1 | RASQSISNRLA |
| NO: 160 | | |
| SEQ ID | AB001638_LV_cdr1 | QASQSISNYLA |
| NO: 161 | | |
| SEQ ID | AB001843_LV_cdr1 | RASQSISSYLN |
| NO: 162 | | |
| SEQ ID | AB001718_LV_cdr2 | KASSLDN |
| NO: 163 | | |
| | AB001744_LV_cdr2 | KASSLDN |
| SEQ ID | AB002022_LV_cdr2 | SASNLET |
| NO: 164 | | |
| SEQ ID | AB001718_LV_cdr3 | QQYERLPL |
| NO: 168 | | |
| SEQ ID | AB001744_LV_cdr3 | QQYNRAPL |
| NO: 169 | | |
| SEQ ID | AB002022_LV_cdr3 | QQYESFPV |
| NO: 170 | | |

TABLE 9
Exemplary DBA-Cytokine Protein Complexes

| | | | | | | | Heavy | Heavy | Heavy |
|---|---|---|---|---|---|---|---|---|---|
| | | | | | | | Chain 1 | Chain 2 | Chain 3 |
| | DBA/ | | | DBA | Therapeutic | 2nd Ab | SEQ ID | SEQ ID | SEQ ID |
| Name | Therapeutic | DBA | Type | domains | domains | domain | NO: | NO: | NO: |
| AF003229 | PD-1/IL-2 | AB001718 | FIG. 2B | 2 | 1 | N/A | 80 | 97 | 114 |
| AF003230 | PD-1/IL-2 | AB001744 | FIG. 2B | 2 | 1 | N/A | 81 | 98 | 115 |
| AF003232 | PD-1/IL-2 | AB002022 | FIG. 2B | 2 | 1 | N/A | 82 | 99 | 116 |
| AF003250 | PD-1/IL-2 | AB001718 | FIG. | 1 | 1 | anti-PD-1 | 83 | 100 | 117 |
| | | | 2A | | | | | | |
| AF003251 | PD-1/IL-2 | AB001744 | FIG. | 1 | 1 | anti-PD-1 | 84 | 101 | 118 |
| | | | 2A | | | | | | |
| AF003253 | PD-1/IL-2 | AB002022 | FIG. | 1 | 1 | anti-PD-1 | 85 | 102 | 119 |
| | | | 2A | | | | | | |

TABLE 10
Sequences of Peptides in TABLE 9

| SEQ ID | | |
|---|---|---|
| NO: | DBA | Sequence |
| SEQ ID | AF003229_Pep1 | APTSSSTKKTQLQLEHLLLDLQMILNGINNYKNPKLTDMLTF |
| NO: 80 | | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLTGGGGSGGGGSGGGGSGGGGSQVQLVQSGAEVKKPG |
| | | ASVKVSCKASGDTFSTYYVHWVRQAPGQGLEWMGIINPSGGG |
| | | TVYAQKFQGRVTMTRDTSTSTVYMELSSLRSEDTAVYYCAAG |
| | | LFIWGQGTLVTVSSAKTTAPSVYPLAPVCGDTTGSSVTLGCL |
| | | VKGYFPEPVTLTWNSGSLSSGVHTFPAVLQSDLYTLSSSVTV |
| | | TSSTWPSQSITCNVAHPASSTKVDKKIEPRGPTIKPCPPCKC |
| | | PAPNAAGGPSVFIFPPKIKDVLMISLSPIVTCVVVDVSEDDP |
| | | DVQISWFVNNVEVHTAQTQTHREDYNSTLRVVSALPIQHQDW |
| | | MSGKEFKCKVNNKDLGAPIERTISKPKGSVRAPQVYVLPPPE |
| | | EEMTKKQVTLTCMVTDFMPEDIYVEWTNNGKTELNYKNTEPV |
| | | LDSDGSYFMYSDLRVEKKNWVERNSYSCSVVHEGLHNHHTTE |
| | | SFSRTPGK |
| SEQ ID | AF003230_Pep1 | APTSSSTKKTQLQLEHLLLDLQMILNGINNYKNPKLTDMLTF |
| NO: 81 | | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLTGGGGSGGGGSGGGGSGGGGSQVQLVQSGAEVKKPG |
| | | ASVKVSCKASGYTFSNYYIHWVRQAPGQGLEWMGIINPSGGG |
| | | TVYAQKFQGRVTMTRDTSTSTVYMELSSLRSEDTAVYYCAAG |
| | | LFIWGQGTLVTVSSAKTTAPSVYPLAPVCGDTTGSSVTLGCL |
| | | VKGYFPEPVTLTWNSGSLSSGVHTFPAVLQSDLYTLSSSVTV |
| | | TSSTWPSQSITCNVAHPASSTKVDKKIEPRGPTIKPCPPCKC |
| | | PAPNAAGGPSVFIFPPKIKDVLMISLSPIVTCVVVDVSEDDP |
| | | DVQISWFVNNVEVHTAQTQTHREDYNSTLRVVSALPIQHQDW |
| | | MSGKEFKCKVNNKDLGAPIERTISKPKGSVRAPQVYVLPPPE |
| | | EEMTKKQVTLTCMVTDFMPEDIYVEWTNNGKTELNYKNTEPV |
| | | LDSDGSYFMYSDLRVEKKNWVERNSYSCSVVHEGLHNHHTTE |
| | | SFSRTPGK |
| SEQ ID | AF003232_Pep1 | APTSSSTKKTQLQLEHLLLDLQMILNGINNYKNPKLTDMLTF |
| NO: 82 | | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLTGGGGSGGGGSGGGGSGGGGSQVQLVQSGAEVKKPG |
| | | ASVKVSCKASGDTFTRHYVHWVRQAPGQGLEWMGIINPSGGY |
| | | ASYAQKFQGRVTMTRDTSTSTVYMELSSLRSEDTAVYYCAAG |
| | | LFIWGQGTLVTVSSAKTTAPSVYPLAPVCGDTTGSSVTLGCL |
| | | VKGYFPEPVTLTWNSGSLSSGVHTFPAVLQSDLYTLSSSVTV |
| | | TSSTWPSQSITCNVAHPASSTKVDKKIEPRGPTIKPCPPCKC |
| | | PAPNAAGGPSVFIFPPKIKDVLMISLSPIVTCVVVDVSEDDP |
| | | DVQISWFVNNVEVHTAQTQTHREDYNSTLRVVSALPIQHQDW |
| | | MSGKEFKCKVNNKDLGAPIERTISKPKGSVRAPQVYVLPPPE |
| | | EEMTKKQVTLTCMVTDFMPEDIYVEWTNNGKTELNYKNTEPV |
| | | LDSDGSYFMYSDLRVEKKNWVERNSYSCSVVHEGLHNHHTTE |
| | | SFSRTPGK |
| SEQ ID | AF003250_Pep1 | APTSSSTKKTQLQLEHLLLDLQMILNGINNYKNPKLTDMLTF |
| NO: 83 | | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLTGGGGSGGGGSGGGGSGGGGSQVQLVQSGAEVKKPG |
| | | ASVKVSCKASGDTFSTYYVHWVRQAPGQGLEWMGIINPSGGG |
| | | TVYAQKFQGRVTMTRDTSTSTVYMELSSLRSEDTAVYYCAAG |
| | | LFIWGQGTLVTVSSAKTTAPSVYPLAPVCGDTTGSSVTLGCL |
| | | VKGYFPEPVTLTWNSGSLSSGVHTFPAVLQSDLYTLSSSVTV |
| | | TSSTWPSQSITCNVAHPASSTKVDKKIEPRGPTIKPCPPCKC |
| | | PAPNAAGGPSVFIFPPKIKDVLMISLSPIVTCVVVDVSEDDP |
| | | DVQISWFVNNVEVHTAQTQTHREDYNSTLRVVSALPIQHQDW |
| | | MSGKEFKCKVNNKDLGAPIERTISKPKGSVRAPQVYVLPPPE |
| | | EEMTKKQVTLTCMVTDFMPEDIYVEWTNNGKTELNYKNTEPV |
| | | LDSDGSYFMYSDLRVEKKNWVERNSYSCSVVHEGLHNHHTTE |
| | | SFSRTPGK |
| SEQ ID | AF003251_Pep1 | APTSSSTKKTQLQLEHLLLDLQMILNGINNYKNPKLTDMLTF |
| NO: 84 | | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLTGGGGSGGGGSGGGGSGGGGSQVQLVQSGAEVKKPG |
| | | ASVKVSCKASGYTFSNYYIHWVRQAPGQGLEWMGIINPSGGG |
| | | TVYAQKFQGRVTMTRDTSTSTVYMELSSLRSEDTAVYYCAAG |
| | | LFIWGQGTLVTVSSAKTTAPSVYPLAPVCGDTTGSSVTLGCL |
| | | VKGYFPEPVTLTWNSGSLSSGVHTFPAVLQSDLYTLSSSVTV |
| | | TSSTWPSQSITCNVAHPASSTKVDKKIEPRGPTIKPCPPCKC |
| | | PAPNAAGGPSVFIFPPKIKDVLMISLSPIVTCVVVDVSEDDP |
| | | DVQISWFVNNVEVHTAQTQTHREDYNSTLRVVSALPIQHQDW |
| | | MSGKEFKCKVNNKDLGAPIERTISKPKGSVRAPQVYVLPPPE |
| | | EEMTKKQVTLTCMVTDFMPEDIYVEWTNNGKTELNYKNTEPV |
| | | LDSDGSYFMYSDLRVEKKNWVERNSYSCSVVHEGLHNHHTTE |
| | | SFSRTPGK |
| SEQ ID | AF003253_Pep1 | APTSSSTKKTQLQLEHLLLDLQMILNGINNYKNPKLTDMLTF |
| NO: 85 | | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLTGGGGSGGGGSGGGGSGGGGSQVQLVQSGAEVKKPG |
| | | ASVKVSCKASGDTFTRHYVHWVRQAPGQGLEWMGIINPSGGY |
| | | ASYAQKFQGRVTMTRDTSTSTVYMELSSLRSEDTAVYYCAAG |
| | | LFIWGQGTLVTVSSAKTTAPSVYPLAPVCGDTTGSSVTLGCL |
| | | VKGYFPEPVTLTWNSGSLSSGVHTFPAVLQSDLYTLSSSVTV |
| | | TSSTWPSQSITCNVAHPASSTKVDKKIEPRGPTIKPCPPCKC |
| | | PAPNAAGGPSVFIFPPKIKDVLMISLSPIVTCVVVDVSEDDP |
| | | DVQISWFVNNVEVHTAQTQTHREDYNSTLRVVSALPIQHQDW |
| | | MSGKEFKCKVNNKDLGAPIERTISKPKGSVRAPQVYVLPPPE |
| | | EEMTKKQVTLTCMVTDFMPEDIYVEWTNNGKTELNYKNTEPV |
| | | LDSDGSYFMYSDLRVEKKNWVERNSYSCSVVHEGLHNHHTTE |
| | | SFSRTPGK |

### Example 3

### Reduced CD8⁺ T Cell STAT5 Phosphorylation by a PD-1/IL-2 Dual Binding Antibody (DBA) Cytokine Complexes

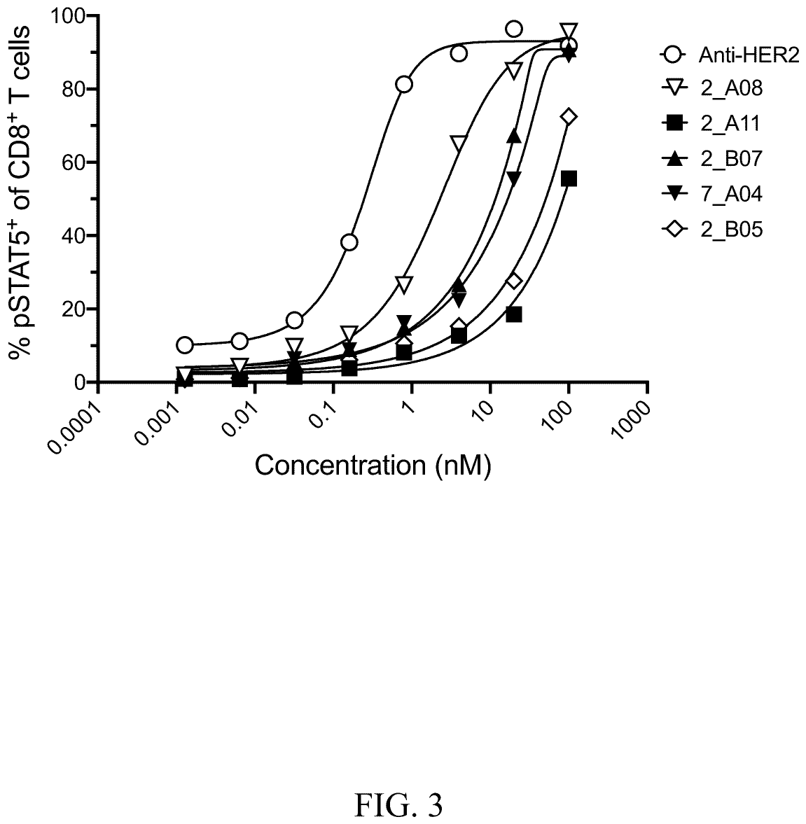

This example describes reduction of IL-2 mediated signaling by addition of PD-1/IL-2 DBA moieties to IL-2 molecules by fusion, as read out using CD8+ T-cell STAT5 phosphorylation. Genes for the PD-1/IL-2 DBAs shown in TABLE 11 were synthesized and expressed in HEK293 as IgG proteins with IL-2 fused to the N-terminus of the heavy or light chain through a linker (Genscript). Although only two of the antibodies blocked IL-2 binding to IL-2RB as scFvs, over 30 of the antibodies were able to reduce IL-2 signaling by a linked IL-2 domain in formats as shown in FIGS. 2D and 2E. An exemplary set of these DBAs were chosen for analysis and compared to a control anti-HER2-IL-2 immunocytokine (TABLE 11 and FIG. 3).

TABLE 11
IgG PD-1/IL-2 DBA protein complexes

| | Heavy Chain Sequence | Light Chain Sequence |
|---|---|---|
| Name | SEQ ID NO | SEQ ID NO |
| Anti-HER2 | (SEQ ID NO: 65) | (SEQ ID NO: 66) |
| | EVQLVESGGGLVQPGGSLRLSCAA | APTSSSTKKTQLQLEHLLLDLQ |
| | SGFNIKDTYIHWVRQAPGKGLEWV | MILNGINNYKNPKLTRMLTFKF |
| | ARIYPTNGYTRYADSVKGRFTISA | YMPKKATELKHLQCLEEELKPL |
| | DTSKNTAYLQMNSLRAEDTAVYYC | EEVLNLAQSKNFHLRPRDLISN |
| | SRWGGDGFYAMDYWGQGTLVTVSS | INVIVLELKGSETTFMCEYADE |
| | AKTTAPSVYPLAPVCGDTTGSSVT | TATIVEFLNRWITFCQSIISTL |
| | LGCLVKGYFPEPVTLTWNSGSLSS | TVPGVGVPGAGVPGVGVPGGGV |
| | GVHTFPAVLQSDLYTLSSSVTVTS | PGVGVPGGGVPGAGVPGGGVPG |
| | STWPSQSITCNVAHPASSTKVDKK | VGVPGAGVPGVGVPGGGDIQMT |
| | IEPRGPTIKPCPPCKCPAPNAAGG | QSPSSLSASVGDRVTITCRASQ |
| | PSVFIFPPKIKDVLMISLSPIVTC | DVNTAVAWYQQKPGKAPKLLIY |
| | VVVDVSEDDPDVQISWFVNNVEVH | SASFLYSGVPSRFSGSRSGTDF |
| | TAQTQTHREDYNSTLRVVSALPIQ | TLTISSLQPEDFATYYCQQHYT |
| | HQDWMSGKEFKCKVNNKDLGAPIE | TPPTFGQGTKVEIKRADAAPTV |
| | RTISKPKGSVRAPQVYVLPPPEEE | SIFPPSSEQLTSGGASVVCFLN |
| | MTKKQVTLTCMVTDFMPEDIYVEW | NFYPKDINVKWKIDGSERQNGV |
| | TNNGKTELNYKNTEPVLDSDGSYF | LNSWTDQDSKDSTYSMSSTLTL |
| | MYSKLRVEKKNWVERNSYSCSVVH | TKDEYERHNSYTCEATHKTSTS |
| | EGLHNHHTTKSFSRTPGK | PIVKSFNRNEC |
| 2-A08 | (SEQ ID NO: 67) | (SEQ ID NO: 68) |
| | QVQLVQSGAEVKKPGASVKVSCKV | APTSSSTKKTQLQLEHLLLDLQ |
| | SGYTFTSYDINWVRQAPGQGLEWM | MILNGINNYKNPKLTRMLTFKF |
| | GWINPNSGDTGYAQKFQGRVTMTR | YMPKKATELKHLQCLEEELKPL |
| | DTSTSTVYMELSSLRSEDTAVYYC | EEVLNLAQSKNFHLRPRDLISN |
| | ARDTGLGYYYGSGDFDYWGQGTLV | INVIVLELKGSETTFMCEYADE |
| | TVSSAKTTAPSVYPLAPVCGDTTG | TATIVEFLNRWITFCQSIISTL |
| | SSVTLGCLVKGYFPEPVTLTWNSG | TVPGVGVPGAGVPGVGVPGGGV |
| | SLSSGVHTFPAVLQSDLYTLSSSV | PGVGVPGGGVPGAGVPGGGVPG |
| | TVTSSTWPSQSITCNVAHPASSTK | VGVPGAGVPGVGVPGGGDIQMT |
| | VDKKIEPRGPTIKPCPPCKCPAPN | QSPSSLSASVGDRVTITCQASQ |
| | AAGGPSVFIFPPKIKDVLMISLSP | DIHNYLNWYQQKPGKAPKLLIY |
| | IVTCVVVDVSEDDPDVQISWFVNN | DVSNLETGVPSRFSGSGSGTDF |
| | VEVHTAQTQTHREDYNSTLRVVSA | TLTISSLQPEDFATYYCQQAIS |
| | LPIQHQDWMSGKEFKCKVNNKDLG | FPLTFGGGTKVEIKRADAAPTV |
| | APIERTISKPKGSVRAPQVYVLPP | SIFPPSSEQLTSGGASVVCFLN |
| | PEEEMTKKQVTLTCMVTDFMPEDI | NFYPKDINVKWKIDGSERQNGV |
| | YVEWTNNGKTELNYKNTEPVLDSD | LNSWTDQDSKDSTYSMSSTLTL |
| | GSYFMYSKLRVEKKNWVERNSYSC | TKDEYERHNSYTCEATHKTSTS |
| | SVVHEGLHNHHTTKSFSRTPGK | PIVKSFNRNEC |
| 2-A11 | (SEQ ID NO: 69) | (SEQ ID NO: 70) |
| | QVQLVQSGAEVKKPGASVKVSCKA | APTSSSTKKTQLQLEHLLLDLQ |
| | SGHTFTRYYMHWVRQAPGQGLEWM | MILNGINNYKNPKLTRMLTFKF |
| | GIINPSGGYATYAQKFQGRVTMTR | YMPKKATELKHLQCLEEELKPL |
| | DTSTSTVYMELSSLRSEDTAVYYC | EEVLNLAQSKNFHLRPRDLISN |
| | ASGWDVWGQGTLVTVSSAKTTAPS | INVIVLELKGSETTFMCEYADE |
| | VYPLAPVCGDTTGSSVTLGCLVKG | TATIVEFLNRWITFCQSIISTL |
| | YFPEPVTLTWNSGSLSSGVHTFPA | TVPGVGVPGAGVPGVGVPGGGV |
| | VLQSDLYTLSSSVTVTSSTWPSQS | PGVGVPGGGVPGAGVPGGGVPG |
| | ITCNVAHPASSTKVDKKIEPRGPT | VGVPGAGVPGVGVPGGGDIQMT |
| | IKPCPPCKCPAPNAAGGPSVFIFP | QSPSSLSASVGDRVTITCRASQ |
| | PKIKDVLMISLSPIVTCVVVDVSE | SINSWLAWYQQKPGKAPKLLIY |
| | DDPDVQISWFVNNVEVHTAQTQTH | ATSTLESGVPSRFSGSGSGTDF |
| | REDYNSTLRVVSALPIQHQDWMSG | TLTISSLQPEDFATYYCQQSYS |
| | KEFKCKVNNKDLGAPIERTISKPK | FPPTFGQGTKVEIKRADAAPTV |
| | GSVRAPQVYVLPPPEEEMTKKQVT | SIFPPSSEQLTSGGASVVCFLN |
| | LTCMVTDFMPEDIYVEWTNNGKTE | NFYPKDINVKWKIDGSERQNGV |
| | LNYKNTEPVLDSDGSYFMYSKLRV | LNSWTDQDSKDSTYSMSSTLTL |
| | EKKNWVERNSYSCSVVHEGLHNHH | TKDEYERHNSYTCEATHKTSTS |
| | TTKSFSRTPGK | PIVKSFNRNEC |
| 2-B05 | (SEQ ID NO: 71) | (SEQ ID NO: 72) |
| | QVQLVQSGAEVKKPGASVKVSCKA | APTSSSTKKTQLQLEHLLLDLQ |
| | SGYTFTNYYIHWVRQAPGQGLEWM | MILNGINNYKNPKLTRMLTFKF |
| | GIINPRAGYTSYALKFQGRVTMTR | YMPKKATELKHLQCLEEELKPL |
| | DTSTSTVYMELSSLRSEDTAVYYC | EEVLNLAQSKNFHLRPRDLISN |
| | AGGWLDWGQGTLVTVSSAKTTAPS | INVIVLELKGSETTFMCEYADE |
| | VYPLAPVCGDTTGSSVTLGCLVKG | TATIVEFLNRWITFCQSIISTL |
| | YFPEPVTLTWNSGSLSSGVHTFPA | TVPGVGVPGAGVPGVGVPGGGV |
| | VLQSDLYTLSSSVTVTSSTWPSQS | PGVGVPGGGVPGAGVPGGGVPG |
| | ITCNVAHPASSTKVDKKIEPRGPT | VGVPGAGVPGVGVPGGGDIQMT |
| | IKPCPPCKCPAPNAAGGPSVFIFP | QSPSSLSASVGDRVTITCRASQ |
| | PKIKDVLMISLSPIVTCVVVDVSE | SISSWLAWYQQKPGKAPKLLIY |
| | DDPDVQISWFVNNVEVHTAQTQTH | AASSLQSGVPSRFSGSGSGTDF |
| | REDYNSTLRVVSALPIQHQDWMSG | TLTISSLQPEDFATYYCQQSFT |
| | KEFKCKVNNKDLGAPIERTISKPK | MPITFGQGTRLEIKRADAAPTV |
| | GSVRAPQVYVLPPPEEEMTKKQVT | SIFPPSSEQLTSGGASVVCFLN |
| | LTCMVTDFMPEDIYVEWTNNGKTE | NFYPKDINVKWKIDGSERQNGV |
| | LNYKNTEPVLDSDGSYFMYSKLRV | LNSWTDQDSKDSTYSMSSTLTL |
| | EKKNWVERNSYSCSVVHEGLHNHH | TKDEYERHNSYTCEATHKTSTS |
| | TTKSFSRTPGK | PIVKSFNRNEC |
| 2-B07 | (SEQ ID NO: 73) | (SEQ ID NO: 74) |
| | QVQLVQSGAEVKKPGASVKVSCKA | APTSSSTKKTQLQLEHLLLDLQ |
| | SGDTFTRHYVHWVRQAPGQGLEWM | MILNGINNYKNPKLTRMLTFKF |
| | GIINPSGGYASYAQKFQGRVTMTR | YMPKKATELKHLQCLEEELKPL |
| | DTSTSTVYMELSSLRSEDTAVYYC | EEVLNLAQSKNFHLRPRDLISN |
| | AAGLFIWGQGTLVTVSSAKTTAPS | INVIVLELKGSETTFMCEYADE |
| | VYPLAPVCGDTTGSSVTLGCLVKG | TATIVEFLNRWITFCQSIISTL |
| | YFPEPVTLTWNSGSLSSGVHTFPA | TVPGVGVPGAGVPGVGVPGGGV |
| | VLQSDLYTLSSSVTVTSSTWPSQS | PGVGVPGGGVPGAGVPGGGVPG |
| | ITCNVAHPASSTKVDKKIEPRGPT | VGVPGAGVPGVGVPGGGDIQMT |
| | IKPCPPCKCPAPNAAGGPSVFIFP | QSPSSLSASVGDRVTITCRASQ |
| | PKIKDVLMISLSPIVTCVVVDVSE | SIGRWLAWYQQKPGKAPKLLIY |
| | DDPDVQISWFVNNVEVHTAQTQTH | SASNLETGVPSRFSGSGSGTDF |
| | REDYNSTLRVVSALPIQHQDWMSG | TLTISSLQPEDFATYYCQQANS |
| | KEFKCKVNNKDLGAPIERTISKPK | FPVTFGPGTKVDIKRADAAPTV |
| | GSVRAPQVYVLPPPEEEMTKKQVT | SIFPPSSEQLTSGGASVVCFLN |
| | LTCMVTDFMPEDIYVEWTNNGKTE | NFYPKDINVKWKIDGSERQNGV |
| | LNYKNTEPVLDSDGSYFMYSKLRV | LNSWTDQDSKDSTYSMSSTLTL |
| | EKKNWVERNSYSCSVVHEGLHNHH | TKDEYERHNSYTCEATHKTSTS |
| | TTKSFSRTPGK | PIVKSFNRNEC |
| 7-A04 | (SEQ ID NO: 75) | (SEQ ID NO: 76) |
| | QVQLVQSGAEVKKPGASVKVSCKA | APTSSSTKKTQLQLEHLLLDLQ |
| | SGYTFTDYYMHWVRQAPGQGLEWM | MILNGINNYKNPKLTRMLTFKF |
| | GIINPRAGYTSYALKFQGRVTMTR | YMPKKATELKHLQCLEEELKPL |
| | DTSTSTVYMELSSLRSEDTAVYYC | EEVLNLAQSKNFHLRPRDLISN |
| | TSGMDVWGQGTLVTVSSAKTTAPS | INVIVLELKGSETTFMCEYADE |
| | VYPLAPVCGDTTGSSVTLGCLVKG | TATIVEFLNRWITFCQSIISTL |
| | YFPEPVTLTWNSGSLSSGVHTFPA | TVPGVGVPGAGVPGVGVPGGGV |
| | VLQSDLYTLSSSVTVTSSTWPSQS | PGVGVPGGGVPGAGVPGGGVPG |
| | ITCNVAHPASSTKVDKKIEPRGPT | VGVPGAGVPGVGVPGGGDIQMT |
| | IKPCPPCKCPAPNAAGGPSVFIFP | QSPSSLSASVGDRVTITCRASQ |
| | PKIKDVLMISLSPIVTCVVVDVSE | SISTWLAWYQQKPGKAPKLLIY |
| | DDPDVQISWFVNNVEVHTAQTQTH | AASSLQSGVPSRFSGSGSGTDF |
| | REDYNSTLRVVSALPIQHQDWMSG | TLTISSLQPEDFATYYCQQSYS |
| | KEFKCKVNNKDLGAPIERTISKPK | FPVTFGQGTKVEIKRADAAPTV |
| | GSVRAPQVYVLPPPEEEMTKKQVT | SIFPPSSEQLTSGGASVVCFLN |
| | LTCMVTDFMPEDIYVEWTNNGKTE | NFYPKDINVKWKIDGSERQNGV |
| | LNYKNTEPVLDSDGSYFMYSKLRV | LNSWTDQDSKDSTYSMSSTLTL |
| | EKKNWVERNSYSCSVVHEGLHNHH | TKDEYERHNSYTCEATHKTSTS |
| | TTKSFSRTPGK | PIVKSFNRNEC |

The PD-1/IL-2 DBA-cytokine complexes were serially diluted in complete RPMI (+10% FBS, 2 mM L-glutamine, sodium pyruvate) and added to a 96-well plate. 2×105 human peripheral blood mononuclear cells (PBMCs) were added to each well and plates were incubated at 37° C. for 20 minutes. An equal volume prewarmed fixation buffer (Biolegend) was then added to each well and plates were incubated at 37° C. for 10 minutes. Cells were then fixed in pre-chilled Perm Buffer III (BD Biosciences) for 30 minutes at 4° C. Cells were washed with FACS wash buffer (PBS+2% FBS, 2 mM EDTA) and stained with fluorophore labeled antibodies directed against CD3, CD4, CD8, (BioLegend) and phospho-STAT5 (BD Biosciences) diluted 1:20 in FACS wash buffer. Cells were incubated 1 hour at 4° C., washed with FACS wash buffer, and analyzed on a SA3800 Spectral Analyzer. In the absence of PD-1, the PD-1/IL-2 DBA/cytokine complexes induced less STAT5 phosphorylation in T cells compared to the monospecific control anti-HER2 IL-2 immunocytokine (FIG. 3).

### Example 4

### General Method: Production of Complexes to Drive PD-1 Dependent IL-2 Activity in Human Cells

This example describes PD-1/IL-2 protein complexes for PD-1 dependent IL-2 activity in human cells, in vitro and in vivo. PD-1/IL-2 protein complexes comprise a PD-1 sensor domain (e.g., an anti-PD-1 antibody or an anti-PD-1 scFv) linked to an IL-2 cytokine therapeutic domain via a linker, where the IL-2 cytokine is a therapeutic. In the absence of PD-1, the PD-1 sensor domain binds the IL-2 therapeutic domain, rendering the IL-2 therapeutic inert. In the presence of PD-1 (e.g., PD-1 is expressed on a cell, such as an immune cell), the PD-1 sensor domain binds PD-1, thereby unbinding the IL-2 therapeutic domain and allowing for IL-2 to exhibit therapeutic activity.

PD-1/IL-2 protein complexes are recombinantly expressed or chemically synthesized. PD-1/IL-2 protein complexes are administered in vitro to a human cell or in vivo to a mouse or to a human subject in need thereof. The human cell is a cell expressing PD-1. Administration to a mouse or to a human subject is performed intravenously, intramuscularly, subcutancously, intradermally, intraperitoneally, or mucosally. In the absence of PD-1, the IL-2 therapeutic domain remains bound to the PD-1 sensor domain and no therapeutic efficacy is observed (e.g., cell activation in vitro and in the subject is unaltered). In the presence of PD-1, the PD-1 sensor domain binds PD-1 and unbinds the IL-2 therapeutic domain. Therapeutic efficacy is observed (e.g., cell activation is observed in vitro and, in the subject, in vivo). The subject has a disease. The disease is cancer. The cell may express PD-1 endogenously or after activation, or following introduction of a gene encoding PD-1. The therapeutic effect may be cell growth, differentiation, activation or induction of IL2-responsive genes. In vitro, if the cell is part of a mixture of cell types, any of these changes may be monitored for a responding cell population in the mixture.

### Example 5

### PD-1/IL-2 DBA Cytokine Complex Induction of STAT5 Phosphorylation in a Lymphocyte Cell Line

This example describes PD-1/IL-2 DBA-cytokine complex induction of STAT5 phosphorylation in a lymphocytic cell line. To assess the dependence of PD-1/IL-2 DBA-cytokine complex activity on binding to PD-1, a PD-1-expressing variant is generated of an IL-2R+ T cell line such as Hut78 or Jurkat E6.1. The PD-1+ and PD-1-variant cell lines are treated with titrating concentrations of a PD-1/IL-2 DBA-cytokine complex of this disclosure, and STAT5 phosphorylation is assessed by phospho-flow, TR-FRET, or other assays for measuring IL-2 signaling.

A HEK 293 IL-2 reporter cell line is engineered to express PD-1. The PD-1+ and PD-1-variant cell lines are treated with titrating concentrations of PD-1/IL-2 DBA-cytokine complexes, and reporter activity is assessed as a measurement of IL-2 signaling. The PD-1/IL-2 DBA-cytokine complex exhibits increased potency on PD-1+ variant cell lines.

### Example 6

### PD-1/IL-2 DBA Cytokine Complex Induction of STAT5 Phosphorylation and Other Markers of Activation, and Proliferation in Primary Lymphocytes

This example describes PD-1/IL-2 DBA-cytokine complex induction of STAT5 phosphorylation and other markers of activation and proliferation in primary lymphocytes. PBMCs are labeled with cell proliferation dye and incubated for 4 days with titrating concentrations of a PD-1/IL-2 DBA-cytokine complex of the present disclosure. PBMCs are stained with antibodies directed against immune cell phenotyping markers to distinguish CD4+ and CD8+ T cells, Treg cells, and natural killer (NK) cells and markers of cell activation, such as CD25. Dye dilution on immune cell subsets is examined by flow cytometry as a measurement of proliferation.

Total T cells are isolated from PBMCs using immunomagnetic negative selection (STEMCELL) and stimulated with plate-bound anti-CD3 and soluble anti-CD28 for 72 hours to induce expression of PD-1. The PD-1+ T cells are incubated for 20 minutes with titrating concentrations of PD-1/IL-2 DBA-cytokine complexes. STAT5 phosphorylation is measured in fixed and permeabilized T cells by flow cytometry. In some experiments, PD-1 may be blocked on T cells with anti-PD-1 prior to treatment with PD-1/IL-2 DBA-cytokine complexes to assess the dependence of PD-1/IL-2 DBA-cytokine complex activity on binding to PD-1. The PD-1/IL-2 DBA-cytokine complex induces minimal STAT5 phosphorylation when PD-1 is blocked, showing activity that is conditional on its ability to bind PD-1.

### Example 7

### In Vivo PD-1/IL-2 DBA Cytokine Complex Signaling in Non-Tumor Peripheral Tissues

This example describes PD-1/IL-2 DBA-cytokine complex pharmacokinetics in the blood of wild-type mice and the signaling of the complex in non-tumor peripheral tissue. The serum half-lives and peripheral tissue activities of PD-1/IL-2 DBA-cytokine complexes and suitable non-regulated controls such as anti-PD-1, anti-HER2-IL-2, or anti-PD-1-IL-2 were measured in mice dosed intravenously (i.v.) with the complexes. Blood, spleens, or both were collected at various timepoints after treatment and stained to identify CD8+ T cells and NK cells.

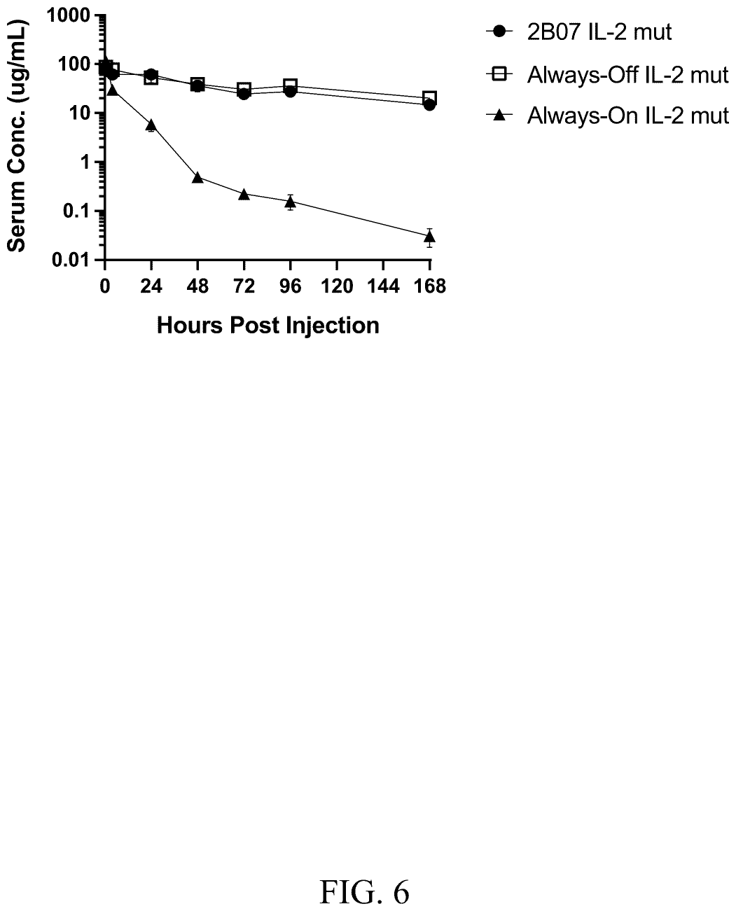

To examine the half-life of PD-1/IL-2 DBA-cytokine complex in circulation, wild-type C57BL/6 mice received a single 2.5 milligrams per kilogram intravenous dose of a PD-1/IL-2 DBA-cytokine complex (2B07 IL-2 mut; SEQ ID NO: 205-206), anti-HER2/IL-2-cytokine complex (Always-on IL-2 mut; SEQ ID NO: 64 and SEQ ID NO: 207), or anti-IL-2/IL-2-cytokine complex (Always-off IL-2 mut; SEQ ID SEQ ID NO: 208-209), as outlined in TABLE 13. Mice were bled via retro-orbital sinus at 30 minutes, 4, 24, 48, 72, 96, and 168 hours post-dosing. The blood was collected into serum separator tubes, and the isolated scrum was frozen at −80° C. until analysis. To determine serum levels of the cytokine complexes, 96-well high-binding ELISA plates were coated with 1 μg/mL rabbit anti-hu IL-2 capture antibody (clone ab9618, Abcam) in carbonate-bicarbonate buffer overnight at 4 C. Plates were washed three times and blocked for 1 hour with SUPERBLOCK™ blocking buffer (Thermo Scientific). Serum samples from the various timepoints and treatment groups were diluted in SUPERBLOCK™, added to the plates, and incubated 1 hr. To detect cytokine complexes, plates were incubated with goat anti-mouse Fc-HRP (Jackson ImmunoRescarch) at 1:5000 in SUPERBLOCK™ for 1 hour. The plates were then washed and developed with TMB substrate. Absorbance (OD) was measured using an ENVISION™ 2105 microplate reader (PerkinElmer) at 450 nm. As shown in FIG. 6, at all timepoints examined the PD-1/IL-2 DBA-cytokine complex was detected at similar serum concentrations as the anti-IL-2/IL-2-cytokine complex. In contrast, the serum concentration of the non-regulated anti-HER2/IL-2-cytokine complex showed a greater decrease in serum concentration over time.

TABLE 12
IgG PD-1/IL-2 DBA and control protein complexes

| Protein | SEQ ID | |
|---|---|---|
| Complexes | NO: | Sequence |
| 2B07 IL-2 | SEQ ID | APASSSTKKTQLQLEHLLLDLQMILNGINNYKNPKLTRMLTAKFAMPKK |
| mut | NO: 205 | ATELKHLQCLEEELKPLEEVLNGAQSKNFHLRPRDLISNINVIVLELKG |
| | | SETTFMCEYADETATIVEFLNRWITFAQSIISTLTGGGGSGGGGSGGGG |
| | | SGGGGSQVQLVQSGAEVKKPGASVKVSCKASGDTFTRHYVHWVRQAPGQ |
| | | GLEWMGIINPSGGYASYAQKFQGRVTMTRDTSTSTVYMELSSLRSEDTA |
| | | VYYCAAGLFIWGQGTLVTVSSAKTTAPSVYPLAPVCGDTTGSSVTLGCL |
| | | VKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNAAGGPSVFIF |
| | | PPKIKDVLMISLSPIVTCVVVDVSEDDPDVQISWFVNNVEVHTAQTQTH |
| | | REDYNSTLRVVSALPIQHQDWMSGKEFKCKVNNKDLGAPIERTISKPKG |
| | | SVRAPQVYVLPPPEEEMTKKQVTLTCMVTDFMPEDIYVEWTNNGKTELN |
| | | YKNTEPVLDSDGSYFMYSKLRVEKKNWVERNSYSCSVVHEGLHNHHTTK |
| | | SFSRTPGK |
| | SEQ ID | DIQMTQSPSSLSASVGDRVTITCRASQSIGRWLAWYQQKPGKAPKLLIY |
| | NO: 206 | SASNLETGVPSRFSGSGSGTDFTLTISSLQPEDFATYYCQQYESFPVTF |
| | | GPGTKVDIKRADAAPTVSIFPPSSEQLTSGGASVVCFLNNFYPKDINVK |
| | | WKIDGSERQNGVLNSWTDQDSKDSTYSMSSTLTLTKDEYERHNSYTCEA |
| | | THKTSTSPIVKSFNRNEC |
| anti- | SEQ ID | DIQMTQSPSSLSASVGDRVTITCRASQDVNTAVAWYQQKPGKAPKLLIY |
| HER2/IL-2- | NO: 64 | SASFLYSGVPSRFSGSRSGTDFTLTISSLQPEDFATYYCQQHYTTPPTF |
| cytokine | | GQGTKVEIKRADAAPTVSIFPPSSEQLTSGGASVVCFLNNFYPKDINVK |
| complex | | WKIDGSERQNGVLNSWTDQDSKDSTYSMSSTLTLTKDEYERHNSYTCEA |
| | | THKTSTSPIVKSFNRNEC |
| | SEQ ID | APASSSTKKTQLQLEHLLLDLQMILNGINNYKNPKLTRMLTAKFAMPKK |
| | NO: 207 | ATELKHLQCLEEELKPLEEVLNGAQSKNFHLRPRDLISNINVIVLELKG |
| | | SETTFMCEYADETATIVEFLNRWITFAQSIISTLTGGGGSGGGGGGGGS |
| | | GGGGSEVQLVESGGGLVQPGGSLRLSCAASGFNIKDTYIHWVRQAPGKG |
| | | LEWVARIYPTNGYTRYADSVKGRFTISADTSKNTAYLQMNSLRAEDTAV |
| | | YYCSRWGGDGFYAMDYWGQGTLVTVSSAKTTAPSVYPLAPVCGDTTGSS |
| | | VTLGCLVKGYFPEPVTLTWNSGSLSSGVHTFPAVLQSDLYTLSSSVTVT |
| | | SSTWPSQSITCNVAHPASSTKVDKKIEPRGPTIKPCPPCKCPAPNAAGG |
| | | PSVFIFPPKIKDVLMISLSPIVTCVVVDVSEDDPDVQISWFVNNVEVHT |
| | | AQTQTHREDYNSTLRVVSALPIQHQDWMSGKEFKCKVNNKDLGAPIERT |
| | | ISKPKGSVRAPQVYVLPPPEEEMTKKQVTLTCMVTDFMPEDIYVEWTNN |
| | | GKTELNYKNTEPVLDSDGSYFMYSKLRVEKKNWVERNSYSCSVVHEGLH |
| | | NHHTTKSFSRTPGK |
| Always-off | SEQ ID | APASSSTKKTQLQLEHLLLDLQMILNGINNYKNPKLTRMLTAKFAMPKK |
| | NO: 208 | ATELKHLQCLEEELKPLEEVLNGAQSKNFHLRPRDLISNINVIVLELKG |
| | | SETTFMCEYADETATIVEFLNRWITFAQSIISTLTGGGGSGGGGSGGGG |
| | | SGGGGSEVQLVESGGGLVKPGGSLRLSCAASGFTFSSYTLAWVRQAPGK |
| | | GLEWVAAIDSSSYTYSPDTVRGRFTISRDNAKNSLYLQMNSLRAEDTAV |
| | | YYCARDSNWDALDYWGQGTLVTVSSAKTTAPSVYPLAPVCGDTTGSSVT |
| | | LGCLVKGYFPEPVTLTWNSGSLSSGVHTFPAVLQSDLYTLSSSVTVTSS |
| | | TWPSQSITCNVAHPASSTKVDKKIEPRGPTIKPCPPCKCPAPNAAGGPS |
| | | VFIFPPKIKDVLMISLSPIVTCVVVDVSEDDPDVQISWFVNNVEVHTAQ |
| | | TQTHREDYNSTLRVVSALPIQHQDWMSGKEFKCKVNNKDLGAPIERTIS |
| | | KPKGSVRAPQVYVLPPPEEEMTKKQVTLTCMVTDFMPEDIYVEWTNNGK |
| | | TELNYKNTEPVLDSDGSYFMYSKLRVEKKNWVERNSYSCSVVHEGLHNH |
| | | HTTKSFSRTPGK |
| IL-2 mut | SEQ ID | DIQMTQSPSSLSASVGDRVSITCKASQNVGTNVGWYQQKPGKAPKALIY |
| | NO: 209 | SASFRYSGVPSRFSGSGSGTDFTLTISSLQPEDFATYFCQQYYTYPYTF |
| | | GGGTKLEIKRADAAPTVSIFPPSSEQLTSGGASVVCFLNNFYPKDINVK |
| | | WKIDGSERQNGVLNSWTDQDSKDSTYSMSSTLTLTKDEYERHNSYTCEA |
| | | THKTSTSPIVKSFNRNEC |

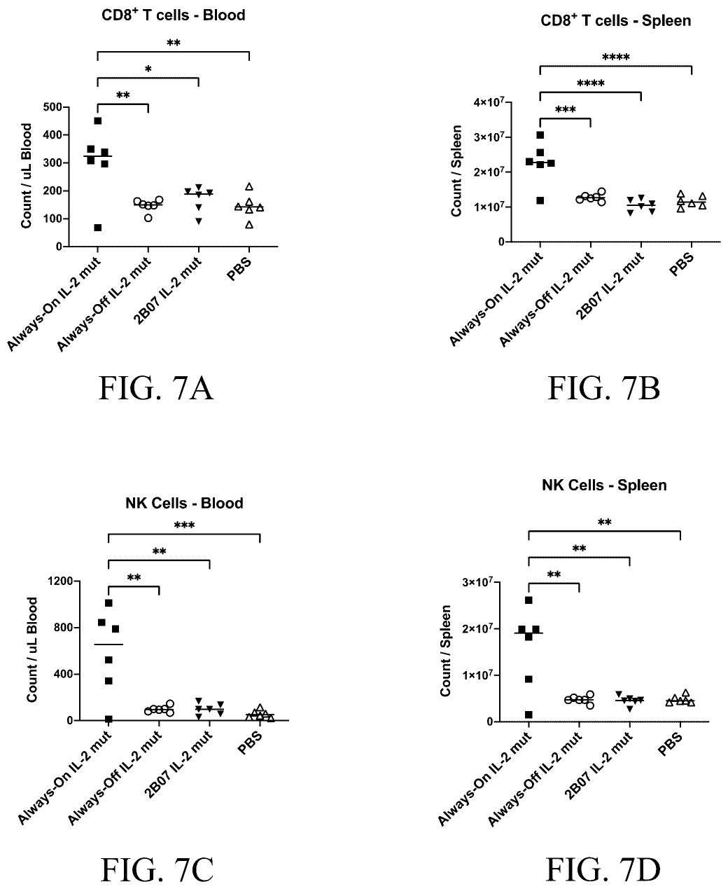

To examine the activity of PD-1/IL-2 DBA-cytokine complexes in peripheral tissues, wild-type C57BL/6 mice received a single 2.5 milligrams per kilogram intravenous dose of PD-1/IL-2 DBA-cytokine complex (2B07 IL-2 mut; SEQ ID NO: 205-206), anti-HER2/IL-2-cytokine complex (Always-on IL-2 mut; SEQ ID NO: 64 and SEQ ID NO: 207), anti-IL-2/IL-2-cytokine complex (Always-off IL-2 mut; SEQ ID NO: 208-209), as shown in TABLE 12 or PBS. Prior to dosing, the presence of intact IL-2 within each IL-2 cytokine complex was confirmed by ELISA as a means of verifying their potential for biological activity. Blood and spleens were collected 5 days following treatment and analyzed by flow cytometry to quantify the number of CD8+ T cells and NK cells per spleen and per microliter of blood. The PD-1/IL-2 DBA-cytokine complex did not induce expansion of CD8 T cells or NK cells, whereas the HER2/IL-2-cytokine complex induced expansion of peripheral CD8+ T cells and NK cells (FIG. 7A-D).

### Example 8

### PD-1/IL-2 DBA Cytokine Complex Modulation of Anti-Tumor Immunity in Syngeneic Tumor Models

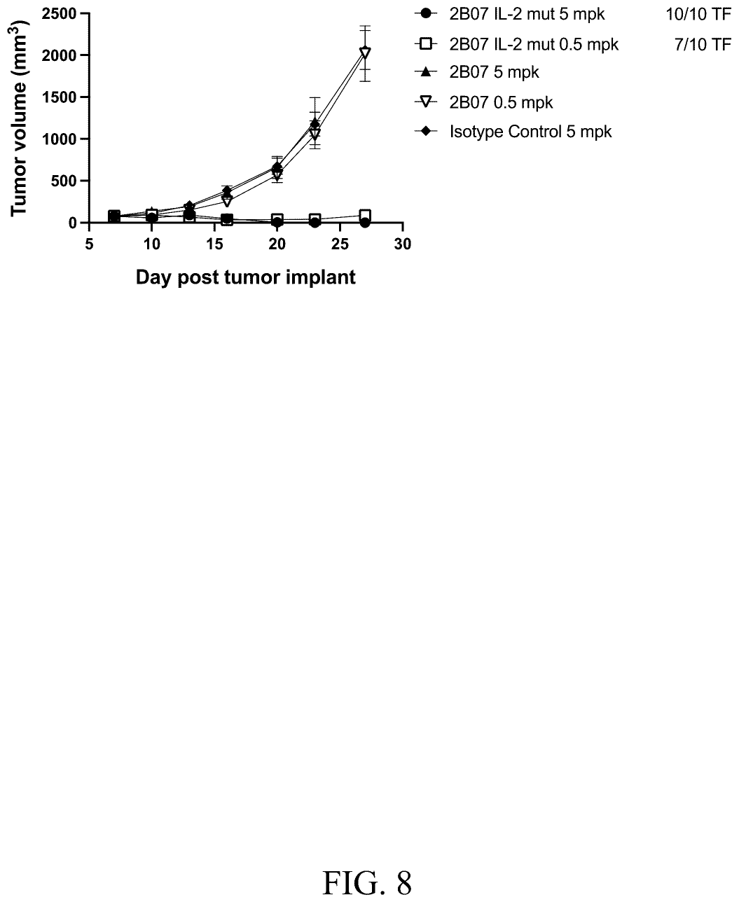

This example describes PD-1/IL-2 DBA-cytokine complex modulation of anti-tumor immunity in a MC38 syngeneic mouse tumor model. A PD-1/IL-2 DBA-cytokine complex was assessed for the ability to drive anti-tumor immunity in vivo. 500,000 MC38 tumor cells were implanted subcutaneously in human PD-1 knock-in mice (GenOway). Tumors were measured twice weekly, and volumes calculated as (Length x Width x Width/2). Mice were randomized into treatment groups, and treatments were initiated when tumors reached a volume of ˜100 mm³. Mice were treated intravenously with PD-1/IL-2 DBA-cytokine complex (2B07 IL-2 mut; SEQ ID NO: 210-212), PD-1/IL-2 DBA lacking IL-2 (2B07; SEQ ID NO: 212-213), or an isotype control (SEQ ID NO: 214-215), as shown in TABLE 13 below, at the indicated doses of 5 or 0.5 milligrams per kilogram on days 7, 10, and 13 post tumor implantation. The PD-1/IL-2 DBA-cytokine complex showed increased tumor growth inhibition compared to either the PD-1/IL-2 DBA lacking IL-2 or the isotype control (FIG. 8).

TABLE 13
IgG PD-1/IL-2 DBA and control protein complexes

| Protein | SEQ ID | |
|---|---|---|
| Complex | NO: | Sequence |
| 2B07 IL-2 | SEQ ID | APASSSTKKTQLQLEHLLLDLQMILNGINNYKNPKLTRMLTAKFAMPKK |
| mut | NO: 210 | ATELKHLQCLEEELKPLEEVLNGAQSKNFHLRPRDLISNINVIVLELKG |
| | | SETTFMCEYADETATIVEFLNRWITFAQSIISTLTGGGGSGGGGSGGGG |
| | | SGGGGSQVQLVQSGAEVKKPGASVKVSCKASGDTFTRYYVHWVRQAPGQ |
| | | GLEWMGIINPSGGYASYAQKFQGRVTMTRDTSTSTVYMELSSLRSEDTA |
| | | VYYCAAGLFIWGQGTLVTVSSAKTTAPSVYPLAPVCGDTTGSSVTLGCL |
| | | VKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNAAGGPSVFIF |
| | | PPKIKDVLMISLSPIVTCVVVDVSEDDPDVQISWFVNNVEVHTAQTQTH |
| | | REDYNSTLRVVSALPIQHQDWMSGKEFKCKVNNKDLGAPIERTISKPKG |
| | | SVRAPQVYVLPPPEEEMTKKQVTLTCMVTDFMPEDIYVEWTNNGKTELN |
| | | YKNTEPVLDSDGSYFMYSDLRVEKKNWVERNSYSCSVVHEGLHNHHTTE |
| | | SFSRTPGK |
| | SEQ ID | QVQLVQSGAEVKKPGASVKVSCKASGDTFTRYYVHWVRQAPGQGLEWMG |
| | NO: 211 | IINPSGGYASYAQKFQGRVTMTRDTSTSTVYMELSSLRSEDTAVYYCAA |
| | | GLFIWGQGTLVTVSSAKTTAPSVYPLAPVCGDTTGSSVTLGCLVKGYFP |
| | | EPVTLTWNSGSLSSGVHTFPAVLQSDLYTLSSSVTVTSSTWPSQSITCN |
| | | VAHPASSTKVDKKIEPRGPTIKPCPPCKCPAPNAAGGPSVFIFPPKIKD |
| | | VLMISLSPIVTCVVVDVSEDDPDVQISWFVNNVEVHTAQTQTHREDYNS |
| | | TLRVVSALPIQHQDWMSGKEFKCKVNNKDLGAPIERTISKPKGSVRAPQ |
| | | VYVLPPPEKEMTKKQVSLTCLVKDFMPEDIYVEWTNNGKTELNYKNTEP |
| | | VLKSDGSYFMYSKLTVEKKNWVERNSYSCSVVHEGLHNHHTTKSFSRTP |
| | | GK |
| | SEQ ID | DIQMTQSPSSLSASVGDRVTITCRASQSIGRYLAWYQQKPGKAPKLLIY |
| | NO: 212 | SASNLETGVPSRFSGSGSGTDFTLTISSLQPEDFATYYCQQYNSFPVTF |
| | | GPGTKVDIKRADAAPTVSIFPPSSEQLTSGGASVVCFLNNFYPKDINVK |
| | | WKIDGSERQNGVLNSWTDQDSKDSTYSMSSTLTLTKDEYERHNSYTCEA |
| | | THKTSTSPIVKSFNRNEC |
| PD-1/IL-2 | SEQ ID | DIQMTQSPSSLSASVGDRVTITCRASQSIGRYLAWYQQKPGKAPKLLIY |
| DBA | NO: 212 | SASNLETGVPSRFSGSGSGTDFTLTISSLQPEDFATYYCQQYNSFPVTF |
| lacking | | GPGTKVDIKRADAAPTVSIFPPSSEQLTSGGASVVCFLNNFYPKDINVK |
| IL-2 | | WKIDGSERQNGVLNSWTDQDSKDSTYSMSSTLTLTKDEYERHNSYTCEA |
| | | THKTSTSPIVKSFNRNEC |
| | SEQ ID | QVQLVQSGAEVKKPGASVKVSCKASGDTFTRYYVHWVRQAPGQGLEWMG |
| | NO: 213 | IINPSGGYASYAQKFQGRVTMTRDTSTSTVYMELSSLRSEDTAVYYCAA |
| | | GLFIWGQGTLVTVSSAKTTAPSVYPLAPVCGDTTGSSVTLGCLVKGYFP |
| | | EPVTLTWNSGSLSSGVHTFPAVLQSDLYTLSSSVTVTSSTWPSQSITCN |
| | | VAHPASSTKVDKKIEPRGPTIKPCPPCKCPAPNAAGGPSVFIFPPKIKD |
| | | VLMISLSPIVTCVVVDVSEDDPDVQISWFVNNVEVHTAQTQTHREDYNS |
| | | TLRVVSALPIQHQDWMSGKEFKCKVNNKDLGAPIERTISKPKGSVRAPQ |
| | | VYVLPPPEEEMTKKQVTLTCMVTDFMPEDIYVEWTNNGKTELNYKNTEP |
| | | VLDSDGSYFMYSKLRVEKKNWVERNSYSCSVVHEGLHNHHTTKSFSRTP |
| | | GK |
| isotype | SEQ ID | EVQLVESGGGLVQPGGSLRLSCAASGFNIKDTYIHWVRQAPGKGLEWVA |
| control | NO: 214 | RIYPTNGYTRYADSVKGRFTISADTSKNTAYLQMNSLRAEDTAVYYCSR |
| | | WGGDGFYAMDYWGQGTLVTVSSAKTTAPSVYPLAPVCGDTTGSSVTLGC |
| | | LVKGYFPEPVTLTWNSGSLSSGVHTFPAVLQSDLYTLSSSVTVTSSTWP |
| | | SQSITCNVAHPASSTKVDKKIEPRGPTIKPCPPCKCPAPNAAGGPSVFI |
| | | FPPKIKDVLMISLSPIVTCVVVDVSEDDPDVQISWFVNNVEVHTAQTQT |
| | | HREDYNSTLRVVSALPIQHQDWMSGKEFKCKVNNKDLGAPIERTISKPK |
| | | GSVRAPQVYVLPPPEEEMTKKQVTLTCMVTDFMPEDIYVEWTNNGKTEL |
| | | NYKNTEPVLDSDGSYFMYSKLRVEKKNWVERNSYSCSVVHEGLHNHHTT |
| | | KSFSRTPGK |
| | SEQ ID | DIQMTQSPSSLSASVGDRVTITCRASQDVNTAVAWYQQKPGKAPKLLIY |
| | NO: 215 | SASFLYSGVPSRFSGSRSGTDFTLTISSLQPEDFATYYCQQHYTTPPTF |
| | | GQGTKVEIKRADAAPTVSIFPPSSEQLTSGGASVVCFLNNFYPKDINVK |
| | | WKIDGSERQNGVLNSWTDQDSKDSTYSMSSTLTLTKDEYERHNSYTCEA |
| | | THKTSTSPIVKSFNRNEC |

### Example 9

### PD-1/IL-2 DBA Cytokine Complex Modulation of Anti-Tumor Immunity in Xenograft/Human Immune Cell Admixture Models

This example describes PD-1/IL-2 DBA-cytokine complex modulation of anti-tumor immunity in xenograft/human immune cell admixture models. To examine the ability of PD-1/IL-2 DBA-cytokine complexes to drive anti-tumor immunity in an in vivo setting, an admixture system is used. Total human PBMCs or a combination of human T cells and monocyte-derived dendritic cells (moDCs) are mixed with human tumor cells (e.g., HPAC, A375, H441) at a 1:4 ratio and co-implanted subcutaneously into the flanks of NSG mice. One day later, treatment with a PD-1/IL-2 DBA-cytokine complex of the present disclosure, or suitable non-regulated controls such as anti-PD-1, anti-HER2-IL-2, or anti-PD-1-IL-2, is initiated. Tumors are measured at least twice weekly and volumes calculated as (Length×Width×Height/2). PD-1/IL-2 DBA-cytokine complexes exhibit increased anti-tumor efficacy compared to anti-PD-1 and anti-HER2-IL-2 and decreased off-tumor activity compared to anti-PD-1-IL-2.

### Example 10

### General Method: In Vitro and In Vivo Characterization of Protein Complexes

This example describes the evaluation of DBA-cytokine complexes for in vitro and in vivo stability. A protein complex of the present disclosure is recombinantly expressed or chemically synthesized. The protein complex includes a sensor domain linked to a therapeutic domain. The linker is a peptide linker. The sensor domain is capable of binding to the therapeutic domain and a marker. In the absence if the marker, the sensor domain binds the therapeutic domain rendering the therapeutic domain unable to bind to its target and unable to exert therapeutic activity. In the presence of the marker, the sensor domain binds the marker rendering the therapeutic domain free to bind to its target and able to exert therapeutic activity.

In vitro, the protein complexes are tested for stability and functionality at baseline or after incubation in conditions of stress, such as elevated temperature, pH changes, oxidative buffers, or serum/plasma, using methods of biophysical characterization to measure fragmentation, unfolding, or aggregation, and/or using methods to test for changes in functional activity. In vivo, the pharmacokinetic properties of the proteins are measured following dosing in a mammal, such as a mouse, rat, or non-human primate, and properties of distribution, clearance and degradation are measured. These measurements are used to engineer or select the optimal therapeutic form of the DBA-protein complex.

### Example 11

### Regulated IL-2 Receptor Signaling by a PD-1/IL-2 Dual Binding Antibody (DBA) Cytokine Complex

This example describes PD-1 regulated IL-2 activity in a HEK-BLUE™ IL-2 reporter cell by PD-1/IL-2 DBA-cytokine complexes. The DBA-cytokine complexes and control antibody-cytokine complexes were produced in three formats shown in FIGS. 2E, 2B AND 2H by expression in mammalian cells using standard protocols. The wells of a 384-well ELISA plate were coated with constant concentration of PD-1-Fc or an IgG1 control protein captured with an anti-Fc antibody (Jackson ImmunoRescarch, Prod. #109-005-098). The cytokine complexes were serially diluted 1:4 for 8 points in growth media from a starting concentration of 6 nM and incubated briefly before addition of the HEK-BLUE™ IL-2 reporter cells.

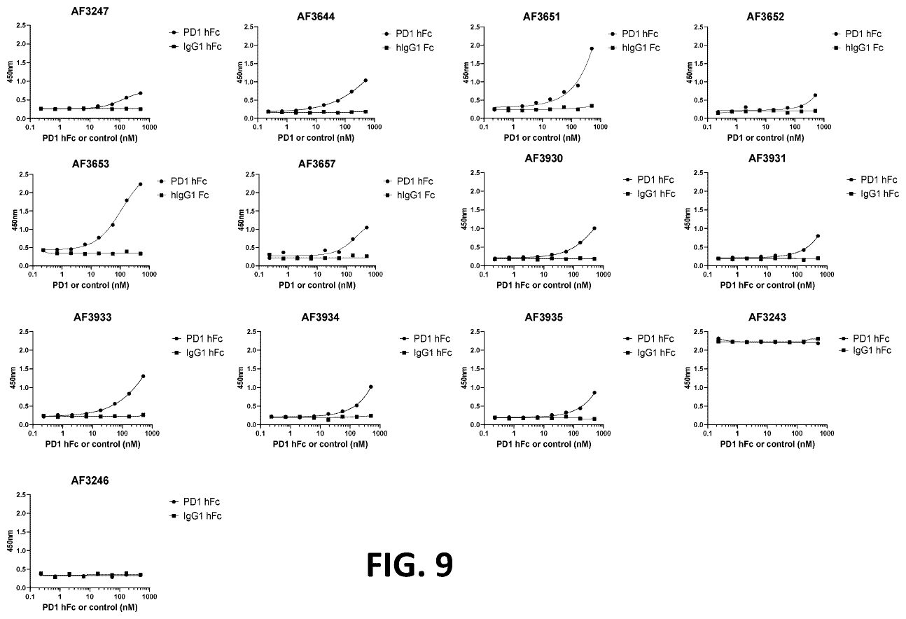

Results with protein complexes comprising the structure shown in FIG. 2E are shown in FIG. 9A-D. As depicted in FIG. 2E, this symmetric format is comprised of one IL-2 linked to each antibody variable domain. The IL-2 activity of the PD-1/IL-2 DBA-IL-2 complex AF4379 comprising SEQ ID NO: 174-175 had an EC50 of 31 PM in the PD-1 coated wells versus 62 pM in the IgG1 coated wells, as shown in FIG. 9A, demonstrating PD-1 dependence. The IL-2 activity of antibody-cytokine complexes AF4377 comprising SEQ ID NO: 64 and 176 (anti-Her2 antibody) and AF4378 comprising SEQ ID NO: 177-178 (anti-IL-2 antibody) was unchanged in the presence of PD-1 (as shown in FIG. 9B and FIG. 9C, respectively), while the IL-2 activity of the anti-PD-1 antibody AF4376 comprising SEQ ID NO: 179-180 is reduced in the presence of PD-1, as shown in FIG. 9D. Sequences of the protein complexes are summarized in TABLE 14 below.

TABLE 14
IgG PD-1/IL-2 DBA with heavy chain IL-2 therapeutic
domains, and control protein complexes

| Protein | | |
|---|---|---|
| Complex | SEQ ID NO: | Sequence |
| AF4379 | SEQ ID NO: | APTSSSTKKTQLQLEHLLLDLQMILNGINNYKNPKLTDMLT |
| | 174 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSQVQLVQSGAEV |
| | | KKPGASVKVSCKASGDTFTRYYVHWVRQAPGQGLEWMGIIN |
| | | PSGGYASYAQKFQGRVTMTRDTSTSTVYMELSSLRSEDTAV |
| | | YYCAAGLFIWGQGTLVTVSSAKTTAPSVYPLAPVCGDTTGS |
| | | SVTLGCLVKGYFPEPVTLTWNSGSLSSGVHTFPAVLQSDLY |
| | | TLSSSVTVTSSTWPSQSITCNVAHPASSTKVDKKIEPRGPT |
| | | IKPCPPCKCPAPNAAGGPSVFIFPPKIKDVLMISLSPIVTC |
| | | VVVDVSEDDPDVQISWFVNNVEVHTAQTQTHREDYNSTLRV |
| | | VSALPIQHQDWMSGKEFKCKVNNKDLGAPIERTISKPKGSV |
| | | RAPQVYVLPPPEEEMTKKQVTLTCMVTDFMPEDIYVEWTNN |
| | | GKTELNYKNTEPVLDSDGSYFMYSKLRVEKKNWVERNSYSC |
| | | SVVHEGLHNHHTTKSFSRTPGK |
| | SEQ ID NO: | DIQMTQSPSSLSASVGDRVTITCRASQSIGRYLAWYQQKPG |
| | 175 | KAPKLLIYSASNLETGVPSRFSGSGSGTDFTLTISSLQPED |
| | | FATYYCQQYNSFPVTFGPGTKVDIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| AF4377 | SEQ ID NO: | DIQMTQSPSSLSASVGDRVTITCRASQDVNTAVAWYQQKPG |
| | 64 | KAPKLLIYSASFLYSGVPSRFSGSRSGTDFTLTISSLQPED |
| | | FATYYCQQHYTTPPTFGQGTKVEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID NO: | APTSSSTKKTQLQLEHLLLDLQMILNGINNYKNPKLTDMLT |
| | 176 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSEVQLVESGGGL |
| | | VQPGGSLRLSCAASGFNIKDTYIHWVRQAPGKGLEWVARIY |
| | | PTNGYTRYADSVKGRFTISADTSKNTAYLQMNSLRAEDTAV |
| | | YYCSRWGGDGFYAMDYWGQGTLVTVSSAKTTAPSVYPLAPV |
| | | CGDTTGSSVTLGCLVKGYFPEPVTLTWNSGSLSSGVHTFPA |
| | | VLQSDLYTLSSSVTVTSSTWPSQSITCNVAHPASSTKVDKK |
| | | IEPRGPTIKPCPPCKCPAPNAAGGPSVFIFPPKIKDVLMIS |
| | | LSPIVTCVVVDVSEDDPDVQISWFVNNVEVHTAQTQTHRED |
| | | YNSTLRVVSALPIQHQDWMSGKEFKCKVNNKDLGAPIERTI |
| | | SKPKGSVRAPQVYVLPPPEEEMTKKQVTLTCMVTDFMPEDI |
| | | YVEWTNNGKTELNYKNTEPVLDSDGSYFMYSKLRVEKKNWV |
| | | ERNSYSCSVVHEGLHNHHTTKSFSRTPGK |
| AF4378 | SEQ ID NO: | APTSSSTKKTQLQLEHLLLDLQMILNGINNYKNPKLTDMLT |
| | 177 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSEVQLVESGGGL |
| | | VKPGGSLRLSCAASGFTFSSYTLAWVRQAPGKGLEWVAAID |
| | | SSSYTYSPDTVRGRFTISRDNAKNSLYLQMNSLRAEDTAVY |
| | | YCARDSNWDALDYWGQGTLVTVSSAKTTAPSVYPLAPVCGD |
| | | TTGSSVTLGCLVKGYFPEPVTLTWNSGSLSSGVHTFPAVLQ |
| | | SDLYTLSSSVTVTSSTWPSQSITCNVAHPASSTKVDKKIEP |
| | | RGPTIKPCPPCKCPAPNAAGGPSVFIFPPKIKDVLMISLSP |
| | | IVTCVVVDVSEDDPDVQISWFVNNVEVHTAQTQTHREDYNS |
| | | TLRVVSALPIQHQDWMSGKEFKCKVNNKDLGAPIERTISKP |
| | | KGSVRAPQVYVLPPPEEEMTKKQVTLTCMVTDFMPEDIYVE |
| | | WTNNGKTELNYKNTEPVLDSDGSYFMYSKLRVEKKNWVERN |
| | | SYSCSVVHEGLHNHHTTKSFSRTPGK |
| | SEQ ID NO: | DIQMTQSPSSLSASVGDRVSITCKASQNVGTNVGWYQQKPG |
| | 178 | KAPKALIYSASFRYSGVPSRFSGSGSGTDFTLTISSLQPED |
| | | FATYFCQQYYTYPYTFGGGTKLEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| AF4376 | SEQ ID NO: | APTSSSTKKTQLQLEHLLLDLQMILNGINNYKNPKLTDMLT |
| | 179 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSQVQLVESGGGV |
| | | VQPGRSLRLDCKASGITFSNSGMHWVRQAPGKGLEWVAVIW |
| | | YDGSKRYYADSVKGRFTISRDNSKNTLFLQMNSLRAEDTAV |
| | | YYCATNDDYWGQGTLVTVSSAKTTAPSVYPLAPVCGDTTGS |
| | | SVTLGCLVKGYFPEPVTLTWNSGSLSSGVHTFPAVLQSDLY |
| | | TLSSSVTVTSSTWPSQSITCNVAHPASSTKVDKKIEPRGPT |
| | | IKPCPPCKCPAPNAAGGPSVFIFPPKIKDVLMISLSPIVTC |
| | | VVVDVSEDDPDVQISWFVNNVEVHTAQTQTHREDYNSTLRV |
| | | VSALPIQHQDWMSGKEFKCKVNNKDLGAPIERTISKPKGSV |
| | | RAPQVYVLPPPEEEMTKKQVTLTCMVTDFMPEDIYVEWTNN |
| | | GKTELNYKNTEPVLDSDGSYFMYSKLRVEKKNWVERNSYSC |
| | | SVVHEGLHNHHTTKSFSRTPGK |
| | SEQ ID NO: | EIVLTQSPATLSLSPGERATLSCRASQSVSSYLAWYQQKPG |
| | 180 | QAPRLLIYDASNRATGIPARFSGSGSGTDFTLTISSLEPED |
| | | FAVYYCQQSSNWPRTFGQGTKVEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |

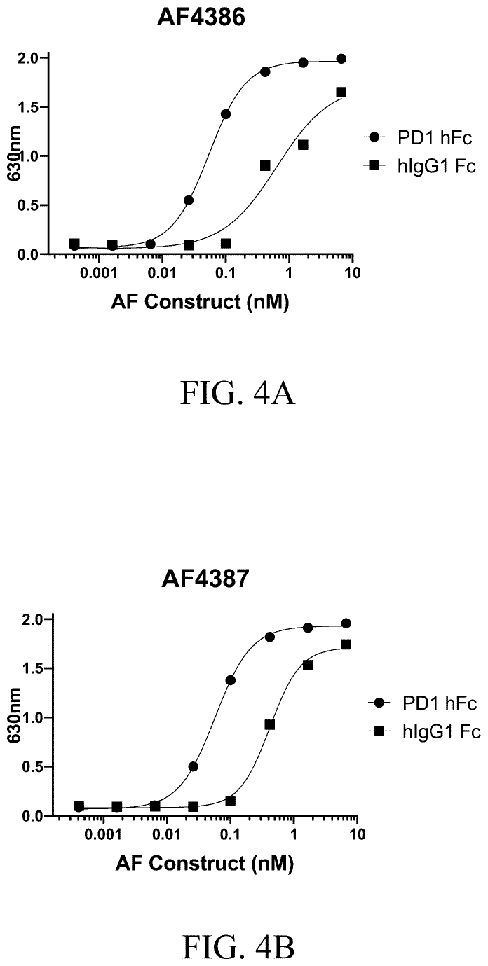

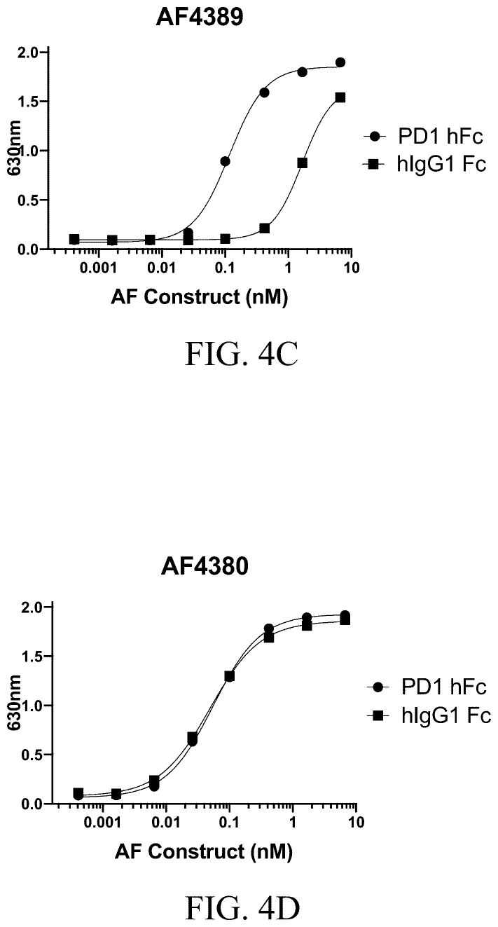

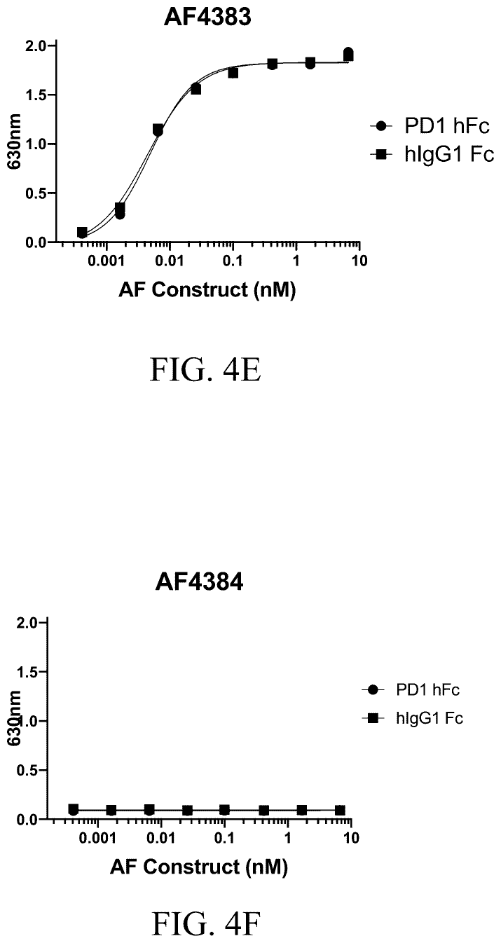

Results with protein complexes comprising the structures depicted in FIG. 2B, are shown in FIGS. 4A-F. This format is composed of an asymmetric complex comprised of two antibody domains with a single IL-2 linked to one of the domains. The IL-2 activity of the PD-1/IL-2 DBA-IL-2 complexes AF4386 (comprising SEQ ID NO: 212 and 181-182, results shown in FIG. 4A), AF4387 (comprising SEQ ID NO: 183-185, results shown in FIG. 4B) and AF4389 (comprising SEQ ID NO: 186-188, results shown in FIG. 4C) had an EC50 of 50 pM, 57 pM and 118 pM respectively in the PD-1 coated wells and 1.79 nM, 419 pM and 1.67 nM respectively in the IgG1 coated wells, demonstrating PD-1 dependence. The IL-2 activity of the anti-PD1 control protein AF4380 (comprising SEQ ID NO: 180, 189-190, results shown in FIG. 4D), the anti-Her2 control protein AF4383 (comprising SEQ ID NO: 64, 191-192, results shown in FIG. 4E), and the anti-IL-2 control protein AF4384 (comprising SEQ ID NO: 178, 193-194, results shown in FIG. 4F) were unchanged. Sequences of the protein complexes are summarized in TABLE 15 below.

TABLE 15
IgG PD-1/IL-2 PDA with single IL-2, and
control protein complexes

| Protein | | |
|---|---|---|
| Complex | SEQ ID NO: | Sequence |
| AF4386 | SEQ ID NO: | APTSSSTKKTQLQLEHLLLDLQMILNGINNYKNPKLTDMLT |
| | 181 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSQVQLVQSGAEV |
| | | KKPGASVKVSCKASGDTFTRYYVHWVRQAPGQGLEWMGIIN |
| | | PSGGYASYAQKFQGRVTMTRDTSTSTVYMELSSLRSEDTAV |
| | | YYCAAGLFIWGQGTLVTVSSAKTTAPSVYPLAPVCGDTTGS |
| | | SVTLGCLVKGYFPEPVTLTWNSGSLSSGVHTFPAVLQSDLY |
| | | TLSSSVTVTSSTWPSQSITCNVAHPASSTKVDKKIEPRGPT |
| | | IKPCPPCKCPAPNAAGGPSVFIFPPKIKDVLMISLSPIVTC |
| | | VVVDVSEDDPDVQISWFVNNVEVHTAQTQTHREDYNSTLRV |
| | | VSALPIQHQDWMSGKEFKCKVNNKDLGAPIERTISKPKGSV |
| | | RAPQVYVLPPPEEEMTKKQVTLTCMVTDFMPEDIYVEWTNN |
| | | GKTELNYKNTEPVLDSDGSYFMYSDLRVEKKNWVERNSYSC |
| | | SVVHEGLHNHHTTESFSRTPGK |
| | SEQ ID NO: | QVQLVQSGAEVKKPGASVKVSCKASGDTFTRYYVHWVRQAP |
| | 182 | GQGLEWMGIINPSGGYASYAQKFQGRVTMTRDTSTSTVYME |
| | | LSSLRSEDTAVYYCAAGLFIWGQGTLVTVSSAKTTAPSVYP |
| | | LAPVCGDTTGSSVTLGCLVKGYFPEPVTLTWNSGSLSSGVH |
| | | TFPAVLQSDLYTLSSSVTVTSSTWPSQSITCNVAHPASSTK |
| | | VDKKIEPRGPTIKPCPPCKCPAPNAAGGPSVFIFPPKIKDV |
| | | LMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPPEKEMTKKQVSLTCLVKDFM |
| | | PEDIYVEWTNNGKTELNYKNTEPVLKSDGSYFMYSKLTVEK |
| | | KNWVERNSYSCSVVHEGLHNHHTTKSFSRTPGGGGSGGGSH |
| | | HHHHH |
| | SEQ ID NO: | DIQMTQSPSSLSASVGDRVTITCRASQSIGRYLAWYQQKPG |
| | 212 | KAPKLLIYSASNLETGVPSRFSGSGSGTDFTLTISSLQPED |
| | | FATYYCQQYNSFPVTFGPGTKVDIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| AF4387 | SEQ ID NO: | APTSSSTKKTQLQLEHLLLDLQMILNGINNYKNPKLTDMLT |
| | 183 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSQVQLVQSGAEV |
| | | KKPGASVKVSCKASGYTFTDYYMHWVRQAPGQGLEWMGIIN |
| | | PRAGYTSYALKFQGRVTMTRDTSTSTVYMELSSLRSEDTAV |
| | | YYCTSGWDVWGQGTLVTVSSAKTTAPSVYPLAPVCGDTTGS |
| | | SVTLGCLVKGYFPEPVTLTWNSGSLSSGVHTFPAVLQSDLY |
| | | TLSSSVTVTSSTWPSQSITCNVAHPASSTKVDKKIEPRGPT |
| | | IKPCPPCKCPAPNAAGGPSVFIFPPKIKDVLMISLSPIVTC |
| | | VVVDVSEDDPDVQISWFVNNVEVHTAQTQTHREDYNSTLRV |
| | | VSALPIQHQDWMSGKEFKCKVNNKDLGAPIERTISKPKGSV |
| | | RAPQVYVLPPPEEEMTKKQVTLTCMVTDFMPEDIYVEWTNN |
| | | GKTELNYKNTEPVLDSDGSYFMYSDLRVEKKNWVERNSYSC |
| | | SVVHEGLHNHHTTESFSRTPGK |
| | SEQ ID NO: | QVQLVQSGAEVKKPGASVKVSCKASGYTFTDYYMHWVRQAP |
| | 184 | GQGLEWMGIINPRAGYTSYALKFQGRVTMTRDTSTSTVYME |
| | | LSSLRSEDTAVYYCTSGWDVWGQGTLVTVSSAKTTAPSVYP |
| | | LAPVCGDTTGSSVTLGCLVKGYFPEPVTLTWNSGSLSSGVH |
| | | TFPAVLQSDLYTLSSSVTVTSSTWPSQSITCNVAHPASSTK |
| | | VDKKIEPRGPTIKPCPPCKCPAPNAAGGPSVFIFPPKIKDV |
| | | LMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPPEKEMTKKQVSLTCLVKDFM |
| | | PEDIYVEWTNNGKTELNYKNTEPVLKSDGSYFMYSKLTVEK |
| | | KNWVERNSYSCSVVHEGLHNHHTTKSFSRTPGGGGSGGGSH |
| | | HHHHH |
| | SEQ ID NO: | DIQMTQSPSSLSASVGDRVTITCRASQSISTWLAWYQQKPG |
| | 185 | KAPKLLIYAASSLDSGVPSRFSGSGSGTDFTLTISSLQPED |
| | | FATYYCQQSYSFPVTFGQGTKVEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| AF4389 | SEQ ID NO: | APTSSSTKKTQLQLEHLLLDLQMILNGINNYKNPKLTDMLT |
| | 186 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSQVQLVQSGAEV |
| | | KKPGASVKVSCKASGHTFTRYYMHWVRQAPGQGLEWMGIIN |
| | | PSGGYATYAQKFQGRVTMTRDTSTSTVYMELSSLRSEDTAV |
| | | YYCASGLFIWGQGTLVTVSSAKTTAPSVYPLAPVCGDTTGS |
| | | SVTLGCLVKGYFPEPVTLTWNSGSLSSGVHTFPAVLQSDLY |
| | | TLSSSVTVTSSTWPSQSITCNVAHPASSTKVDKKIEPRGPT |
| | | IKPCPPCKCPAPNAAGGPSVFIFPPKIKDVLMISLSPIVTC |
| | | VVVDVSEDDPDVQISWFVNNVEVHTAQTQTHREDYNSTLRV |
| | | VSALPIQHQDWMSGKEFKCKVNNKDLGAPIERTISKPKGSV |
| | | RAPQVYVLPPPEEEMTKKQVTLTCMVTDFMPEDIYVEWTNN |
| | | GKTELNYKNTEPVLDSDGSYFMYSDLRVEKKNWVERNSYSC |
| | | SVVHEGLHNHHTTESFSRTPGK |
| | SEQ ID NO: | QVQLVQSGAEVKKPGASVKVSCKASGHTFTRYYMHWVRQAP |
| | 187 | GQGLEWMGIINPSGGYATYAQKFQGRVTMTRDTSTSTVYME |
| | | LSSLRSEDTAVYYCASGLFIWGQGTLVTVSSAKTTAPSVYP |
| | | LAPVCGDTTGSSVTLGCLVKGYFPEPVTLTWNSGSLSSGVH |
| | | TFPAVLQSDLYTLSSSVTVTSSTWPSQSITCNVAHPASSTK |
| | | VDKKIEPRGPTIKPCPPCKCPAPNAAGGPSVFIFPPKIKDV |
| | | LMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPPEKEMTKKQVSLTCLVKDFM |
| | | PEDIYVEWTNNGKTELNYKNTEPVLKSDGSYFMYSKLTVEK |
| | | KNWVERNSYSCSVVHEGLHNHHTTKSFSRTPGGGGSGGGSH |
| | | HHHHH |
| | SEQ ID NO: | DIQMTQSPSSLSASVGDRVTITCRASQSINSWLAWYQQKPG |
| | 188 | KAPKLLIYATSTLESGVPSRFSGSGSGTDFTLTISSLQPED |
| | | FATYYCQQYYRFPVTFGQGTKVEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| AF4380 | SEQ ID NO: | EIVLTQSPATLSLSPGERATLSCRASQSVSSYLAWYQQKPG |
| | 180 | QAPRLLIYDASNRATGIPARFSGSGSGTDFTLTISSLEPED |
| | | FAVYYCQQSSNWPRTFGQGTKVEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID NO: | APTSSSTKKTQLQLEHLLLDLQMILNGINNYKNPKLTDMLT |
| | 189 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSQVQLVESGGGV |
| | | VQPGRSLRLDCKASGITFSNSGMHWVRQAPGKGLEWVAVIW |
| | | YDGSKRYYADSVKGRFTISRDNSKNTLFLQMNSLRAEDTAV |
| | | YYCATNDDYWGQGTLVTVSSAKTTAPSVYPLAPVCGDTTGS |
| | | SVTLGCLVKGYFPEPVTLTWNSGSLSSGVHTFPAVLQSDLY |
| | | TLSSSVTVTSSTWPSQSITCNVAHPASSTKVDKKIEPRGPT |
| | | IKPCPPCKCPAPNAAGGPSVFIFPPKIKDVLMISLSPIVTC |
| | | VVVDVSEDDPDVQISWFVNNVEVHTAQTQTHREDYNSTLRV |
| | | VSALPIQHQDWMSGKEFKCKVNNKDLGAPIERTISKPKGSV |
| | | RAPQVYVLPPPEEEMTKKQVTLTCMVTDFMPEDIYVEWTNN |
| | | GKTELNYKNTEPVLDSDGSYFMYSDLRVEKKNWVERNSYSC |
| | | SVVHEGLHNHHTTESFSRTPGK |
| | SEQ ID NO: | QVQLVESGGGVVQPGRSLRLDCKASGITFSNSGMHWVRQAP |
| | 190 | GKGLEWVAVIWYDGSKRYYADSVKGRFTISRDNSKNTLFLQ |
| | | MNSLRAEDTAVYYCATNDDYWGQGTLVTVSSAKTTAPSVYP |
| | | LAPVCGDTTGSSVTLGCLVKGYFPEPVTLTWNSGSLSSGVH |
| | | TFPAVLQSDLYTLSSSVTVTSSTWPSQSITCNVAHPASSTK |
| | | VDKKIEPRGPTIKPCPPCKCPAPNAAGGPSVFIFPPKIKDV |
| | | LMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPPEKEMTKKQVSLTCLVKDFM |
| | | PEDIYVEWTNNGKTELNYKNTEPVLKSDGSYFMYSKLTVEK |
| | | KNWVERNSYSCSVVHEGLHNHHTTKSFSRTPGGGGSGGGSH |
| | | HHHHH |
| AF4383 | SEQ ID NO: | DIQMTQSPSSLSASVGDRVTITCRASQDVNTAVAWYQQKPG |
| | 64 | KAPKLLIYSASFLYSGVPSRFSGSRSGTDFTLTISSLQPED |
| | | FATYYCQQHYTTPPTFGQGTKVEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID NO: | APTSSSTKKTQLQLEHLLLDLQMILNGINNYKNPKLTDMLT |
| | 191 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSEVQLVESGGGL |
| | | VQPGGSLRLSCAASGFNIKDTYIHWVRQAPGKGLEWVARIY |
| | | PTNGYTRYADSVKGRFTISADTSKNTAYLQMNSLRAEDTAV |
| | | YYCSRWGGDGFYAMDYWGQGTLVTVSSAKTTAPSVYPLAPV |
| | | CGDTTGSSVTLGCLVKGYFPEPVTLTWNSGSLSSGVHTFPA |
| | | VLQSDLYTLSSSVTVTSSTWPSQSITCNVAHPASSTKVDKK |
| | | IEPRGPTIKPCPPCKCPAPNAAGGPSVFIFPPKIKDVLMIS |
| | | LSPIVTCVVVDVSEDDPDVQISWFVNNVEVHTAQTQTHRED |
| | | YNSTLRVVSALPIQHQDWMSGKEFKCKVNNKDLGAPIERTI |
| | | SKPKGSVRAPQVYVLPPPEEEMTKKQVTLTCMVTDFMPEDI |
| | | YVEWTNNGKTELNYKNTEPVLDSDGSYFMYSDLRVEKKNWV |
| | | ERNSYSCSVVHEGLHNHHTTESFSRTPGK |
| | SEQ ID NO: | EVQLVESGGGLVQPGGSLRLSCAASGFNIKDTYIHWVRQAP |
| | 192 | GKGLEWVARIYPTNGYTRYADSVKGRFTISADTSKNTAYLQ |
| | | MNSLRAEDTAVYYCSRWGGDGFYAMDYWGQGTLVTVSSAKT |
| | | TAPSVYPLAPVCGDTTGSSVTLGCLVKGYFPEPVTLTWNSG |
| | | SLSSGVHTFPAVLQSDLYTLSSSVTVTSSTWPSQSITCNVA |
| | | HPASSTKVDKKIEPRGPTIKPCPPCKCPAPNAAGGPSVFIF |
| | | PPKIKDVLMISLSPIVTCVVVDVSEDDPDVQISWFVNNVEV |
| | | HTAQTQTHREDYNSTLRVVSALPIQHQDWMSGKEFKCKVNN |
| | | KDLGAPIERTISKPKGSVRAPQVYVLPPPEKEMTKKQVSLT |
| | | CLVKDFMPEDIYVEWTNNGKTELNYKNTEPVLKSDGSYFMY |
| | | SKLTVEKKNWVERNSYSCSVVHEGLHNHHTTKSFSRTPGGG |
| | | GSGGGSHHHHHH |
| AF4384 | SEQ ID NO: | DIQMTQSPSSLSASVGDRVSITCKASQNVGTNVGWYQQKPG |
| | 178 | KAPKALIYSASFRYSGVPSRFSGSGSGTDFTLTISSLQPED |
| | | FATYFCQQYYTYPYTFGGGTKLEIKRADAAPTVSIFPPSSE |
| | | QLTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID NO: | APTSSSTKKTQLQLEHLLLDLQMILNGINNYKNPKLTDMLT |
| | 193 | FEFYMPKKATELKHLQCLERELKPLEEVLNLAQSKNFHLRP |
| | | RDLISNINVIVLELKGSETTFMCEYADETATIVEFLNRWIT |
| | | FCQSIISTLTGGGGSGGGGSGGGGSGGGGSEVQLVESGGGL |
| | | VKPGGSLRLSCAASGFTFSSYTLAWVRQAPGKGLEWVAAID |
| | | SSSYTYSPDTVRGRFTISRDNAKNSLYLQMNSLRAEDTAVY |
| | | YCARDSNWDALDYWGQGTLVTVSSAKTTAPSVYPLAPVCGD |
| | | TTGSSVTLGCLVKGYFPEPVTLTWNSGSLSSGVHTFPAVLQ |
| | | SDLYTLSSSVTVTSSTWPSQSITCNVAHPASSTKVDKKIEP |
| | | RGPTIKPCPPCKCPAPNAAGGPSVFIFPPKIKDVLMISL |
| | SEQ ID NO: | SPIVTCVVVDVSEDDPDVQISWFVNNVEVHTAQTQTHREDY |
| | 194 | NSTLRVVSALPIQHQDWMSGKEFKCKVNNKDLGAPIERTIS |
| | | KPKGSVRAPQVYVLPPPEEEMTKKQVTLTCMVTDFMPEDIY |
| | | VEWTNNGKTELNYKNTEPVLDSDGSYFMYSDLRVEKKNWVE |
| | | RNSYSCSVVHEGLHNHHTTESFSRTPGKEVQLVESGGGLVK |
| | | PGGSLRLSCAASGFTFSSYTLAWVRQAPGKGLEWVAAIDSS |
| | | SYTYSPDTVRGRFTISRDNAKNSLYLQMNSLRAEDTAVYYC |
| | | DARSNWDALDYWGQGTLVTVSSAKTTAPSVYPLAPVCGDTT |
| | | GSSVTLGCLVKGYFPEPVTLTWNSGSLSSGVHTFPAVLQSD |
| | | LYTLSSSVTVTSSTWPSQSITCNVAHPASSTKVDKKIEPRG |
| | | PTIKPCPPCKCPAPNAAGGPSVFIFPPKIKDVLMISLSPIV |
| | | TCVVVDVSEDDPDVQISWFVNNVEVHTAQTQTHREDYNSTL |
| | | RVVSALPIQHQDWMSGKEFKCKVNNKDLGAPIERTISKPKG |
| | | SVRAPQVYVLPPPEKEMTKKQVSLTCLVKDFMPEDIYVEWT |
| | | NNGKTELNYKNTEPVLKSDGSYFMYSKLTVEKKNWVERNSY |
| | | SCSVVHEGLHNHHTTKSFSRTPGGGGSGGGSHHHHHH |

Results with protein complexes comprising the structures depicted in FIG. 2H are shown in FIGS. 5A-H. As depicted in FIG. 2H, these complexes are asymmetric and comprised of two identical monospecific Fab arms with a single IL-2 attached to one Fc domain by flexible linker and a single scFv attached to the other Fc domain by a flexible linker. The active PD-1/IL-2 DBA complexes, AF4403 comprising SEQ ID NO: 180, 195,199 and AF4404 comprising SEQ ID NO: 180, 196, 199, are composed of anti-PD-1 domains in the Fab arms and a PD-1/IL-2 DBA scFv on the Fc arm. The control antibody-cytokine complexes are composed of a) antibody-cytokine complexes with an irrelevant antibody on the Fab arms with the DBA scFv on the Fc (AF4395 comprising SEQ ID NO: 64, 197, 202 and AF4396 comprising SEQ ID NO: 64, 198, 202), b) antibody-cytokine complexes with a non-DBA scFv on the Fc arm (AF4400 comprising SEQ ID NO: 180, 199-200 and AF4401 comprising SEQ ID NO: 180, 199, 201), and c) antibody-cytokine complexes with non-DBA antibodies in both the Fab and scFv domains (AF4392 comprising SEQ ID NO: 64, 202-203 and AF4393 comprising SEQ ID NO: 64, 202, 204). As shown in FIGS. 5B and 5D, the IL-2 activity of the DBA-cytokine complexes AF4403 and AF4404 had an EC50 of 31 PM and 26 PM respectively in the PD-1 coated wells and 62 pM and 64 pM respectively in the control wells, demonstrating PD-1 dependence of the IL-2 activity. None of the control proteins AF4395, AF4396, AF4400, AF4401, AF4392 and AF4393 described above showed a lower EC50 on PD-1 coated wells than on wells coated with the IgG1 protein, as shown in FIGS. 5A, 5C and 5E-H. Sequences of the protein complexes are summarized in TABLE 16 below.

TABLE 16
IgG PD-1 with C-terminal scFv and IL-2, and control protein complexes

| Protein | SEQ ID | |
|---|---|---|
| Complex | NO: | Sequence |
| AF4403 | SEQ ID | EIVLTQSPATLSLSPGERATLSCRASQSVSSYLAWYQQKPG |
| | NO: 180 | QAPRLLIYDASNRATGIPARFSGSGSGTDFTLTISSLEPEDFA |
| | | VYYCQQSSNWPRTFGQGTKVEIKRADAAPTVSIFPPSSEQL |
| | | TSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | QVQLVESGGGVVQPGRSLRLDCKASGITFSNSGMHWVRQ |
| | NO: 195 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEKEMTKKQVSLTC |
| | | LVKDFMPEDIYVEWTNNGKTELNYKNTEPVLKSDGSYFM |
| | | YSKLTVEKKNWVERNSYSCSVVHEGLHNHHTTKSFSRTPG |
| | | GGGGSGGGGSGGGGSGGGGSQVQLVQSGAEVKKPGASV |
| | | KVSCKASGDTFTRYYVHWVRQAPGQGLEWMGIINPSGGY |
| | | ASYAQKFQGRVTMTRDTSTSTVYMELSSLRSEDTAVYYC |
| | | AAGLFIWGQGTLVTVSSASGGGGSGGGGSGGGGSHASDIQ |
| | | MTQSPSSLSASVGDRVTITCRASQSIGRYLAWYQQKPGKA |
| | | PKLLIYSASNLETGVPSRFSGSGSGTDFTLTISSLQPEDFATY |
| | | YCQQYNSFPVTFGPGTKVDIKGGGSGGGSHHHHHH |
| | SEQ ID | QVQLVESGGGVVQPGRSLRLDCKASGITFSNSGMHWVRQ |
| | NO: 199 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEEEMTKKQVTLTC |
| | | MVTDFMPEDIYVEWTNNGKTELNYKNTEPVLDSDGSYFM |
| | | YSDLRVEKKNWVERNSYSCSVVHEGLHNHHTTESFSRTPG |
| | | GGGGSGGGGSGGGGSAPTSSSTKKTQLQLEHLLLDLQMIL |
| | | NGINNYKNPKLTDMLTFEFYMPKKATELKHLQCLERELKP |
| | | LEEVLNLAQSKNFHLRPRDLISNINVIVLELKGSETTFMCEY |
| | | ADETATIVEFLNRWITFCQSIISTLT |
| AF4404 | SEQ ID | EIVLTQSPATLSLSPGERATLSCRASQSVSSYLAWYQQKPG |
| | NO: 180 | QAPRLLIYDASNRATGIPARFSGSGSGTDFTLTISSLEPEDFA |
| | | VYYCQQSSNWPRTFGQGTKVEIKRADAAPTVSIFPPSSEQL |
| | | TSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | QVQLVESGGGVVQPGRSLRLDCKASGITFSNSGMHWVRQ |
| | NO: 196 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEKEMTKKQVSLTC |
| | | LVKDFMPEDIYVEWTNNGKTELNYKNTEPVLKSDGSYFM |
| | | YSKLTVEKKNWVERNSYSCSVVHEGLHNHHTTKSFSRTPG |
| | | GGGGSGGGGSGGGGSGGGGSQVQLVQSGAEVKKPGASV |
| | | KVSCKASGYTFTDYYMHWVRQAPGQGLEWMGIINPRAG |
| | | YTSYALKFQGRVTMTRDTSTSTVYMELSSLRSEDTAVYYC |
| | | TSGWDVWGQGTLVTVSSASGGGGSGGGGSGGGGSHASDI |
| | | QMTQSPSSLSASVGDRVTITCRASQSISTWLAWYQQKPGK |
| | | APKLLIYAASSLDSGVPSRFSGSGSGTDFTLTISSLQPEDFAT |
| | | YYCQQSYSFPVTFGQGTKVEIKGGGSGGGSHHHHHH |
| | SEQ ID | QVQLVESGGGVVQPGRSLRLDCKASGITFSNSGMHWVRQ |
| | NO: 199 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEEEMTKKQVTLTC |
| | | MVTDFMPEDIYVEWTNNGKTELNYKNTEPVLDSDGSYFM |
| | | YSDLRVEKKNWVERNSYSCSVVHEGLHNHHTTESFSRTPG |
| | | GGGGSGGGGSGGGGSAPTSSSTKKTQLQLEHLLLDLQMIL |
| | | NGINNYKNPKLTDMLTFEFYMPKKATELKHLQCLERELKP |
| | | LEEVLNLAQSKNFHLRPRDLISNINVIVLELKGSETTFMCEY |
| | | ADETATIVEFLNRWITFCQSIISTLT |
| AF4395 | SEQ ID | DIQMTQSPSSLSASVGDRVTITCRASQDVNTAVAWYQQKP |
| | NO: 64 | GKAPKLLIYSASFLYSGVPSRFSGSRSGTDFTLTISSLQPEDF |
| | | ATYYCQQHYTTPPTFGQGTKVEIKRADAAPTVSIFPPSSEQ |
| | | LTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | EVQLVESGGGLVQPGGSLRLSCAASGFNIKDTYIHWVRQA |
| | NO: 197 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEKEMT |
| | | KKQVSLTCLVKDFMPEDIYVEWTNNGKTELNYKNTEPVL |
| | | KSDGSYFMYSKLTVEKKNWVERNSYSCSVVHEGLHNHHT |
| | | TKSFSRTPGGGGGSGGGGSGGGGSGGGGSQVQLVQSGAE |
| | | VKKPGASVKVSCKASGDTFTRYYVHWVRQAPGQGLEWM |
| | | GIINPSGGYASYAQKFQGRVTMTRDTSTSTVYMELSSLRSE |
| | | DTAVYYCAAGLFIWGQGTLVTVSSASGGGGSGGGGSGGG |
| | | GSHASDIQMTQSPSSLSASVGDRVTITCRASQSIGRYLAWY |
| | | QQKPGKAPKLLIYSASNLETGVPSRFSGSGSGTDFTLTISSL |
| | | QPEDFATYYCQQYNSFPVTFGPGTKVDIKGGGSGGGSHHH |
| | | HHH |
| | SEQ ID | EVQLVESGGGLVQPGGSLRLSCAASGFNIKDTYIHWVRQA |
| | NO: 202 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEEEMTK |
| | | KQVTLTCMVTDFMPEDIYVEWTNNGKTELNYKNTEPVLD |
| | | SDGSYFMYSDLRVEKKNWVERNSYSCSVVHEGLHNHHTT |
| | | ESFSRTPGGGGGSGGGGSGGGGSAPTSSSTKKTQLQLEHLL |
| | | LDLQMILNGINNYKNPKLTDMLTFEFYMPKKATELKHLQC |
| | | LERELKPLEEVLNLAQSKNFHLRPRDLISNINVIVLELKGSE |
| | | TTFMCEYADETATIVEFLNRWITFCQSIISTLT |
| AF4396 | SEQ ID | DIQMTQSPSSLSASVGDRVTITCRASQDVNTAVAWYQQKP |
| | NO: 64 | GKAPKLLIYSASFLYSGVPSRFSGSRSGTDFTLTISSLQPEDF |
| | | ATYYCQQHYTTPPTFGQGTKVEIKRADAAPTVSIFPPSSEQ |
| | | LTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | EVQLVESGGGLVQPGGSLRLSCAASGFNIKDTYIHWVRQA |
| | NO: 198 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEKEMT |
| | | KKQVSLTCLVKDFMPEDIYVEWTNNGKTELNYKNTEPVL |
| | | KSDGSYFMYSKLTVEKKNWVERNSYSCSVVHEGLHNHHT |
| | | TKSFSRTPGGGGGSGGGGSGGGGSGGGGSQVQLVQSGAE |
| | | VKKPGASVKVSCKASGYTFTDYYMHWVRQAPGQGLEWM |
| | | GIINPRAGYTSYALKFQGRVTMTRDTSTSTVYMELSSLRSE |
| | | DTAVYYCTSGWDVWGQGTLVTVSSASGGGGSGGGGSGG |
| | | GGSHASDIQMTQSPSSLSASVGDRVTITCRASQSISTWLAW |
| | | YQQKPGKAPKLLIYAASSLDSGVPSRFSGSGSGTDFTLTISS |
| | | LQPEDFATYYCQQSYSFPVTFGQGTKVEIKGGGSGGGSHH |
| | | HHHH |
| | SEQ ID | EVQLVESGGGLVQPGGSLRLSCAASGFNIKDTYIHWVRQA |
| | NO: 202 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEEEMTK |
| | | KQVTLTCMVTDFMPEDIYVEWTNNGKTELNYKNTEPVLD |
| | | SDGSYFMYSDLRVEKKNWVERNSYSCSVVHEGLHNHHTT |
| | | ESFSRTPGGGGGSGGGGSGGGGSAPTSSSTKKTQLQLEHLL |
| | | LDLQMILNGINNYKNPKLTDMLTFEFYMPKKATELKHLQC |
| | | LERELKPLEEVLNLAQSKNFHLRPRDLISNINVIVLELKGSE |
| | | TTFMCEYADETATIVEFLNRWITFCQSIISTLT |
| AF4400 | SEQ ID | EIVLTQSPATLSLSPGERATLSCRASQSVSSYLAWYQQKPG |
| | NO: 180 | QAPRLLIYDASNRATGIPARFSGSGSGTDFTLTISSLEPEDFA |
| | | VYYCQQSSNWPRTFGQGTKVEIKRADAAPTVSIFPPSSEQL |
| | | TSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | QVQLVESGGGVVQPGRSLRLDCKASGITFSNSGMHWVRQ |
| | NO: 199 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEEEMTKKQVTLTC |
| | | MVTDFMPEDIYVEWTNNGKTELNYKNTEPVLDSDGSYFM |
| | | YSDLRVEKKNWVERNSYSCSVVHEGLHNHHTTESFSRTPG |
| | | GGGGSGGGGSGGGGSAPTSSSTKKTQLQLEHLLLDLQMIL |
| | | NGINNYKNPKLTDMLTFEFYMPKKATELKHLQCLERELKP |
| | | LEEVLNLAQSKNFHLRPRDLISNINVIVLELKGSETTFMCEY |
| | | ADETATIVEFLNRWITFCQSIISTLT |
| | SEQ ID | QVQLVESGGGVVQPGRSLRLDCKASGITFSNSGMHWVRQ |
| | NO: 200 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEKEMTKKQVSLTC |
| | | LVKDFMPEDIYVEWTNNGKTELNYKNTEPVLKSDGSYFM |
| | | YSKLTVEKKNWVERNSYSCSVVHEGLHNHHTTKSFSRTPG |
| | | GGGGSGGGGSGGGGSGGGGSEVQLVESGGGLVQPGGSLR |
| | | LSCAASGFNIKDTYIHWVRQAPGKGLEWVARIYPTNGYTR |
| | | YADSVKGRFTISADTSKNTAYLQMNSLRAEDTAVYYCSR |
| | | WGGDGFYAMDYWGQGTLVTVSSASGGGGSGGGGSGGG |
| | | GSHASDIQMTQSPSSLSASVGDRVTITCRASQDVNTAVAW |
| | | YQQKPGKAPKLLIYSASFLYSGVPSRFSGSRSGTDFTLTISS |
| | | LQPEDFATYYCQQHYTTPPTFGQGTKVEIKGGGSGGGSHH |
| | | HHHH |
| AF4401 | SEQ ID | EIVLTQSPATLSLSPGERATLSCRASQSVSSYLAWYQQKPG |
| | NO: 180 | QAPRLLIYDASNRATGIPARFSGSGSGTDFTLTISSLEPEDFA |
| | | VYYCQQSSNWPRTFGQGTKVEIKRADAAPTVSIFPPSSEQL |
| | | TSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | QVQLVESGGGVVQPGRSLRLDCKASGITFSNSGMHWVRQ |
| | NO: 199 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEEEMTKKQVTLTC |
| | | MVTDFMPEDIYVEWTNNGKTELNYKNTEPVLDSDGSYFM |
| | | YSDLRVEKKNWVERNSYSCSVVHEGLHNHHTTESFSRTPG |
| | | GGGGSGGGGSGGGGSAPTSSSTKKTQLQLEHLLLDLQMIL |
| | | NGINNYKNPKLTDMLTFEFYMPKKATELKHLQCLERELKP |
| | | LEEVLNLAQSKNFHLRPRDLISNINVIVLELKGSETTFMCEY |
| | | ADETATIVEFLNRWITFCQSIISTLT |
| | SEQ ID | QVQLVESGGGVVQPGRSLRLDCKASGITFSNSGMHWVRQ |
| | NO: 201 | APGKGLEWVAVIWYDGSKRYYADSVKGRFTISRDNSKNT |
| | | LFLQMNSLRAEDTAVYYCATNDDYWGQGTLVTVSSAKTT |
| | | APSVYPLAPVCGDTTGSSVTLGCLVKGYFPEPVTLTWNSG |
| | | SLSSGVHTFPAVLQSDLYTLSSSVTVTSSTWPSQSITCNVAH |
| | | PASSTKVDKKIEPRGPTIKPCPPCKCPAPNAAGGPSVFIFPPK |
| | | IKDVLMISLSPIVTCVVVDVSEDDPDVQISWFVNNVEVHTA |
| | | QTQTHREDYNSTLRVVSALPIQHQDWMSGKEFKCKVNNK |
| | | DLGAPIERTISKPKGSVRAPQVYVLPPPEKEMTKKQVSLTC |
| | | LVKDFMPEDIYVEWTNNGKTELNYKNTEPVLKSDGSYFM |
| | | YSKLTVEKKNWVERNSYSCSVVHEGLHNHHTTKSFSRTPG |
| | | GGGGSGGGGSGGGGSGGGGSEVQLVESGGGLVKPGGSLR |
| | | LSCAASGFTFSSYTLAWVRQAPGKGLEWVAAIDSSSYTYS |
| | | PDTVRGRFTISRDNAKNSLYLQMNSLRAEDTAVYYCARDS |
| | | NWDALDYWGQGTLVTVSSASGGGGSGGGGSGGGGSHAS |
| | | DIQMTQSPSSLSASVGDRVSITCKASQNVGTNVGWYQQKP |
| | | GKAPKALIYSASFRYSGVPSRFSGSGSGTDFTLTISSLQPED |
| | | FATYFCQQYYTYPYTFGGGTKLEIKGGGSGGGSHHHHHH |
| AF4392 | SEQ ID | DIQMTQSPSSLSASVGDRVTITCRASQDVNTAVAWYQQKP |
| | NO: 64 | GKAPKLLIYSASFLYSGVPSRFSGSRSGTDFTLTISSLQPEDF |
| | | ATYYCQQHYTTPPTFGQGTKVEIKRADAAPTVSIFPPSSEQ |
| | | LTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | EVQLVESGGGLVQPGGSLRLSCAASGFNIKDTYIHWVRQA |
| | NO: 202 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEEEMTK |
| | | KQVTLTCMVTDFMPEDIYVEWTNNGKTELNYKNTEPVLD |
| | | SDGSYFMYSDLRVEKKNWVERNSYSCSVVHEGLHNHHTT |
| | | ESFSRTPGGGGGSGGGGSGGGGSAPTSSSTKKTQLQLEHLL |
| | | LDLQMILNGINNYKNPKLTDMLTFEFYMPKKATELKHLQC |
| | | LERELKPLEEVLNLAQSKNFHLRPRDLISNINVIVLELKGSE |
| | | TTFMCEYADETATIVEFLNRWITFCQSIISTLT |
| | SEQ ID | EVQLVESGGGLVQPGGSLRLSCAASGFNIKDTYIHWVRQA |
| | NO: 203 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEKEMT |
| | | KKQVSLTCLVKDFMPEDIYVEWTNNGKTELNYKNTEPVL |
| | | KSDGSYFMYSKLTVEKKNWVERNSYSCSVVHEGLHNHHT |
| | | TKSFSRTPGGGGGSGGGGSGGGGSGGGGSEVQLVESGGGL |
| | | VQPGGSLRLSCAASGFNIKDTYIHWVRQAPGKGLEWVARI |
| | | YPTNGYTRYADSVKGRFTISADTSKNTAYLQMNSLRAEDT |
| | | AVYYCSRWGGDGFYAMDYWGQGTLVTVSSASGGGGSGG |
| | | GGSGGGGSHASDIQMTQSPSSLSASVGDRVTITCRASQDV |
| | | NTAVAWYQQKPGKAPKLLIYSASFLYSGVPSRESGSRSGT |
| | | DFTLTISSLQPEDFATYYCQQHYTTPPTFGQGTKVEIKGGG |
| | | SGGGSHHHHHH |
| AF4393 | SEQ ID | DIQMTQSPSSLSASVGDRVTITCRASQDVNTAVAWYQQKP |
| | NO: 64 | GKAPKLLIYSASFLYSGVPSRFSGSRSGTDFTLTISSLQPEDF |
| | | ATYYCQQHYTTPPTFGQGTKVEIKRADAAPTVSIFPPSSEQ |
| | | LTSGGASVVCFLNNFYPKDINVKWKIDGSERQNGVLNSWT |
| | | DQDSKDSTYSMSSTLTLTKDEYERHNSYTCEATHKTSTSPI |
| | | VKSFNRNEC |
| | SEQ ID | EVQLVESGGGLVQPGGSLRLSCAASGFNIKDTYIHWVRQA |
| | NO: 202 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEEEMTK |
| | | KQVTLTCMVTDFMPEDIYVEWTNNGKTELNYKNTEPVLD |
| | | SDGSYFMYSDLRVEKKNWVERNSYSCSVVHEGLHNHHTT |
| | | ESFSRTPGGGGGSGGGGSGGGGSAPTSSSTKKTQLQLEHLL |
| | | LDLQMILNGINNYKNPKLTDMLTFEFYMPKKATELKHLQC |
| | | LERELKPLEEVLNLAQSKNFHLRPRDLISNINVIVLELKGSE |
| | | TTFMCEYADETATIVEFLNRWITFCQSIISTLT |
| | SEQ ID | EVQLVESGGGLVQPGGSLRLSCAASGFNIKDTYIHWVRQA |
| | NO: 204 | PGKGLEWVARIYPTNGYTRYADSVKGRFTISADTSKNTAY |
| | | LQMNSLRAEDTAVYYCSRWGGDGFYAMDYWGQGTLVT |
| | | VSSAKTTAPSVYPLAPVCGDTTGSSVTLGCLVKGYFPEPVT |
| | | LTWNSGSLSSGVHTFPAVLQSDLYTLSSSVTVTSSTWPSQSI |
| | | TCNVAHPASSTKVDKKIEPRGPTIKPCPPCKCPAPNAAGGP |
| | | SVFIFPPKIKDVLMISLSPIVTCVVVDVSEDDPDVQISWFVN |
| | | NVEVHTAQTQTHREDYNSTLRVVSALPIQHQDWMSGKEF |
| | | KCKVNNKDLGAPIERTISKPKGSVRAPQVYVLPPPEKEMT |
| | | KKQVSLTCLVKDFMPEDIYVEWTNNGKTELNYKNTEPVL |
| | | KSDGSYFMYSKLTVEKKNWVERNSYSCSVVHEGLHNHHT |
| | | TKSFSRTPGGGGGSGGGGSGGGGSGGGGSEVQLVESGGGL |
| | | VKPGGSLRLSCAASGFTFSSYTLAWVRQAPGKGLEWVAAI |
| | | DSSSYTYSPDTVRGRFTISRDNAKNSLYLQMNSLRAEDTA |
| | | VYYCARDSNWDALDYWGQGTLVTVSSASGGGGSGGGGS |
| | | GGGGSHASDIQMTQSPSSLSASVGDRVSITCKASQNVGTN |
| | | VGWYQQKPGKAPKALIYSASFRYSGVPSRFSGSGSGTDFT |
| | | LTISSLQPEDFATYFCQQYYTYPYTFGGGTKLEIKGGGSGG |
| | | GSHHHHHH |

### Example 12

### Binding of Additional Dual Binding Antibodies to PD1 and IL2

Having observed the effectiveness of dual binding antibodies (DBAs) in previous examples, additional dual binding antibody scFvs were generated (see TABLE 17, TABLE 18, and TABLE 19 below). This example describes the measurement of binding of PD1 and IL2 to these dual binding antibody scFvs. The scFv DNA sequence for each clone was synthesized as a GBLOCK® (Integrated DNA Technologies, Inc.) with a T7 promoter, a translation initiation site, the coding sequence for the scFv sequence with cMyc and V5 tags, and a T7 terminator sequence. Proteins from each of the GBLOCK® fragments were expressed using a cell-free transcription/translation system (Cosmo Bio USA, Inc., PUREFREX® 2.1, Product #GFK-PF213 with DS Supplement, Prod. #GFK-PF005). The scFv samples were subjected to ELISA analysis to detect PD1 and IL2 binding. In these experiments, wells of a 384-well plate were coated with an anti-V5 antibody (Sv5-Pk1, BioRad) at lug/ml overnight at 4 degrees. After washing, wells were blocked with SUPERBLOCK™ (ThermoFisher, 37515) followed by addition of saturating levels of scFvs in SUPERBLOCK™. After washing, antigens were added and plates incubated for one hour. To detect PD1 binding, a biotinylated recombinant PD1 was used (PD1-HisAvi, Acro Biosystems, PD1-H82E4). To detect IL2 binding, IL2 (R&D, 202-IL) was preincubated with biotinylated-anti-IL2 mAb (mab202, biotinylated using standard methods) at a ratio of 2:1 in SUPERBLOCK™ buffer. Biotinylated antigens were detected using streptavidin HRP using standard methods. Varying amounts of labelled test antigen were added to show binding and to estimate relative affinities of the different scFvs. TABLE 17A-C shows the EC50 values for PD1 and IL2 binding for three sets of scFvs, demonstrating dual binding of these antibodies.

The data indicate that all the antibodies in TABLE 17A-C except for the control antibodies AB000694, AB000719, AB000880 are able to effectively bind both PD1 and IL2.

TABLE 17A
Binding affinities for additional DBAs to PD1 and IL2 according
to the disclosure as assessed by ELISA in a first experiment

| | Protein | | |
|---|---|---|---|
| antibodyID | name | PD1 EC50 | IL2 EC50 |
| AB002293_2B07v2 | AF003676 | 0.2 | 6 |
| AB002353_2B07v9 | AF003736 | 0.9 | 30 |
| AB002342_2B07v5 | AF003725 | 0.2 | 7 |
| AB002328_2B07v4 | AF003711 | 0.2 | 0.4 |
| AB002326_2B07v3 | AF003709 | 0.2 | 30 |
| AB002345_2B07v6 | AF003728 | 0.2 | 60 |
| AB002348_2B07v8 | AF003731 | 0.3 | 100 |
| AB002022_2B07v1 | AF002829 | 0.6 | 10 |
| AB002347_2B07v7 | AF003730 | 0.1 | 8 |

TABLE 17B
Binding affinities for additional DBAs to PD1 and IL2 according
to the disclosure as assessed by ELISA in a second experiment

| | Protein | | |
|---|---|---|---|
| antibodyID | name | PD1 EC50 | IL2 EC50 |
| AB000694 (anti- | AF005039 | 0.7 | — |
| PD1 control) | | | |
| AB000719 (anti- | AF005040 | 0.4 | — |
| PD1 control) | | | |
| AB000880 (anti- | AF000327 | 4 | — |
| PD1 control) | | | |
| AB003021_2A11v7 | AF005103 | 0.6 | 40 |
| AB002417_2A11v4 | AF005148 | 4 | 50 |
| AB002413_2A11v3 | AF005147 | 2 | 2 |
| AB002427_2A11v5 | AF005149 | 3 | 0.9 |
| AB003200_2B05v1 | AF005131 | 0.5 | weak |
| AB003209_2B05v4 | AF005140 | 0.4 | weak |
| AB003201_2B05v2 | AF005132 | 0.5 | weak |
| AB003202_2B05v3 | AF005133 | 0.5 | weak |
| AB003155_2B07v10 | AF005042 | 0.7 | 0.4 |
| AB002328_2B07v4 | AF005041 | 2 | 1 |
| AB003174_2B07v11 | AF005062 | 0.6 | 7 |
| AB003180_2B07v13 | AF005068 | 0.6 | 0.8 |
| AB003183_2B07v14 | AF005071 | 0.3 | 40 |
| AB003178_2B07v12 | AF005066 | 1 | 0.3 |
| AB003007_7A04v6 | AF005087 | 1 | 10 |
| AB002365_7A04v2 | AF005095 | 1 | 8 |
| AB003012_7A04v7 | AF005092 | 1 | 7 |
| AB003005_7A04v5 | AF005085 | 4 | 3 |
| AB002864_2A11v6 | AF005116 | 0.4 | 60 |

TABLE 17C
Binding affinities for additional DBAs to PD1 and IL2 according
to the disclosure as assessed by ELISA in a third experiment

| | Protein | | |
|---|---|---|---|
| antibodyID | name | PD1 EC50 | IL2 EC50 |
| AB000880 (anti- | AF000327 | 8 | — |
| PD1 control) | | | |
| AB002360_7A04v1 | AF003765 | 1 | 70 |
| AB002365_7A04v2 | AF003770 | 1 | 30 |
| AB002370_7A04v3 | AF003775 | 0.7 | 30 |
| AB002381_7A04v4 | AF003786 | 0.7 | 30 |
| AB002410_2A11v2 | AF003815 | 3 | 100 |
| AB002413_2A11v3 | AF003818 | 3 | 3 |
| AB002417_2A11v4 | AF003822 | 5 | 400 |
| AB002427_2A11v5 | AF003832 | 3 | 0.8 |
| AB002022_2B07v1 | AF002829 | 5 | 30 |
| AB001938 | AF002745 | 10 | 30 |

TABLE 18
Sequences of VH and VL regions for DBAs described in Example 12

| | | | | VH SEQ | VL SEQ |
|---|---|---|---|---|---|
| ID | antibodyID | VH | VL | ID NO: | ID NO: |
| AB000694_ | AB000694 | QVQLVESGGGVVQPGRSLRLD | EIVLTQSPATLSLSPGERATLSCRASQS | 329 | 330 |
| nivo | | CKASGITFSNSGMHWVRQAPG | VSSYLAWYQQKPGQAPRLLIYDASNR | | |
| | | KGLEWVAVIWYDGSKRYYADS | ATGIPARFSGSGSGTDFTLTISSLEPED | | |
| | | VKGRFTISRDNSKNTLFLQMNS | FAVYYCQQSSNWPRTFGQGTKVEIK | | |
| | | LRAEDTAVYYCATNDDYWGQ | | | |
| | | GTLVTVSS | | | |
| AB000880_ | AB000880 | QVQLVQSGAEVKKPGASVKVS | DIVMTQSPDSLAVSLGERATINCKAS | 331 | 332 |
| PD1_R04_C10 | | CKASGYTFTTYYMHWVRQAPG | QSLLHSSSNKNYLAWYQQKPGQPPK | | |
| | | QGLEWMGIINPSGGGTLYAQKF | LLIYWASTRESGVPDRFSGSGSGTDFT | | |
| | | QGRVTMTRDTSTSTVYMELSSL | LTISSLQAEDVAVYYCQQYYSTPITFG | | |
| | | RSEDTAVYYCAAGLFIWGQGTT | PGTKVDIK | | |
| | | VTVSS | | | |
| AB002022_ | AB002022 | QVQLVQSGAEVKKPGASVKVS | DIQMTQSPSSLSASVGDRVTITCRASQ | 333 | 334 |
| 2B07v1 | | CKASGDTFTRHYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYESFPVTFGPGTKVDIK | | |
| | | RSEDTAVYYCAAGLFIWGQGTL | | | |
| | | VTVSS | | | |
| AB002293_ | AB002293 | QVQLVQSGAEVKKPGASVKVS | DIQMTQSPSSLSASVGDRVTITCRASQ | 335 | 336 |
| 2B07v2 | | CKASGDTFTRYYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNSFPVTFGPGTKVDIK | | |
| | | RSEDTAVYYCAAGLFIWGQGTL | | | |
| | | VTVSS | | | |
| AB002326_ | AB002326 | QVQLVQSGAEVKKPGASVKVS | DIQMTQSPSSLSASVGDRVTITCRASQ | 337 | 338 |
| 2B07v3 | | CKASGDTFTRYYVHWVRQAPG | SIGSWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNSFPVTFGPGTKVDIK | | |
| | | RSEDTAVYYCAAGLFIWGQGTL | | | |
| | | VTVSS | | | |
| AB002328_ | AB002328 | QVQLVQSGAEVKKPGASVKVS | DIQMTQSPSSLSASVGDRVTITCRASQ | 339 | 340 |
| 2B07v4 | | CKASGDTFTRYYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNRFPVTFGPGTKVDIK | | |
| | | RSEDTAVYYCAAGLFIWGQGTL | | | |
| | | VTVSS | | | |
| AB002342_ | AB002342 | QVQLVQSGAEVKKPGASVKVS | DIQMTQSPSSLSASVGDRVTITCRASQ | 341 | 342 |
| 2B07v5 | | CKASGDTFTRYYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYERFPVTFGPGTKVDIK | | |
| | | RSEDTAVYYCAAGLFIWGQGTL | | | |
| | | VTVSS | | | |
| AB002345_ | AB002345 | QVQLVQSGAEVKKPGASVKVS | DIQMTQSPSSLSASVGDRVTITCRASQ | 343 | 344 |
| 2B07v6 | | CKASGDTFTRYYVHWVRQAPG | SIGRYLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNSFPVTFGPGTKVDIK | | |
| | | RSEDTAVYYCAAGLFIWGQGTL | | | |
| | | VTVSS | | | |
| AB002347_ | AB002347 | QVQLVQSGAEVKKPGASVKVS | DIQMTQSPSSLSASVGDRVTITCRASQ | 345 | 346 |
| 2B07v7 | | CKASGDTFTRYYVHWVRQAPG | SIGRYLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNRFPVTFGPGTKVDIK | | |
| | | RSEDTAVYYCAAGLFIWGQGTL | | | |
| | | VTVSS | | | |
| AB002348_ | AB002348 | QVQLVQSGAEVKKPGASVKVS | DIQMTQSPSSLSASVGDRVTITCRASQ | 347 | 348 |
| 2B07v8 | | CKASGDTFTRYYVHWVRQAPG | SIGRYLAWYQQKPGKAPKLLIYYASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNSFPVTFGPGTKVDIK | | |
| | | RSEDTAVYYCAAGLFIWGQGTL | | | |
| | | VTVSS | | | |
| AB002353_ | AB002353 | QVQLVQSGAEVKKPGASVKVS | DIQMTQSPSSLSASVGDRVTITCRASQ | 349 | 350 |
| 2B07v9 | | CKASGDTFTRYYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYESFPVTFGPGTKVDIK | | |
| | | RSEDTAVYYCAAGLFIWGQGTL | | | |
| | | VTVSS | | | |
| AB002360_ | AB002360 | QVQLVQSGAEVKKPGASVKVS | DIQMTQSPSSLSASVGDRVTITCRASQ | 351 | 352 |
| 7A04v1 | | CKASGYTFTDYYMHWVRQAPG | SISTWLAWYQQKPGKAPKLLIYAASS | | |
| | | QGLEWMGIINPRAGYTSYALKF | LDSGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWDVWGQGT | | | |
| | | LVTVSS | | | |
| AB002365_ | AB002365 | QVQLVQSGAEVKKPGASVKVS | DIQMTQSPSSLSASVGDRVTITCRASQ | 353 | 354 |
| 7A04v2 | | CKASGYTFTRYYMHWVRQAPG | SISTWLAWYQQKPGKAPKLLIYAASS | | |
| | | QGLEWMGIINPRAGYTSYALKF | LDSGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWDVWGQGT | | | |
| | | LVTVSS | | | |
| AB002370_ | AB002370 | QVQLVQSGAEVKKPGASVKVS | DIQMTQSPSSLSASVGDRVTITCRASQ | 355 | 356 |
| 7A04v3 | | CKASGYTFTTYYMHWVRQAPG | SISTWLAWYQQKPGKAPKLLIYAASS | | |
| | | QGLEWMGIINPRAGYTSYALKF | LDSGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWDVWGQGT | | | |
| | | LVTVSS | | | |
| AB002381_ | AB002381 | QVQLVQSGAEVKKPGASVKVS | DIQMTQSPSSLSASVGDRVTITCRASQ | 357 | 358 |
| 7A04v4 | | CKASGDTFTDYYMHWVRQAPG | SISTWLAWYQQKPGKAPKLLIYAASS | | |
| | | QGLEWMGIINPRAGYTSYALKF | LDSGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWDVWGQGT | | | |
| | | LVTVSS | | | |
| AB002410_ | AB002410 | QVQLVQSGAEVKKPGASVKVS | DIQMTQSPSSLSASVGDRVTITCRASQ | 359 | 360 |
| 2A11v2 | | CKASGHTFTRYYMHWVRQAPG | SINSWLAWYQQKPGKAPKLLIYDTST | | |
| | | QGLEWMGIINPSGGYATYAQKF | LESGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCASGWFVWGQGT | | | |
| | | LVTVSS | | | |
| AB002413_ | AB002413 | QVQLVQSGAEVKKPGASVKVS | DIQMTQSPSSLSASVGDRVTITCRASQ | 361 | 362 |
| 2A11v3 | | CKASGHTFTRYYMHWVRQAPG | SINSWLAWYQQKPGKAPKLLIYATST | | |
| | | QGLEWMGIINPSGGYATYAQKF | LESGVPSRFSGSGSGTDFTLTISSLQPE | | |
| | | QGRVTMTRDTSTSTVYMELSSL | DFATYYCQQYYRFPVTFGQGTKVEIK | | |
| | | RSEDTAVYYCASGLFIWGQGTL | | | |
| | | VTVSS | | | |
| AB002417_ | AB002417 | QVQLVQSGAEVKKPGASVKVS | DIQMTQSPSSLSASVGDRVTITCRASQ | 363 | 364 |
| 2A11v4 | | CKASGHTFTRYYMHWVRQAPG | SINSWLAWYQQKPGKAPKLLIYATST | | |
| | | QGLEWMGIINPSGGYATYAQKF | LESGVPSRFSGSGSGTDFTLTISSLQPE | | |
| | | QGRVTMTRDTSTSTVYMELSSL | DFATYYCQQYYSFPVTFGQGTKVEIK | | |
| | | RSEDTAVYYCASGLFIWGQGTL | | | |
| | | VTVSS | | | |
| AB002427_ | AB002427 | QVQLVQSGAEVKKPGASVKVS | DIQMTQSPSSLSASVGDRVTITCRASQ | 365 | 366 |
| 2A11v5 | | CKASGHTFTRYYMHWVRQAPG | SINSWLAWYQQKPGKAPKLLIYATST | | |
| | | QGLEWMGIINPSGGYATYAQKF | LESGVPSRFSGSGSGTDFTLTISSLQPE | | |
| | | QGRVTMTRDTSTSTVYMELSSL | DFATYYCQQSYRFPVTFGQGTKVEIK | | |
| | | RSEDTAVYYCASGLFIWGQGTL | | | |
| | | VTVSS | | | |
| AB002520_ | AB002520 | QVQLVQSGAEVKKPGASVKVS | DIQMTQSPSSLSASVGDRVTITCRASQ | 367 | 368 |
| 7A04v8 | | CKASGYTFTDYYMHWVRQAPG | SISTWLAWYQQKPGKAPKLLIYAASS | | |
| | | QGLEWMGIINPRAGYTSYALKF | LDSGVPSRFSGSGSGTDFTLTISSLQPE | | |
| | | QGRVTMTRDTSTSTVYMELSSL | DFATYYCQQSYRFPVTFGQGTKVEIK | | |
| | | RSEDTAVYYCTSGWDVWGQGT | | | |
| | | LVTVSS | | | |
| AB002521_ | AB002521 | QVQLVQSGAEVKKPGASVKVS | DIQMTQSPSSLSASVGDRVTITCRASQ | 369 | 370 |
| 2A11v1 | | CKASGHTFTRYYMHWVRQAPG | SINSWLAWYQQKPGKAPKLLIYATST | | |
| | | QGLEWMGIINPSGGYATYAQKF | LESGVPSRFSGSGSGTDFTLTISSLQPE | | |
| | | QGRVTMTRDTSTSTVYMELSSL | DFATYYCQQSYRFPVTFGQGTKVEIK | | |
| | | RSEDTAVYYCASGLDIWGQGTL | | | |
| | | VTVSS | | | |
| AB002829_ | AB002829 | QVQLVQSGAEVKKPGASVKVS | DIVMTQSPLSLPVTPGEPASISCRSSQS | 371 | 372 |
| knd_A08 | | CKASGYIFNGYDIHWVRQAPGQ | LLHSNGYNYLDWYLQKPGQSPQLLIY | | |
| | | GLEWMGWMNPDNGNTGLAQK | LGSNRASGVPDRFSGSGSGTDFTLKIS | | |
| | | FQGRVTITADESTSTAYMELSS | RVEAEDVGVYYCMQGTHWPPTFGQ | | |
| | | LRSEDTAVYYCARGMATRFPYY | GTKVEIK | | |
| | | YYGMDVWGQGTLVTVSS | | | |
| AB002864_ | AB002864 | QVQLVQSGAEVKKPGASVKVS | DIQMTQSPSSLSASVGDRVTITCRASQ | 373 | 374 |
| 2A11v6 | | CKASGHTFTRYYMHWVRQAPG | SINSWLAWYQQKPGKAPKLLIYATST | | |
| | | QGLEWMGVINPSGGYATYAQK | LESGVPSRFSGSGSGTDFTLTISSLQPE | | |
| | | FQGRVTMTRDTSTSTVYMELSS | DFATYYCQQSYSSPVTFGQGTKVEIK | | |
| | | LRSEDTAVYYCASGWDVWGQ | | | |
| | | GTLVTVSS | | | |
| AB003005_ | AB003005 | QVQLVQSGAEVKKPGASVKVS | DIQMTQSPSSLSASVGDRVTITCRASQ | 375 | 376 |
| 7A04v5 | | CKASGYTFTRYYMHWVRQAPG | SISTWLAWYQQKPGKAPKLLIYAASS | | |
| | | QGLEWMGIIQPRAGYTSYALKF | LDEGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWDVWGQGT | | | |
| | | LVTVSS | | | |
| AB003007_ | AB003007 | QVQLVQSGAEVKKPGASVKVS | DIQMTQSPSSLSASVGDRVTITCRASQ | 377 | 378 |
| 7A04v6 | | CKASGYTFTRYYMHWVRQAPG | SISTWLAWYQQKPGKAPKLLIYAASS | | |
| | | QGLEWMGIINPRAGYTSYALKF | LDEGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWEVWGQGT | | | |
| | | LVTVSS | | | |
| AB003012_ | AB003012 | QVQLVQSGAEVKKPGASVKVS | DIQMTQSPSSLSASVGDRVTITCRASQ | 379 | 380 |
| 7A04v7 | | CKASGYTFTRYYMHWVRQAPG | SISTWLAWYQQKPGKAPKLLIYAASS | | |
| | | QGLEWMGIINPRAGYTSYALKF | LDEGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWDVWGQGT | | | |
| | | LVTVSS | | | |
| AB003021_ | AB003021 | QVQLVQSGAEVKKPGASVKVS | DIQMTQSPSSLSASVGDRVTITCRASQ | 381 | 382 |
| 2A11v7 | | CKASGFTFTRYYMHWVRQAPG | SISSWLAWYQQKPGKAPKLLIYATST | | |
| | | QGLEWMGVINPSGGYATYAQK | LESGVPSRFSGSGSGTDFTLTISSLQPE | | |
| | | FQGRVTMTRDTSTSTVYMELSS | DFATYYCQQSYSSPVTFGQGTKVEIK | | |
| | | LRSEDTAVYYCASGWDVWGQ | | | |
| | | GTLVTVSS | | | |
| AB003155_ | AB003155 | QVQLVQSGAEVKKPGASVKVS | DIQMTQSPSSLSASVGDRVTITCRASQ | 383 | 384 |
| 2B07v10 | | CKASGETFTRYYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNRFPVTFGPGTKVDIK | | |
| | | RSEDTAVYYCAAGLFIWGQGTL | | | |
| | | VTVSS | | | |
| AB003174_ | AB003174 | QVQLVQSGAEVKKPGASVKVS | DIQMTQSPSSLSASVGDRVTITCRASQ | 385 | 386 |
| 2B07v11 | | CKASGDTFTRYYVHWVRQAPG | SIGRELAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNRFPVTFGPGTKVDIK | | |
| | | RSEDTAVYYCAAGLFIWGQGTL | | | |
| | | VTVSS | | | |
| AB003178_ | AB003178 | QVQLVQSGAEVKKPGASVKVS | DIQMTQSPSSLSASVGDRVTITCRASQ | 387 | 388 |
| 2B07v12 | | CKASGDEFTRYYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNRFPVTFGPGTKVDIK | | |
| | | RSEDTAVYYCAAGLFIWGQGTL | | | |
| | | VTVSS | | | |
| AB003180_ | AB003180 | QVQLVQSGAEVKKPGASVKVS | DIQMTQSPSSLSASVGDRVTITCRASQ | 389 | 390 |
| 2B07v13 | | CKASGDEFTRYYVHWVRQAPG | SIGRWLAWYQQKPGKAPKLLIYSASN | | |
| | | QGLEWMGIQNPSGGYASYAQK | LETGVPSRFSGSGSGTDFTLTISSLQPE | | |
| | | FQGRVTMTRDTSTSTVYMELSS | DFATYYCQQYNRFPVTFGPGTKVDIK | | |
| | | LRSEDTAVYYCAAGLFIWGQGT | | | |
| | | LVTVSS | | | |
| AB003183_ | AB003183 | QVQLVQSGAEVKKPGASVKVS | DIQMTQSPSSLSASVGDRVTITCRASQ | 391 | 392 |
| 2B07v14 | | CKASGDEFTRYYVHWVRQAPG | SIGRELAWYQQKPGKAPKLLIYDASN | | |
| | | QGLEWMGIQNPSGGYASYAQK | LETGVPSRFSGSGSGTDFTLTISSLQPE | | |
| | | FQGRVTMTRDTSTSTVYMELSS | DFATYYCQQYNRFPVTFGPGTKVDIK | | |
| | | LRSEDTAVYYCAAGLFIWGQGT | | | |
| | | LVTVSS | | | |
| AB003200_ | AB003200 | QVQLVQSGAEVKKPGASVKVS | DIQMTQSPSSLSASVGDRVTITCRISQS | 393 | 394 |
| 2B05v1 | | CKASGETFTNYYIHWVRQAPG | ISSWLAWYQQKPGKAPKLLIYSASSL | | |
| | | QGLEWMGVINPRAGYTSYALK | QSGVPSRFSGSGSGTDFTLTISSLQPED | | |
| | | FQGRVTMTRDTSTSTVYMELSS | FATYYCQQSFTSPITFGQGTRLEIK | | |
| | | LRSEDTAVYYCAGGWEDWGQ | | | |
| | | GTLVTVSS | | | |
| AB003201_ | AB003201 | QVQLVQSGAEVKKPGASVKVS | DIQMTQSPSSLSASVGDRVTITCRISQS | 395 | 396 |
| 2B05v2 | | CKASGGTFTNYYIHWVRQAPG | ISSWLAWYQQKPGKAPKLLIYSASSL | | |
| | | QGLEWMGIIDPRAGYTSYALKF | QSGVPSRFSGSGSGTDFTLTISSLQPED | | |
| | | QGRVTMTRDTSTSTVYMELSSL | FATYYCQQSFTSPITFGQGTRLEIK | | |
| | | RSEDTAVYYCAGGWEDWGQG | | | |
| | | TLVTVSS | | | |
| AB003202_ | AB003202 | QVQLVQSGAEVKKPGASVKVS | DIQMTQSPSSLSASVGDRVTITCRISQS | 397 | 398 |
| 2B05v3 | | CKASGSTFTNYYIHWVRQAPGQ | ISSFLAWYQQKPGKAPKLLIYSASSLQ | | |
| | | GLEWMGIIDPRAGYTSYALKFQ | SGVPSRFSGSGSGTDFTLTISSLQPEDF | | |
| | | GRVTMTRDTSTSTVYMELSSLR | ATYYCQQSFTSPITFGQGTRLEIK | | |
| | | SEDTAVYYCAGGWEDWGQGT | | | |
| | | LVTVSS | | | |
| AB003209_ | AB003209 | QVQLVQSGAEVKKPGASVKVS | DIQMTQSPSSLSASVGDRVTITCRISQS | 399 | 400 |
| 2B05v4 | | CKASGETFTNYYIHWVRQAPG | ISSFLAWYQQKPGKAPKLLIYSASSLQ | | |
| | | QGLEWMGIIDPRAGYTSYALKF | SGVPSRFSGSGSGTDFTLTISSLQPEDF | | |
| | | QGRVTMTRDTSTSTVYMELSSL | ATYYCQQSFTSPITFGQGTRLEIK | | |
| | | RSEDTAVYYCAGGWEDWGQG | | | |
| | | TLVTVSS | | | |
| AB003232_ | AB003232 | QVQLVQSGAEVKKPGASVKVS | DIQMTQSPSSLSASVGDRVTITCRASQ | 401 | 402 |
| 2B05v5 | | CKASGTTFTNYYIHWVRQAPG | SISSWLAWYQQKPGKAPKLLIYSASS | | |
| | | QGLEWMGVINPRAGYTSYALK | LQSGVPSRFSGSGSGTDFTLTISSLQPE | | |
| | | FQGRVTMTRDTSTSTVYMELSS | DFATYYCQQSVTMPVTFGQGTRLEIK | | |
| | | LRSEDTAVYYCTGGWLDWGQ | | | |
| | | GTLVTVSS | | | |
| AB003422_ | AB003422 | QVQLVQSGAEVKKPGASVKVS | DIQMTQSPSSLSASVGDRVTITCRASQ | 403 | 404 |
| 2B05v5 | | CKASGYTFTNYYIHWVRQAPG | RISSWLAWYQQKPGKAPKLLIYDASS | | |
| | | QGLEWMGVINPRAGYTSYALK | LQSGVPSRFSGSGSGTDFTLTISSLQPE | | |
| | | FQGRVTMTRDTSTSTVYMELSS | DFATYYCQQSFTMPVTFGQGTRLEIK | | |
| | | LRSEDTAVYYCAGGWLDWGQ | | | |
| | | GTLVTVSS | | | |
| AB003465_ | AB003465 | QVQLVQSGAEVKKPGASVKVS | DIQMTQSPSSLSASVGDRVTITCRASQ | 405 | 406 |
| 2A11v9 | | CKASGFTFTRYYMHWVRQAPG | SISSWLAWYQQKPGKAPKLLIYATST | | |
| | | QGLEWMGVINPSGGYATYAQK | LESGVPSRFSGSGSGTDFTLTISSLQPE | | |
| | | FQGRVTMTRDTSTSTVYMELSS | DFATYYCQQSYSFPPTFGQGTKVEIK | | |
| | | LRSEDTAVYYCASGWDVWGQ | | | |
| | | GTLVTVSS | | | |
| AB003466_ | AB003466 | QVQLVQSGAEVKKPGASVKVS | DIQMTQSPSSLSASVGDRVTITCRASQ | 407 | 408 |
| 2B07v10 | | CKASGETFTRYYVHWVRQAPG | SIGRWLAWYQQKPGKAPKLLIYSASN | | |
| | | QGLEWMGIINPSGGYASYAQKF | LETGVPSRFSGSGSGTDFTLTISSLQPE | | |
| | | QGRVTMTRDTSTSTVYMELSSL | DFATYYCQQYNSFPVTFGPGTKVDIK | | |
| | | RSEDTAVYYCAAGLFIWGQGTL | | | |
| | | VTVSS | | | |
| AB003467_ | AB003467 | QVQLVQSGAEVKKPGASVKVS | DIQMTQSPSSLSASVGDRVTITCRASQ | 409 | 410 |
| 7A04v9 | | CKASGYTFTRYYMHWVRQAPG | SISTWLAWYQQKPGKAPKLLIYAASS | | |
| | | QGLEWMGIINPRAGYTSYALKF | LDTGVPSRFSGSGSGTDFTLTISSLQPE | | |
| | | QGRVTMTRDTSTSTVYMELSSL | DFATYYCQQSYSFPVTFGQGTKVEIK | | |
| | | RSEDTAVYYCTSGWDVWGQGT | | | |
| | | LVTVSS | | | |
| AB003470_ | AB003470 | QVQLVQSGAEVKKPGSSVKVS | DIQMTQSPSSLSASVGDRVTITCRASQ | 411 | 412 |
| knd_A04 | | CKASGYTFTAYYIHWVRQAPG | TISRYLNWYQQKPGKAPKLLIYAASS | | |
| | | QGLEFMGWIHPYSGGTNYAQK | LQSGVPSRFSGSGSGTDFTLTISSLQPE | | |
| | | FQGRVTITADESTSTAYMELSS | DFATYYCQQSYSTPRTFGQGTKLEIK | | |
| | | LRSEDTAVYYCAIGYYYGKEDY | | | |
| | | WGQGTLVTVSS | | | |

TABLE 19
Sequences of V_{H} and V_{L} CDRs for DBAs described in Example 12

| | | | | | | | | V_{H}_ | V_{H}_ | V_{H}_ | V_{L}_ | V_{L}_ | V_{L}_ |
|---|---|---|---|---|---|---|---|---|---|---|---|---|---|
| | | | | | | | | cdr1 | cdr2 | cdr3 | Cdr1 | Cdr2 | Cdr3 |
| | antibody | V_{H}_ | V_{H}_ | V_{H}_ | V_{L}_ | V_{L}_ | V_{L}_ | SEQ ID | SEQ ID | SEQ ID | SEQ ID | SEQ ID | SEQ ID |
| ID | ID | cdr1 | cdr2 | cdr3 | cdr1 | cdr2 | cdr3 | NO: | NO: | NO: | NO: | NO: | NO: |
| AB002022_ | AB00 | GDTFTR | IINPSGGYASY | AAGLFI | RASQSIGRW | SASN | QQYES | 431 | 432 | 433 | 434 | 435 | 436 |
| 2B07v1 | 2022 | HYVH | AQKFQG | | LA | LET | FPV | | | | | | |
| AB002293_ | AB00 | GDTFTR | IINPSGGYASY | AAGLFI | RASQSIGRW | SASN | QQYN | 437 | 438 | 439 | 440 | 441 | 442 |
| 2B07v2 | 2293 | YYVH | AQKFQG | | LA | LET | SFPV | | | | | | |
| AB002326_ | AB00 | GDTFTR | IINPSGGYASY | AAGLFI | RASQSIGSW | SASN | QQYN | 443 | 444 | 445 | 446 | 447 | 448 |
| 2B07v3 | 2326 | YYVH | AQKFQG | | LA | LET | SFPV | | | | | | |
| AB002328_ | AB00 | GDTFTR | IINPSGGYASY | AAGLFI | RASQSIGRW | SASN | QQYN | 449 | 450 | 451 | 452 | 453 | 454 |
| 2B07v4 | 2328 | YYVH | AQKFQG | | LA | LET | RFPV | | | | | | |
| AB002342_ | AB00 | GDTFTR | IINPSGGYASY | AAGLFI | RASQSIGRW | SASN | QQYE | 455 | 456 | 457 | 458 | 459 | 460 |
| 2B07v5 | 2342 | YYVH | AQKFQG | | LA | LET | RFPV | | | | | | |
| AB002345_ | AB00 | GDTFTR | IINPSGGYASY | AAGLFI | RASQSIGRY | SASN | QQYN | 461 | 462 | 463 | 464 | 465 | 466 |
| 2B07v6 | 2345 | YYVH | AQKFQG | | LA | LET | SFPV | | | | | | |
| AB002347_ | AB00 | GDTFTR | IINPSGGYASY | AAGLFI | RASQSIGRY | SASN | QQYN | 467 | 468 | 469 | 470 | 471 | 472 |
| 2B07v7 | 2347 | YYVH | AQKFQG | | LA | LET | RFPV | | | | | | |
| AB002348_ | AB00 | GDTFTR | IINPSGGYASY | AAGLFI | RASQSIGRY | YAS | QQYN | 473 | 474 | 475 | 476 | 477 | 478 |
| 2B07v8 | 2348 | YYVH | AQKFQG | | LA | NLET | SFPV | | | | | | |
| AB002353_ | AB00 | GDTFTR | IINPSGGYASY | AAGLFI | RASQSIGRW | SASN | QQYES | 479 | 480 | 481 | 482 | 483 | 484 |
| 2B07v9 | 2353 | YYVH | AQKFQG | | LA | LET | FPV | | | | | | |
| AB002360_ | AB00 | GYTFTD | IINPRAGYTSY | TSGWDV | RASQSISTW | AASS | QQSYS | 485 | 486 | | 488 | 489 | 490 |
| 7A04v1 | 2360 | YYMH | ALKFQG | | LA | LDS | FPV | | | 487 | | | |
| AB002365_ | AB00 | GYTFTR | IINPRAGYTSY | TSGWDV | RASQSISTW | AASS | QQSYS | 491 | 492 | 493 | 494 | 495 | 496 |
| 7A04v2 | 2365 | YYMH | ALKFQG | | LA | LDS | FPV | | | | | | |
| AB002370_ | AB00 | GYTFTT | IINPRAGYTSY | TSGWDV | RASQSISTW | AASS | QQSYS | 497 | 498 | 499 | 500 | 501 | 502 |
| 7A04v3 | 2370 | YYMH | ALKFQG | | LA | LDS | FPV | | | | | | |
| AB002381_ | AB00 | GDTFTD | IINPRAGYTSY | TSGWDV | RASQSISTW | AASS | QQSYS | 503 | 504 | 505 | 506 | 507 | 508 |
| 7A04v4 | 2381 | YYMH | ALKFQG | | LA | LDS | FPV | | | | | | |
| AB002410_ | AB00 | GHTFTR | IINPSGGYATY | ASGWFV | RASQSINSW | DTST | QQSYS | 509 | 510 | 511 | 512 | 513 | 514 |
| 2A11v2 | 2410 | YYMH | AQKFQG | | LA | LES | FPV | | | | | | |
| AB002413_ | AB00 | GHTFTR | IINPSGGYATY | ASGLFI | RASQSINSW | ATST | QQYY | 515 | 516 | 517 | 518 | 519 | 520 |
| 2A11v3 | 2413 | YYMH | AQKFQG | | LA | LES | RFPV | | | | | | |
| AB002417_ | AB00 | GHTFTR | IINPSGGYATY | ASGLFI | RASQSINSW | ATST | QQYY | 521 | 522 | 523 | 524 | 525 | 526 |
| 2A11v4 | 2417 | YYMH | AQKFQG | | LA | LES | SFPV | | | | | | |
| AB002427_ | AB00 | GHTFTR | IINPSGGYATY | ASGLFI | RASQSINSW | ATST | QQSY | 527 | 528 | 529 | 530 | 531 | 532 |
| 2A11v5 | 2427 | YYMH | AQKFQG | | LA | LES | RFPV | | | | | | |
| AB002520_ | AB00 | GYTFTD | IINPRAGYTSY | TSGWDV | RASQSISTW | AASS | QQSY | 533 | 534 | 535 | 536 | 537 | 538 |
| 7A04v8 | 2520 | YYMH | ALKFQG | | LA | LDS | RFPV | | | | | | |
| AB002521_ | AB00 | GHTFTR | IINPSGGYATY | ASGLDI | RASQSINSW | ATST | QQSY | 539 | 540 | 541 | 542 | 543 | 544 |
| 2A11v1 | 2521 | YYMH | AQKFQG | | LA | LES | RFPV | | | | | | |
| AB002829_ | AB00 | GYIFNG | WMNPDNGNT | ARGMA | RSSQSLLHS | LGSN | MQGT | 545 | 546 | 547 | 548 | 549 | 550 |
| knd_A08 | 2829 | YDIH | GLAQKFQG | TRFPY | NGYNYLD | RAS | HWPP | | | | | | |
| | | | | YYYGM | | | | | | | | | |
| | | | | DV | | | | | | | | | |
| AB002864_ | AB00 | GHTFTR | VINPSGGYAT | ASGWDV | RASQSINSW | ATST | QQSYS | 551 | 552 | 553 | 554 | 555 | 556 |
| 2A11v6 | 2864 | YYMH | YAQKFQG | | LA | LES | SPV | | | | | | |
| AB003005_ | AB00 | GYTFTR | IIQPRAGYTSY | TSGWDV | RASQSISTW | AASS | QQSYS | 557 | 558 | 559 | 560 | 561 | 562 |
| 7A04v5 | 3005 | YYMH | ALKFQG | | LA | LDE | FPV | | | | | | |
| AB003007_ | AB00 | GYTFTR | IINPRAGYTSY | TSGWEV | RASQSISTW | AASS | QQSYS | 563 | 564 | 565 | 566 | 567 | 568 |
| 7A04v6 | 3007 | YYMH | ALKFQG | | LA | LDE | FPV | | | | | | |
| AB003012_ | AB00 | GYTFTR | IINPRAGYTSY | TSGWDV | RASQSISTW | AASS | QQSYS | 569 | 570 | 571 | 572 | 573 | 574 |
| 7A04v7 | 3012 | YYMH | ALKFQG | | LA | LDE | FPV | | | | | | |
| AB003021_ | AB00 | GFTFTR | VINPSGGYAT | ASGWDV | RASQSISSW | ATST | QQSYS | 575 | 576 | 577 | 578 | 579 | 580 |
| 2A11v7 | 3021 | YYMH | YAQKFQG | | LA | LES | SPV | | | | | | |
| AB003155_ | AB00 | GETFTR | IINPSGGYASY | AAGLFI | RASQSIGRW | SASN | QQYN | 581 | 582 | 583 | 584 | 585 | 586 |
| 2B07v10 | 3155 | YYVH | AQKFQG | | LA | LET | RFPV | | | | | | |
| AB003174_ | AB00 | GDTFTR | IINPSGGYASY | AAGLFI | RASQSIGRE | SASN | QQYN | 587 | 588 | 589 | 590 | 591 | 592 |
| 2B07v11 | 3174 | YYVH | AQKFQG | | LA | LET | RFPV | | | | | | |
| AB003178_ | AB00 | GDEFTR | IINPSGGYASY | AAGLFI | RASQSIGRW | SASN | QQYN | 593 | 594 | 595 | 596 | 597 | 598 |
| 2B07v12 | 3178 | YYVH | AQKFQG | | LA | LET | RFPV | | | | | | |
| AB003180_ | AB00 | GDEFTR | IQNPSGGYASY | AAGLFI | RASQSIGRW | SASN | QQYN | 599 | 600 | 601 | 602 | 603 | 604 |
| 2B07v13 | 3180 | YYVH | AQKFQG | | LA | LET | RFPV | | | | | | |
| AB003183_ | AB00 | GDEFTR | IQNPSGGYASY | AAGLFI | RASQSIGRE | DAS | QQYN | 605 | 606 | 607 | 608 | 609 | 610 |
| 2B07v14 | 3183 | YYVH | AQKFQG | | LA | NLET | RFPV | | | | | | |
| AB003200_ | AB00 | GETFTN | VINPRAGYTSY | AGGWED | RISQSISSW | SASS | QQSFT | 611 | 612 | 613 | 614 | 615 | 616 |
| 2B05v1 | 3200 | YYIH | ALKFQG | | LA | LQS | SPI | | | | | | |
| AB003201_ | AB00 | GGTFTN | IIDPRAGYTSY | AGGWED | RISQSISSW | SASS | QQSFT | 617 | 618 | 619 | 620 | 621 | 622 |
| 2B05v2 | 3201 | YYIH | ALKFQG | | AL | LQS | SPI | | | | | | |
| AB003202_ | AB00 | GSTFTN | IIDPRAGYTSY | AGGWED | RISQSISSF | SASS | QQSFT | 623 | 624 | 625 | 626 | 627 | 628 |
| 2B05v3 | 3202 | YYIH | ALKFQG | | LA | LQS | SPI | | | | | | |
| AB003209_ | AB00 | GETFTN | IIDPRAGYTSY | AGGWED | RISQSISSF | SASS | QQSFT | 629 | 630 | 631 | 632 | 633 | 634 |
| 2B05v4 | 3209 | YYIH | ALKFQG | | LA | LQS | SPI | | | | | | |
| AB003232_ | AB00 | GTTFTN | VINPRAGYTSY | TGGWLD | RASQSISSW | SASS | QQSVT | 635 | 636 | 637 | 638 | 639 | 640 |
| 2B05v5 | 3232 | YYIH | ALKFQG | | LA | LQS | MPV | | | | | | |
| AB003422_ | AB00 | GYTFTN | VINPRAGYTSY | AGGWLD | RASQRISSW | DASS | QQSFT | 641 | 642 | 643 | 644 | 645 | 646 |
| 2B05v5 | 3422 | YYIH | ALKFQG | | LA | LQS | MPV | | | | | | |
| AB003465_ | AB00 | GFTFTR | VINPSGGYAT | ASGWDV | RASQSISSW | ATST | QQSYS | 647 | 648 | 649 | 650 | 651 | 652 |
| 2A11v9 | 3465 | YYMH | YAQKFQG | | LA | LES | FPP | | | | | | |
| AB003466_ | AB00 | GETFTR | IINPSGGYASY | AAGLFI | RASQSIGRW | SASN | QQYN | 653 | 654 | 655 | 656 | 657 | 658 |
| 2B07v10 | 3466 | YYVH | AQKFQG | | LA | LET | SFPV | | | | | | |
| AB003467_ | AB00 | GYTFTR | IINPRAGYTSY | TSGWDV | RASQSISTW | AASS | QQSYS | 659 | 660 | 661 | 662 | 663 | 664 |
| 7A04v9 | 3467 | YYMH | ALKFQG | | LA | LDT | FPV | | | | | | |
| AB003470_ | AB00 | GYTFTA | WIHPYSGGTN | AIGYY | RASQTISRY | AASS | QQSYS | 665 | 666 | 667 | 668 | 669 | 670 |
| knd_A04 | 3470 | YYIH | YAQKFQG | YGKFD | LN | LQS | TPR | | | | | | |
| | | | | Y | | | | | | | | | |

### Example 13

### Regulated IL-2 Receptor Binding by PD-1/IL-2 Cytokine Complexes Containing Additional Dual Binding Antibody (DBA) Domains In Vitro

This example describes PD-1 regulated IL-2 receptor binding by PD-1/IL-2 DBA-cytokine complexes including the additional DBA binding elements described in Example 12. Anti PD-1/IL-2 DBA-cytokine complexes were analyzed along with suitable non-regulated controls such as anti-PD-1, anti Her2, anti PDL-1 or anti IL-2 cytokine complexes. The DBA-cytokine complexes and control antibody-cytokine complexes were produced in two formats: (1) “Symmetric” immunocytokine (shown in FIG. 2E); or (2) “Asymmetric” immunocytokine (shown in FIG. 2B) by expression in mammalian cells and purified using standard protocols. An ELISA assay was performed with a constant amount of the antibody-cytokine construct coated on each well probed with biotinylated IL-2 receptor beta gamma heterodimer Fc (IL-2RBG; Acro Cat #: ILG-H5254) in the presence of varying amounts of PD-1-Fc or hIgG1-Fc. To perform the assay, 384-well ELISA plates were coated with anti-Fc antibody at 1 micrograms/ml in 100 mM bicarbonate solution pH 9.0 overnight at 4° C. and washed twice with SUPERBLOCK™. Antibody-cytokine complexes were then added to each well at a constant concentration of 6 nM and allowed to incubate for 1 hour and washed three times in PBS plus 0.05% TWEEN® 20 (PBST). Titrated concentrations of PD-1 Fc or an IgG1 control Fc were added and allowed to incubate for 15 minutes before the addition of a constant amount of biotinylated IL-2RBG at 10 nM. The plates were incubated for an additional 30 minutes, washed and biotinylated IL-2RBG detection was performed using streptavidin-HRP and standard ELISA protocols.

### Symmetric Designs

Results with protein complexes comprising the structure shown in FIG. 2E are shown in FIG. 9. As depicted in FIG. 2E, this symmetric format is comprised of one IL-2 linked to each antibody variable domain. As can be seen for the behavior of these constructs in FIG. 9, IL-2RBG binding increased in a dose dependent manner with the addition of PD-1 Fc (but not with the addition of negative control hIgG1 Fc protein) for the DBA-cytokine complexes AF3247, AF3644, AF3651, AF3652, AF3653, AF3657, AF3930, AF3931, AF3933, AF3934, and AF3935 comprising the peptide IDs noted in TABLE 35 below (the sequences of the referenced peptides can be found in Table 2A). IL-2RBG binding for the control monospecific antibody-cytokine complexes including anti Her2 (AF3243) and anti IL-2 (AF3246) did not change with the addition of PD-1 Fc protein.

TABLE 20
Multiprotein Components of “Symmetric” Immunocytokine
designs and controls tested in Example 13

| Protein | | | | |
|---|---|---|---|---|
| Complex | peptide1 | peptide2 | peptide3 | Antibody1 |
| AF003232 | PEP003639 | PEP003648 | PEP003642 | AB002022_2B07v1 |
| AF003740 | PEP004243 | PEP004247 | PEP004159 | AB002293_2B07v2 |
| AF003747 | PEP004243 | PEP004247 | PEP004162 | AB002345_2B07v6 |
| AF003749 | PEP004243 | PEP004247 | PEP004163 | AB002348_2B07v8 |
| AF003753 | PEP004243 | PEP004247 | PEP003642 | AB002353_2B07v9 |
| AF003941 | PEP004251 | PEP004443 | PEP000169 | AB001054_PDL1_DB02_D10 |
| | | | | control |
| AF003945 | PEP004243 | PEP004438 | PEP004421 | AB002326_2B07v3 |
| AF003947 | PEP004243 | PEP004438 | PEP004420 | AB002342_2B07v5 |
| AF003951 | PEP004433 | PEP004442 | PEP004427 | AB002360_7A04v1 |
| AF003952 | PEP004435 | PEP004445 | PEP004427 | AB002365_7A04v2 |
| AF003953 | PEP004436 | PEP004446 | PEP004427 | AB002370_7A04v3 |
| AF003955 | PEP004431 | PEP004440 | PEP004423 | AB002413_2A11v3 |
| AF003956 | PEP004433 | PEP004442 | PEP004426 | AB002520_7A04v8 |
| AF003243 | PEP003655 | PEP000113 | | AB001203_trastuzumab control |
| AF003246 | PEP003654 | PEP003641 | | AB001923_Anti-IL2 control |

### Asymmetric Designs

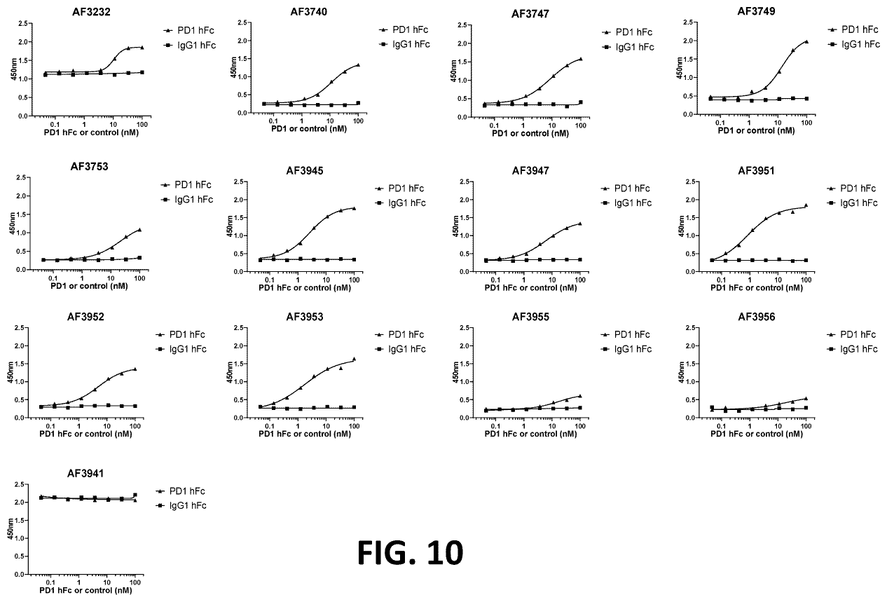

Results with protein complexes comprising the structures depicted in FIG. 2B (“asymmetric” immunocytokine design) and described in TABLE 21 below are shown in FIG. 10. This format is composed of an asymmetric complex comprised of two antibody domains with a single IL-2 linked to one of the domains. IL-2RBG binding increased in a dose dependent manner with the addition of PD-1 Fc but not with the addition of an IgG1 control Fc protein for the DBA-cytokine complexes AF3232, AF3740, AF3747, AF3749, AF3753, AF3945, AF3947, AF3951, AF3952, AF3953, AF3955, and AF3956 comprising the peptide IDs noted in TABLE 21 below (the sequences of the referenced peptides can be found in Table 2A). IL-2RBG binding for the control monospecific antibody-cytokine complex anti PDL-1 (AF3941) did not change with the addition of PD-1 Fc protein.

TABLE 21
Multiprotein Components of “Asymmetric” Immunocytokine
designs and controls tested in Example 13

| Protein | | | | |
|---|---|---|---|---|
| Complex | peptide1 | peptide2 | peptide2 | Antibody |
| AF003232 | PEP003639 | PEP003648 | PEP003642 | AB002022_2B07v1 |
| AF003740 | PEP004243 | PEP004247 | PEP004159 | AB002293_2B07v2 |
| AF003747 | PEP004243 | PEP004247 | PEP004162 | AB002345_2B07v6 |
| AF003749 | PEP004243 | PEP004247 | PEP004163 | AB002348_2B07v8 |
| AF003753 | PEP004243 | PEP004247 | PEP003642 | AB002353_2B07v9 |
| AF003945 | PEP004243 | PEP004438 | PEP004421 | AB002326_2B07v3 |
| AF003947 | PEP004243 | PEP004438 | PEP004420 | AB002342_2B07v5 |
| AF003951 | PEP004433 | PEP004442 | PEP004427 | AB002360_7A04v1 |
| AF003952 | PEP004435 | PEP004445 | PEP004427 | AB002365_7A04v2 |
| AF003953 | PEP004436 | PEP004446 | PEP004427 | AB002370_7A04v3 |
| AF003955 | PEP004431 | PEP004440 | PEP004423 | AB002413_2A11v3 |
| AF003956 | PEP004433 | PEP004442 | PEP004426 | AB002520_7A04v8 |
| AF003941 | PEP004251 | PEP004443 | PEP000169 | AB001054_PDL1_DB02_D10 |
| | | | | control |

### Example 14

### Inhibition of Two Forms of IL-2 Binding to IL-2RBG to PD-1/IL-2 Cytokine Complexes In Vitro

This example describes two forms of IL-2 in PD-1/IL-2 DBA-cytokine complexes binding to IL-2RBG. Antibody-cytokine complexes in the format depicted in FIG. 2H were generated with wild-type (WT) IL-2 or IL-2 3× (an IL-2 variant with reduced binding to IL-2Ralpha and having R38D, K43E, and E61R mutations, see e.g. Vazquez-Lombardi et al. Nat Commun. 8:15371 2017, which is incorporated by reference in its entirety herein). The DBA-cytokine complexes and control antibody-cytokine complexes were produced by expression in mammalian cells and purified using standard protocols. To examine the ability of DBA binding domains to block IL-2 from binding to IL-2RBG, an ELISA assay was performed with a constant amount of the antibody-cytokine construct coated on each well probed with biotinylated IL-2 receptor beta gamma heterodimer Fc (IL-2RBG; Acro Cat #: ILG-H5254. In these experiments, 384-well ELISA plates were coated with anti-Fc antibody (Jackson ImmunoResearch) at 1 micrograms/ml in 100 mM bicarbonate solution pH 9.0 overnight at 4° C. and washed twice with SUPERBLOCK™ (ThermoFisher, 37515). Antibody-cytokine complexes were then added to each well at a constant concentration of 6 nM and allowed to incubate for 1 hour and washed three times in PBS plus 0.05% TWEEN® 20 (PBST). Titrating concentrations of biotinylated IL-2RBG were added and the plates were incubated for an additional 45 minutes. After washing, the biotinylated IL-2RBG detection was performed using streptavidin-HRP and standard ELISA protocols.

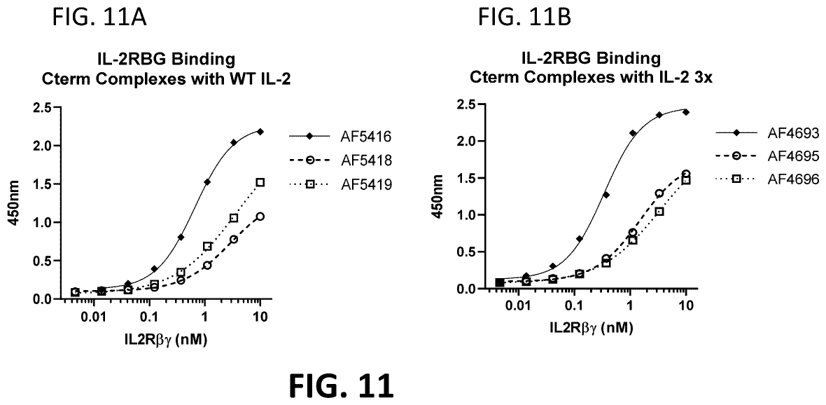

Results with protein complexes described in TABLE 22 below are shown in FIG. 11. As depicted in FIG. 2H, these complexes are asymmetric and comprised of two identical monospecific Fab arms with a single IL-2 (either WT or 3×) attached to one Fc domain by flexible linker and a single scFv attached to the other Fc domain by a flexible linker. The antibody-cytokine complexes comprise an anti-PD-1 domain in the Fab arms and a PD-1/IL-2 DBA scFv on the Fc arm. The control antibody-cytokine complexes comprise the same anti-PD-1 domain in the Fab arms and an anti HER2 monospecific scFv on the Fc arm. In FIG. 11 panel A, the antibody-cytokine complexes contain the WT IL-2 form on the Fc domain and in FIG. 11 panel B, the antibody-cytokine complexes contain the 3×IL-2 form on the Fc. As shown in FIG. 11 panel A, the PD-1/IL-2 DBA complexes AF5418 and AF5419 have reduced IL-2RBG binding compared to the anti Her2 non DBA control complex AF5416 demonstrating the ability of the DBA domains to block receptor binding to WT IL-2. As shown in FIG. 11 panel B, the PD-1/IL-2 DBA complexes AF4695 and AF4696 have reduced IL-2RBG binding compared to the anti Her2 non DBA control complex AF4693 demonstrating the ability of the DBA domains to block receptor binding to IL-2 3×.

TABLE 22
Multiprotein Components of “Cterm” Immunocytokine designs and controls tested in Example 14

| Peptide | | | | | |
|---|---|---|---|---|---|
| ID | peptide1 | peptide2 | peptide3 | Antibody1 | Antibody2 |
| AF004693 | PEP004967 | PEP005090 | PEP004729 | AB001203_trastuzumab | AB000694_PD1_nivolumab |
| | | | | control | control |
| AF004695 | PEP004967 | PEP005091 | PEP004729 | AB002328_2B07v4 | AB000694_PD1_nivolumab |
| | | | | | control |
| AF004696 | PEP004967 | PEP005092 | PEP004729 | AB002365_7A04v2 | AB000694_PD1_nivolumab |
| | | | | | control |
| AF005416 | PEP006177 | PEP005090 | PEP004729 | AB001203_trastuzumab | AB000694_PD1_nivolumab |
| | | | | control | control |
| AF005418 | PEP006177 | PEP005091 | PEP004729 | AB002328_2B07v4 | AB000694_PD1_nivolumab |
| | | | | | control |
| AF005419 | PEP006177 | PEP005092 | PEP004729 | AB002365_7A04v2 | AB000694_PD1_nivolumab |
| | | | | | control |

### Example 15

Regulated IL-2 Receptor Signaling by PD-1/IL-2 Dual Binding Antibody (DBA) Cytokine Complexes in Cellular Assays

This example describes PD-1 regulated IL-2 activity in a HEK-BLUE™ IL-2 reporter cell by PD-1/IL-2 DBA-cytokine complexes. Anti PD-1/IL-2 DBA-cytokine complexes were analyzed along with suitable non-regulated controls such as anti Her2, anti PDL-1 or anti IL-2 cytokine complexes. The DBA-cytokine complexes and control antibody-cytokine complexes were produced in five formats shown in FIGS. 2B, 2D, 2E, 2G, 2H, and 2I by expression in mammalian cells and purified using standard protocols; the peptide components of these complexes are outlined in TABLE 23 below (where sequences of individual components can be found in Table 2A). A cell-based reporter assay was performed for each of the five formats in the presence of varying amounts of PD-1-Fc or hIgG1-Fc.

TABLE 23
Multiprotein Components of Immunocytokine designs and controls tested in Example 15

| Protein | Format | | | | | |
|---|---|---|---|---|---|---|
| Complex | (Figure) | Chain 1 | Chain 2 | Chain 3 | Antibody1 | Antibody2 |
| AF003232 | 2B | PEP003639 | PEP003648 | PEP003642 | AB002022_2B07v1 | — |
| AF003744 | 2B | PEP004243 | PEP004247 | PEP004158 | AB002328_2B07v4 | — |
| AF003747 | 2B | PEP004243 | PEP004247 | PEP004162 | AB002345_2B07v6 | — |
| AF003941 | 2B | PEP004251 | PEP004443 | PEP000169 | AB001054_PDL1_{—} | — |
| | | | | | DB02_D10 control | |
| AF003946 | 2B | PEP004243 | PEP004438 | PEP004158 | AB002328_2B07v4 | — |
| AF003948 | 2B | PEP004243 | PEP004438 | PEP004162 | AB002345_2B07v6 | — |
| AF003952 | 2B | PEP004435 | PEP004445 | PEP004427 | AB002365_7A04v2 | — |
| AF003955 | 2B | PEP004431 | PEP004440 | PEP004423 | AB002413_2A11v3 | — |
| AF003956 | 2B | PEP004433 | PEP004442 | PEP004426 | AB002520_7A04v8 | — |
| AF003345 | 2D | PEP003791 | PEP003786 | | AB002022_2B07v1 | — |
| AF003243 | 2E | PEP003655 | PEP000113 | | AB001203_{—} | — |
| | | | | | trastuzumab control | |
| AF003246 | 2E | PEP003654 | PEP003641 | | AB001923_Anti-IL2 | — |
| | | | | | control | |
| AF003247 | 2E | PEP003657 | PEP003642 | | AB002022_2B07v1 | — |
| AF003341 | 2E | PEP003780 | PEP003782 | | AB002022_2B07v1 | — |
| AF003644 | 2E | PEP004153 | PEP004159 | | AB002293_2B07v2 | — |
| AF003651 | 2E | PEP004153 | PEP004162 | | AB002345_2B07v6 | — |
| AF003657 | 2E | PEP004153 | PEP003642 | | AB002353_2B07v9 | — |
| AF003934 | 2E | PEP004418 | PEP004427 | | AB002365_7A04v2 | — |
| AF003873 | 2G | PEP004361 | PEP003642 | | AB002022_2B07v1 | AB000880_PD1_{—} |
| | | | | | | 4C10 control |
| AF003876 | 2G | PEP004363 | PEP004158 | | AB002328_2B07v4 | AB000880_PD1_{—} |
| | | | | | | 4C10 control |
| AF003877 | 2G | PEP004363 | PEP004162 | | AB002345_2B07v6 | AB000880_PD1_{—} |
| | | | | | | 4C10 control |
| AF003864 | 2H | PEP003626 | PEP004356 | PEP000243 | AB001923_Anti-IL2 | AB000881_PD1_{—} |
| | | | | | control | control |
| AF003871 | 2H | PEP004360 | PEP000113 | PEP004360 | AB001203_{—} | AB000880_PD1_{—} |
| | | | | | trastuzumab control | 4C10 control |
| AF003872 | 2H | PEP004359 | PEP003641 | PEP004359 | AB001923_Anti-IL2 | AB000880_PD1_{—} |
| | | | | | control | 4C10 control |
| AF003913 | 2H | PEP003626 | PEP004395 | PEP000243 | AB002022_2B07v1 | AB000881_PD1_{—} |
| | | | | | | control |
| AF003918 | 2H | PEP003626 | PEP004398 | PEP000243 | AB002328_2B07v4 | AB000881_PD1_{—} |
| | | | | | | control |
| AF003923 | 2H | PEP003626 | PEP004406 | PEP000243 | AB002360_7A04v1 | AB000881_PD1_{—} |
| | | | | | | control |
| AF003927 | 2H | PEP003626 | PEP004404 | PEP000243 | AB002413_2A11v3 | AB000881_PD1_{—} |
| | | | | | | control |
| AF004502 | 2H | PEP005089 | PEP005090 | PEP004729 | AB000694_PD1_{—} | AB001203_{—} |
| | | | | | nivolumab control | trastuzumab control |
| AF004503 | 2H | PEP003628 | PEP005088 | PEP000113 | AB001203_{—} | AB001203_{—} |
| | | | | | trastuzumab control | trastuzumab control |
| AF004504 | 2H | PEP005089 | PEP005091 | PEP004729 | AB002328_2B07v4 | AB000694_PD1_{—} |
| | | | | | | nivolumab control |
| AF004505 | 2H | PEP005089 | PEP005092 | PEP004729 | AB002365_7A04v2 | AB000694_PD1_{—} |
| | | | | | | nivolumab control |
| AF004892 | 2H | PEP000288 | PEP005631 | PEP000244 | AB002328_2B07v4 | AB000881_PD1_{—} |
| | | | | | | control |
| AF004893 | 2H | PEP000288 | PEP005634 | PEP000244 | AB002365_7A04v2 | AB000881_PD1_{—} |
| | | | | | | control |
| AF003632 | 2I | PEP004130 | PEP004129 | PEP000244 | AB000881_PD1_{—} | — |
| | | | | | control | |
| AF003634 | 2I | PEP004134 | PEP004133 | PEP003782 | AB002022_2B07v1 | — |

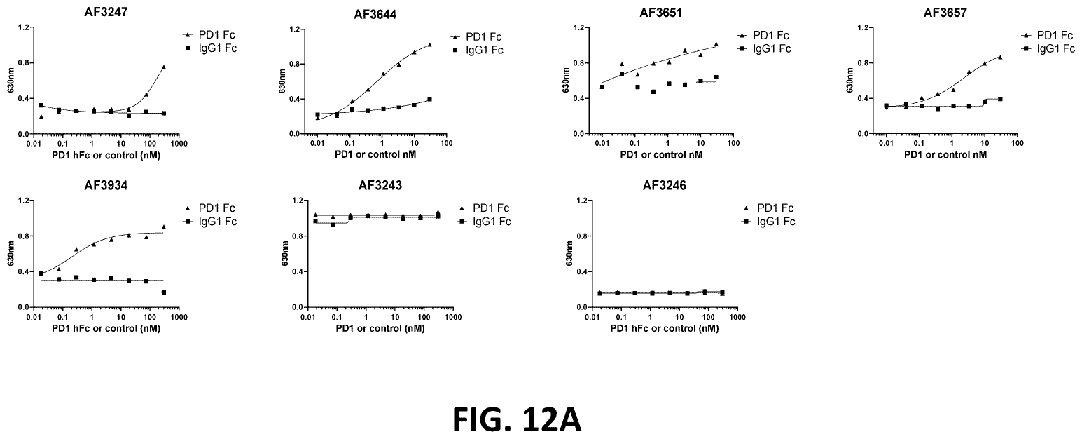

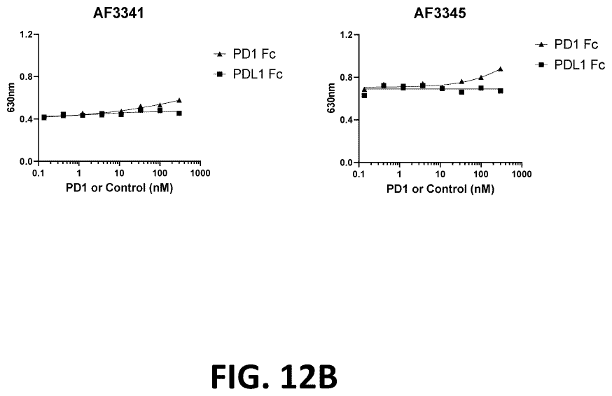

In the experiments shown in FIGS. 12A, 12B, 13A and 25, the antibody-cytokine complexes were diluted to a final concentration of 100 pM (a concentration previously shown to have a strong reporter signal for the always on control but little to no signal for the always off control) into wells of a 384 well TC treated plate (Corning 3701) in complete DMEM (+10% FBS, 2 mM L-glutamine, sodium pyruvate) along with titrated concentrations of PD-1 Fc or an IgG1 control Fc. After a 15-minute incubation HEK-BLUE™ IL-2 reporter cells (12,500 cells) were added to each well and incubated overnight. Five microliters from each well was transferred to a new plate containing 45 microliters of QUANTI-BLUE™ solution (Invivogen Product #rep-qbs). After 30 to 60 minutes the absorbance at 630 nm was determined using a Perkin-Elmer ENVISION™.

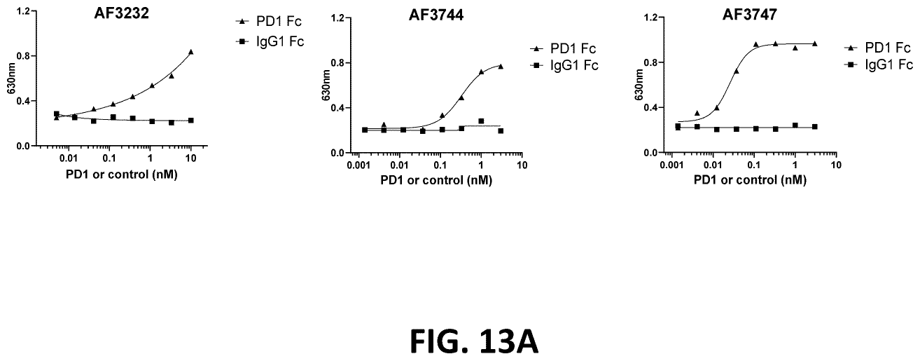

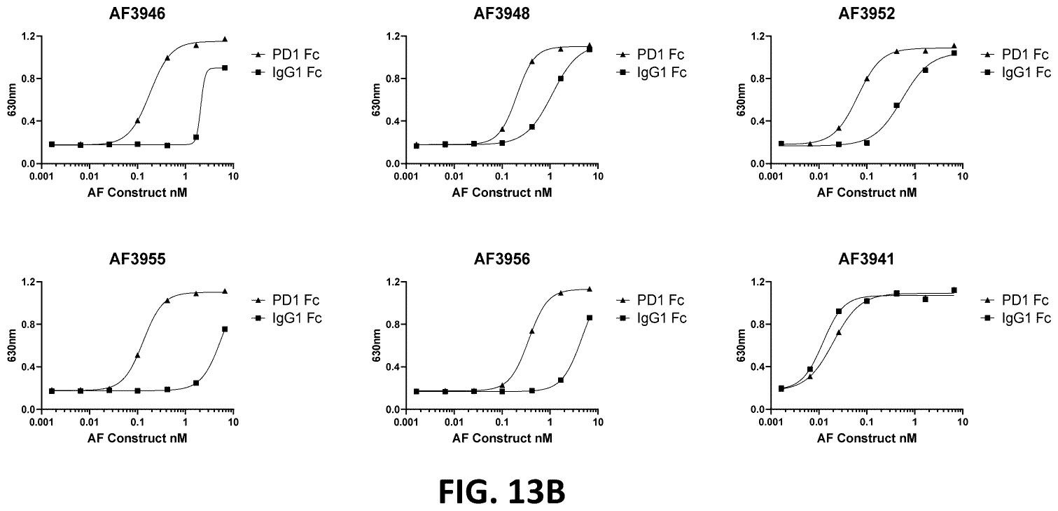

In an alternative experiment format shown in FIGS. 13B, 14A, 14B, 20 and 22, the wells of a 384-well ELISA plate were coated with constant concentration of PD-1-Fc or an IgG1 Fc control protein captured with an anti-Fc antibody (Jackson ImmunoResearch, Prod. #109-005-098). The cytokine complexes were serially diluted 1:4 for 8 points in growth media from a starting concentration of 6 nM and incubated briefly before addition of the HEK-BLUE™ IL-2 reporter cells.

Results with protein complexes comprising the structure shown in FIG. 2E are shown in FIGS. 12A and 12B. As depicted in FIG. 2E, this symmetric format is comprised of one IL-2 linked to each antibody heavy chain variable domain. IL-2 activity increased in a dose dependent manner with the addition of PD-1 Fc but not with the addition of hIgG1 Fc protein for the DBA-cytokine complexes AF3247, AF3644, AF3651, AF3657, and AF3934. IL-2 activity for the control anti-Her2 AF3243 and anti-IL-2 AF3246 monospecific antibody-cytokine complexes did not change with the addition of PD-1 Fc protein. The symmetric format in FIG. 12B is similar to the DBA-cytokine complexes in FIG. 12A however the IL-2 is conjugated to the heavy chain variable domain for AF3341 and the light chain variable domain for AF3345. Both symmetric formats demonstrate increased IL-2 activity with the addition of PD-1 Fc but not with the addition of hIgG1 Fc.

Results with protein complexes comprising the structures depicted in FIG. 2B are shown in FIGS. 13A and 13B. This format is composed of an asymmetric complex comprised of two antibody domains with a single IL-2 linked to one of the domains. In FIG. 13A, IL-2 activity increased in a dose dependent manner with the addition of PD-1 Fc but not with the addition of an IgG1 control Fc protein for the DBA-cytokine complexes AF3232, AF3744, and AF3747. In FIG. 13B, results with the asymmetric constructs in the alternative assay format where PD-1 Fc or hIgG1 Fc is captured to the plate are shown. The DBA-cytokine complexes AF3946, AF3948, AF3952, AF3955 and AF3956 demonstrate increased IL-2 activity in the wells coated with PD-1 compared to wells coated with hIgG1 Fc. IL-2 activity for the control anti-PDL-1 monospecific antibody-cytokine complex AF3941 did not change with the addition of PD-1 Fc protein.

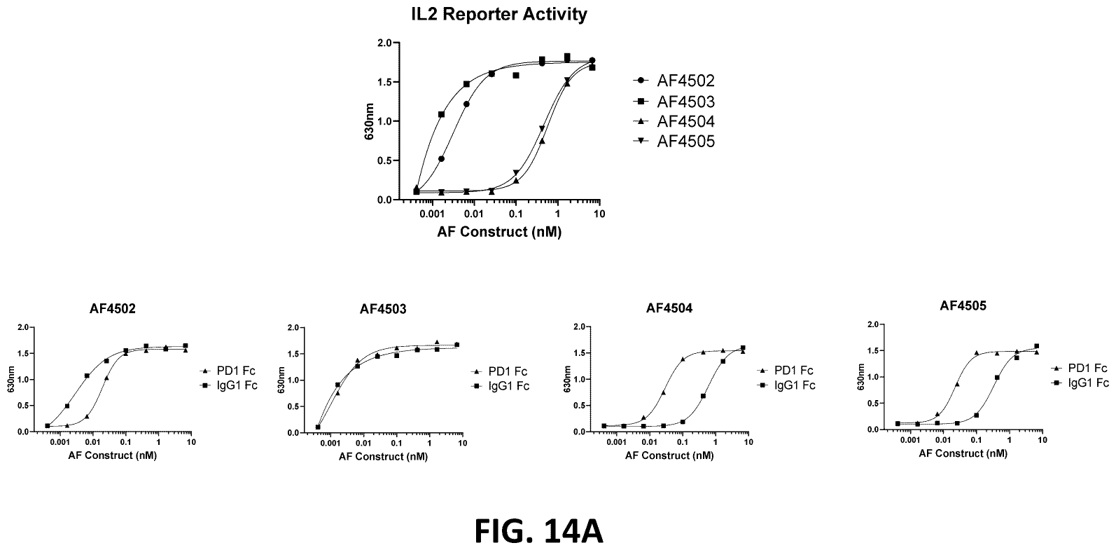

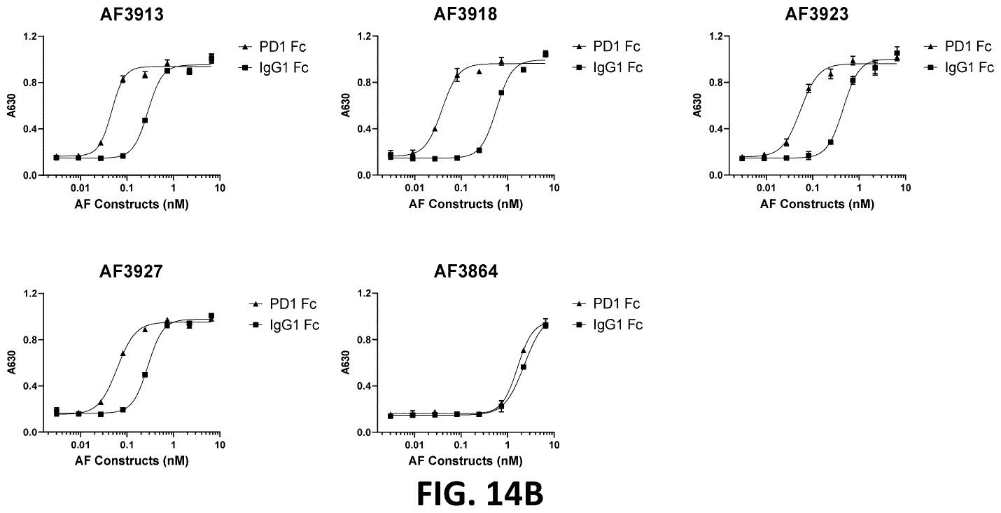

Results with protein complexes comprising the structures depicted in FIG. 2H are shown in FIGS. 14A and 14B. As depicted in FIG. 2H, these complexes are asymmetric and comprised of two identical monospecific Fab arms with a single IL-2 attached to one Fc domain by flexible linker and a single scFv attached to the other Fc domain by a flexible linker.

In FIG. 14A, the PD-1/IL-2 DBA complexes are composed of an anti-PD-1 domain in the Fab arms (a PD1-nivolumab control) and a PD-1/IL-2 DBA scFv on the Fc arm. The control antibody-cytokine complexes are composed of the same anti-PD-1 domain in the Fab arm and a non-DBA scFv on the Fc arm. When titrating amounts of the cytokine complexes are added to the cells, the PD-1/IL-2 DBA containing cytokine complexes AF4504 and AF4505 in the upper graph show decreased reporter activation compared to equimolar amounts of the control anti-HER2 IL-2 immunocytokines AF4502 and AF4503. The same titrating amounts of the cytokine complexes were added to wells coated with PD-1 Fc or human IgG1 Fc control in the lower graphs. Both AF4504 and AF4505 demonstrate increased IL-2 activity in the PD-1 coated wells compared to the wells coated with the human IgG1 Fc. IL-2 activity for the control anti-HER2 IL-2 immunocytokines AF4502 and AF4503 did not change in the wells coated with PD1 Fc compared to the hIgG1 Fc control. In FIG. 14B, the PD-1/IL-2 DBA complexes are composed of a different anti-PD-1 domain in the Fab arms (AB000881_PD1_control) and PD-1/IL-2 DBA scFvs on the Fc arm. The PD-1/IL-2 DBA containing cytokine complexes AF3913, AF3918, AF3923 and AF3927 demonstrate increased IL-2 activity in the wells coated with PD-1 Fc compared to wells coated with hIgG1 Fc. IL-2 activity for the anti-IL-2 non-DBA scFv control antibody-cytokine complex AF3864 did not change in the wells coated with PD-1.

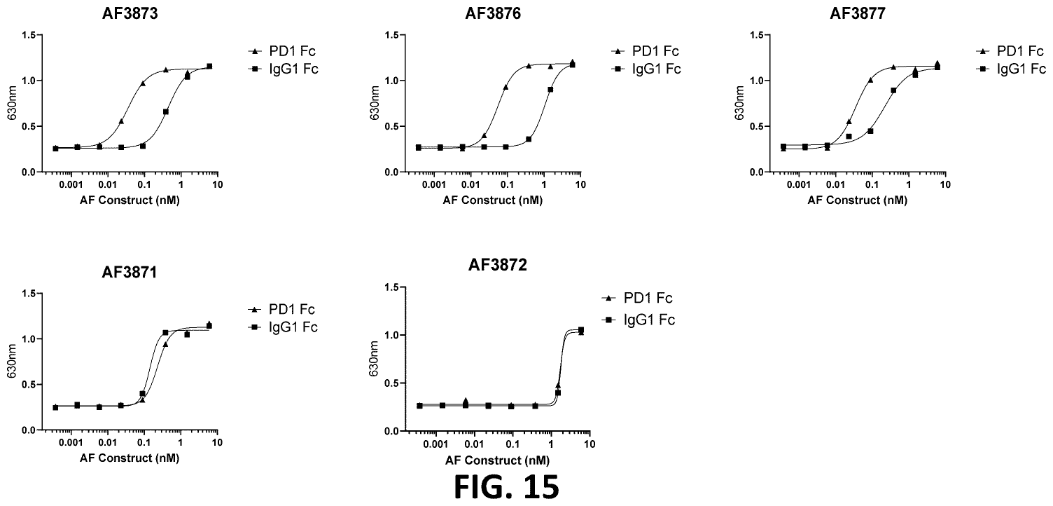

Results with protein complexes comprising the structure depicted in FIG. 2G are shown in FIG. 15. As depicted in FIG. 2G, this symmetric format is comprised of one IL-2 linked to each antibody heavy chain variable domain in the Fab arm and one scFv attached to each Fc domain. The antibody-cytokine complexes are composed of a PD-1/IL-2 DBA in the Fabs arms and an anti-PD-1 scFv (AB000880_PD1_4C10_control) on the Fc domain. The control antibody-cytokine complexes are composed of the same anti-PD-1 domain in the Fc domain and a non-DBA in the Fab arm. AF3871 has the non-DBA anti-Her2 antibody on the Fab arm and AF3872 has the non-DBA anti-IL-2 antibody on the Fab arm. The PD-1/IL-2 DBA containing complexes AF3873, AF3876 and AF3877 demonstrate increased IL-2 reporter activity in the wells coated with PD-1 Fc compared to the wells coated with hIgG1. IL-2 activity for the two control antibody-cytokine complexes AF3871 and AF3872 did not change in the wells coated with PD-1 Fc.

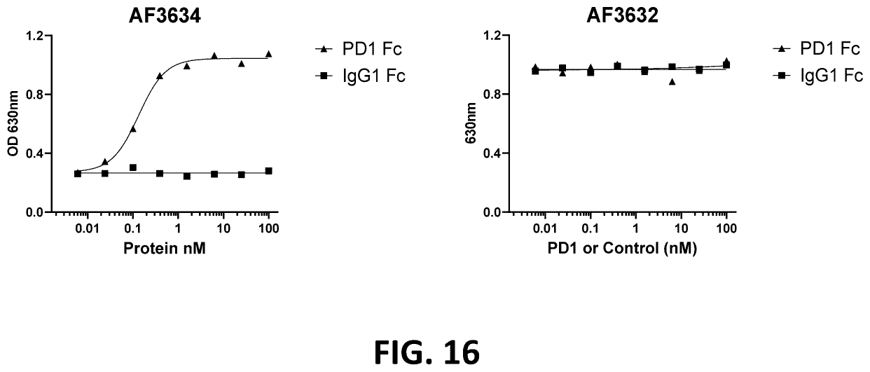

Results with protein complexes comprising the structure depicted in FIG. 2I are shown in FIG. 16. As depicted in FIG. 2I, these complexes are asymmetric and comprised of a PD-1/IL-2 DBA in the Fab arms with a single IL-2 attached to one Fc domain by flexible linker. The hinge region of the antibody is a hybrid of the hinge sequence of an IgG1 and IgG3 with the disulfide bridges removed to provide increased flexibility between the Fab arm and the IL-2 cytokine on the Fc domain. The control antibody-cytokine complex is composed of a monospecific anti-PD-1 domain in the Fab arms and the same IL-2 on the Fc domain. IL-2 activity increased in a dose dependent manner with the addition of PD-1 Fc but not with the addition of hIgG1 Fc protein for the DBA-cytokine complex AF3634. IL-2 activity for the control anti-PD-1 monospecific antibody-cytokine complex AF3632 did not change with the addition of PD-1 Fc protein.

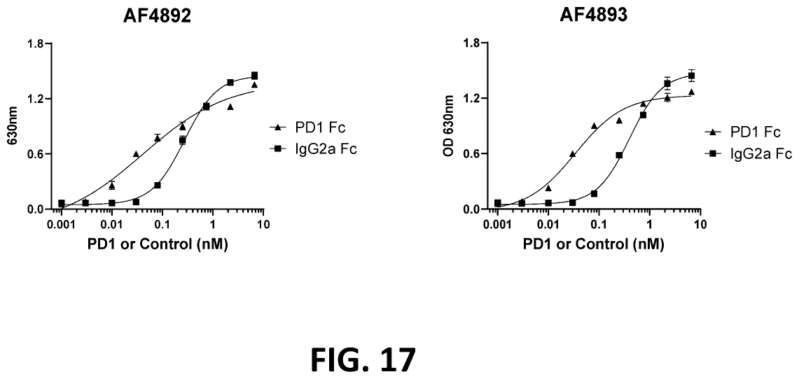

Results with protein complexes comprising the structures depicted in FIG. 2H are shown in FIG. 17. As depicted in FIG. 2H) and similar to the constructs in FIGS. 14A and 14B, these complexes are asymmetric and comprised of two identical monospecific Fab arms with a single IL-2 attached to one Fc domain by flexible linker and a single scFv attached to the other Fc domain by a flexible linker. Unique to the constructs in FIG. 17, the Fc portion of the construct is a human IgG1 isotype. The two constructs depicted in FIG. 17 are composed of an anti-PD-1 domain in the Fab arms (AB000881_PD1_control) and a PD-1/IL-2 DBA scFv on the Fc arm. Both PD-1/IL2 DBA containing cytokine complexes AF4892 and AF4893 demonstrate increased IL-2 activity in the wells coated with PD-1 Fc compared to wells coated with mIgG2a Fc control.

### Example 16

### Regulated IL-2 3× Receptor Signaling by PD-1/IL-2 3× Dual Binding Antibody (DBA) Cytokine Complexes In Vitro

This example describes PD-1 regulated IL-2 3× (an IL-2 variant with reduced binding to IL-2Ralpha, Lombardi et al, 2017) activity in a HEK-BLUE™ IL-2 reporter cell by PD-1/IL-2 3×DBA-cytokine complexes. Anti PD-1/IL-2 3×DBA-cytokine complexes were analyzed along with suitable non-regulated controls such as anti Her2, anti PD-1 or anti IL-2 cytokine complexes. The DBA-cytokine complexes and control antibody-cytokine complexes were produced in two formats shown in FIGS. 2B and 2H by expression in mammalian cells and purified using standard protocols. A cell-based reporter assay was performed for each of the two formats in the presence of plate bound PD-1-Fc or hIgG1-Fc. 384-well ELISA plates (Corning 3700) were coated with anti-Fc antibody (Jackson ImmunoResearch) at 1 micrograms/ml in 100 mM bicarbonate solution pH 9.0 overnight at 4° C. and washed twice with SUPERBLOCK™ (ThermoFisher). PD1-Fc or IgG1 control Fc were then added to each well at a constant concentration of 6 nM and allowed to incubate for 1 hour and washed three times in PBS plus 0.05% TWEEN® 20 (PBST). The antibody-cytokine complexes were serially diluted 1:4 for 8 points in complete DMEM (+10% FBS, 2 mM L-glutamine, sodium pyruvate) from a starting concentration of 6 nM. After a 15-minute incubation HEK-BLUE™ IL-2 reporter cells (12,500 cells) were added to each well and incubated overnight. Five microliters from each well were transferred to a new plate containing 45 microliters of QUANTI-BLUE™ solution (Invivogen Product #rep-qbs). After 30 to 60 minutes the absorbance at 630 nm was determined using a Perkin-Elmer ENVISION™.

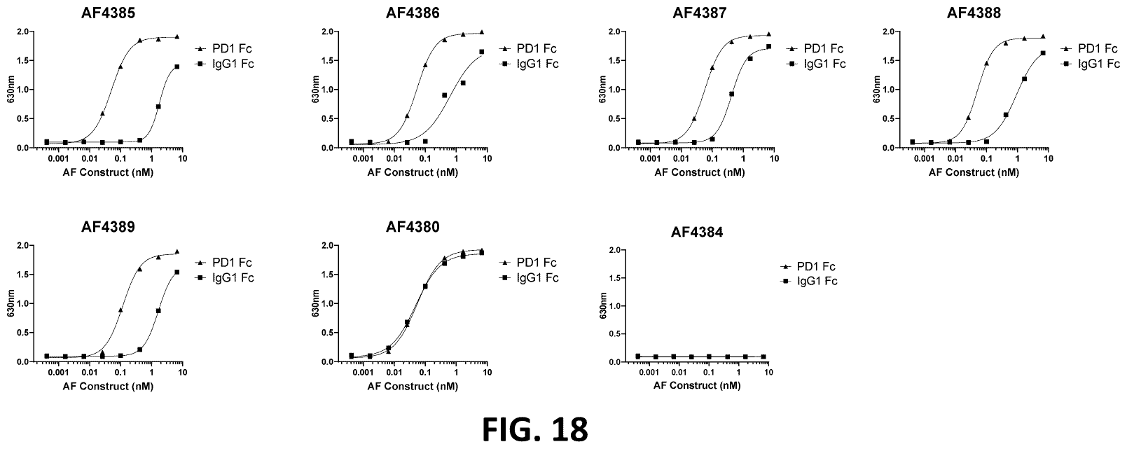

Results with protein complexes comprising the structures depicted in FIG. 2B are shown in FIG. 18. This format is composed of an asymmetric complex comprised of two antibody domains with a single IL-2 3× linked to one of the domains. The DBA-cytokine complexes AF4385, AF4386, AF4387, AF4388, and AF4389 demonstrate increased IL-2 3× activity in the wells coated with PD-1 Fc compared to wells coated with hIgG1 Fc. IL-2 3× activity for the control anti-PD-1 monospecific antibody-cytokine complex AF4380 and the anti-IL-2 monospecific antibody-cytokine complex AF4384 did not change with the addition of PD-1 Fc protein.

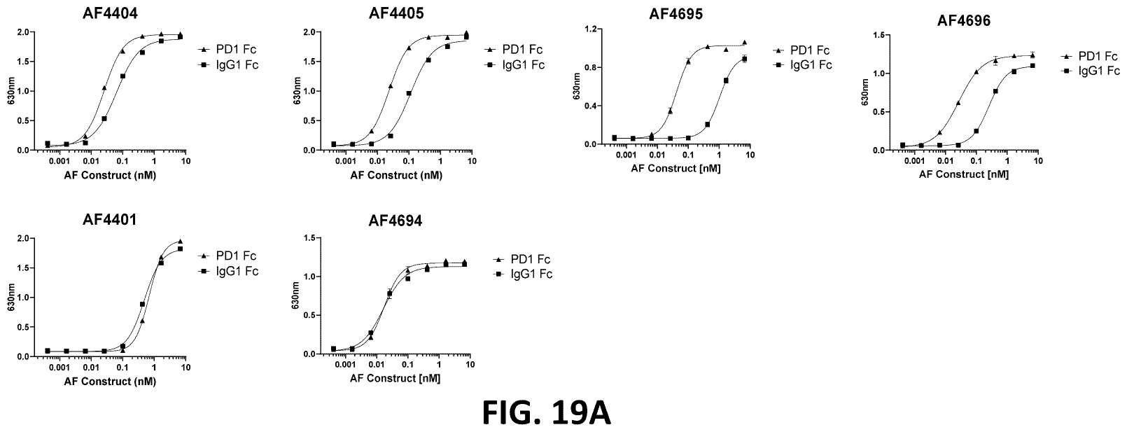

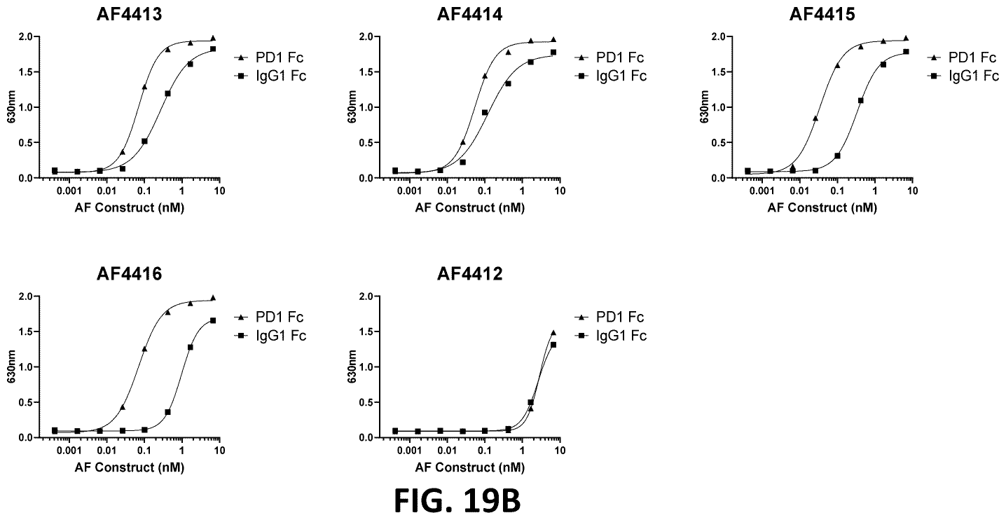

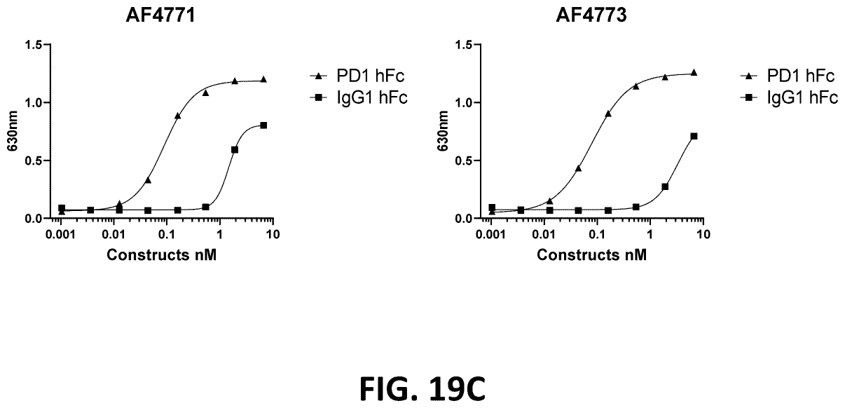

Results with protein complexes comprising the structures depicted in FIG. 2H are shown in FIGS. 19A, 19B, and 19C. As depicted in FIG. 2H, these complexes are asymmetric and comprise two identical monospecific Fab arms with a single IL-2 3× attached to one Fc domain by flexible linker and a single scFv attached to the other Fc domain by a flexible linker.

In FIG. 19A, the PD-1/IL-2 DBA complexes are composed of an anti-PD-1 domain in the Fab arms (AB000694_nivo) and a PD-1/IL-2 DBA scFv on the Fc arm. The control antibody-cytokine complexes are composed of the same anti-PD-1 domain in the Fab arms and a non-DBA scFv on the Fc arm. The PD-1/IL-2 3×DBA containing cytokine complexes AF4404, AF4405, AF4695 and AF4696 demonstrate increased IL-2 3× activity in the wells coated with PD-1 Fc compared to wells coated with hIgG1 Fc. IL-2 3× activity for the anti-IL-2 non-DBA scFv control antibody-cytokine complex AF4401 and the anti-Her2 non-DBA scFv control antibody-cytokine complex AF4694 did not change in the wells coated with PD-1 Fc. In FIG. 19B, the PD-1/IL-2 DBA complexes are composed of a different anti-PD-1 domain in the Fab arms (AB000880_PD1_R04_C10,) and PD-1/IL-2 DBA scFvs on the Fc arm. The PD-1/IL-2 3×DBA containing cytokine complexes AF4413, AF4414, AF4415 and AF4416 demonstrate increased IL-2 3× activity in the wells coated with PD-1 Fc compared to wells coated with hIgG1 Fc. IL-2 3× activity for the anti-IL-2 non-DBA scFv control antibody-cytokine complex AF4412 did not change in the wells coated with PD-1 Fc. In FIG. 19C, the PD-1/IL-2 DBA complexes are composed of anti-PD-1 domains in the Fab arms that do not block PDL-1 binding to PD-1 nor block the binding of nivolumab to PD-1. Both PD-1/IL-2 DBA containing complexes AF4771 and AF4773 demonstrate increased IL-2 3× activity in the wells coated with PD-1 Fc compared to wells coated with hIgG1 Fc.

TABLE 24
Multiprotein Components of Immunocytokine designs and controls tested in Example 16

| Protein | Format | | | | | |
|---|---|---|---|---|---|---|
| Complex | (Figure) | Chain 1 | Chain 2 | Chain 3 | Antibody1 | Antibody2 |
| AF004385 | 2B | PEP004957 | PEP004438 | PEP004420 | AB002342_{—} | — |
| | | | | | 2B07v5 | |
| AF004386 | 2B | PEP004957 | PEP004438 | PEP004162 | AB002345_{—} | — |
| | | | | | 2B07v6 | |
| AF004387 | 2B | PEP004959 | PEP004442 | PEP004427 | AB002360_{—} | — |
| | | | | | 7A04v1 | |
| AF004388 | 2B | PEP004960 | PEP004446 | PEP004427 | AB002370_{—} | — |
| | | | | | 7A04v3 | |
| AF004389 | 2B | PEP004958 | PEP004440 | PEP004423 | AB002413_{—} | — |
| | | | | | 2A11v3 | |
| AF004404 | 2H | PEP004967 | PEP004974 | PEP004729 | AB002360_{—} | AB000694_{—} |
| | | | | | 7A04v1 | PD1_nivolumab |
| | | | | | | control |
| AF004405 | 2H | PEP004967 | PEP004973 | PEP004729 | AB002413_{—} | AB000694_{—} |
| | | | | | 2A11v3 | PD1_nivolumab |
| | | | | | | control |
| AF004413 | 2H | PEP004978 | PEP004981 | PEP000245 | AB002342_{—} | AB000880_{—} |
| | | | | | 2B07v5 | PD1_4C10 |
| | | | | | | control |
| AF004414 | 2H | PEP004978 | PEP004982 | PEP000245 | AB002345_{—} | AB000880_{—} |
| | | | | | 2B07v6 | PD1_4C10 |
| | | | | | | control |
| AF004415 | 2H | PEP004978 | PEP004984 | PEP000245 | AB002360_{—} | AB000880_{—} |
| | | | | | 7A04v1 | PD1_4C10 |
| | | | | | | control |
| AF004416 | 2H | PEP004978 | PEP004983 | PEP000245 | AB002413_{—} | AB000880_{—} |
| | | | | | 2A11v3 | PD1_4C10 |
| | | | | | | control |
| AF004695 | 2H | PEP004967 | PEP005091 | PEP004729 | AB002328_{—} | AB000694_{—} |
| | | | | | 2B07v4 | PD1_nivolumab |
| | | | | | | control |
| AF004696 | 2H | PEP004967 | PEP005092 | PEP004729 | AB002365_{—} | AB000694_{—} |
| | | | | | 7A04v2 | PD1_nivolumab |
| | | | | | | control |
| AF004771 | 2H | PEP005470 | PEP005471 | PEP005469 | AB002328_{—} | AB002829_{—} |
| | | | | | 2B07v4 | knd_A04_PD1 |
| | | | | | | antibody |
| AF004773 | 2H | PEP005474 | PEP005475 | PEP005473 | AB002328_{—} | AB003470_{—} |
| | | | | | 2B07v4 | knd_A08_PD1 |
| | | | | | | antibody |

### Example 17

### Regulated IL-2 Receptor Signaling by PD-1/IL-2 Dual Binding Antibody (DBA) Cytokine Complexes with Variable Linker Lengths in Cells

This example describes PD-1 regulated IL-2 activity in a HEK-BLUE™ IL-2 reporter cell model by PD-1/IL-2 DBA-cytokine complexes with varying linker lengths. The DBA-cytokine complexes were produced in the format shown in FIG. 2B where the Glycine-Serine (GS) linker connecting the IL-2 cytokine to the DBA domain is varied from 5 GS repeats to 25 GS repeats. The DBA-cytokine complexes were expressed in mammalian cells and purified using standard protocols. A cell-based reporter assay was performed in the presence of plate bound PD-1-Fc or hIgG1-Fc. In this experiment, 384-well ELISA plates (Corning 3700) were coated with anti-Fc antibody (Jackson ImmunoResearch) at 1 micrograms/ml in 100 mM bicarbonate solution pH 9.0 overnight at 4° C. and washed twice with SUPERBLOCK™ (ThermoFisher). PD1-Fc or IgG1 control Fc were then added to each well at a constant concentration of 6 nM and allowed to incubate for 1 hour and washed three times in PBS plus 0.05% TWEEN® 20 (PBST). The antibody-cytokine complexes were serially diluted 1:4 for 8 points in complete DMEM (+10% FBS, 2 mM L-glutamine, sodium pyruvate) from a starting concentration of 6 nM. After a 15-minute incubation HEK-BLUE™ IL-2 reporter cells (12,500 cells) were added to each well and incubated overnight. Five microliters from each well were transferred to a new plate containing 45 microliters of QUANTI-BLUE™ solution (Invivogen Product #rep-qbs). After 30 to 60 minutes the absorbance at 630 nm was determined using a Perkin-Elmer ENVISION™.

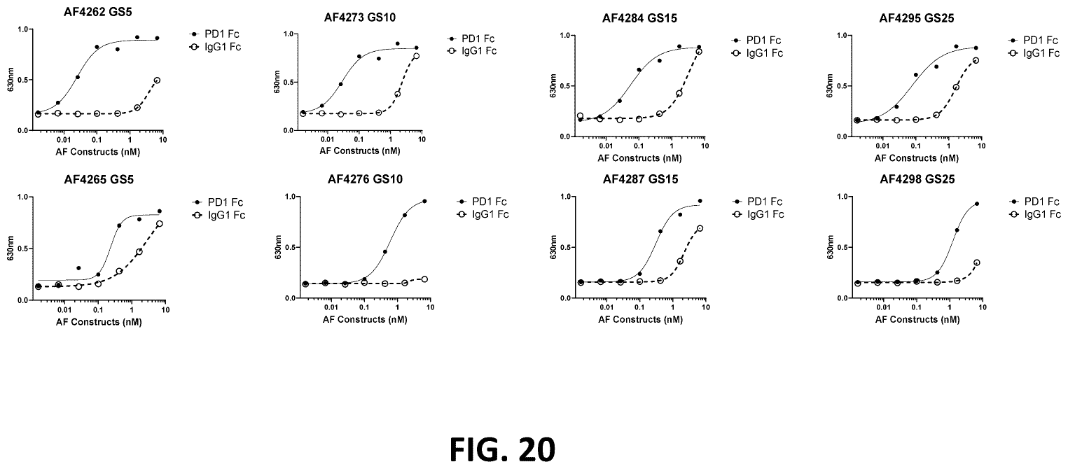

Results with the protein complexes depicted in FIG. 2B with varied linker lengths, are shown in FIG. 20. The asymmetric complex is comprised of two antibody domains with a single IL-2 linked to one of the domains. Varying linker lengths were chosen from GS5-GS25 to test for linker length dependence on PD-1 regulation. The cytokine complexes containing the PD-1/IL-2 DBA domain 2B07 variants in AF4262, AF4273, AF4284, and AF4295 all demonstrated increased IL-2 activity in the wells coated with PD-1 Fc compared to wells coated with hIgG1 Fc. Similar results were observed in the cytokine complexes containing the PD-1/IL-2 DBA domain 7A04 variants in AF4265, AF4276, AF4287 and AF4298. These data demonstrate that PD-1 regulation is possible with multiple cytokine antibody linker lengths varying from GS5 to GS25.

### Example 18

### PD-1-Dependent Induction of STAT5 Phosphorylation by PD-1/IL-2 DBA-Cytokine Complexes in Human Primary CD8+ T Cells

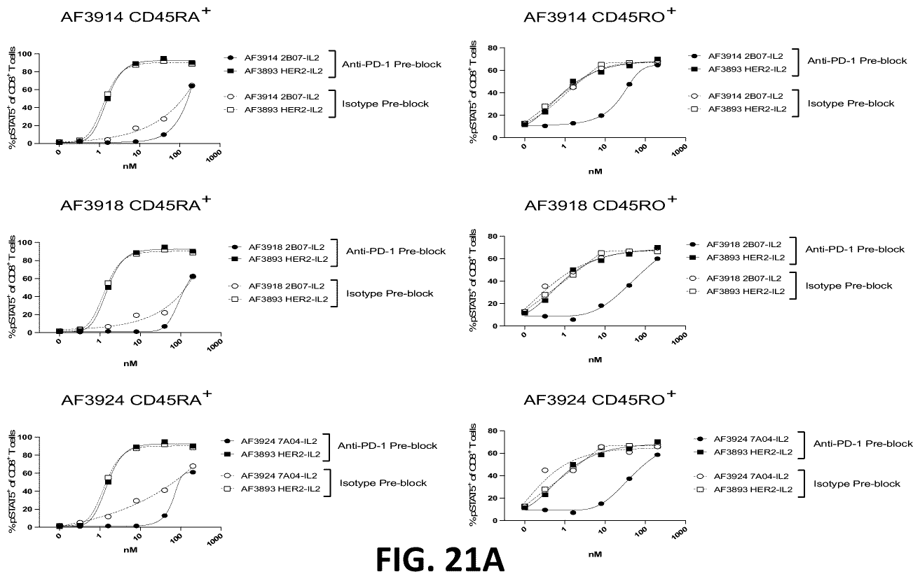

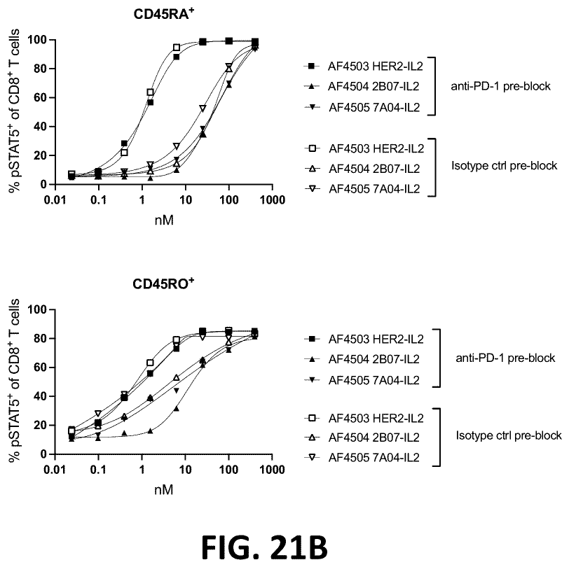

This example describes PD-1/IL-2 DBA-cytokine complex induction of STAT5 phosphorylation in primary human CD8+ T cells. The DBA-cytokine complexes were produced in the format shown in FIG. 2H. CD8+ T cells were isolated from human PBMCs using immunomagnetic negative selection (STEMCELL) and stimulated with plate-bound anti-CD3 and soluble anti-CD28 for 72 hours to induce expression of PD-1. The stimulated CD8+ T cells were incubated 1 hour with an anti-PD-1 blocking antibody or and isotype control antibody. Titrating concentrations of PD-1/IL-2 DBA cytokine complex (PD-1-regulated IL-2) or anti-HER2/IL-2-cytokine complex (Always-on IL-2) were then added to the CD8+ T cells and incubated at 37 C for 20 minutes. The CD8+ T cells were fixed with Perm Buffer III (BD Biosciences), washed, and stained with antibodies directed against CD8, CD45RA, CD45RO, and pSTAT5. STAT5 phosphorylation within the CD45RA+ and CD45RO+ T cell populations was assessed by flow cytometry. The results of this experiment are depicted in FIGS. 21A and 21B. In CD8+CD45RA+ T cells, which are largely PD-1 negative, the PD-1/IL-2 DBA cytokine complex induces a lower frequency of STAT5 phosphorylation-positive CD8+ T cells compared to the non-regulated anti-HER2/IL-2-cytokine complex control. In CD8+CD45RO+ T cells, which are largely PD-1 positive, the PD-1/IL-2 DBA cytokine complex induces an equivalent frequency of STAT5 phosphorylation-positive CD8+ T cells compared to the non-regulated anti-HER2/IL-2-cytokine complex control. Furthermore, CD8+CD45RO+ T cells that were pre-treated with an anti-PD-1 blocking antibody had a lower frequency of STAT5 phosphorylation-positive cells following treatment with the PD-1/IL-2 DBA cytokine complex showing the dependence of activity on PD-1 binding. Taken together, these data show that PD-1/IL-2 DBA cytokine complex shows decreased activity on PD-1 negative cells. On PD-1 positive cells, PD-1/IL-2 DBA cytokine complex activity is diminished by PD-1 blockade.

### Example 19

### PD-1/IL-2 DBA-Cytokine Complex Modulation of Human T Cell Activation in a Mixed Lymphocyte Reaction

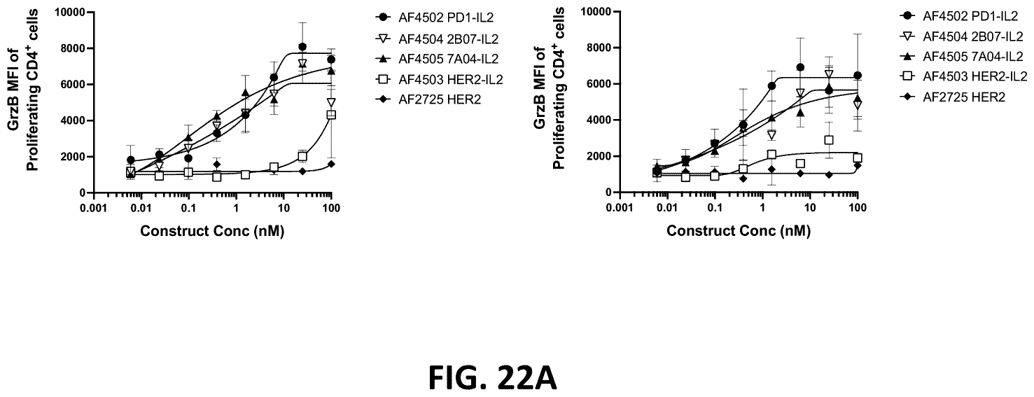

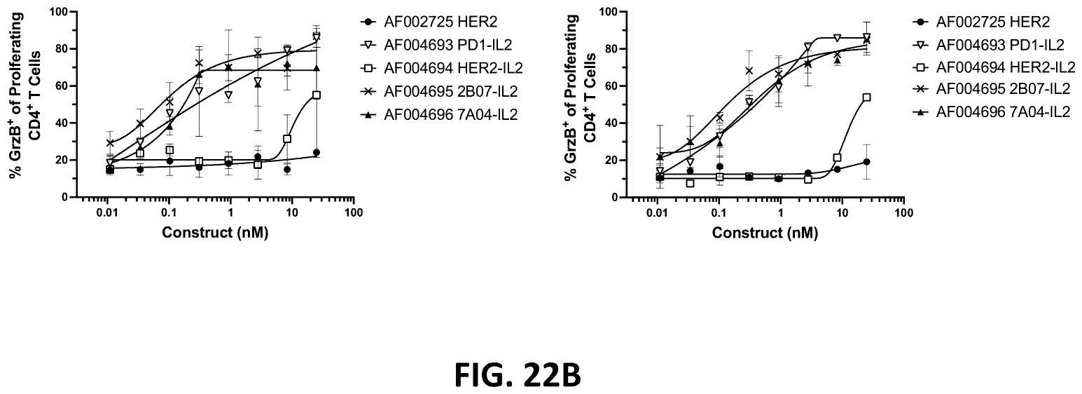

This example describes PD-1/IL-2 DBA-cytokine complex modulation of human CD4+ T cell activation in a mixed lymphocyte reaction (MLR) model. In this model, we assessed the activation of T cells against foreign antigen-presenting cells and the ability of our immunocytokine constructs to modulate that activation. CD4+CD25− T cells were isolated from human PBMCs using immunomagnetic negative selection (STEMCELL) and labeled with CELLTRACE™ Violet proliferation dye (ThermoFisher) following the manufactures protocol. To generate monocyte-derived dendritic cells (MDDCs), monocytes were isolated from PBMCs of a different donor using immunomagnetic negative selection (STEMCELL) and cultured in the presence of GM-CSF (100 ng/mL) and IL-4 (50 ng/ml). The culture media was replaced after 3 days, and MDDCs were collected on day 7. 10,000 MDDCs were added to each well of a 96-well round-bottom plate followed by the addition of 50,000 proliferation dye-labeled CD4+ T cells. A dilution series of each immunocytokine complex was generated and added to the cell cultures. After 5 days at 37 C, cells were stained with a LIVE/DEAD™ viability dye (ThermoFisher) and then then incubated in fixation/permeabilization buffer (BD Biosciences) for 20 minutes at 4 C. Cells were then stained with fluorophore-conjugated antibodies directed against CD4 and granzyme B (BD Bioscience), and the frequency of granzyme B-expressing cells amongst proliferating CD4+ T cells was assessed by flow cytometry. The results of this experiment are depicted in FIG. 22A and FIG. 22B. While HER2-IL2 induced minimal specific activation of the T cells (as expected because the T cells do not carry the HER2 molecule), the non-regulated and regulated PD1-IL2 constructs were able to stimulate the T cell activation, as indicated by dose-dependent granzyme B expression. These data demonstrate the PD-1 binding-dependent activity of the regulated PD1-IL2 complexes (via their ability to activate T-cells). Furthermore, the degree of granzyme B induction observed was comparable between the regulated and non-regulated PD1-IL2 complexes, indicating that the addition of the regulating moieties to IL2 does not diminish the activity of IL2 under properly permissive conditions.

### Example 20

### PD-1/IL-2 DBA-Cytokine Complex Modulation of Anti-Tumor Immunity in a Syngenetic Tumor Model

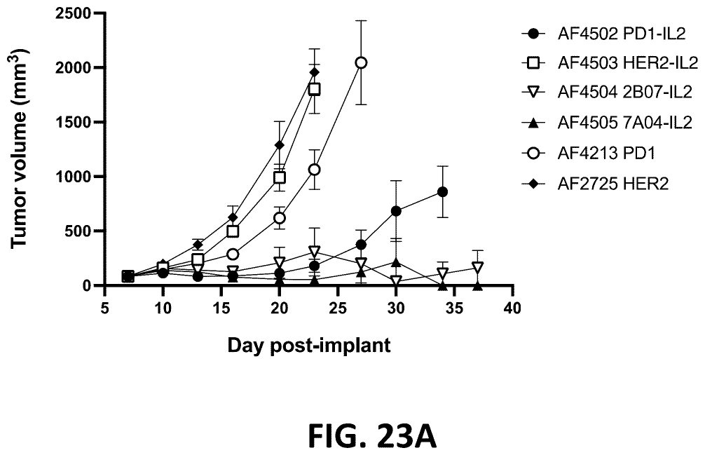

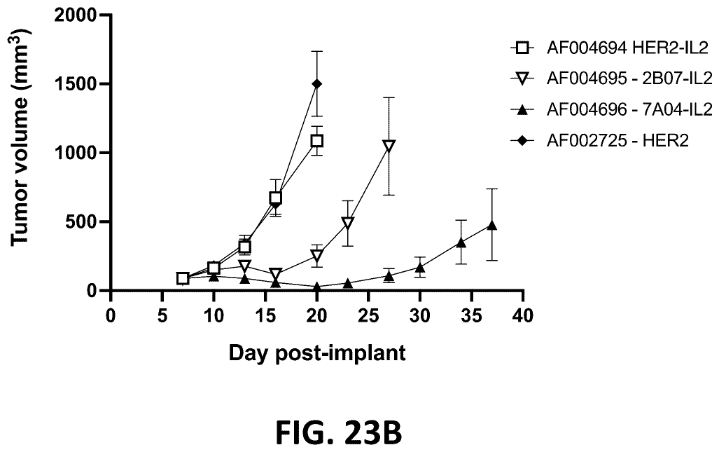

This example describes PD-1/IL-2 DBA-cytokine complex modulation of anti-tumor immunity in the MC38 syngeneic mouse tumor model. PD-1/IL-2 DBA-cytokine complex was assessed for the ability to drive anti-tumor immunity in vivo. 500,000 MC38 tumor cells were implanted subcutaneously in human PD-1 knock-in mice (GenOway). Tumors were measured twice weekly, and volumes calculated as (Length×Width×Width/2). Mice were randomized into treatment groups, and therapy initiated when tumors reached a volume of ˜100 mm³. Mice were treated intravenously with PD-1/IL-2 DBA-cytokine complex, non-regulated anti-PD1-IL2, anti-HER2-IL2, anti-PD-1, or anti-HER2 at 0.5 milligrams per kilogram of body weight on days 7, 10, and 13 post tumor implantation. The results of this experiment are presented in FIGS. 23A and 23B. The PD-1/IL-2 DBA-cytokine complexes showed comparable tumor growth inhibition compared to the non-regulated PD1-IL2 and superior tumor growth inhibition compared to anti-PD1 and anti-HER antibodies.

### Example 21

### PD1-IL2 Enhancement of T Cell Bispecific Engager Activity (Prophetic)

PD-1-regulated immunocytokines can be generated in which the PD1-regulated IL2 is used to enhance the activity of T cell bispecific antibodies (TCBs). In this example, PD1-IL2 TCBs can be generated in which a PD1-IL2 DBA scFv is fused to the C-term of one heavy chain and an IL-2 variant is fused to the C-term of the opposing heavy chain of a TCB. The N-term variable regions of the PD1-IL2 TCB can be directed against CD3 and a tumor associated antigen such as PSMA, HER2, CD20, or against CD3 and an irrelevant antigen. To assess PD1-IL2 TCB activity, human T cells are isolated from fresh PBMCs and co-cultured with tumor cell lines expressing various levels of tumor-associated antigens. Titrating concentrations of naked TCBs or PD1-IL2 TCBs are added to the T cell: tumor cell co-cultures. Tumor killing as well as T cell activation and cytokine production is assessed at various timepoints.

### Example 22

### PD-1-Regulated IL-2 Activity Targeted to T Cell-Associated Antigens (Prophetic)

PD-1-regulated immunocytokines can be generated in which the PD1-regulated IL2 is targeted to T cells using any T cell marker. For example, PD1-IL2 DBA scFv can be fused to the C-term of one heavy chain and an IL-2 variant can be fused to the C-term of the opposing heavy chain of an antibody directed against a T cell-expressed marker including but not limited to CD28, CD28H, OX40, GITR, CD137, CD27, HVEM, CTLA-4, PD-1, TIM-3, BTLA, VISTA, LAG-3, TIGIT, CD244, ICOS, CD40L, CD4, CD8, KLRG1, FasL, and CD7. T cells can be activated under various conditions to induce the expression of a given T cell marker. Titrating concentrations of PD1-IL2 DBA-containing immunocytokines directed against the marker of interest or an irrelevant marker may then be added. STAT5 phosphorylation may then be assessed as a measurement of targeted IL-2 activity. In some experiments, a blocking antibody against the marker of interest may be added prior to treatment with the PD1-IL2 DBA-containing immunocytokine to show specificity.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A complex comprising:
(a) a therapeutic domain comprising an IL-2 peptide;
(b) a linker; and
(c) a sensor domain comprising an antibody, wherein said sensor domain is configured to bind PD-1 and IL-2 in a mutually exclusive manner,
wherein the therapeutic domain is linked to the sensor domain by the linker.

2. The complex of claim 1, wherein:
(a) the sensor domain is configured: (i) to bind IL-2 in the absence of PD-1; and (ii) to not bind IL-2 in the presence of PD-1:
(b) the antibody is an antibody fragment or an antibody derivative;
(c) the sensor domain comprises a single dual binding antibody (DBA) configured to bind PD-1 and IL-2;
(d) the complex comprises an Fc domain; and/or
(e) the IL-2 peptide comprises a wild-type human IL-2 peptide.

3-4. (canceled)

5. The complex of claim 1, wherein the sensor domain comprises a single DBA configured to bind PD-1 and IL-2, wherein:
(a) the DBA comprises a heavy chain CDR3 having at least 80% identity to any one of SEQ ID NOs: 11-20, 154-156, 168-173, 114-119, 415, 421, 433, 439, 445, 451, 457, 463, 469, 475, 481, 487, 493, 499, 505, 511, 517, 523, 529, 535, 541, 547, 553, 559, 565, 571, 577, 583, 589, 595, 601, 607, 613, 619, 625, 631, 637, 643, 649, 655, 661, and 667;
(b) the DBA comprises a heavy chain CDR1, CDR2, or CDR3 comprising a sequence having at least 80% identity to any of the sequences recited in Table 3, Table 7, Table 8, and Table 19; or
(c) the DBA comprises a V_{H} or a V_{L} comprising a sequence having at least 80% identity to any of the sequences recited in Table 18.

6-8. (canceled)

9. The complex of claim 2, wherein the complex comprises an Fc domain, and wherein:
(a) the Fc domain is homodimeric or heterodimeric; and/or
(b) the Fc domain comprises: (i) a first polypeptide comprising a knob mutation and (ii) a second polypeptide comprising a hole mutation.

10-11. (canceled)

12. The complex of claim 9, wherein:
(a) the knob mutation or the hole mutation comprises mutations of any one of following pairs of residues relative to IgG: 366 and 407; 405 and 394; and 407 and 366; and/or
(b) the knob mutation comprises an arginine residue, a phenylalanine residue, a tyrosine residue, or a tryptophan residue; and the hole mutation comprises an alanine residue, a serine residue, a threonine residue, or a valine residue.

13. (canceled)

14. The complex of claim 1, wherein the complex comprises a sensor domain comprising a full-length DBA, wherein:
(a) the IL-2 peptide is linked to an N-terminus of a heavy chain of said full-length DBA or wherein the IL-2 peptide is linked to an N-terminus of a light chain of said full-length DBA; or
(b) the IL-2 peptide is linked to a C-terminus of a heavy chain of said full-length DBA.

15. (canceled)

16. The complex of claim 1, wherein the complex comprises:
(a) a first polypeptide according to N-[IL-2]-[linker]-[V_{H}]-[C_{H}]-[hinge]-Fc-C; and
a second polypeptide according to N-[V_{L}]-[C_{L}]-C, or
(b) a first polypeptide according to N-[V_{H}]-[C_{H}]-[hinge]-Fc-C; and
a second polypeptide according to N-[IL-2]-[linker]-[V_{L}]-[C_{L}]-C,
wherein N—denotes a peptide N-terminus, C—denotes a peptide C-terminus, [linker] denotes said linker, V_{H} indicates a heavy chain variable domain of said DBA, C_{H} indicates a heavy chain constant domain of an immunoglobulin, V_{L} denotes a light chain variable domain of said DBA, [hinge] denotes a hinge region of an immunoglobulin, Fc denotes an Fc region of an immunoglobulin, and CL denotes a light chain constant domain of an immunoglobulin.

17. The complex of claim 16, wherein the complex comprises any one of AF003345, AF003243, AF003246, AF003247, AF003341, AF003644, AF003651, AF003657, or and AF003934.

18. The complex of claim 1, wherein the complex comprises:
(a) a first polypeptide according to N-[IL-2]-[linker]-[V_{H}]-[C_{H}]-[hinge]-Fc[knob]-C;
a second polypeptide according to N-[V_{L}]-[C_{L}]-C; and
a third polypeptide according to N-[V_{H}]-[C_{H}]-[hinge]-Fc[hole]-C, or
(b) a first polypeptide according to N-[IL-2]-[linker]-[V_{H}]-[C_{H}]-[hinge]-Fc[hole]-C;
a second polypeptide according to N-[V_{L}]-[C_{L}]-C; and
a third polypeptide according to N-[V_{H}]-[C_{H}]-[hinge]-Fc[knob]-C,
wherein N—denotes a peptide N-terminus, C—denotes a peptide C-terminus, [linker] denotes said linker, V_{H} indicates a heavy chain variable domain of said DBA, C_{H} indicates a heavy chain constant domain of an immunoglobulin, V_{L} denotes a light chain variable domain of said DBA, [hinge] denotes a hinge region of an immunoglobulin, Fc[knob] denotes an Fc of an immunoglobulin comprising a knob mutation, Fc[hole] denotes an Fc region of an immunoglobulin comprising a hole mutation, and C_{L} denotes a light chain constant domain of an immunoglobulin.

19. The complex of claim 18, wherein:
(a) the knob mutation or the hole mutation comprises mutations of any one of following pairs of residues relative to IgG: 366 and 407; 405 and 394; and 407 and 366; and/or
(b) the complex comprises any one of AF003229, AF003230, AF003232, AF003740, AF003747 AF003749, AF003753, AF003945, AF003947, AF003951, AF003952, AF003953, AF003955, AF003956, and AF003941.

20. (canceled)

21. The complex of claim 1, wherein the complex comprises:
(a) a first polypeptide according to N-[IL-2]-[linker]-[V_{H}]-[C_{H}]-[hinge]-Fc-[scFv]-C; and
(b) a second polypeptide according to N-[V_{L}]-[C_{L}]-C,
wherein N-denotes a peptide N-terminus, C-denotes a peptide C-terminus, [linker] denotes said linker, V_{H} indicates a heavy chain variable domain of an anti-PD-1 monoselective antibody, C_{H} indicates a heavy chain constant domain of an immunoglobulin, V_{L} denotes a light chain variable domain of an anti-PD-1 monoselective antibody, [hinge] denotes a hinge region of an immunoglobulin, Fc denotes an Fc region of an immunoglobulin, C_{L} denotes a light chain constant domain of an immunoglobulin, and [scFv] denotes an scFv comprising V_{H} and V_{L} domains of said DBA.

22. The complex of claim 21, wherein:
(a) said scFv comprising V_{H} and V_{L} domains of said DBA is oriented according to N-[V_{H}]-[linker2]-[V_{L}]-C;
(b) said scFv comprising V_{H} and V_{L} domains of said DBA comprises:
(i) a V_{H} domain comprising a sequence having at least 80% identity to a V_{H} domain of any one of AB002022 2B07v1, AB002328 2B07v4, AB002360 7A04v1, AB002413 2A11v3. AB002342 2B07v5, AB002345 2B07v6, and AB002365 7A04v2; or
a V_{L} domain comprising a sequence having at least 80% identity to a V_{L} domain of any one of AB002022 2B07v1, AB002328 2B07v4, AB002360 7A04v1, AB002413 2A11v3. AB002342 2B07v5, AB002345 2B07v6, and AB002365 7A04v2; and/or
(ii) heavy chain CDRs of any one of AB002022_2B07v1, AB002328 2B07v4, AB002360 7A04v1, AB002413 2A11v3, AB002342 2B07v5, AB002345 2B07v6, and AB002365 7A04v2; or
light chain CDRs of any one of AB002022 2B07v1, AB002328 2B07v4, AB002360 7A04v1, AB002413 2A11v3, AB002342 2B07v5, AB002345 2B07v6, and AB002365 7A04v2; and/or
(c) the complex comprises any one of AF003864, AF003871, AF003872, AF003913, AF003918. AF003923, AF003927, AF004502, AF004503, AF004504, AF004505, AF004892, and AF004893.

23-25. (canceled)

26. The complex of claim 1, wherein the complex comprises:
(a) a first polypeptide according to N-[V_{H}]-[C_{H}]-[hinge]-Fc[knob]-[linker]-[IL-2]-C;
a second polypeptide according to N-[V_{L}]-[C_{L}]-C; and
a third polypeptide according to N-[V_{H}]-[C_{H}]-[hinge]-Fc[hole]-[linker]-[scFv]-C, or
(b) a first polypeptide according to N-[V_{H}]-[C_{H}]-[hinge]-Fc[hole]-[linker]-[IL-2]-C;
a second polypeptide according to N-[V_{L}]-[C_{L}]-C; and
a third polypeptide according to N-[V_{H}]-[C_{H}]-[hinge]-Fc[knob]-[linker]-[scFv]-C;
wherein N-denotes a peptide N-terminus, C-denotes a peptide C-terminus, [linker] denotes said linker, V_{H} indicates a heavy chain variable domain of said DBA, C_{H} indicates a heavy chain constant domain of an immunoglobulin, V_{L} denotes a light chain variable domain of said DBA, [hinge] denotes a hinge region of an immunoglobulin, Fc[knob] denotes an Fc of an immunoglobulin comprising a knob mutation, Fc[hole] denotes an Fc region of an immunoglobulin comprising a hole mutation, C_{L} denotes a light chain constant domain of an immunoglobulin, and [scFv] denotes an scFv of said DBA.

27. The complex of claim 26, wherein the complex comprises any one of AF004693, AF004695, AF004696, AF005416, AF005418, and AF005419.

28. The complex of claim 1, wherein the complex comprises:
(a) a first polypeptide according to N-[V_{H}]-[C_{H}]-[het-hinge]-Fc[knob]-[linker]-[IL-2]-C;
a second polypeptide according to N-[V_{L}]-[C_{L}]-C; and
a third polypeptide according to N-[V_{H}]-[C_{H}]-[het-hinge]-Fc[hole]-C, or
(b) a first polypeptide according to N-[V_{H}]-[C_{H}]-[het-hinge]-Fc[hole]-[linker]-[IL-2]-C;
a second polypeptide according to N-[V_{L}]-[C_{L}]-C; and
a third polypeptide according to N-[V_{H}]-[C_{H}]-[het-hinge]-Fc[knob]-C,
wherein N-denotes a peptide N-terminus, C-denotes a peptide C-terminus, [linker] denotes said linker, V_{H} indicates a heavy chain variable domain of said DBA, C_{H} indicates a heavy chain constant domain of an immunoglobulin, V_{L} denotes a light chain variable domain of said DBA, [het hinge] denotes a hinge region heterologous to said Fc region, Fc[knob] denotes an Fc of an immunoglobulin comprising a knob mutation, Fc[hole] denotes an Fc region of an immunoglobulin comprising a hole mutation, and C_{L} denotes a light chain constant domain of an immunoglobulin.

29. The complex of claim 28, wherein:
(a) said hinge region heterologous to said Fc region is: (i) a hinge region derived from an IgG3 antibody, or (ii) a G4S-based linker; and/or
(b) said complex comprises AF003632 or AF003634.

30-31. (canceled)

32. The complex of claim 2, wherein the IL-2 peptide comprises a sequence having at least about 80% sequence identity to human IL-2.

33. The complex of claim 32, wherein the complex comprises any one of AF003232, AF003243, AF003246, AF003247, AF003341, AF003345, AF003632, AF003634, AF003644, AF003651, AF003652, AF003653, AF003657, AF003740, AF003744, AF003747, AF003749, AF003753, AF003864, AF003873, AF003876, AF003877, AF003913, AF003918, AF003923, AF003927, AF003930, AF003931, AF003933, AF003934, AF003935, AF003941, AF003945, AF003946, AF003947, AF003948, AF003951, AF003952, AF003953, AF003955, AF003956, AF004262, AF004265, AF004273, AF004276, AF004284, AF004287, AF004295, AF004298, AF004385, AF004386, AF004387, AF004388, AF004389, AF004404, AF004405, AF004413, AF004414, AF004415, AF004416, AF004504, AF004505, AF004693, AF004695, AF004696, AF004771, AF004773, AF004892, er and AF004893.

34. A method of enhancing T-cell reactivity to heterologous cells, comprising administering the complex of claim 1 to a subject in need thereof.

35. The method of claim 34, wherein the heterologous cells are cancer cells.

36. A method of treating a subject in need thereof, the method comprising administering the complex of claim 1 to the subject in need thereof.

37. The method of claim 36, wherein:
(a) the administering comprises intravenous, intramuscular, or subcutaneous administration;
(b) the subject in need thereof has cancer;
(c) the therapeutic domain treats the subject in need thereof; and/or
(d) the subject in need thereof is a mammal.

38-40. (canceled)

41. The method of claim 37, wherein the subject in need thereof is a human.

42. A composition comprising a recombinant nucleic acid encoding the complex of claim 1.

43. A pharmaceutical composition comprising the complex of claim 1 and a pharmaceutically acceptable excipient.


SEQUENCE LISTING (800 sequences)

SEQ ID NO: 1-10 - skipped (10 entries)

SEQ ID NO: 11 - protein, 8 aa, synthetic construct
CAAGLFIW

SEQ ID NO: 12 - protein, 8 aa, synthetic construct
CAGGWLDW

SEQ ID NO: 13 - protein, 13 aa, synthetic construct
CARDHLGGSYQPW

SEQ ID NO: 14 - protein, 28 aa, synthetic construct
CARDLVGVSPGINYVPRYYYYYYGMDVW

SEQ ID NO: 15 - protein, 19 aa, synthetic construct
CARDTGLGYYYGSGDFDYW

SEQ ID NO: 16 - protein, 15 aa, synthetic construct
CARSGYSYGYYFDYW

SEQ ID NO: 17 - protein, 13 aa, synthetic construct
CARTGGYPAIDSW

SEQ ID NO: 18 - protein, 8 aa, synthetic construct
CASGWDVW

SEQ ID NO: 19 - protein, 12 aa, synthetic construct
CASSPLQWVDVW

SEQ ID NO: 20 - protein, 8 aa, synthetic construct
CTSGMDVW

SEQ ID NO: 21-30 - skipped (10 entries)

SEQ ID NO: 31 - protein, 251 aa, synthetic construct
QVQLVQSGAEVKKPGVSVKVSCKASGYTFPRSYIHWVRQAPGQGLEWMGWINPHSGDTYYAQNFQGRVTMTRDTSTSTVYMELSSLRSEDTAVYYCARDTGLGYYYGSGDFDYWGQGTLVTVSSASGGGGSGGGGSGGGGSHASDIQMTQSPSSLSASVGDRVTITCRASQSISRYLNWYQQKPGKAPKLLIYTASSLQSGVPSRFSGSGSGTDFTLTISSLQPEDFATYYCQQANRFPLTFGPGTKVDIK

SEQ ID NO: 32 - protein, 251 aa, synthetic construct
QVQLVQSGAEVKKPGASVKVSCKASGYTFPRYHIHWVRQAPGQGLEWMGMINPSGGTTTYAQKFQGRVTMTRDTSTSTVYMELSSLRSEDTAVYYCARDTGLGYYYGSGDFDYWGQGTLVTVSSASGGGGSGGGGSGGGGSHASDIQMTQSPSSLSASVGDRVTITCRASQSISSWLAWYQQKPGKAPKLLIYAASSLQSGVPSRFSGSGSGTDFTLTISSLQPEDFATYYCQQSHSFPLTFGGGTKVEIK

SEQ ID NO: 33 - protein, 245 aa, synthetic construct
QVQLVQSGAEVKKPGASVKVSCKASGYTFTRYYIHWVRQAPGQGLEWMGWINAYNGDTNYAQKLQGRVTMTRDTSTSTVYMELSSLRSEDTAVYYCARDSYYYDSFDYWGQGTLVTVSSASGGGGSGGGGSGGGGSHASDIQMTQSPSSLSASVGDRVTITCRASQTITDWLAWYQQKPGKAPKLLIYGASNLQGGVPSRFSGSGSGTDFTLTISSLQPEDFATYYCQQYYSSWTFGQGTKVEIK

SEQ ID NO: 34 - protein, 246 aa, synthetic construct
QVQLVQSGAEVKKPGASVKVSCKASGYTFTSYYMHWVRQAPGQGLEWMGIINPSDGSTTYAQSFQGRVTMTRDTSTSTVYMELSSLRSEDTAVYYCASGWDVWGQGTLVTVSSASGGGGSGGGGSGGGGSHASDIVMTQSPDSLAVSLGERATINCKSSQSVFSSANNKNYLAWYQQKPGQPPKLLIYWASTRESGVPDRFSGSGSGTDFTLTISSLQAEDVAVYYCQQYFGTPVTFGGGTKVEIK

SEQ ID NO: 35-50 - skipped (16 entries)

SEQ ID NO: 51 - protein, 443 aa, synthetic construct
QVQLVQSGAEVKKPGASVKVSCKASGYTFSTYYIHWVRQAPGQGLEWMGIINPSGGGTVYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 407 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTVPGVGVPGAGVPGVGVPGGGVPGVGVPGGGVPGAGVPGGGVPGVGVPGAGVPGVGVPGGGDIQMTQSPSSLSASVGDRVTITCRASQYISSGLAWYQQKPGKAPKLLIYKASSLDNGVPSRFSGSGSGTDFTLTISSLQPEDFATYYCQQYERLPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 596 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGYTFSTYYIHWVRQAPGQGLEWMGIINPSGGGTVYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 54 - protein, 214 aa, synthetic construct
DIQMTQSPSSLSASVGDRVTITCRASQYISSGLAWYQQKPGKAPKLLIYKASSLENGVPSRFSGSGSGTDFTLTISSLQPEDFATYYCQQYERLPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 55-63 - skipped (9 entries)

SEQ ID NO: 64 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 66 - protein, 407 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTVPGVGVPGAGVPGVGVPGGGVPGVGVPGGGVPGAGVPGGGVPGVGVPGAGVPGVGVPGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 67 - protein, 454 aa, synthetic construct
QVQLVQSGAEVKKPGASVKVSCKVSGYTFTSYDINWVRQAPGQGLEWMGWINPNSGDTGYAQKFQGRVTMTRDTSTSTVYMELSSLRSEDTAVYYCARDTGLGYYYGSGDFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 68 - protein, 407 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTVPGVGVPGAGVPGVGVPGGGVPGVGVPGGGVPGAGVPGGGVPGVGVPGAGVPGVGVPGGGDIQMTQSPSSLSASVGDRVTITCQASQDIHNYLNWYQQKPGKAPKLLIYDVSNLETGVPSRFSGSGSGTDFTLTISSLQPEDFATYYCQQAISFPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 69 - protein, 443 aa, synthetic construct
QVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 70 - protein, 407 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTVPGVGVPGAGVPGVGVPGGGVPGVGVPGGGVPGAGVPGGGVPGVGVPGAGVPGVGVPGGGDIQMTQSPSSLSASVGDRVTITCRASQSINSWLAWYQQKPGKAPKLLIYATSTLESGVPSRFSGSGSGTDFTLTISSLQPEDFATYYCQQSYSFPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 71 - protein, 443 aa, synthetic construct
QVQLVQSGAEVKKPGASVKVSCKASGYTFTNYYIHWVRQAPGQGLEWMGIINPRAGYTSYALKFQGRVTMTRDTSTSTVYMELSSLRSEDTAVYYCAGGWLDWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 72 - protein, 407 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTVPGVGVPGAGVPGVGVPGGGVPGVGVPGGGVPGAGVPGGGVPGVGVPGAGVPGVGVPGGGDIQMTQSPSSLSASVGDRVTITCRASQSISSWLAWYQQKPGKAPKLLIYAASSLQSGVPSRFSGSGSGTDFTLTISSLQPEDFATYYCQQSFTMPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 73 - protein, 443 aa, synthetic construct
QVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 74 - protein, 407 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTVPGVGVPGAGVPGVGVPGGGVPGVGVPGGGVPGAGVPGGGVPGVGVPGAGVPGVGVPGGGDIQMTQSPSSLSASVGDRVTITCRASQSIGRWLAWYQQKPGKAPKLLIYSASNLETGVPSRFSGSGSGTDFTLTISSLQPEDFATYYCQQANSFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 75 - protein, 443 aa, synthetic construct
QVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 76 - protein, 407 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTVPGVGVPGAGVPGVGVPGGGVPGVGVPGGGVPGAGVPGGGVPGVGVPGAGVPGVGVPGGGDIQMTQSPSSLSASVGDRVTITCRASQSISTWLAWYQQKPGKAPKLLIYAASSLQSGVPSRFSGSGSGTDFTLTISSLQPEDFATYYCQQSYSFPVTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 77 - protein, 443 aa, synthetic construct
QVQLVQSGAEVKKPGASVKVSCKASGYTFTTYYVHWVRQAPGQGLEWMGIINPSGGSTSYAQNFQGRVTMTRDTSTSTVYMELSSLRSEDTAVYYCASGWDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 78 - protein, 485 aa, synthetic construct
QVQLVQSGAEVKKPGASVKVSCKASGYTFTTYYVHWVRQAPGQGLEWMGIINPSGGSTSYAQNFQGRVTMTRDTSTSTVYMELSSLRSEDTAVYYCASGWDVWGQGTTVTVSSASGGGGSGGGGSGGGGSHASEIVMTQSPATLSVSPGERATLSCRASQSVNTYLAWYQQKPGQAPRLLIYGASTRATGIPARFSGSGSGTEFTLTISSLQSEDFAVYYCQQYGSSPVTFGQGTRLEIKPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 79 - protein, 367 aa, synthetic construct
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSEIVMTQSPATLSVSPGERATLSCRASQSVNTYLAWYQQKPGQAPRLLIYGASTRATGIPARFSGSGSGTEFTLTISSLQSEDFAVYYCQQYGSSPVTFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 80 - protein, 596 aa, synthetic construct
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGDTFSTYYVHWVRQAPGQGLEWMGIINPSGGGTVYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 81 - protein, 596 aa, synthetic construct
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGYTFSNYYIHWVRQAPGQGLEWMGIINPSGGGTVYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 82 - protein, 596 aa, synthetic construct
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 83 - protein, 596 aa, synthetic construct; identical to SEQ ID NO: 80

SEQ ID NO: 84 - protein, 596 aa, synthetic construct; identical to SEQ ID NO: 81

SEQ ID NO: 85 - protein, 596 aa, synthetic construct; identical to SEQ ID NO: 82

SEQ ID NO: 86-126 - skipped (41 entries)

SEQ ID NO: 127 - protein, 113 aa, synthetic construct
QVQLVQSGAEVKKPGASVKVSCKASGDTFSTYYVHWVRQAPGQGLEWMGIINPSGGGTVYAQKFQGRVTMTRDTSTSTVYMELSSLRSEDTAVYYCAAGLFIWGQGTLVTVSS

SEQ ID NO: 128 - protein, 113 aa, synthetic construct
QVQLVQSGAEVKKPGASVKVSCKASGYTFSNYYIHWVRQAPGQGLEWMGIINPSGGGTVYAQKFQGRVTMTRDTSTSTVYMELSSLRSEDTAVYYCAAGLFIWGQGTLVTVSS

SEQ ID NO: 129 - protein, 113 aa, synthetic construct
QVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSS

SEQ ID NO: 130-134 - skipped (5 entries)

SEQ ID NO: 135 - protein, 107 aa, synthetic construct
DIQMTQSPSSLSASVGDRVTITCRASQYISSGLAWYQQKPGKAPKLLIYKASSLDNGVPSRFSGSGSGTDFTLTISSLQPEDFATYYCQQYERLPLTFGGGTKVEIK

SEQ ID NO: 136 - protein, 107 aa, synthetic construct
DIQMTQSPSSLSASVGDRVTITCRASQSIGTGLAWYQQKPGKAPKLLIYKASSLDNGVPSRFSGSGSGTDFTLTISSLQPEDFATYYCQQYNRAPLTFGGGTKVEIK

SEQ ID NO: 137 - protein, 107 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYESFPVTFGPGTKVDIK

SEQ ID NO: 138-141 - skipped (4 entries)

SEQ ID NO: 142 - protein, 10 aa, synthetic construct
GDTFSTYYVH

SEQ ID NO: 143-145 - skipped (3 entries)

SEQ ID NO: 146 - protein, 10 aa, synthetic construct
GYTFSGYYIH

SEQ ID NO: 147 - protein, 10 aa, synthetic construct
GYTFSNYYVH

SEQ ID NO: 148 - protein, 17 aa, synthetic construct
IINPSGGGTVYAQKFQG

SEQ ID NO: 149 - protein, 17 aa, synthetic construct
IINPSGGYASYAQKFQG

SEQ ID NO: 150-153 - skipped (4 entries)

SEQ ID NO: 154 - protein, 6 aa, synthetic construct
AAGLFI

SEQ ID NO: 155-156 - skipped (2 entries)

SEQ ID NO: 157 - protein, 11 aa, synthetic construct
RASQYISSGLA

SEQ ID NO: 158 - protein, 11 aa, synthetic construct
RASQSIGTGLA

SEQ ID NO: 159 - protein, 11 aa, synthetic construct
RASQSIGRWLA

SEQ ID NO: 160 - protein, 11 aa, synthetic construct
RASQSISNRLA

SEQ ID NO: 161 - protein, 11 aa, synthetic construct
QASQSISNYLA

SEQ ID NO: 162 - protein, 11 aa, synthetic construct
RASQSISSYLN

SEQ ID NO: 163 - protein, 7 aa, synthetic construct
KASSLDN

SEQ ID NO: 164 - protein, 7 aa, synthetic construct
SASNLET

SEQ ID NO: 165-167 - skipped (3 entries)

SEQ ID NO: 168 - protein, 8 aa, synthetic construct
QQYERLPL

SEQ ID NO: 169 - protein, 8 aa, synthetic construct
QQYNRAPL

SEQ ID NO: 170 - protein, 8 aa, synthetic construct
QQYESFPV

SEQ ID NO: 171-173 - skipped (3 entries)

SEQ ID NO: 174 - protein, 596 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 175 - protein, 214 aa, synthetic construct
DIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 176 - protein, 603 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 177 - protein, 600 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSEVQLVESGGGLVKPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 178 - protein, 214 aa, synthetic construct
DIQMTQSPSSLSASVGDRVSITCKASQNVGTNVGWYQQKPGKAPKALIYSASFRYSGVPSRFSGSGSGTDFTLTISSLQPEDFATYFCQQYYTYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 179 - protein, 596 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 180 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 181 - protein, 596 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 182 - protein, 456 aa, synthetic construct
QVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 183 - protein, 596 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 184 - protein, 456 aa, synthetic construct
QVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 185 - protein, 214 aa, synthetic construct
DIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 186 - protein, 596 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 187 - protein, 456 aa, synthetic construct
QVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 188 - protein, 214 aa, synthetic construct
DIQMTQSPSSLSASVGDRVTITCRASQSINSWLAWYQQKPGKAPKLLIYATSTLESGVPSRFSGSGSGTDFTLTISSLQPEDFATYYCQQYYRFPVTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 189 - protein, 596 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 190 - protein, 456 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 191 - protein, 603 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 192 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 193 - protein, 600 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSEVQLVESGGGLVKPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 194 - protein, 460 aa, synthetic construct
EVQLVESGGGLVKPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 195 - protein, 716 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIKGGGSGGGSHHHHHH

SEQ ID NO: 196 - protein, 716 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKGGGSGGGSHHHHHH

SEQ ID NO: 197 - protein, 723 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIKGGGSGGGSHHHHHH

SEQ ID NO: 198 - protein, 723 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKGGGSGGGSHHHHHH

SEQ ID NO: 199 - protein, 590 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLT

SEQ ID NO: 200 - protein, 723 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASGGGGSGGGGSGGGGSHASDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGSGGGSHHHHHH

SEQ ID NO: 201 - protein, 720 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSEVQLVESGGGLVKPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTLVTVSSASGGGGSGGGGSGGGGSHASDIQMTQSPSSLSASVGDRVSITCKASQNVGTNVGWYQQKPGKAPKALIYSASFRYSGVPSRFSGSGSGTDFTLTISSLQPEDFATYFCQQYYTYPYTFGGGTKLEIKGGGSGGGSHHHHHH

SEQ ID NO: 202 - protein, 597 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLT

SEQ ID NO: 203 - protein, 730 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASGGGGSGGGGSGGGGSHASDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGSGGGSHHHHHH

SEQ ID NO: 204 - protein, 727 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSEVQLVESGGGLVKPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTLVTVSSASGGGGSGGGGSGGGGSHASDIQMTQSPSSLSASVGDRVSITCKASQNVGTNVGWYQQKPGKAPKALIYSASFRYSGVPSRFSGSGSGTDFTLTISSLQPEDFATYFCQQYYTYPYTFGGGTKLEIKGGGSGGGSHHHHHH

SEQ ID NO: 205 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 206 - protein, 214 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYESFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 207 - protein, 603 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 208 - protein, 600 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSEVQLVESGGGLVKPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 209 - protein, 214 aa, synthetic construct; identical to SEQ ID NO: 178

SEQ ID NO: 210 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 211 - protein, 443 aa, synthetic construct
QVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGK

SEQ ID NO: 212 - protein, 214 aa, synthetic construct; identical to SEQ ID NO: 175

SEQ ID NO: 213 - protein, 443 aa, synthetic construct
QVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 214 - protein, 450 aa, synthetic construct; identical to SEQ ID NO: 65

SEQ ID NO: 215 - protein, 214 aa, synthetic construct; identical to SEQ ID NO: 64

SEQ ID NO: 216-328 - skipped (113 entries)

SEQ ID NO: 329 - protein, 113 aa, synthetic construct [sequence omitted]

SEQ ID NO: 330 - protein, 107 aa, synthetic construct [sequence omitted]

SEQ ID NO: 331 - protein, 113 aa, synthetic construct
QVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSS

SEQ ID NO: 332 - protein, 113 aa, synthetic construct
DIVMTQSPDSLAVSLGERATINCKASQSLLHSSSNKNYLAWYQQKPGQPPKLLIYWASTRESGVPDRFSGSGSGTDFTLTISSLQAEDVAVYYCQQYYSTPITFGPGTKVDIK

SEQ ID NO: 333 - protein, 113 aa, synthetic construct; identical to SEQ ID NO: 129

SEQ ID NO: 334 - protein, 107 aa, synthetic construct; identical to SEQ ID NO: 137

SEQ ID NO: 335 - protein, 113 aa, synthetic construct
QVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSS

SEQ ID NO: 336 - protein, 107 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYNSFPVTFGPGTKVDIK

SEQ ID NO: 337 - protein, 113 aa, synthetic construct; identical to SEQ ID NO: 335

SEQ ID NO: 338 - protein, 107 aa, synthetic construct
DIQMTQSPSSLSASVGDRVTITCRASQSIGSWLAWYQQKPGKAPKLLIYSASNLETGVPSRFSGSGSGTDFTLTISSLQPEDFATYYCQQYNSFPVTFGPGTKVDIK

SEQ ID NO: 339 - protein, 113 aa, synthetic construct; identical to SEQ ID NO: 335

SEQ ID NO: 340 - protein, 107 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIK

SEQ ID NO: 341 - protein, 113 aa, synthetic construct; identical to SEQ ID NO: 335

SEQ ID NO: 342 - protein, 107 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYERFPVTFGPGTKVDIK

SEQ ID NO: 343 - protein, 113 aa, synthetic construct; identical to SEQ ID NO: 335

SEQ ID NO: 344 - protein, 107 aa, synthetic construct
DIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIK

SEQ ID NO: 345 - protein, 113 aa, synthetic construct; identical to SEQ ID NO: 335

SEQ ID NO: 346 - protein, 107 aa, synthetic construct
DIQMTQSPSSLSASVGDRVTITCRASQSIGRYLAWYQQKPGKAPKLLIYSASNLETGVPSRFSGSGSGTDFTLTISSLQPEDFATYYCQQYNRFPVTFGPGTKVDIK

SEQ ID NO: 347 - protein, 113 aa, synthetic construct; identical to SEQ ID NO: 335

SEQ ID NO: 348 - protein, 107 aa, synthetic construct
DIQMTQSPSSLSASVGDRVTITCRASQSIGRYLAWYQQKPGKAPKLLIYYASNLETGVPSRFSGSGSGTDFTLTISSLQPEDFATYYCQQYNSFPVTFGPGTKVDIK

SEQ ID NO: 349 - protein, 113 aa, synthetic construct; identical to SEQ ID NO: 335

SEQ ID NO: 350 - protein, 107 aa, synthetic construct; identical to SEQ ID NO: 137

SEQ ID NO: 351 - protein, 113 aa, synthetic construct
QVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSS

SEQ ID NO: 352 - protein, 107 aa, synthetic construct
DIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIK

SEQ ID NO: 353 - protein, 113 aa, synthetic construct
QVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSS

SEQ ID NO: 354 - protein, 107 aa, synthetic construct; identical to SEQ ID NO: 352

SEQ ID NO: 355 - protein, 113 aa, synthetic construct
QVQLVQSGAEVKKPGASVKVSCKASGYTFTTYYMHWVRQAPGQGLEWMGIINPRAGYTSYALKFQGRVTMTRDTSTSTVYMELSSLRSEDTAVYYCTSGWDVWGQGTLVTVSS

SEQ ID NO: 356 - protein, 107 aa, synthetic construct; identical to SEQ ID NO: 352

SEQ ID NO: 357 - protein, 113 aa, synthetic construct
QVQLVQSGAEVKKPGASVKVSCKASGDTFTDYYMHWVRQAPGQGLEWMGIINPRAGYTSYALKFQGRVTMTRDTSTSTVYMELSSLRSEDTAVYYCTSGWDVWGQGTLVTVSS

SEQ ID NO: 358 - protein, 107 aa, synthetic construct; identical to SEQ ID NO: 352

SEQ ID NO: 359 - protein, 113 aa, synthetic construct
QVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGWFVWGQGTLVTVSS

SEQ ID NO: 360 - protein, 107 aa, synthetic construct
DIQMTQSPSSLSASVGDRVTITCRASQSINSWLAWYQQKPGKAPKLLIYDTSTLESGVPSRFSGSGSGTDFTLTISSLQPEDFATYYCQQSYSFPVTFGQGTKVEIK

SEQ ID NO: 361 - protein, 113 aa, synthetic construct
QVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSS

SEQ ID NO: 362 - protein, 107 aa, synthetic construct
DIQMTQSPSSLSASVGDRVTITCRASQSINSWLAWYQQKPGKAPKLLIYATSTLESGVPSRFSGSGSGTDFTLTISSLQPEDFATYYCQQYYRFPVTFGQGTKVEIK

SEQ ID NO: 363 - protein, 113 aa, synthetic construct; identical to SEQ ID NO: 361

SEQ ID NO: 364 - protein, 107 aa, synthetic construct
DIQMTQSPSSLSASVGDRVTITCRASQSINSWLAWYQQKPGKAPKLLIYATSTLESGVPSRFSGSGSGTDFTLTISSLQPEDFATYYCQQYYSFPVTFGQGTKVEIK

SEQ ID NO: 365 - protein, 113 aa, synthetic construct; identical to SEQ ID NO: 361

SEQ ID NO: 366 - protein, 107 aa, synthetic construct
DIQMTQSPSSLSASVGDRVTITCRASQSINSWLAWYQQKPGKAPKLLIYATSTLESGVPSRFSGSGSGTDFTLTISSLQPEDFATYYCQQSYRFPVTFGQGTKVEIK

SEQ ID NO: 367 - protein, 113 aa, synthetic construct; identical to SEQ ID NO: 351

SEQ ID NO: 368 - protein, 107 aa, synthetic construct
DIQMTQSPSSLSASVGDRVTITCRASQSISTWLAWYQQKPGKAPKLLIYAASSLDSGVPSRFSGSGSGTDFTLTISSLQPEDFATYYCQQSYRFPVTFGQGTKVEIK

SEQ ID NO: 369 - protein, 113 aa, synthetic construct
QVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLDIWGQGTLVTVSS

SEQ ID NO: 370 - protein, 107 aa, synthetic construct; identical to SEQ ID NO: 366

SEQ ID NO: 371 - protein, 124 aa, synthetic construct
QVQLVQSGAEVKKPGASVKVSCKASGYIFNGYDIHWVRQAPGQGLEWMGWMNPDNGNTGLAQKFQGRVTITADESTSTAYMELSSLRSEDTAVYYCARGMATRFPYYYYGMDVWGQGTLVTVSS

SEQ ID NO: 372 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPPTFGQGTKVEIK

SEQ ID NO: 373 - protein, 113 aa, synthetic construct
QVQLVQSGAEVKKPGASVKVSCKASGHTFTRYYMHWVRQAPGQGLEWMGVINPSGGYATYAQKFQGRVTMTRDTSTSTVYMELSSLRSEDTAVYYCASGWDVWGQGTLVTVSS

SEQ ID NO: 374 - protein, 107 aa, synthetic construct
DIQMTQSPSSLSASVGDRVTITCRASQSINSWLAWYQQKPGKAPKLLIYATSTLESGVPSRFSGSGSGTDFTLTISSLQPEDFATYYCQQSYSSPVTFGQGTKVEIK

SEQ ID NO: 375 - protein, 113 aa, synthetic construct
QVQLVQSGAEVKKPGASVKVSCKASGYTFTRYYMHWVRQAPGQGLEWMGIIQPRAGYTSYALKFQGRVTMTRDTSTSTVYMELSSLRSEDTAVYYCTSGWDVWGQGTLVTVSS

SEQ ID NO: 376 - protein, 107 aa, synthetic construct
DIQMTQSPSSLSASVGDRVTITCRASQSISTWLAWYQQKPGKAPKLLIYAASSLDEGVPSRFSGSGSGTDFTLTISSLQPEDFATYYCQQSYSFPVTFGQGTKVEIK

SEQ ID NO: 377 - protein, 113 aa, synthetic construct
QVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWEVWGQGTLVTVSS

SEQ ID NO: 378 - protein, 107 aa, synthetic construct; identical to SEQ ID NO: 376

SEQ ID NO: 379 - protein, 113 aa, synthetic construct; identical to SEQ ID NO: 353

SEQ ID NO: 380 - protein, 107 aa, synthetic construct; identical to SEQ ID NO: 376

SEQ ID NO: 381 - protein, 113 aa, synthetic construct
QVQLVQSGAEVKKPGASVKVSCKASGFTFTRYYMHWVRQAPGQGLEWMGVINPSGGYATYAQKFQGRVTMTRDTSTSTVYMELSSLRSEDTAVYYCASGWDVWGQGTLVTVSS

SEQ ID NO: 382 - protein, 107 aa, synthetic construct
DIQMTQSPSSLSASVGDRVTITCRASQSISSWLAWYQQKPGKAPKLLIYATSTLESGVPSRFSGSGSGTDFTLTISSLQPEDFATYYCQQSYSSPVTFGQGTKVEIK

SEQ ID NO: 383 - protein, 113 aa, synthetic construct
QVQLVQSGAEVKKPGASVKVSCKASGETFTRYYVHWVRQAPGQGLEWMGIINPSGGYASYAQKFQGRVTMTRDTSTSTVYMELSSLRSEDTAVYYCAAGLFIWGQGTLVTVSS

SEQ ID NO: 384 - protein, 107 aa, synthetic construct; identical to SEQ ID NO: 340

SEQ ID NO: 385 - protein, 113 aa, synthetic construct; identical to SEQ ID NO: 335

SEQ ID NO: 386 - protein, 107 aa, synthetic construct
DIQMTQSPSSLSASVGDRVTITCRASQSIGRELAWYQQKPGKAPKLLIYSASNLETGVPSRFSGSGSGTDFTLTISSLQPEDFATYYCQQYNRFPVTFGPGTKVDIK

SEQ ID NO: 387 - protein, 113 aa, synthetic construct
QVQLVQSGAEVKKPGASVKVSCKASGDEFTRYYVHWVRQAPGQGLEWMGIINPSGGYASYAQKFQGRVTMTRDTSTSTVYMELSSLRSEDTAVYYCAAGLFIWGQGTLVTVSS

SEQ ID NO: 388 - protein, 107 aa, synthetic construct; identical to SEQ ID NO: 340

SEQ ID NO: 389 - protein, 113 aa, synthetic construct
QVQLVQSGAEVKKPGASVKVSCKASGDEFTRYYVHWVRQAPGQGLEWMGIQNPSGGYASYAQKFQGRVTMTRDTSTSTVYMELSSLRSEDTAVYYCAAGLFIWGQGTLVTVSS

SEQ ID NO: 390 - protein, 107 aa, synthetic construct; identical to SEQ ID NO: 340

SEQ ID NO: 391 - protein, 113 aa, synthetic construct; identical to SEQ ID NO: 389

SEQ ID NO: 392 - protein, 107 aa, synthetic construct
DIQMTQSPSSLSASVGDRVTITCRASQSIGRELAWYQQKPGKAPKLLIYDASNLETGVPSRFSGSGSGTDFTLTISSLQPEDFATYYCQQYNRFPVTFGPGTKVDIK

SEQ ID NO: 393 - protein, 113 aa, synthetic construct
QVQLVQSGAEVKKPGASVKVSCKASGETFTNYYIHWVRQAPGQGLEWMGVINPRAGYTSYALKFQGRVTMTRDTSTSTVYMELSSLRSEDTAVYYCAGGWEDWGQGTLVTVSS

SEQ ID NO: 394 - protein, 107 aa, synthetic construct
DIQMTQSPSSLSASVGDRVTITCRISQSISSWLAWYQQKPGKAPKLLIYSASSLQSGVPSRFSGSGSGTDFTLTISSLQPEDFATYYCQQSFTSPITFGQGTRLEIK

SEQ ID NO: 395 - protein, 113 aa, synthetic construct
QVQLVQSGAEVKKPGASVKVSCKASGGTFTNYYIHWVRQAPGQGLEWMGIIDPRAGYTSYALKFQGRVTMTRDTSTSTVYMELSSLRSEDTAVYYCAGGWEDWGQGTLVTVSS

SEQ ID NO: 396 - protein, 107 aa, synthetic construct; identical to SEQ ID NO: 394

SEQ ID NO: 397 - protein, 113 aa, synthetic construct
QVQLVQSGAEVKKPGASVKVSCKASGSTFTNYYIHWVRQAPGQGLEWMGIIDPRAGYTSYALKFQGRVTMTRDTSTSTVYMELSSLRSEDTAVYYCAGGWEDWGQGTLVTVSS

SEQ ID NO: 398 - protein, 107 aa, synthetic construct
DIQMTQSPSSLSASVGDRVTITCRISQSISSFLAWYQQKPGKAPKLLIYSASSLQSGVPSRFSGSGSGTDFTLTISSLQPEDFATYYCQQSFTSPITFGQGTRLEIK

SEQ ID NO: 399 - protein, 113 aa, synthetic construct
QVQLVQSGAEVKKPGASVKVSCKASGETFTNYYIHWVRQAPGQGLEWMGIIDPRAGYTSYALKFQGRVTMTRDTSTSTVYMELSSLRSEDTAVYYCAGGWEDWGQGTLVTVSS

SEQ ID NO: 400 - protein, 107 aa, synthetic construct; identical to SEQ ID NO: 398

SEQ ID NO: 401 - protein, 113 aa, synthetic construct
QVQLVQSGAEVKKPGASVKVSCKASGTTFTNYYIHWVRQAPGQGLEWMGVINPRAGYTSYALKFQGRVTMTRDTSTSTVYMELSSLRSEDTAVYYCTGGWLDWGQGTLVTVSS

SEQ ID NO: 402 - protein, 107 aa, synthetic construct
DIQMTQSPSSLSASVGDRVTITCRASQSISSWLAWYQQKPGKAPKLLIYSASSLQSGVPSRFSGSGSGTDFTLTISSLQPEDFATYYCQQSVTMPVTFGQGTRLEIK

SEQ ID NO: 403 - protein, 113 aa, synthetic construct
QVQLVQSGAEVKKPGASVKVSCKASGYTFTNYYIHWVRQAPGQGLEWMGVINPRAGYTSYALKFQGRVTMTRDTSTSTVYMELSSLRSEDTAVYYCAGGWLDWGQGTLVTVSS

SEQ ID NO: 404 - protein, 107 aa, synthetic construct
DIQMTQSPSSLSASVGDRVTITCRASQRISSWLAWYQQKPGKAPKLLIYDASSLQSGVPSRFSGSGSGTDFTLTISSLQPEDFATYYCQQSFTMPVTFGQGTRLEIK

SEQ ID NO: 405 - protein, 113 aa, synthetic construct; identical to SEQ ID NO: 381

SEQ ID NO: 406 - protein, 107 aa, synthetic construct
DIQMTQSPSSLSASVGDRVTITCRASQSISSWLAWYQQKPGKAPKLLIYATSTLESGVPSRFSGSGSGTDFTLTISSLQPEDFATYYCQQSYSFPPTFGQGTKVEIK

SEQ ID NO: 407 - protein, 113 aa, synthetic construct; identical to SEQ ID NO: 383

SEQ ID NO: 408 - protein, 107 aa, synthetic construct; identical to SEQ ID NO: 336

SEQ ID NO: 409 - protein, 113 aa, synthetic construct; identical to SEQ ID NO: 353

SEQ ID NO: 410 - protein, 107 aa, synthetic construct
DIQMTQSPSSLSASVGDRVTITCRASQSISTWLAWYQQKPGKAPKLLIYAASSLDTGVPSRFSGSGSGTDFTLTISSLQPEDFATYYCQQSYSFPVTFGQGTKVEIK

SEQ ID NO: 411 - protein, 118 aa, synthetic construct
QVQLVQSGAEVKKPGSSVKVSCKASGYTFTAYYIHWVRQAPGQGLEFMGWIHPYSGGTNYAQKFQGRVTITADESTSTAYMELSSLRSEDTAVYYCAIGYYYGKFDYWGQGTLVTVSS

SEQ ID NO: 412 - protein, 107 aa, synthetic construct
DIQMTQSPSSLSASVGDRVTITCRASQTISRYLNWYQQKPGKAPKLLIYAASSLQSGVPSRFSGSGSGTDFTLTISSLQPEDFATYYCQQSYSTPRTFGQGTKLEIK

SEQ ID NO: 413-430 - skipped (18 entries)

SEQ ID NO: 431 - protein, 10 aa, synthetic construct
GDTFTRHYVH

SEQ ID NO: 432 - protein, 17 aa, synthetic construct
IINPSGGYASYAQKFQG

SEQ ID NO: 433 - protein, 6 aa, synthetic construct
AAGLFI

SEQ ID NO: 434 - protein, 11 aa, synthetic construct
RASQSIGRWLA

SEQ ID NO: 435 - protein, 7 aa, synthetic construct
SASNLET

SEQ ID NO: 436 - protein, 8 aa, synthetic construct
QQYESFPV

SEQ ID NO: 437 - protein, 10 aa, synthetic construct
GDTFTRYYVH

SEQ ID NO: 438 - protein, 17 aa, synthetic construct
IINPSGGYASYAQKFQG

SEQ ID NO: 439 - protein, 6 aa, synthetic construct
AAGLFI

SEQ ID NO: 440 - protein, 11 aa, synthetic construct
RASQSIGRWLA

SEQ ID NO: 441 - protein, 7 aa, synthetic construct
SASNLET

SEQ ID NO: 442 - protein, 8 aa, synthetic construct
QQYNSFPV

SEQ ID NO: 443 - protein, 10 aa, synthetic construct
GDTFTRYYVH

SEQ ID NO: 444 - protein, 17 aa, synthetic construct
IINPSGGYASYAQKFQG

SEQ ID NO: 445 - protein, 6 aa, synthetic construct
AAGLFI

SEQ ID NO: 446 - protein, 11 aa, synthetic construct
RASQSIGSWLA

SEQ ID NO: 447 - protein, 7 aa, synthetic construct
SASNLET

SEQ ID NO: 448 - protein, 8 aa, synthetic construct
QQYNSFPV

SEQ ID NO: 449 - protein, 10 aa, synthetic construct
GDTFTRYYVH

SEQ ID NO: 450 - protein, 17 aa, synthetic construct
IINPSGGYASYAQKFQG

SEQ ID NO: 451 - protein, 6 aa, synthetic construct
AAGLFI

SEQ ID NO: 452 - protein, 11 aa, synthetic construct
RASQSIGRWLA

SEQ ID NO: 453 - protein, 7 aa, synthetic construct
SASNLET

SEQ ID NO: 454 - protein, 8 aa, synthetic construct
QQYNRFPV

SEQ ID NO: 455 - protein, 10 aa, synthetic construct
GDTFTRYYVH

SEQ ID NO: 456 - protein, 17 aa, synthetic construct
IINPSGGYASYAQKFQG

SEQ ID NO: 457 - protein, 6 aa, synthetic construct
AAGLFI

SEQ ID NO: 458 - protein, 11 aa, synthetic construct
RASQSIGRWLA

SEQ ID NO: 459 - protein, 7 aa, synthetic construct
SASNLET

SEQ ID NO: 460 - protein, 8 aa, synthetic construct
QQYERFPV

SEQ ID NO: 461 - protein, 10 aa, synthetic construct
GDTFTRYYVH

SEQ ID NO: 462 - protein, 17 aa, synthetic construct
IINPSGGYASYAQKFQG

SEQ ID NO: 463 - protein, 6 aa, synthetic construct
AAGLFI

SEQ ID NO: 464 - protein, 11 aa, synthetic construct
RASQSIGRYLA

SEQ ID NO: 465 - protein, 7 aa, synthetic construct
SASNLET

SEQ ID NO: 466 - protein, 8 aa, synthetic construct
QQYNSFPV

SEQ ID NO: 467 - protein, 10 aa, synthetic construct
GDTFTRYYVH

SEQ ID NO: 468 - protein, 17 aa, synthetic construct
IINPSGGYASYAQKFQG

SEQ ID NO: 469 - protein, 6 aa, synthetic construct
AAGLFI

SEQ ID NO: 470 - protein, 11 aa, synthetic construct
RASQSIGRYLA

SEQ ID NO: 471 - protein, 7 aa, synthetic construct
SASNLET

SEQ ID NO: 472 - protein, 8 aa, synthetic construct
QQYNRFPV

SEQ ID NO: 473 - protein, 10 aa, synthetic construct
GDTFTRYYVH

SEQ ID NO: 474 - protein, 17 aa, synthetic construct
IINPSGGYASYAQKFQG

SEQ ID NO: 475 - protein, 6 aa, synthetic construct
AAGLFI

SEQ ID NO: 476 - protein, 11 aa, synthetic construct
RASQSIGRYLA

SEQ ID NO: 477 - protein, 7 aa, synthetic construct
YASNLET

SEQ ID NO: 478 - protein, 8 aa, synthetic construct
QQYNSFPV

SEQ ID NO: 479 - protein, 10 aa, synthetic construct
GDTFTRYYVH

SEQ ID NO: 480 - protein, 17 aa, synthetic construct
IINPSGGYASYAQKFQG

SEQ ID NO: 481 - protein, 6 aa, synthetic construct
AAGLFI

SEQ ID NO: 482 - protein, 11 aa, synthetic construct
RASQSIGRWLA

SEQ ID NO: 483 - protein, 7 aa, synthetic construct
SASNLET

SEQ ID NO: 484 - protein, 8 aa, synthetic construct
QQYESFPV

SEQ ID NO: 485 - protein, 10 aa, synthetic construct
GYTFTDYYMH

SEQ ID NO: 486 - protein, 17 aa, synthetic construct
IINPRAGYTSYALKFQG

SEQ ID NO: 487 - protein, 6 aa, synthetic construct
TSGWDV

SEQ ID NO: 488 - protein, 11 aa, synthetic construct
RASQSISTWLA

SEQ ID NO: 489 - protein, 7 aa, synthetic construct
AASSLDS

SEQ ID NO: 490 - protein, 8 aa, synthetic construct
QQSYSFPV

SEQ ID NO: 491 - protein, 10 aa, synthetic construct
GYTFTRYYMH

SEQ ID NO: 492 - protein, 17 aa, synthetic construct
IINPRAGYTSYALKFQG

SEQ ID NO: 493 - protein, 6 aa, synthetic construct
TSGWDV

SEQ ID NO: 494 - protein, 11 aa, synthetic construct
RASQSISTWLA

SEQ ID NO: 495 - protein, 7 aa, synthetic construct
AASSLDS

SEQ ID NO: 496 - protein, 8 aa, synthetic construct
QQSYSFPV

SEQ ID NO: 497 - protein, 10 aa, synthetic construct
GYTFTTYYMH

SEQ ID NO: 498 - protein, 17 aa, synthetic construct
IINPRAGYTSYALKFQG

SEQ ID NO: 499 - protein, 6 aa, synthetic construct
TSGWDV

SEQ ID NO: 500 - protein, 11 aa, synthetic construct
RASQSISTWLA

SEQ ID NO: 501 - protein, 7 aa, synthetic construct
AASSLDS

SEQ ID NO: 502 - protein, 8 aa, synthetic construct
QQSYSFPV

SEQ ID NO: 503 - protein, 10 aa, synthetic construct
GDTFTDYYMH

SEQ ID NO: 504 - protein, 17 aa, synthetic construct
IINPRAGYTSYALKFQG

SEQ ID NO: 505 - protein, 6 aa, synthetic construct
TSGWDV

SEQ ID NO: 506 - protein, 11 aa, synthetic construct
RASQSISTWLA

SEQ ID NO: 507 - protein, 7 aa, synthetic construct
AASSLDS

SEQ ID NO: 508 - protein, 8 aa, synthetic construct
QQSYSFPV

SEQ ID NO: 509 - protein, 10 aa, synthetic construct
GHTFTRYYMH

SEQ ID NO: 510 - protein, 17 aa, synthetic construct
IINPSGGYATYAQKFQG

SEQ ID NO: 511 - protein, 6 aa, synthetic construct
ASGWFV

SEQ ID NO: 512 - protein, 11 aa, synthetic construct
RASQSINSWLA

SEQ ID NO: 513 - protein, 7 aa, synthetic construct
DTSTLES

SEQ ID NO: 514 - protein, 8 aa, synthetic construct
QQSYSFPV

SEQ ID NO: 515 - protein, 10 aa, synthetic construct
GHTFTRYYMH

SEQ ID NO: 516 - protein, 17 aa, synthetic construct
IINPSGGYATYAQKFQG

SEQ ID NO: 517 - protein, 6 aa, synthetic construct
ASGLFI

SEQ ID NO: 518 - protein, 11 aa, synthetic construct
RASQSINSWLA

SEQ ID NO: 519 - protein, 7 aa, synthetic construct
ATSTLES

SEQ ID NO: 520 - protein, 8 aa, synthetic construct
QQYYRFPV

SEQ ID NO: 521 - protein, 10 aa, synthetic construct
GHTFTRYYMH

SEQ ID NO: 522 - protein, 17 aa, synthetic construct
IINPSGGYATYAQKFQG

SEQ ID NO: 523 - protein, 6 aa, synthetic construct
ASGLFI

SEQ ID NO: 524 - protein, 11 aa, synthetic construct
RASQSINSWLA

SEQ ID NO: 525 - protein, 7 aa, synthetic construct
ATSTLES

SEQ ID NO: 526 - protein, 8 aa, synthetic construct
QQYYSFPV

SEQ ID NO: 527 - protein, 10 aa, synthetic construct
GHTFTRYYMH

SEQ ID NO: 528 - protein, 17 aa, synthetic construct
IINPSGGYATYAQKFQG

SEQ ID NO: 529 - protein, 6 aa, synthetic construct
ASGLFI

SEQ ID NO: 530 - protein, 11 aa, synthetic construct
RASQSINSWLA

SEQ ID NO: 531 - protein, 7 aa, synthetic construct
ATSTLES

SEQ ID NO: 532 - protein, 8 aa, synthetic construct
QQSYRFPV

SEQ ID NO: 533 - protein, 10 aa, synthetic construct
GYTFTDYYMH

SEQ ID NO: 534 - protein, 17 aa, synthetic construct
IINPRAGYTSYALKFQG

SEQ ID NO: 535 - protein, 6 aa, synthetic construct
TSGWDV

SEQ ID NO: 536 - protein, 11 aa, synthetic construct
RASQSISTWLA

SEQ ID NO: 537 - protein, 7 aa, synthetic construct
AASSLDS

SEQ ID NO: 538 - protein, 8 aa, synthetic construct
QQSYRFPV

SEQ ID NO: 539 - protein, 10 aa, synthetic construct
GHTFTRYYMH

SEQ ID NO: 540 - protein, 17 aa, synthetic construct
IINPSGGYATYAQKFQG

SEQ ID NO: 541 - protein, 6 aa, synthetic construct
ASGLDI

SEQ ID NO: 542 - protein, 11 aa, synthetic construct
RASQSINSWLA

SEQ ID NO: 543 - protein, 7 aa, synthetic construct
ATSTLES

SEQ ID NO: 544 - protein, 8 aa, synthetic construct
QQSYRFPV

SEQ ID NO: 545 - protein, 10 aa, synthetic construct
GYIFNGYDIH

SEQ ID NO: 546 - protein, 17 aa, synthetic construct
WMNPDNGNTGLAQKFQG

SEQ ID NO: 547 - protein, 17 aa, synthetic construct
ARGMATRFPYYYYGMDV

SEQ ID NO: 548 - protein, 16 aa, synthetic construct
RSSQSLLHSNGYNYLD

SEQ ID NO: 549 - protein, 7 aa, synthetic construct
LGSNRAS

SEQ ID NO: 550 - protein, 8 aa, synthetic construct
MQGTHWPP

SEQ ID NO: 551 - protein, 10 aa, synthetic construct
GHTFTRYYMH

SEQ ID NO: 552 - protein, 17 aa, synthetic construct
VINPSGGYATYAQKFQG

SEQ ID NO: 553 - protein, 6 aa, synthetic construct
ASGWDV

SEQ ID NO: 554 - protein, 11 aa, synthetic construct
RASQSINSWLA

SEQ ID NO: 555 - protein, 7 aa, synthetic construct
ATSTLES

SEQ ID NO: 556 - protein, 8 aa, synthetic construct
QQSYSSPV

SEQ ID NO: 557 - protein, 10 aa, synthetic construct
GYTFTRYYMH

SEQ ID NO: 558 - protein, 17 aa, synthetic construct
IIQPRAGYTSYALKFQG

SEQ ID NO: 559 - protein, 6 aa, synthetic construct
TSGWDV

SEQ ID NO: 560 - protein, 11 aa, synthetic construct
RASQSISTWLA

SEQ ID NO: 561 - protein, 7 aa, synthetic construct
AASSLDE

SEQ ID NO: 562 - protein, 8 aa, synthetic construct
QQSYSFPV

SEQ ID NO: 563 - protein, 10 aa, synthetic construct
GYTFTRYYMH

SEQ ID NO: 564 - protein, 17 aa, synthetic construct
IINPRAGYTSYALKFQG

SEQ ID NO: 565 - protein, 6 aa, synthetic construct
TSGWEV

SEQ ID NO: 566 - protein, 11 aa, synthetic construct
RASQSISTWLA

SEQ ID NO: 567 - protein, 7 aa, synthetic construct
AASSLDE

SEQ ID NO: 568 - protein, 8 aa, synthetic construct
QQSYSFPV

SEQ ID NO: 569 - protein, 10 aa, synthetic construct
GYTFTRYYMH

SEQ ID NO: 570 - protein, 17 aa, synthetic construct
IINPRAGYTSYALKFQG

SEQ ID NO: 571 - protein, 6 aa, synthetic construct
TSGWDV

SEQ ID NO: 572 - protein, 11 aa, synthetic construct
RASQSISTWLA

SEQ ID NO: 573 - protein, 7 aa, synthetic construct
AASSLDE

SEQ ID NO: 574 - protein, 8 aa, synthetic construct
QQSYSFPV

SEQ ID NO: 575 - protein, 10 aa, synthetic construct
GFTFTRYYMH

SEQ ID NO: 576 - protein, 17 aa, synthetic construct
VINPSGGYATYAQKFQG

SEQ ID NO: 577 - protein, 6 aa, synthetic construct
ASGWDV

SEQ ID NO: 578 - protein, 11 aa, synthetic construct
RASQSISSWLA

SEQ ID NO: 579 - protein, 7 aa, synthetic construct
ATSTLES

SEQ ID NO: 580 - protein, 8 aa, synthetic construct
QQSYSSPV

SEQ ID NO: 581 - protein, 10 aa, synthetic construct
GETFTRYYVH

SEQ ID NO: 582 - protein, 17 aa, synthetic construct
IINPSGGYASYAQKFQG

SEQ ID NO: 583 - protein, 6 aa, synthetic construct
AAGLFI

SEQ ID NO: 584 - protein, 11 aa, synthetic construct
RASQSIGRWLA

SEQ ID NO: 585 - protein, 7 aa, synthetic construct
SASNLET

SEQ ID NO: 586 - protein, 8 aa, synthetic construct
QQYNRFPV

SEQ ID NO: 587 - protein, 10 aa, synthetic construct
GDTFTRYYVH

SEQ ID NO: 588 - protein, 17 aa, synthetic construct
IINPSGGYASYAQKFQG

SEQ ID NO: 589 - protein, 6 aa, synthetic construct
AAGLFI

SEQ ID NO: 590 - protein, 11 aa, synthetic construct
RASQSIGRELA

SEQ ID NO: 591 - protein, 7 aa, synthetic construct
SASNLET

SEQ ID NO: 592 - protein, 8 aa, synthetic construct
QQYNRFPV

SEQ ID NO: 593 - protein, 10 aa, synthetic construct
GDEFTRYYVH

SEQ ID NO: 594 - protein, 17 aa, synthetic construct
IINPSGGYASYAQKFQG

SEQ ID NO: 595 - protein, 6 aa, synthetic construct
AAGLFI

SEQ ID NO: 596 - protein, 11 aa, synthetic construct
RASQSIGRWLA

SEQ ID NO: 597 - protein, 7 aa, synthetic construct
SASNLET

SEQ ID NO: 598 - protein, 8 aa, synthetic construct
QQYNRFPV

SEQ ID NO: 599 - protein, 10 aa, synthetic construct
GDEFTRYYVH

SEQ ID NO: 600 - protein, 17 aa, synthetic construct
IQNPSGGYASYAQKFQG

SEQ ID NO: 601 - protein, 6 aa, synthetic construct
AAGLFI

SEQ ID NO: 602 - protein, 11 aa, synthetic construct
RASQSIGRWLA

SEQ ID NO: 603 - protein, 7 aa, synthetic construct
SASNLET

SEQ ID NO: 604 - protein, 8 aa, synthetic construct
QQYNRFPV

SEQ ID NO: 605 - protein, 10 aa, synthetic construct
GDEFTRYYVH

SEQ ID NO: 606 - protein, 17 aa, synthetic construct
IQNPSGGYASYAQKFQG

SEQ ID NO: 607 - protein, 6 aa, synthetic construct
AAGLFI

SEQ ID NO: 608 - protein, 11 aa, synthetic construct
RASQSIGRELA

SEQ ID NO: 609 - protein, 7 aa, synthetic construct
DASNLET

SEQ ID NO: 610 - protein, 8 aa, synthetic construct
QQYNRFPV

SEQ ID NO: 611 - protein, 10 aa, synthetic construct
GETFTNYYIH

SEQ ID NO: 612 - protein, 17 aa, synthetic construct
VINPRAGYTSYALKFQG

SEQ ID NO: 613 - protein, 6 aa, synthetic construct
AGGWED

SEQ ID NO: 614 - protein, 11 aa, synthetic construct
RISQSISSWLA

SEQ ID NO: 615 - protein, 7 aa, synthetic construct
SASSLQS

SEQ ID NO: 616 - protein, 8 aa, synthetic construct
QQSFTSPI

SEQ ID NO: 617 - protein, 10 aa, synthetic construct
GGTFTNYYIH

SEQ ID NO: 618 - protein, 17 aa, synthetic construct
IIDPRAGYTSYALKFQG

SEQ ID NO: 619 - protein, 6 aa, synthetic construct
AGGWED

SEQ ID NO: 620 - protein, 11 aa, synthetic construct
RISQSISSWLA

SEQ ID NO: 621 - protein, 7 aa, synthetic construct
SASSLQS

SEQ ID NO: 622 - protein, 8 aa, synthetic construct
QQSFTSPI

SEQ ID NO: 623 - protein, 10 aa, synthetic construct
GSTFTNYYIH

SEQ ID NO: 624 - protein, 17 aa, synthetic construct
IIDPRAGYTSYALKFQG

SEQ ID NO: 625 - protein, 6 aa, synthetic construct
AGGWED

SEQ ID NO: 626 - protein, 11 aa, synthetic construct
RISQSISSFLA

SEQ ID NO: 627 - protein, 7 aa, synthetic construct
SASSLQS

SEQ ID NO: 628 - protein, 8 aa, synthetic construct
QQSFTSPI

SEQ ID NO: 629 - protein, 10 aa, synthetic construct
GETFTNYYIH

SEQ ID NO: 630 - protein, 17 aa, synthetic construct
IIDPRAGYTSYALKFQG

SEQ ID NO: 631 - protein, 6 aa, synthetic construct
AGGWED

SEQ ID NO: 632 - protein, 11 aa, synthetic construct
RISQSISSFLA

SEQ ID NO: 633 - protein, 7 aa, synthetic construct
SASSLQS

SEQ ID NO: 634 - protein, 8 aa, synthetic construct
QQSFTSPI

SEQ ID NO: 635 - protein, 10 aa, synthetic construct
GTTFTNYYIH

SEQ ID NO: 636 - protein, 17 aa, synthetic construct
VINPRAGYTSYALKFQG

SEQ ID NO: 637 - protein, 6 aa, synthetic construct
TGGWLD

SEQ ID NO: 638 - protein, 11 aa, synthetic construct
RASQSISSWLA

SEQ ID NO: 639 - protein, 7 aa, synthetic construct
SASSLQS

SEQ ID NO: 640 - protein, 8 aa, synthetic construct
QQSVTMPV

SEQ ID NO: 641 - protein, 10 aa, synthetic construct
GYTFTNYYIH

SEQ ID NO: 642 - protein, 17 aa, synthetic construct
VINPRAGYTSYALKFQG

SEQ ID NO: 643 - protein, 6 aa, synthetic construct
AGGWLD

SEQ ID NO: 644 - protein, 11 aa, synthetic construct
RASQRISSWLA

SEQ ID NO: 645 - protein, 7 aa, synthetic construct
DASSLQS

SEQ ID NO: 646 - protein, 8 aa, synthetic construct
QQSFTMPV

SEQ ID NO: 647 - protein, 10 aa, synthetic construct
GFTFTRYYMH

SEQ ID NO: 648 - protein, 17 aa, synthetic construct
VINPSGGYATYAQKFQG

SEQ ID NO: 649 - protein, 6 aa, synthetic construct
ASGWDV

SEQ ID NO: 650 - protein, 11 aa, synthetic construct
RASQSISSWLA

SEQ ID NO: 651 - protein, 7 aa, synthetic construct
ATSTLES

SEQ ID NO: 652 - protein, 8 aa, synthetic construct
QQSYSFPP

SEQ ID NO: 653 - protein, 10 aa, synthetic construct
GETFTRYYVH

SEQ ID NO: 654 - protein, 17 aa, synthetic construct
IINPSGGYASYAQKFQG

SEQ ID NO: 655 - protein, 6 aa, synthetic construct
AAGLFI

SEQ ID NO: 656 - protein, 11 aa, synthetic construct
RASQSIGRWLA

SEQ ID NO: 657 - protein, 7 aa, synthetic construct
SASNLET

SEQ ID NO: 658 - protein, 8 aa, synthetic construct
QQYNSFPV

SEQ ID NO: 659 - protein, 10 aa, synthetic construct
GYTFTRYYMH

SEQ ID NO: 660 - protein, 17 aa, synthetic construct
IINPRAGYTSYALKFQG

SEQ ID NO: 661 - protein, 6 aa, synthetic construct
TSGWDV

SEQ ID NO: 662 - protein, 11 aa, synthetic construct
RASQSISTWLA

SEQ ID NO: 663 - protein, 7 aa, synthetic construct
AASSLDT

SEQ ID NO: 664 - protein, 8 aa, synthetic construct
QQSYSFPV

SEQ ID NO: 665 - protein, 10 aa, synthetic construct
GYTFTAYYIH

SEQ ID NO: 666 - protein, 17 aa, synthetic construct
WIHPYSGGTNYAQKFQG

SEQ ID NO: 667 - protein, 11 aa, synthetic construct
AIGYYYGKFDY

SEQ ID NO: 668 - protein, 11 aa, synthetic construct
RASQTISRYLN

SEQ ID NO: 669 - protein, 7 aa, synthetic construct
AASSLQS

SEQ ID NO: 670 - protein, 8 aa, synthetic construct
QQSYSTPR

SEQ ID NO: 671 - protein, 214 aa, synthetic construct; identical to SEQ ID NO: 64

SEQ ID NO: 672 - protein, 214 aa, synthetic construct
DIQMTQSPSSLSASVGDRVTITCRASQSISSWLAWYQQKPGKAPKLLIYAASTLQSGVPSRFSGSGSGTDFTLTISSLQPEDFATYYCQQSYSTPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 673 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 674 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 675 - protein, 220 aa, synthetic construct
DIVMTQSPDSLAVSLGERATINCKASQSLLHSSSNKNYLAWYQQKPGQPPKLLIYWASTRESGVPDRFSGSGSGTDFTLTISSLQAEDVAVYYCQQYYSTPITFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 676 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 677 - protein, 292 aa, synthetic construct
MSTSTEQKLISEEDLQVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSASGGGGSGGGGSGGGGSHASDIVMTQSPDSLAVSLGERATINCKASQSLLHSSSNKNYLAWYQQKPGQPPKLLIYWASTRESGVPDRFSGSGSGTDFTLTISSLQAEDVAVYYCQQYYSTPITFGPGTKVDIKGSGLNDIFEAQKIEWHEGKPIPNPLLGLDST

SEQ ID NO: 678 - protein, 269 aa, synthetic construct
MSTSTEQKLISEEDLQVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYESFPVTFGPGTKVDIKGKPIPNPLLGLDST

SEQ ID NO: 679 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 680 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 681 - protein, 214 aa, synthetic construct; identical to SEQ ID NO: 178

SEQ ID NO: 682 - protein, 214 aa, synthetic construct; identical to SEQ ID NO: 206

SEQ ID NO: 683 - protein, 456 aa, synthetic construct
QVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 684 - protein, 600 aa, synthetic construct; identical to SEQ ID NO: 208

SEQ ID NO: 685 - protein, 603 aa, synthetic construct; identical to SEQ ID NO: 207

SEQ ID NO: 686 - protein, 596 aa, synthetic construct; identical to SEQ ID NO: 205

SEQ ID NO: 687 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 688 - protein, 214 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYESFPVTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 689 - protein, 367 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSDIQMTQSPSSLSASVGDRVTITCRASQSIGRWLAWYQQKPGKAPKLLIYSASNLETGVPSRFSGSGSGTDFTLTISSLQPEDFATYYCQQYESFPVTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 690 - protein, 443 aa, synthetic construct
QVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 691 - protein, 491 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEPKSSDTPPPSPRSPEPKSSDTPPPSPRSPEPKSCDTPPPCPRAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 692 - protein, 640 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEPKSSDTPPPSPRSPEPKSSDTPPPSPRSPEPKSCDTPPPCPRAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 693 - protein, 484 aa, synthetic construct
QVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEPKSSDTPPPSPRSPEPKSSDTPPPSPRSPEPKSCDTPPPCPRAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 694 - protein, 633 aa, synthetic construct
QVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEPKSSDTPPPSPRSPEPKSSDTPPPSPRSPEPKSCDTPPPCPRAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 695 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 696 - protein, 214 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 697 - protein, 214 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYNSFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 698 - protein, 214 aa, synthetic construct
DIQMTQSPSSLSASVGDRVTITCRASQSIGRYLAWYQQKPGKAPKLLIYSASNLETGVPSRFSGSGSGTDFTLTISSLQPEDFATYYCQQYNRFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 699 - protein, 214 aa, synthetic construct; identical to SEQ ID NO: 175

SEQ ID NO: 700 - protein, 214 aa, synthetic construct
DIQMTQSPSSLSASVGDRVTITCRASQSIGRYLAWYQQKPGKAPKLLIYYASNLETGVPSRFSGSGSGTDFTLTISSLQPEDFATYYCQQYNSFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 701 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYERFPVTFGPGTKVDIKAAAGSGSEQKLISEEDLGKPIPNPLLGLDST

SEQ ID NO: 702 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYESFPVTFGPGTKVDIKAAAGSGSEQKLISEEDLGKPIPNPLLGLDST

SEQ ID NO: 703 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIKAAAGSGSEQKLISEEDLGKPIPNPLLGLDST

SEQ ID NO: 704 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYNSFPVTFGPGTKVDIKAAAGSGSEQKLISEEDLGKPIPNPLLGLDST

SEQ ID NO: 705 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRYLAWYQQKPGKAPKLLIYSASNLETGVPSRFSGSGSGTDFTLTISSLQPEDFATYYCQQYNRFPVTFGPGTKVDIKAAAGSGSEQKLISEEDLGKPIPNPLLGLDST

SEQ ID NO: 706 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIKAAAGSGSEQKLISEEDLGKPIPNPLLGLDST

SEQ ID NO: 707 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRYLAWYQQKPGKAPKLLIYYASNLETGVPSRFSGSGSGTDFTLTISSLQPEDFATYYCQQYNSFPVTFGPGTKVDIKAAAGSGSEQKLISEEDLGKPIPNPLLGLDST

SEQ ID NO: 708 - protein, 276 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGSWLAWYQQKPGKAPKLLIYSASNLETGVPSRFSGSGSGTDFTLTISSLQPEDFATYYCQQYNSFPVTFGPGTKVDIKAAAGSGSEQKLISEEDLGKPIPNPLLGLDST

SEQ ID NO: 709 - protein, 596 aa, synthetic construct; identical to SEQ ID NO: 210

SEQ ID NO: 710 - protein, 443 aa, synthetic construct; identical to SEQ ID NO: 211

SEQ ID NO: 711 - protein, 605 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGYTFTKNYMHWVRQAPGQGLEWLGWVSPDSGYTGYAQKFQGRVTMTRDTSTSTVYMELSSLRSEDTAVYYCTTDLLSLELDDAFDIWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 712 - protein, 276 aa, synthetic construct
MSTSTQVQLVQSGAEVKKPGASVKVSCKASGDTFTDYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKAAAGSGSEQKLISEEDLGKPIPNPLLGLDST

SEQ ID NO: 713 - protein, 276 aa, synthetic construct
MSTSTQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSASGGGGSGGGGSGGGGSHASDIQMTQSPSSLSASVGDRVTITCRASQSINSWLAWYQQKPGKAPKLLIYATSTLESGVPSRFSGSGSGTDFTLTISSLQPEDFATYYCQQSYRFPVTFGQGTKVEIKAAAGSGSEQKLISEEDLGKPIPNPLLGLDST

SEQ ID NO: 714 - protein, 276 aa, synthetic construct
MSTSTQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSASGGGGSGGGGSGGGGSHASDIQMTQSPSSLSASVGDRVTITCRASQSINSWLAWYQQKPGKAPKLLIYATSTLESGVPSRFSGSGSGTDFTLTISSLQPEDFATYYCQQYYRFPVTFGQGTKVEIKAAAGSGSEQKLISEEDLGKPIPNPLLGLDST

SEQ ID NO: 715 - protein, 276 aa, synthetic construct
MSTSTQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSASGGGGSGGGGSGGGGSHASDIQMTQSPSSLSASVGDRVTITCRASQSINSWLAWYQQKPGKAPKLLIYATSTLESGVPSRFSGSGSGTDFTLTISSLQPEDFATYYCQQYYSFPVTFGQGTKVEIKAAAGSGSEQKLISEEDLGKPIPNPLLGLDST

SEQ ID NO: 716 - protein, 276 aa, synthetic construct
MSTSTQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGWFVWGQGTLVTVSSASGGGGSGGGGSGGGGSHASDIQMTQSPSSLSASVGDRVTITCRASQSINSWLAWYQQKPGKAPKLLIYDTSTLESGVPSRFSGSGSGTDFTLTISSLQPEDFATYYCQQSYSFPVTFGQGTKVEIKAAAGSGSEQKLISEEDLGKPIPNPLLGLDST

SEQ ID NO: 717 - protein, 276 aa, synthetic construct
MSTSTQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKAAAGSGSEQKLISEEDLGKPIPNPLLGLDST

SEQ ID NO: 718 - protein, 276 aa, synthetic construct
MSTSTQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKAAAGSGSEQKLISEEDLGKPIPNPLLGLDST

SEQ ID NO: 719 - protein, 276 aa, synthetic construct
MSTSTQVQLVQSGAEVKKPGASVKVSCKASGYTFTTYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKAAAGSGSEQKLISEEDLGKPIPNPLLGLDST

SEQ ID NO: 720 - protein, 727 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSEVQLVESGGGLVKPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTLVTVSSASGGGGSGGGGSGGGGSHASDIQMTQSPSSLSASVGDRVSITCKASQNVGTNVGWYQQKPGKAPKALIYSASFRYSGVPSRFSGSGSGTDFTLTISSLQPEDFATYFCQQYYTYPYTFGGGTKLEIKGGGSGGGSHHHHHH

SEQ ID NO: 721 - protein, 861 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGGGGSGGGGSGGGGSGGGGSQVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSASGGGGSGGGGSGGGGSHASDIVMTQSPDSLAVSLGERATINCKASQSLLHSSSNKNYLAWYQQKPGQPPKLLIYWASTRESGVPDRFSGSGSGTDFTLTISSLQAEDVAVYYCQQYYSTPITFGPGTKVDIK

SEQ ID NO: 722 - protein, 861 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGGGGSGGGGSGGGGSGGGGSQVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSASGGGGSGGGGSGGGGSHASDIVMTQSPDSLAVSLGERATINCKASQSLLHSSSNKNYLAWYQQKPGQPPKLLIYWASTRESGVPDRFSGSGSGTDFTLTISSLQAEDVAVYYCQQYYSTPITFGPGTKVDIK

SEQ ID NO: 723 - protein, 723 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRHYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYESFPVTFGPGTKVDIKGGGSGGGSHHHHHH

SEQ ID NO: 724 - protein, 723 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIKGGGSGGGSHHHHHH

SEQ ID NO: 725 - protein, 723 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSASGGGGSGGGGSGGGGSHASDIQMTQSPSSLSASVGDRVTITCRASQSINSWLAWYQQKPGKAPKLLIYATSTLESGVPSRFSGSGSGTDFTLTISSLQPEDFATYYCQQYYRFPVTFGQGTKVEIKGGGSGGGSHHHHHH

SEQ ID NO: 726 - protein, 723 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKGGGSGGGSHHHHHH

SEQ ID NO: 727 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 728 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 729 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGYTFTTYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 730 - protein, 214 aa, synthetic construct
DIQMTQSPSSLSASVGDRVTITCRASQSIGRWLAWYQQKPGKAPKLLIYSASNLETGVPSRFSGSGSGTDFTLTISSLQPEDFATYYCQQYERFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 731 - protein, 214 aa, synthetic construct
DIQMTQSPSSLSASVGDRVTITCRASQSIGSWLAWYQQKPGKAPKLLIYSASNLETGVPSRFSGSGSGTDFTLTISSLQPEDFATYYCQQYNSFPVTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 732 - protein, 214 aa, synthetic construct; identical to SEQ ID NO: 188

SEQ ID NO: 733 - protein, 214 aa, synthetic construct
DIQMTQSPSSLSASVGDRVTITCRASQSISTWLAWYQQKPGKAPKLLIYAASSLDSGVPSRFSGSGSGTDFTLTISSLQPEDFATYYCQQSYRFPVTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 734 - protein, 214 aa, synthetic construct; identical to SEQ ID NO: 185

SEQ ID NO: 735 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 736 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 737 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 738 - protein, 596 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSQVQLVQSGAEVKKPGASVKVSCKASGYTFTTYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 739 - protein, 456 aa, synthetic construct; identical to SEQ ID NO: 182

SEQ ID NO: 740 - protein, 456 aa, synthetic construct; identical to SEQ ID NO: 187

SEQ ID NO: 741 - protein, 456 aa, synthetic construct; identical to SEQ ID NO: 184

SEQ ID NO: 742 - protein, 465 aa, synthetic construct
QVQLVQSGAEVKKPGASVKVSCKASGYTFTKNYMHWVRQAPGQGLEWLGWVSPDSGYTGYAQKFQGRVTMTRDTSTSTVYMELSSLRSEDTAVYYCTTDLLSLELDDAFDIWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 743 - protein, 456 aa, synthetic construct
QVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 744 - protein, 456 aa, synthetic construct
QVQLVQSGAEVKKPGASVKVSCKASGYTFTTYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGSGGGSHHHHHH

SEQ ID NO: 745 - protein, 214 aa, synthetic construct; identical to SEQ ID NO: 180

SEQ ID NO: 746 - protein, 581 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 747 - protein, 581 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 748 - protein, 443 aa, synthetic construct
QVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGK

SEQ ID NO: 749 - protein, 586 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 750 - protein, 586 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 751 - protein, 591 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 752 - protein, 591 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 753 - protein, 601 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 754 - protein, 601 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSGGGGSGGGGSGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 755 - protein, 596 aa, synthetic construct; identical to SEQ ID NO: 181

SEQ ID NO: 756 - protein, 596 aa, synthetic construct; identical to SEQ ID NO: 186

SEQ ID NO: 757 - protein, 596 aa, synthetic construct; identical to SEQ ID NO: 183

SEQ ID NO: 758 - protein, 596 aa, synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTGGGGSGGGGSGGGGSGGGGSQVQLVQSGAEVKKPGASVKVSCKASGYTFTTYYMHWVRQAPGQGLEWMGIINPRAGYTSYALKFQGRVTMTRDTSTSTVYMELSSLRSEDTAVYYCTSGWDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 759 - protein, 590 aa, synthetic construct; identical to SEQ ID NO: 199

SEQ ID NO: 760 - protein, 716 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSASGGGGSGGGGSGGGGSHASDIQMTQSPSSLSASVGDRVTITCRASQSINSWLAWYQQKPGKAPKLLIYATSTLESGVPSRFSGSGSGTDFTLTISSLQPEDFATYYCQQYYRFPVTFGQGTKVEIKGGGSGGGSHHHHHH

SEQ ID NO: 761 - protein, 716 aa, synthetic construct; identical to SEQ ID NO: 196

SEQ ID NO: 762 - protein, 590 aa, synthetic construct
QVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLT

SEQ ID NO: 763 - protein, 716 aa, synthetic construct
QVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYERFPVTFGPGTKVDIKGGGSGGGSHHHHHH

SEQ ID NO: 764 - protein, 716 aa, synthetic construct
QVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRYLAWYQQKPGKAPKLLIYSASNLETGVPSRFSGSGSGTDFTLTISSLQPEDFATYYCQQYNSFPVTFGPGTKVDIKGGGSGGGSHHHHHH

SEQ ID NO: 765 - protein, 716 aa, synthetic construct
QVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSASGGGGSGGGGSGGGGSHASDIQMTQSPSSLSASVGDRVTITCRASQSINSWLAWYQQKPGKAPKLLIYATSTLESGVPSRFSGSGSGTDFTLTISSLQPEDFATYYCQQYYRFPVTFGQGTKVEIKGGGSGGGSHHHHHH

SEQ ID NO: 766 - protein, 716 aa, synthetic construct
QVQLVQSGAEVKKPGASVKVSCKASGYTFTTYYMHWVRQAPGQGLEWMGIINPSGGGTLYAQKFQGRVTMTRDTSTSTVYMELSSLRSEDTAVYYCAAGLFIWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGYTFTDYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKGGGSGGGSHHHHHH

SEQ ID NO: 767 - protein, 590 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 768 - protein, 709 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASGGGGSGGGGSGGGGSHASDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 769 - protein, 702 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIK

SEQ ID NO: 770 - protein, 702 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIK

SEQ ID NO: 771 - protein, 214 aa, synthetic construct
DIQMTQSPSSLSASVGDRVTITCRASQTISRYLNWYQQKPGKAPKLLIYAASSLQSGVPSRFSGSGSGTDFTLTISSLQPEDFATYYCQQSYSTPRTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 772 - protein, 595 aa, synthetic construct
QVQLVQSGAEVKKPGSSVKVSCKASGYTFTAYYIHWVRQAPGQGLEFMGWIHPYSGGTNYAQKFQGRVTITADESTSTAYMELSSLRSEDTAVYYCAIGYYYGKFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLT

SEQ ID NO: 773 - protein, 707 aa, synthetic construct
QVQLVQSGAEVKKPGSSVKVSCKASGYTFTAYYIHWVRQAPGQGLEFMGWIHPYSGGTNYAQKFQGRVTITADESTSTAYMELSSLRSEDTAVYYCAIGYYYGKFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIK

SEQ ID NO: 774 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 775 - protein, 601 aa, synthetic construct
QVQLVQSGAEVKKPGASVKVSCKASGYIFNGYDIHWVRQAPGQGLEWMGWMNPDNGNTGLAQKFQGRVTITADESTSTAYMELSSLRSEDTAVYYCARGMATRFPYYYYGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLT

SEQ ID NO: 776 - protein, 713 aa, synthetic construct
QVQLVQSGAEVKKPGASVKVSCKASGYIFNGYDIHWVRQAPGQGLEWMGWMNPDNGNTGLAQKFQGRVTITADESTSTAYMELSSLRSEDTAVYYCARGMATRFPYYYYGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIK

SEQ ID NO: 777 - protein, 708 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSGGGGSQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSASGGGGSGGGGSGGGGSHASDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIK

SEQ ID NO: 778 - protein, 708 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSGGGGSQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSASGGGGSGGGGSGGGGSHASDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIK

SEQ ID NO: 779 - protein, 288 aa, synthetic construct
MSTSTQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASGGGGSGGGGSGGGGSHASEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKEQKLISEEDLGSGLNDIFEAQKIEWHEGKPIPNPLLGLDSTNA

SEQ ID NO: 780 - protein, 299 aa, synthetic construct
MSTSTQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASGGGGSGGGGSGGGGSHASEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKEQKLISEEDLGSGLNDIFEAQKIEWHEGKPIPNPLLGLDSTNA

SEQ ID NO: 781 - protein, 286 aa, synthetic construct
MSTSTQVQLVQSGAEVKKPGASVKVSCKASGDEFTRYYVHWVRQAPGQGLEWMGIINPSGGYASYAQKFQGRVTMTRDTSTSTVYMELSSLRSEDTAVYYCAAGLFIWGQGTLVTVSSGGGGSGGGGSGGGGSGGGGSDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIKEQKLISEEDLGSGLNDIFEAQKIEWHEGKPIPNPLLGLDST

SEQ ID NO: 782 - protein, 286 aa, synthetic construct
MSTSTQVQLVQSGAEVKKPGASVKVSCKASGDEFTRYYVHWVRQAPGQGLEWMGIQNPSGGYASYAQKFQGRVTMTRDTSTSTVYMELSSLRSEDTAVYYCAAGLFIWGQGTLVTVSSGGGGSGGGGSGGGGSGGGGSDIQMTQSPSSLSASVGDRVTITCRASQSIGRELAWYQQKPGKAPKLLIYDASNLETGVPSRFSGSGSGTDFTLTISSLQPEDFATYYCQQYNRFPVTFGPGTKVDIKEQKLISEEDLGSGLNDIFEAQKIEWHEGKPIPNPLLGLDST

SEQ ID NO: 783 - protein, 286 aa, synthetic construct
MSTSTQVQLVQSGAEVKKPGASVKVSCKASGDEFTRYYVHWVRQAPGQGLEWMGIQNPSGGYASYAQKFQGRVTMTRDTSTSTVYMELSSLRSEDTAVYYCAAGLFIWGQGTLVTVSSGGGGSGGGGSGGGGSGGGGSDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIKEQKLISEEDLGSGLNDIFEAQKIEWHEGKPIPNPLLGLDST

SEQ ID NO: 784 - protein, 286 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSGGGGSGGGGSGGGGSGGGGSDIQMTQSPSSLSASVGDRVTITCRASQSIGRELAWYQQKPGKAPKLLIYSASNLETGVPSRFSGSGSGTDFTLTISSLQPEDFATYYCQQYNRFPVTFGPGTKVDIKEQKLISEEDLGSGLNDIFEAQKIEWHEGKPIPNPLLGLDST

SEQ ID NO: 785 - protein, 286 aa, synthetic construct
MSTSTQVQLVQSGAEVKKPGASVKVSCKASGDTFTRYYVHWVRQAPGQGLEWMGIINPSGGYASYAQKFQGRVTMTRDTSTSTVYMELSSLRSEDTAVYYCAAGLFIWGQGTLVTVSSGGGGSGGGGSGGGGSGGGGSDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIKEQKLISEEDLGSGLNDIFEAQKIEWHEGKPIPNPLLGLDST

SEQ ID NO: 786 - protein, 286 aa, synthetic construct
MSTSTQVQLVQSGAEVKKPGASVKVSCKASGETFTRYYVHWVRQAPGQGLEWMGIINPSGGYASYAQKFQGRVTMTRDTSTSTVYMELSSLRSEDTAVYYCAAGLFIWGQGTLVTVSSGGGGSGGGGSGGGGSGGGGSDIQMTQSPSSLSASVGDRVTITCRASQSIGRWLAWYQQKPGKAPKLLIYSASNLETGVPSRFSGSGSGTDFTLTISSLQPEDFATYYCQQYNRFPVTFGPGTKVDIKEQKLISEEDLGSGLNDIFEAQKIEWHEGKPIPNPLLGLDST

SEQ ID NO: 787 - protein, 286 aa, synthetic construct
MSTSTQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSGGGGSGGGGSGGGGSGGGGSDIQMTQSPSSLSASVGDRVTITCRASQSISTWLAWYQQKPGKAPKLLIYAASSLDEGVPSRFSGSGSGTDFTLTISSLQPEDFATYYCQQSYSFPVTFGQGTKVEIKEQKLISEEDLGSGLNDIFEAQKIEWHEGKPIPNPLLGLDST

SEQ ID NO: 788 - protein, 286 aa, synthetic construct
MSTSTQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWDVWGQGTLVTVSSGGGGSGGGGSGGGGSGGGGSDIQMTQSPSSLSASVGDRVTITCRASQSISTWLAWYQQKPGKAPKLLIYAASSLDSGVPSRFSGSGSGTDFTLTISSLQPEDFATYYCQQSYSFPVTFGQGTKVEIKEQKLISEEDLGSGLNDIFEAQKIEWHEGKPIPNPLLGLDST

SEQ ID NO: 789 - protein, 286 aa, synthetic construct
MSTSTQVQLVQSGAEVKKPGASVKVSCKASGYTFTRYYMHWVRQAPGQGLEWMGIINPRAGYTSYALKFQGRVTMTRDTSTSTVYMELSSLRSEDTAVYYCTSGWEVWGQGTLVTVSSGGGGSGGGGSGGGGSGGGGSDIQMTQSPSSLSASVGDRVTITCRASQSISTWLAWYQQKPGKAPKLLIYAASSLDEGVPSRFSGSGSGTDFTLTISSLQPEDFATYYCQQSYSFPVTFGQGTKVEIKEQKLISEEDLGSGLNDIFEAQKIEWHEGKPIPNPLLGLDST

SEQ ID NO: 790 - protein, 286 aa, synthetic construct
MSTSTQVQLVQSGAEVKKPGASVKVSCKASGYTFTRYYMHWVRQAPGQGLEWMGIIQPRAGYTSYALKFQGRVTMTRDTSTSTVYMELSSLRSEDTAVYYCTSGWDVWGQGTLVTVSSGGGGSGGGGSGGGGSGGGGSDIQMTQSPSSLSASVGDRVTITCRASQSISTWLAWYQQKPGKAPKLLIYAASSLDEGVPSRFSGSGSGTDFTLTISSLQPEDFATYYCQQSYSFPVTFGQGTKVEIKEQKLISEEDLGSGLNDIFEAQKIEWHEGKPIPNPLLGLDST

SEQ ID NO: 791 - protein, 286 aa, synthetic construct
MSTSTQVQLVQSGAEVKKPGASVKVSCKASGETFTNYYIHWVRQAPGQGLEWMGIIDPRAGYTSYALKFQGRVTMTRDTSTSTVYMELSSLRSEDTAVYYCAGGWEDWGQGTLVTVSSGGGGSGGGGSGGGGSGGGGSDIQMTQSPSSLSASVGDRVTITCRISQSISSFLAWYQQKPGKAPKLLIYSASSLQSGVPSRFSGSGSGTDFTLTISSLQPEDFATYYCQQSFTSPITFGQGTRLEIKEQKLISEEDLGSGLNDIFEAQKIEWHEGKPIPNPLLGLDST

SEQ ID NO: 792 - protein, 286 aa, synthetic construct
MSTSTQVQLVQSGAEVKKPGASVKVSCKASGETFTNYYIHWVRQAPGQGLEWMGVINPRAGYTSYALKFQGRVTMTRDTSTSTVYMELSSLRSEDTAVYYCAGGWEDWGQGTLVTVSSGGGGSGGGGSGGGGSGGGGSDIQMTQSPSSLSASVGDRVTITCRISQSISSWLAWYQQKPGKAPKLLIYSASSLQSGVPSRFSGSGSGTDFTLTISSLQPEDFATYYCQQSFTSPITFGQGTRLEIKEQKLISEEDLGSGLNDIFEAQKIEWHEGKPIPNPLLGLDST

SEQ ID NO: 793 - protein, 286 aa, synthetic construct
MSTSTQVQLVQSGAEVKKPGASVKVSCKASGFTFTRYYMHWVRQAPGQGLEWMGVINPSGGYATYAQKFQGRVTMTRDTSTSTVYMELSSLRSEDTAVYYCASGWDVWGQGTLVTVSSGGGGSGGGGSGGGGSGGGGSDIQMTQSPSSLSASVGDRVTITCRASQSISSWLAWYQQKPGKAPKLLIYATSTLESGVPSRFSGSGSGTDFTLTISSLQPEDFATYYCQQSYSSPVTFGQGTKVEIKEQKLISEEDLGSGLNDIFEAQKIEWHEGKPIPNPLLGLDST

SEQ ID NO: 794 - protein, 286 aa, synthetic construct
MSTSTQVQLVQSGAEVKKPGASVKVSCKASGGTFTNYYIHWVRQAPGQGLEWMGIIDPRAGYTSYALKFQGRVTMTRDTSTSTVYMELSSLRSEDTAVYYCAGGWEDWGQGTLVTVSSGGGGSGGGGSGGGGSGGGGSDIQMTQSPSSLSASVGDRVTITCRISQSISSWLAWYQQKPGKAPKLLIYSASSLQSGVPSRFSGSGSGTDFTLTISSLQPEDFATYYCQQSFTSPITFGQGTRLEIKEQKLISEEDLGSGLNDIFEAQKIEWHEGKPIPNPLLGLDST

SEQ ID NO: 795 - protein, 286 aa, synthetic construct
MSTSTQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSGGGGSGGGGSGGGGSGGGGSDIQMTQSPSSLSASVGDRVTITCRASQSINSWLAWYQQKPGKAPKLLIYATSTLESGVPSRFSGSGSGTDFTLTISSLQPEDFATYYCQQSYRFPVTFGQGTKVEIKEQKLISEEDLGSGLNDIFEAQKIEWHEGKPIPNPLLGLDST

SEQ ID NO: 796 - protein, 286 aa, synthetic construct
MSTSTQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSGGGGSGGGGSGGGGSGGGGSDIQMTQSPSSLSASVGDRVTITCRASQSINSWLAWYQQKPGKAPKLLIYATSTLESGVPSRFSGSGSGTDFTLTISSLQPEDFATYYCQQYYRFPVTFGQGTKVEIKEQKLISEEDLGSGLNDIFEAQKIEWHEGKPIPNPLLGLDST

SEQ ID NO: 797 - protein, 286 aa, synthetic construct
MSTSTQVQLVQSGAEVKKPGASVKVSCKASGHTFTRYYMHWVRQAPGQGLEWMGIINPSGGYATYAQKFQGRVTMTRDTSTSTVYMELSSLRSEDTAVYYCASGLFIWGQGTLVTVSSGGGGSGGGGSGGGGSGGGGSDIQMTQSPSSLSASVGDRVTITCRASQSINSWLAWYQQKPGKAPKLLIYATSTLESGVPSRFSGSGSGTDFTLTISSLQPEDFATYYCQQYYSFPVTFGQGTKVEIKEQKLISEEDLGSGLNDIFEAQKIEWHEGKPIPNPLLGLDST

SEQ ID NO: 798 - protein, 286 aa, synthetic construct
MSTSTQVQLVQSGAEVKKPGASVKVSCKASGHTFTRYYMHWVRQAPGQGLEWMGVINPSGGYATYAQKFQGRVTMTRDTSTSTVYMELSSLRSEDTAVYYCASGWDVWGQGTLVTVSSGGGGSGGGGSGGGGSGGGGSDIQMTQSPSSLSASVGDRVTITCRASQSINSWLAWYQQKPGKAPKLLIYATSTLESGVPSRFSGSGSGTDFTLTISSLQPEDFATYYCQQSYSSPVTFGQGTKVEIKEQKLISEEDLGSGLNDIFEAQKIEWHEGKPIPNPLLGLDST

SEQ ID NO: 799 - protein, 286 aa, synthetic construct
MSTSTQVQLVQSGAEVKKPGASVKVSCKASGSTFTNYYIHWVRQAPGQGLEWMGIIDPRAGYTSYALKFQGRVTMTRDTSTSTVYMELSSLRSEDTAVYYCAGGWEDWGQGTLVTVSSGGGGSGGGGSGGGGSGGGGSDIQMTQSPSSLSASVGDRVTITCRISQSISSFLAWYQQKPGKAPKLLIYSASSLQSGVPSRFSGSGSGTDFTLTISSLQPEDFATYYCQQSFTSPITFGQGTRLEIKEQKLISEEDLGSGLNDIFEAQKIEWHEGKPIPNPLLGLDST

SEQ ID NO: 800 - protein, 590 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT